(19) United States
(12) Patent Application Publication
(10) Pub. No.: US 2024/0207267 A1
(43) Pub. Date: Jun. 27, 2024

(54) (R)-Glutarimide CRBN Ligands and Methods of Use

(71) Applicant: BeiGene Switzerland GmbH, Basel (CH)

(72) Inventors: Bailin Lei, Beijing (CN); Huaqing Liu, Beijing (CN); Songzhe Han, Beijing (CN); Changxin Huo, Beijing (CN); Zhiwei Wang, Beijing (CN)

(73) Assignee: BeiGene Switzerland GmbH, Basel (CH)

(21) Appl. No.: 18/391,154

(22) Filed: Dec. 20, 2023

Related U.S. Application Data
(63) Continuation of application No. PCT/CN2022/100017, filed on Jun. 21, 2022.

(30) Foreign Application Priority Data
Jun. 21, 2021 (WO) PCTCN2021101281
Dec. 30, 2021 (WO) PCTCN2021142802

(51) Int. Cl. A61K 31/506; A61K 47/54; A61P 35/00

(52) U.S. Cl. CPC A61K 31/506; A61K 47/545; A61P 35/00

(57) ABSTRACT

Disclosed herein are compounds for binding and modulating the activity of cereblon (CRBN), and methods of use. The present invention also provides compounds that can be used as synthetic intermediates in the preparation of bifunctional compounds for use in targeted protein degradation. The present compounds are thus useful for the treatment or prophylaxis of tumors and cancers.

### FIELD OF THE INVENTION

Disclosed herein are compounds for binding and modulating the activity of cereblon (CRBN), and methods of use. The present invention also provides compounds that can be used as synthetic intermediates in the preparation of bifunctional compounds for use in targeted protein degradation. The present compounds are thus useful for the treatment or prophylaxis tumors and cancer.

### BACKGROUND OF THE INVENTION

Proteolysis targeting chimera (PROTAC) consists of two covalently linked protein-binding molecules: one capable of engaging an E3 ubiquitin ligase, and another that binds to the protein of interest (POI) a target meant for degradation (Sakamoto K M et al., Proc. Natl. Acad. Sci. 2001, 98: 8554-9.; Sakamoto K. M. et al., Methods Enzymol. 2005; 399:833-847.). Rather than inhibiting the target protein's enzymatic activity, recruitment of the E3 ligase to the specific unwanted proteins results in ubiquitination and subsequent degradation of the target protein by the proteasome. The whole process of ubiquitination and proteasomal degradation is known as the ubiquitin-proteasome pathway (UPP) (Ardley H. et al., Essays Biochem. 2005, 41, 15-30; Komander D. et al., Biochem. 2012, 81, 203-229; Grice G. L. et al., Cell Rep. 2015, 12, 545-553; Swatek K. N. et al., Cell Res. 2016, 26, 399-422). Proteasomes are protein complexes which degrade unneeded, misfolded or abnormal proteins into small peptides to maintain the health and productivity of the cells. Ubiquitin ligases, also called an E3 ubiquitin ligase, directly catalyze the transfer of ubiquitin from the E2 to the target protein for degradation. Although the human genome encodes over 600 putative E3 ligases, only a limited number of E3 ubiquitin ligases have been widely applied by small molecule PROTAC technology: cereblon (CRBN), Von Hippel-Lindau (VHL), mouse double minute 2 homologue (MDM2) cellular inhibitor of apoptosis protein (cIAP) (Philipp O. et al., Chem. Biol. 2017, 12, 2570-2578), recombinant Human Ring Finger Protein 114 (RNF114) (Spradlin, J. N. et al. Nat. Chem. Biol. 2019, 15, 747-755) and DDB1 And CUL4 Associated Factor 16 (DCAF16) (Zhang, X. et al. Nat. Chem. Biol. 2019, 15, 737-746). Cereblon (CRBN) forms an E3 ubiquitin ligase complex with damaged DNA binding protein 1 (DDB1) and Cullin-4A (CUL4A) to ubiquitinate a number of other proteins followed by the degradation via proteasomes. (Yi-An Chen, et al., Scientific Reports 2015, 5, 1-13). Immunomodulatory drugs (IMiDs), including thalidomide, lenalidomide, and pomalidomide, function as monovalent promoters of PPIs by binding to the cereblon (CRBN) subunit of the CRL4A^{CRBN} E3 ligase complex and recruiting neosubstrate proteins. (Matyskiela, M. E. et al., Nat Chem Biol 2018, 14, 981-987.) As a consequence, the ability of thalidomide, and its derivatives, to recruit CRBN has been widely applied in proteolysis-targeting chimeras (PROTACs) related studies (Christopher T. et al. ACS Chem. Biol. 2019, 14, 342-347.; Honorine L. et al, ACS Cent. Sci. 2016, 2, 927-934). These new findings regarding the role of CRBN in IMiD action stimulated intense investigation of CRBN's downstream factors involved in maintaining regular function of a cell and its use as a substrate receptor in which the proteins recognized by CRBN might be ubiquitinated and degraded by proteasomes.

There is a need for new compounds, compositions and uses thereof that bind to E3 ligase protein cereblon for the treatment of serious diseases. There is also a need for new compounds that may be in the preparation of bifunctional molecules that are used in the degradation of proteins.

### SUMMARY OF THE INVENTION

One objective of the present invention is to provide compounds and derivatives formed by conjugating target protein moieties with E3 ligase Ligand moieties, which function to recruit targeted proteins to E3 ubiquitin ligase for degradation, and methods of preparation and uses thereof.

Aspect 1. A compound of Formula (I):

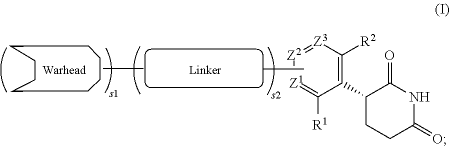

or a pharmaceutically acceptable salt thereof, or a deuterated analog thereof, or a prodrug thereof, wherein:
Warhead is a targeting moiety that binds to a target protein; wherein the target protein is a mediator of a disease in a subject;
Linker is a divalent chemical group that connects the Warhead moiety and the

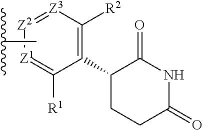

s1 is 0 or 1;
s2 is 0 or 1;
Z¹, Z² and Z³ are each independently N or CR^{Z}, provided that Z¹, Z² and Z³ are not N at the same time;
R^{Z}, at each occurrence, is independently selected from hydrogen, halogen, —C₁₋₈alkyl, —NR^{Za}R^{Zb}, —OR^{Za}, —SR^{Za}, C₃-C₈cycloalkyl, 3- to 8-membered heterocyclyl or CN; each of —C₁₋₈alkyl, C₃-C₈cycloalkyl, 3- to 8-membered heterocyclyl is optionally substituted with at least one R^{Zc};
the

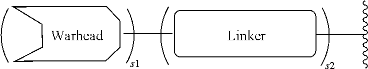

moiety is linked to the

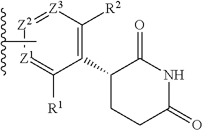

moiety via any one of Z¹, Z² or Z³ which is CR^{Z} and R^{Z} is hydrogen;
R^{Za} and R^{Zb} are each independently selected from hydrogen, —C₁-C₈alkyl, C₃-C₈cycloalkyl, 3- to 8-membered heterocyclyl, C₆-C₁₂aryl, or 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl is optionally substituted with at least one substituent R^{Zd}.
R^{Zc} and R^{Zd} are each independently halogen, hydroxy, —C₁₋₈alkoxy, 3- to 8-membered heterocyclyl, C₆-C₁₂aryl, or 5- to 12-membered heteroaryl;
R¹ and R² are each independently selected from halogen, —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl, 5- to 12-membered heteroaryl, —CN, —SO₂R^{1a}, —SO₂NR^{1a}R^{1b}, —COR^{1a}, —CO₂R^{1a}, —CONR^{1a}R^{1b}, —NR^{1a}R^{1b}, —NR^{1a}COR^{1b}, —NR^{1a}CO₂R^{1b}, or —NR^{1a}SO₂R^{1b}; each of —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}COR^{1d}, —NR^{1c}CO₂R^{1d}, or —NR^{1c}SO₂R^{1d};
R^{1a}, R^{1b}, R^{1c} and R^{1d} are each independently hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl.

Aspect 2. The compound of aspect 1, wherein at most one of Z¹, Z² and Z³ is N.

Aspect 3. The compound of any one of aspects 1-2, wherein Z¹, Z² and Z³ are each independently CR^{Z}.

Aspect 4. The compound of any one of aspects 1-3, wherein R^{Z}, at each occurrence, is independently selected from hydrogen, —F, —Cl, —Br, —I, methyl, ethyl, propyl, butyl, pentyl, hexyl, heptyl, octyl, —NR^{Za}R^{Zb}, —OR^{Za}, —SR^{Za}, cyclopropyl, cyclobutyl, cyclopentyl, cyclohexyl, cycloheptyl, cyclooctyl, 3- to 8-membered heterocyclyl, phenyl, 5- to 12-membered heteroaryl, or CN; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one R^{Zc};
R^{Za} and R^{Zb} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{Zd};
R^{Zc} and R^{Zd} are each independently —F, —Cl, —Br, —I, hydroxy, methyl, ethyl, propyl, butyl, pentyl, hexyl, heptyl, octyl, —C₁₋₈alkoxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 5. The compound of any one of aspects 1-4, wherein R^{Z} is selected from H, —CH₃, —C₂H₅, F, —CH₂F, —CHF₂, —CF₃, —OCH₃, —OC₂H₅, —C₃H₇, —OCH₂F, —OCHF₂, —OCH₂CF₃, —OCF₃, —SCF₃, —CF₃ or —CH(OH)CH₃.

Aspect 6. The compound of any one of aspects 1-5, wherein R¹ and R² are each independently selected from F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{1a}, —SO₂NR^{1a}R^{1b}, —COR^{1a}, —CO₂R^{1a}, —CONR^{1a}R^{1b}, —NR^{1a}R^{1b}, —NR^{1a}COR^{1b}, —NR^{1a}CO₂R^{1b}, or —NR^{1a}SO₂R^{1b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}COR^{1d}, —NR^{1c}CO₂R^{1d}, or —NR^{1c}SO₂R^{1d};
R^{1a}, R^{1b}, R^{1c} and R^{1d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5- to 12-membered heteroaryl.

Aspect 7. The compound of any one of aspects 1-6, wherein R¹ and R² are each independently selected from F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CN, —CH₂F, —CHF₂, —CF₃, —OCH₂F, —OCHF₂, —OCH₂CF₃, —OCF₃, —SCF₃, or phenyl.

Aspect 8. The compound of any one of aspects 1-7, wherein the compound is Formula (II)

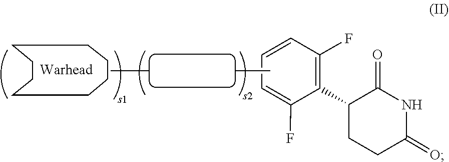

wherein Warhead and Linker are defined as aspect 1.

Aspect 9. The compound of any one of aspects 1-8, wherein Linker is

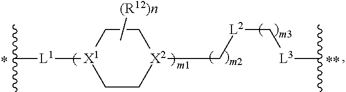

wherein * refers to the position attached to the

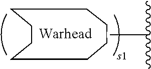

moiety, and ** refers to the position attached to the

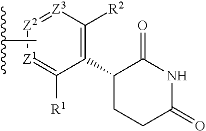

moiety;
L¹ is selected from a single bond, —O—, —SO₂—, —C(O)—, —NR^{L1a}—, —C₃-C₈cycloalkylene-, *^{L1}-O—C₁₋₈alkylene-**^{L1}, *^{L1}-C₁₋₈alkylene-O-**^{L1}, *^{L1}-SO₂—C₁₋₈alkylene**^{L1}, *^{L1}-C₁₋₈alkylene-SO₂-**^{L1}, *^{L1}-CO—C₁₋₈alkylene-**^{L1}, *^{L1}-C₁₋₈alkylene-CO-**^{L1}, *^{L1}-NR^{L1a}—C₁₋₈alkylene-**^{L1}, *^{L1}-C₁₋₈alkylene-NR^{L1a}-**^{L1}, *^{L1}-NR^{L1a}C(O)-**^{L1}, *^{L1}-C(O)NR^{L1a}-**^{L1}, —C₁₋₈alkylene-, —C₂₋₈alkenylene-, —C₂₋₈-alkynylene-, —[OC(CR^{L1a}R^{L1b})ₘ₄]ₘ₅—,

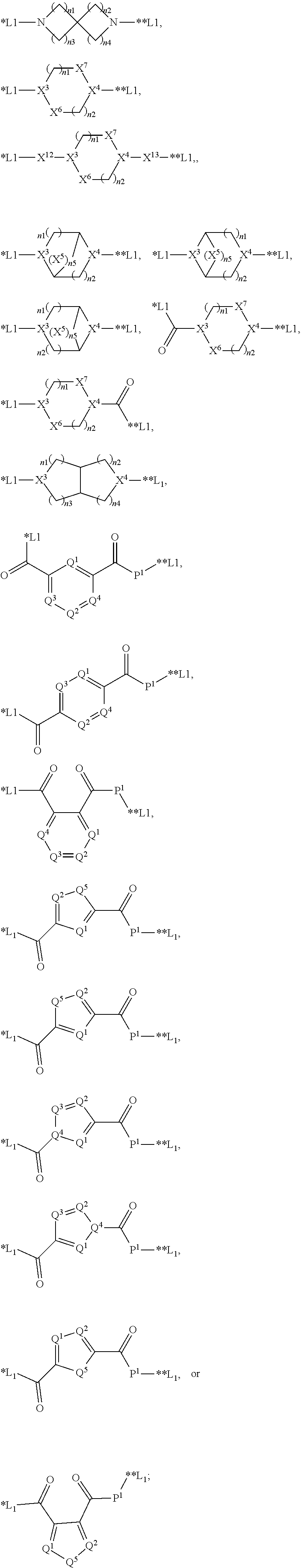

wherein each of said —C₃-C₈cycloalkylene-, *^{L1}-O—C₁₋₈alkylene-**^{L1}, *^{L1}-C₁₋₈alkylene-O-**^{L1}, *^{L1}-SO₂—C₁₋₈alkylene-**^{L1}, *^{L1}-C₁₋₈alkylene-SO₂-**^{L1}, *^{L1}-CO—C₁₋₈alkylene-**^{L1}, *^{L1}-C₁₋₈alkylene-CO-**^{L1}, *^{L1}-NR^{L1a}—C₁₋₈alkylene-**^{L1}, *^{L1}-C₁₋₈alkylene-NR^{L1a}-**^{L1}, —C₁₋₈alkylene-, —C₂₋₈alkenylene-, —C₂₋₈alkynylene-,

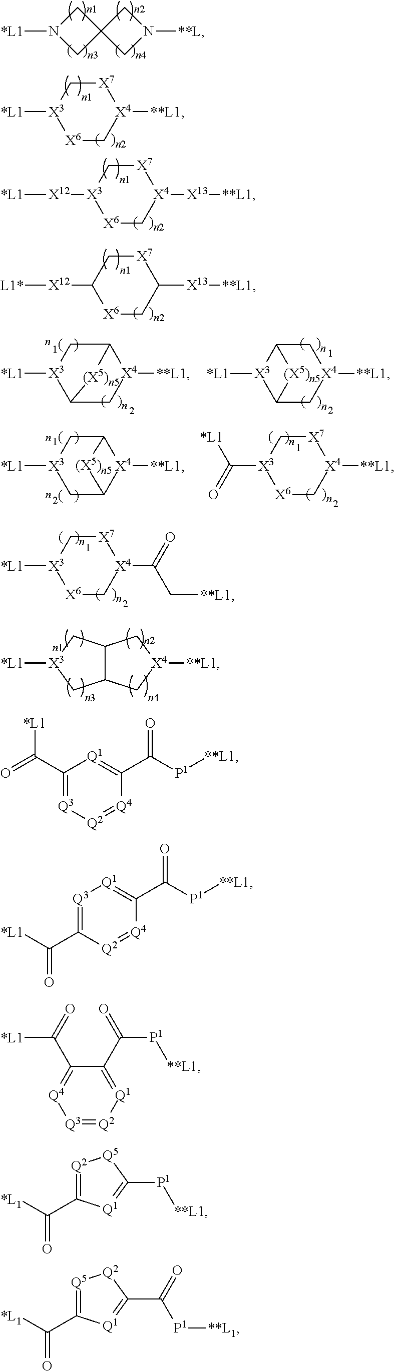

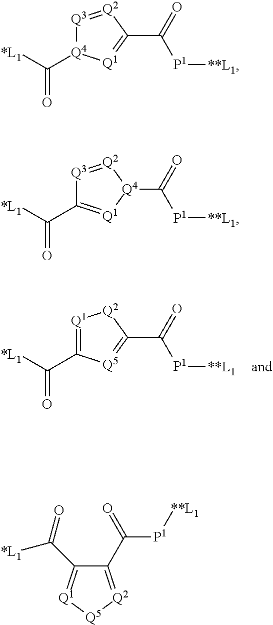

is optionally substituted with at least one R^{L1c};
wherein *^{L1} refers to the position attached to the

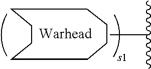

moiety, and **^{L1} refers to the position attached to the

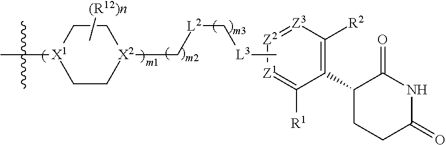

moiety;
R^{L1a} and R^{L1b} are each independently selected from hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{L1d};
each of said R^{L1c} and R^{L1d} are independently oxo, halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, or —C₁₋₈alkyl;
L² is selected from a single bond, —O—, —SO₂—, —CO—, —NR^{L2a}—, —C₃-C₈cycloalkylene-, *^{L2}-O—C₁₋₈alkylene-**^{L2}, *^{L2}-C₁₋₈alkylene-O-**^{L2}, *^{L2}-SO₂—C₁₋₈alkylene-**^{L2}, *^{L2}-C₁₋₈alkylene-SO₂-**^{L2}, *^{L2}-CO—C₁₋₈alkylene-**^{L2}, *^{L2}-C₁₋₈alkylene-CO-**^{L2}, *^{L2}-NR^{L2a}—C₁₋₈alkylene-**^{L2}, *^{L2}-C₁₋₈alkylene-NR^{L2a}-**^{L2}, *^{L2}-NR^{L2a}C(O)NR^{L2a}-**^{L2}, *^{L2}-C(O)NR^{L2a}-**^{L2}, —C₁₋₈alkylene-, —C₂₋₈alkenylene-, —C₂₋₈alkynylene, —[O(CR^{L2a}R^{L2b})ₘ₄]ₘ₅—,

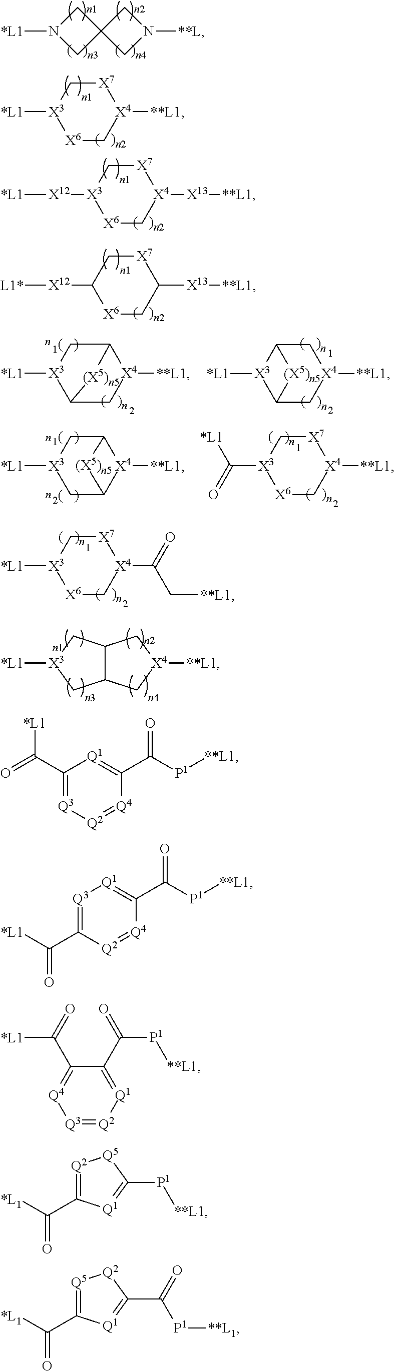

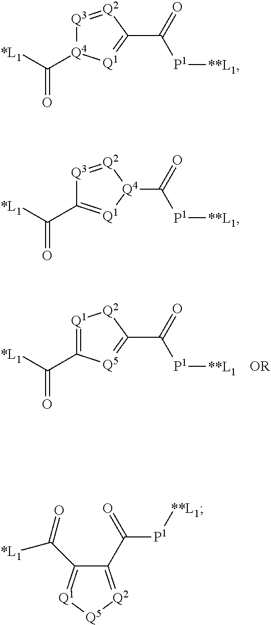

wherein each of said —C₃-C₈cycloalkylene-, *^{L2}-O—C₁₋₈alkylene-**^{L2}, *^{L2}-C₁₋₈alkylene-O-**^{L2}, *^{L2}-SO₂—C₁₋₈alkylene-**^{L2}, *^{L2}-C₁₋₈alkylene-SO₂-**^{L2}, *^{L2}-CO—C₁₋₈alkylene-**^{L2}, *^{L2}-C₁₋₈alkylene-CO-**^{L2}, *^{L2}-NR^{L2a}—C₁₋₈alkylene-**^{L2}, *^{L2}-C₁₋₈alkylene-NR^{L2a}-**^{L2}, —C₁₋₈alkylene-, —C₂₋₈alkenylene-, —C₂₋₈alkynylene-,

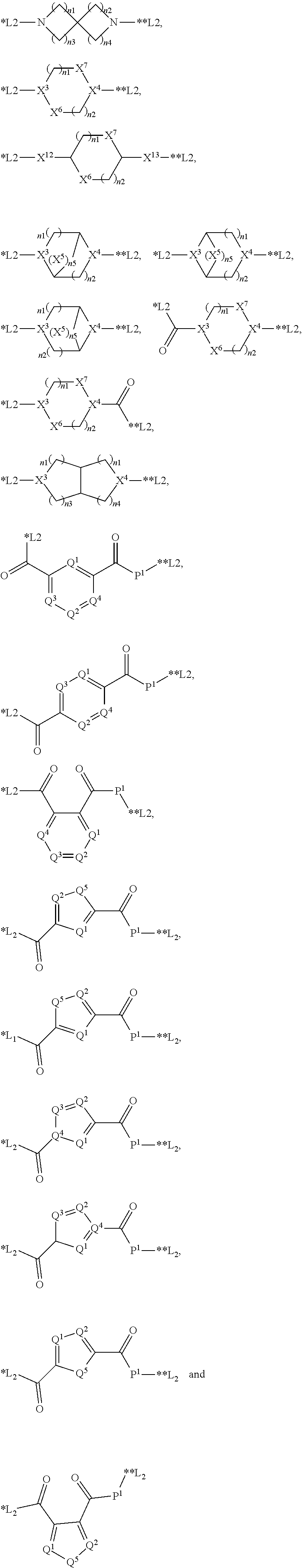

is optionally substituted with at least one substituent R^{L2c};
wherein *^{L2} refers to the position attached to the

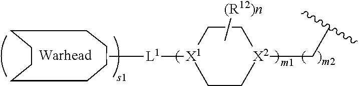

moiety, and **^{L2} refers to the position attached to the

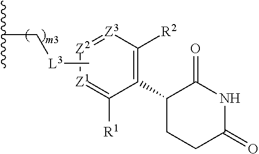

moiety;
R^{L2a} and R^{L2b} are each independently selected from hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{L2d};
each of said R^{L2c} and R^{L2d} are independently oxo, halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl; or
two R^{L2c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁₋₈alkyl;
L³ is selected from a single bond, —O—, —SO₂—, —CO—, —NR^{L3a}—, —C₃-C₈cycloalkylene-, *^{L3}-O—C₁₋₈alkylene-**^{L3}, *^{L3}-C₁₋₈alkylene-O-**^{L3}, *^{L3}-SO₂—C₁₋₈alkylene-**^{L3}, *^{L3}-C₁₋₈alkylene-SO₂-**^{L3}, *^{L3}-CO—C₁₋₈alkylene-**^{L3}, *^{L3}-C₁₋₈alkylene-CO-**^{L3}, *^{L3}-NR^{L3a}C₁₋₈alkylene-**^{L3}, *^{L3}-C₁₋₈alkylene-NR^{L3a}-**^{L3}, *^{L3}-NR^{L3a}C(O)**^{L3}, *^{L3}-C(O)NR^{L3a}-**^{L3}, —C₁₋₈alkylene-, —C₂₋₈alkenylene-, —C₂₋₈alkynylene-, —[O(CR^{L3a}R^{L3b})ₘ₄]ₘ₅—,

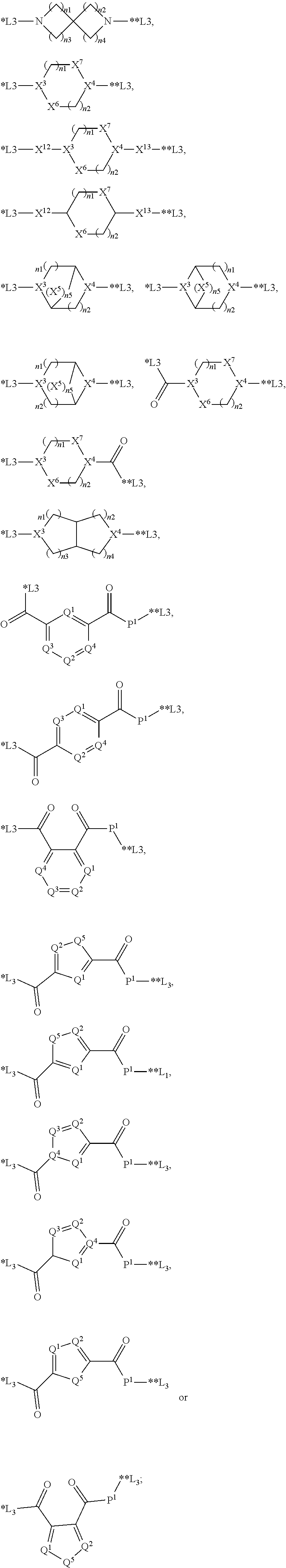

wherein each of said —C₃-C₈cycloalkylene-, *^{L3}-O—C₁₋₈alkylene-**^{L3}, *^{L3}-C₁₋₈alkylene-O-**^{L3}, *^{L3}-SO₂—C₁₋₈alkylene-**^{L3}, *^{L3}-C₁₋₈alkylene-SO₂-**^{L3}, *^{L3}-CO—C₁₋₈alkylene-**^{L3}, *^{L3}-C₁₋₈alkylene-CO-**^{L3}, *^{L3}-NR^{L3a}—C₁₋₈alkylene-**^{L3}, *^{L3}-C₁₋₈alkylene-NR^{L3a}-**^{L3}, —C₁₋₈alkylene-, —C₂₋₈alkenylene-, —C₂₋₈alkynylene-,
is optionally substituted with at least one substituent R^{L3c}; wherein *^{L3} refers to the position attached to the

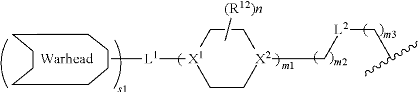

moiety, and; **^{L3} refers to the position attached to the

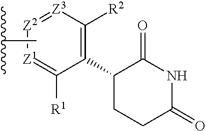

moiety;
R^{L3a} and R^{L3b} are each independently selected from hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{L3d};
each of said R^{L3c} and R^{L3d} are independently oxo, halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl; or
two R^{L3c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, or —C₁₋₈alkyl;
R¹² is independently selected from hydrogen, halogen, —C₁₋₈alkyl, —NR^{12a}R^{12b}, —OR^{12a}, —C₃-C₈cycloalkyl, 3- to 8-membered heterocyclyl, —C₆-C₁₂aryl, 5- to 12-membered heteroaryl, oxo, or —CN; each of —C₁₋₈alkyl, C₃-C₈cycloalkyl, 3- to 8-membered heterocyclyl, C₆-C₁₂aryl, or 5- to 12-membered heteroaryl is optionally substituted with at least one substituent R^{12c}; or
two R¹² together with the carbon atoms to which they are attached, form a 3- to 12-membered ring, said ring comprising 0-3 heteroatoms independently selected from nitrogen, oxygen or sulfur; said ring is optionally substituted with at least one substituent R^{12c};
R^{12a} and R^{12b} are each independently selected from hydrogen, —C₁₋₈alkyl, —C₂₋₈alkenyl, —C₂₋₈alkynyl, —C₃-C₈cycloalkyl, 3- to 8-membered heterocyclyl, —C₆-C₁₂aryl or 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{12d}; or
R^{12c} and R^{12d} are each independently halogen, hydroxy, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl or 5- to 12-membered heteroaryl;
X¹, X², X³ and X⁴ are each independently selected from —CR^{a}, or N;
X⁵, X⁶ and X⁷ are each independently selected from —NR^{a}—, —O—, —S— and —CR^{a}R^{b}—;
X¹² and X¹³ are each independently selected from —C(O)—, —NR^{a}— and —O—;
Q¹, Q², Q³ and Q⁴ are each independently selected from CR^{a} or N;
Q⁵ is each independently selected from —O—, —NR^{a}—, —CR^{a}R^{b}—, —S— or —C(O)—;
P¹ is a single bond, —O—, —NR^{a}—, —CR^{a}R^{b}—, —S—, —SO— or —SO₂—;
at each occurrence, Ra and Rb are each independently selected from hydrogen, hydroxy, halogen, CN, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl or 5- to 12-membered heteroaryl, each of said —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl or 5- to 12-membered heteroaryl; or
R^{a} and R^{b} together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁₋₈alkyl, —C₂₋₈alkenyl, —C₂₋₈alkynyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl;
m₁ is 0 or 1;
m₂ and m3 is 0, 1, 2, 3, 4, 5, 6, 7 or 8;
m₄ and m₅ are each independently 0, 1, 2 or 3;
n, n₁, n₂, n₃, n₄ and n₅ are each independently 0, 1, 2 or 3.

Aspect 10. The compound of aspect 9, wherein L¹ is selected from a single bond, —C₁₋₈alkylene-(preferably —CH₂—, —C₂H₄—, —C₃H₆—), —CO—, —O—, —N(CH₃)—, —NH—,

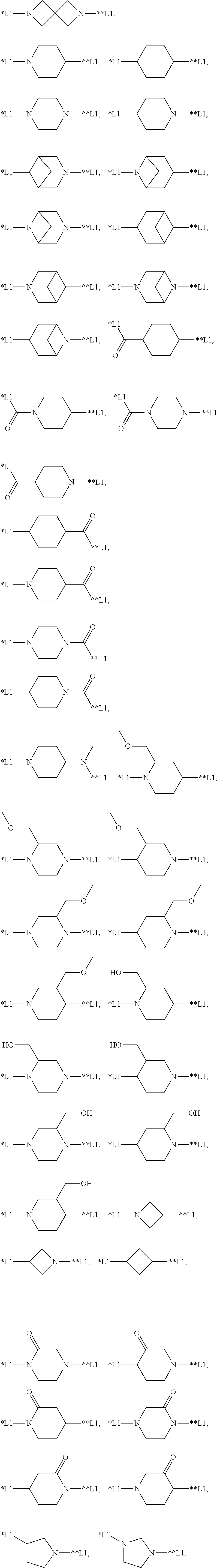

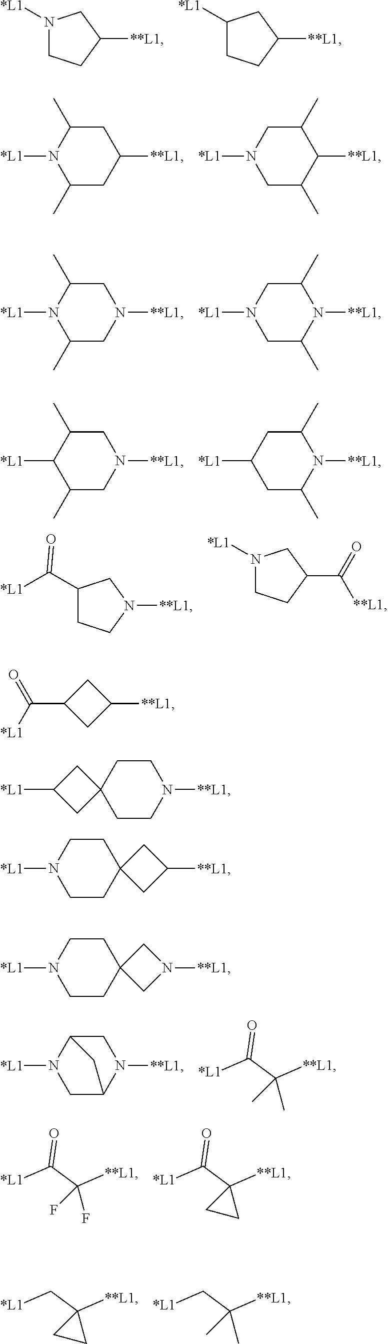

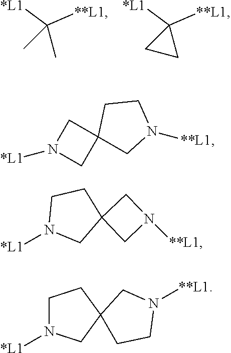

Aspect 11. The compound of any one of aspects 9-10, wherein X¹ and X² are each independently selected from —CR^{a} or N;
R^{a} is selected from hydrogen, —F, —Cl, —Br, —I, CN, methyl, ethyl, methoxy, ethoxy, or cyclopropyl, wherein each of said methyl, ethyl, methoxy, ethoxy, and cyclopropyl is optionally substituted with at least one substituent —F, —Cl, —Br, —I, hydroxy, methyl, ethyl (preferably, X¹ and X² are each independently selected from CH, C(F), C(CH₃) or N);
m₁=1 or 0;
R¹² is hydrogen, oxo, methoxymethyl, hydroxymethyl, —CN or —CH₃.

Aspect 12. The compound of any one of aspects 9-11, wherein m₁ is 1; preferably,

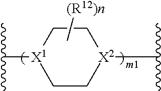

moiety is

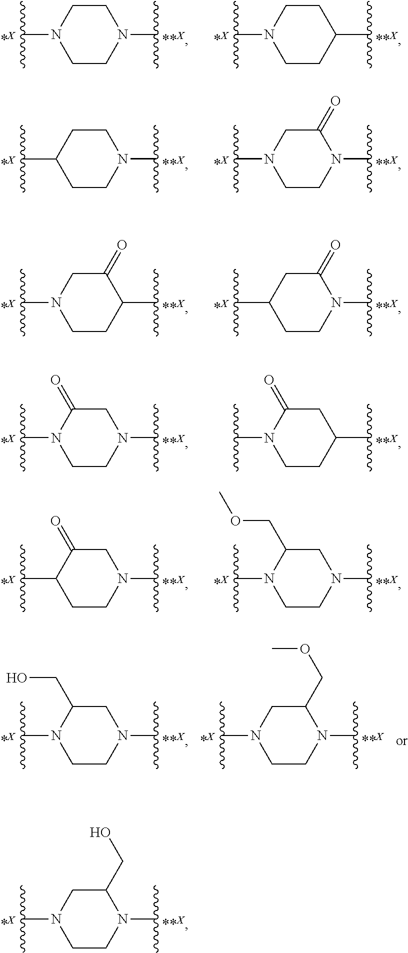

wherein *^{X} refers to the position attached to

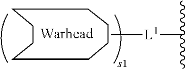

moiety, and **^{X} refers to the position attached to the

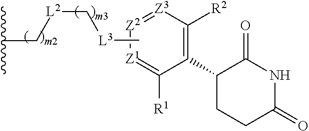

moiety.

Aspect 13. The compound of any one of aspects 9-12, wherein m₁ is 1,

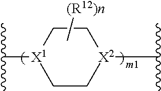

moiety is

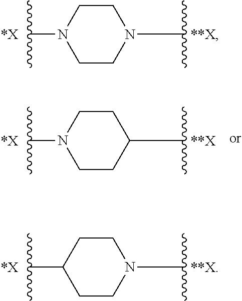

Aspect 14. The compound of any one of aspects 9-13, wherein L² is selected from a single bond, —C₁₋₈alkylene- (preferably —CH₂—, —C₂H₄—, —C₃H₆—), —CO—, —O—, —N(CH₃)—, —NH—,

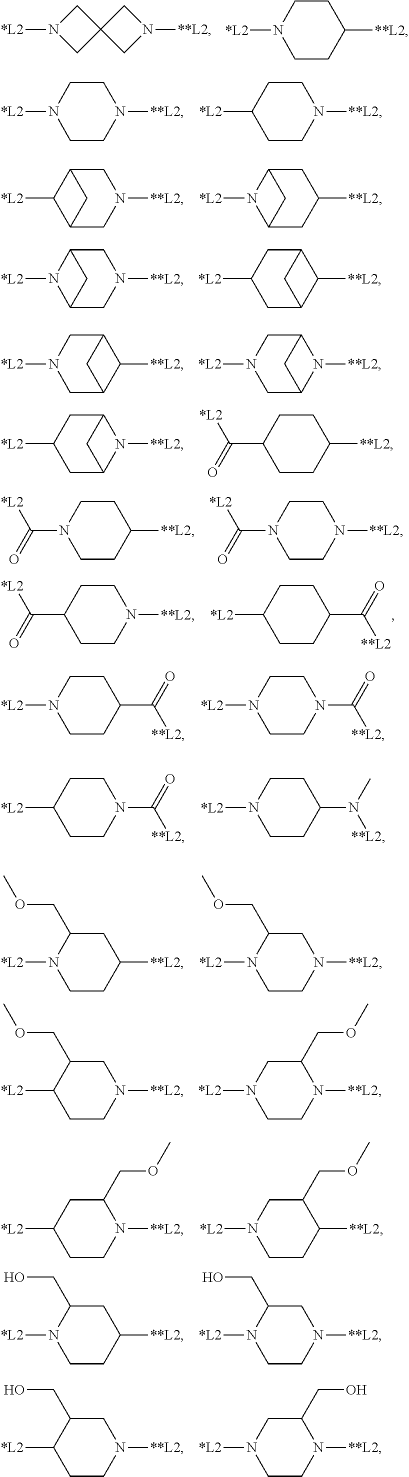

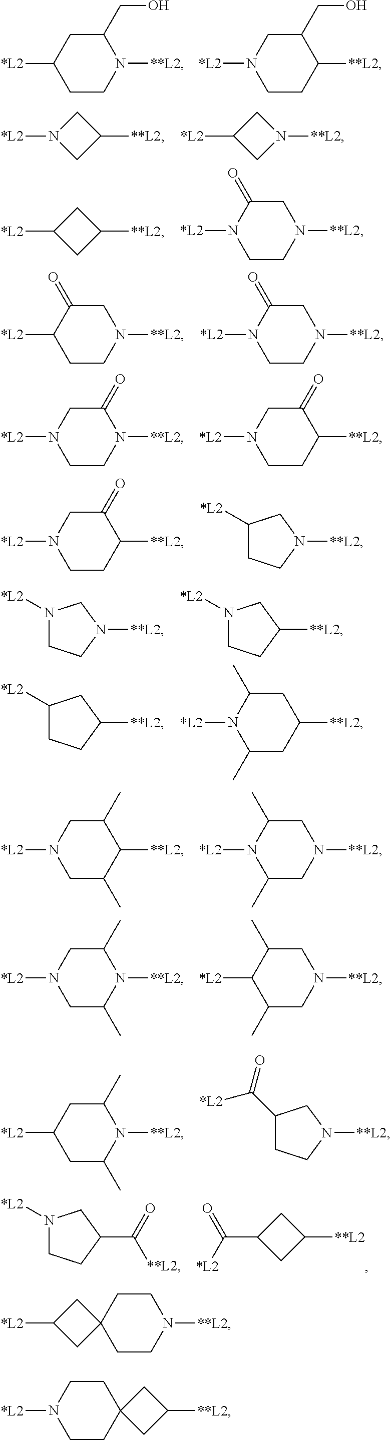

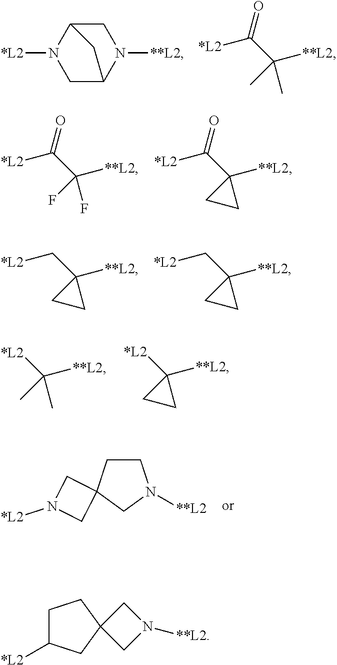

Aspect 15. The compound of any one of aspects 9-14, wherein L³ is selected from single bond, —C₁₋₈alkylene-(preferably —CH₂—, —C₂H₄—, —H₆—), —CO—, —O—, —N(CH₃)—, —NH—,

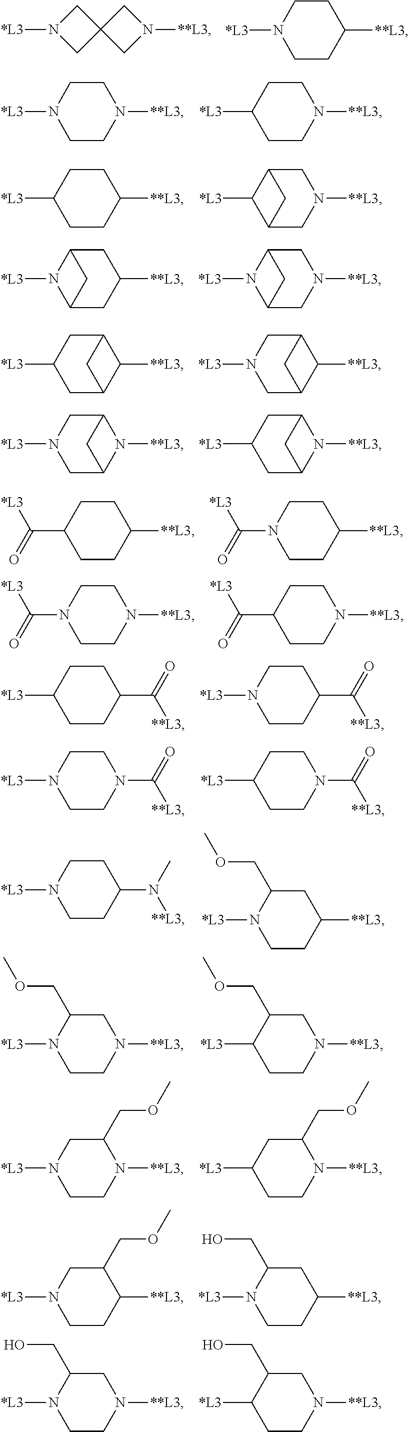

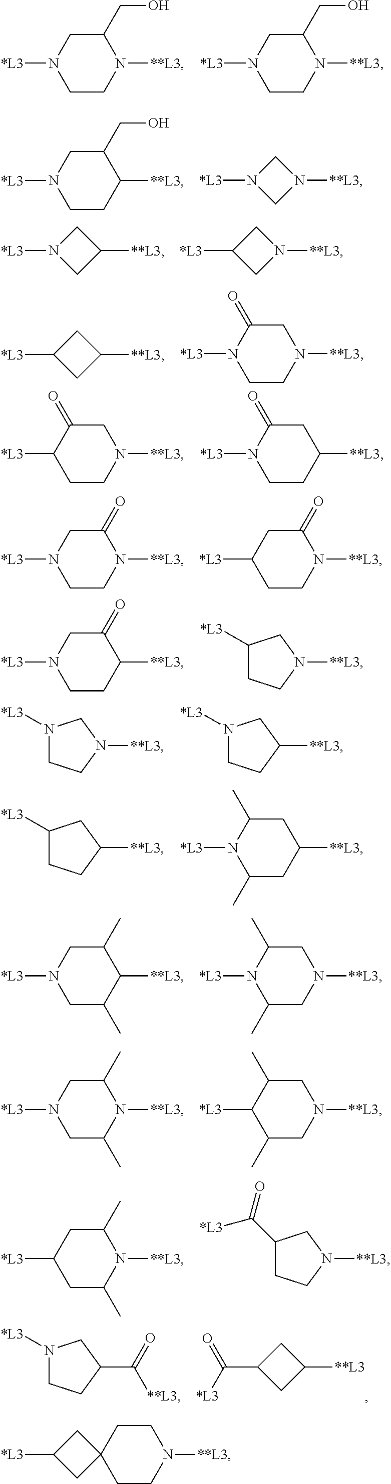

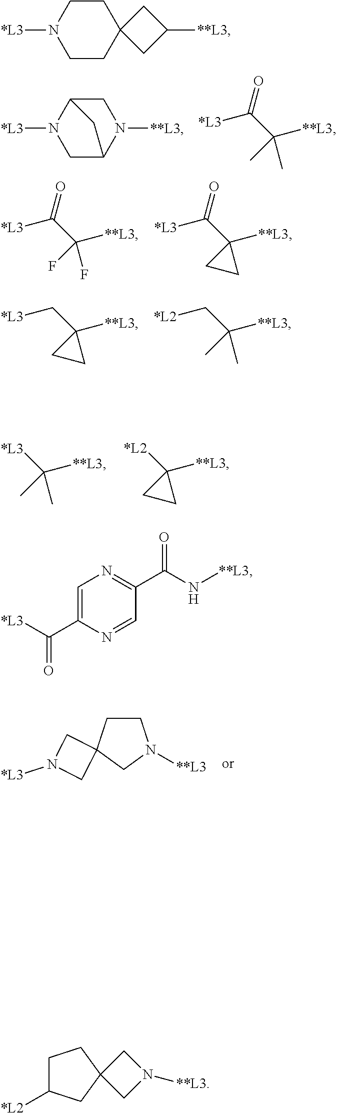

Aspect 16. The compound of any one of aspects 9-15, wherein L² is a single bond; or L³ is a single bond; or L² is a single bond and L³ is a single bond.

Aspect 17. The compound of any one of aspects 9-16, wherein

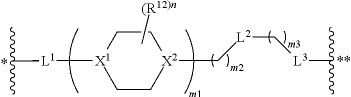

is selected from

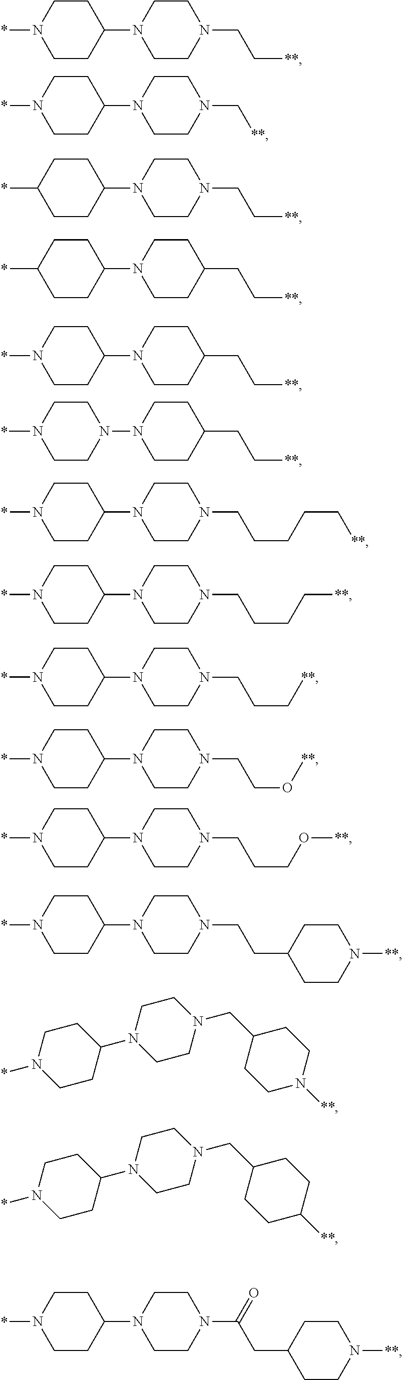

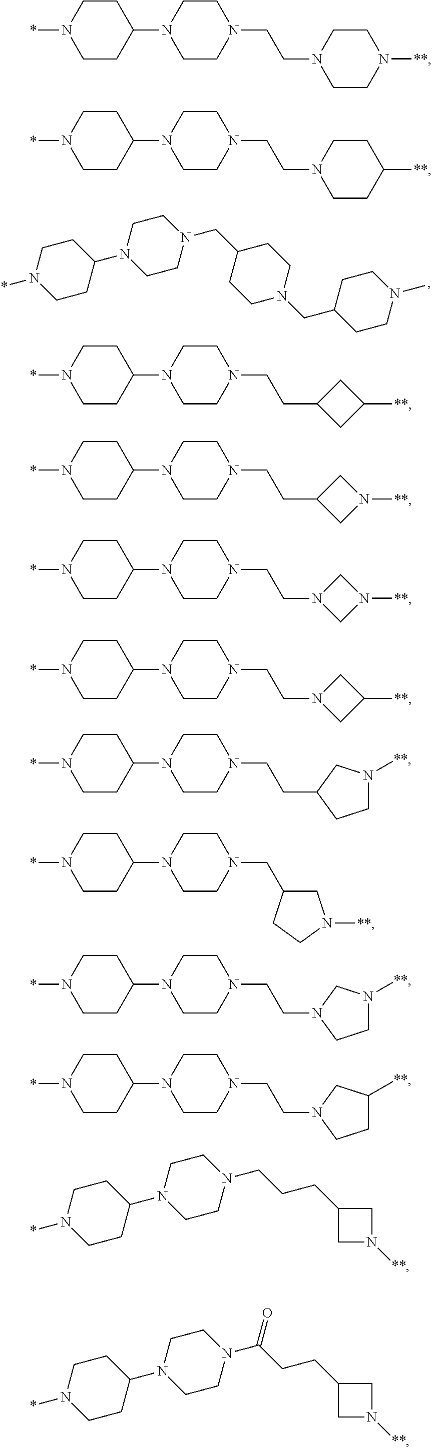

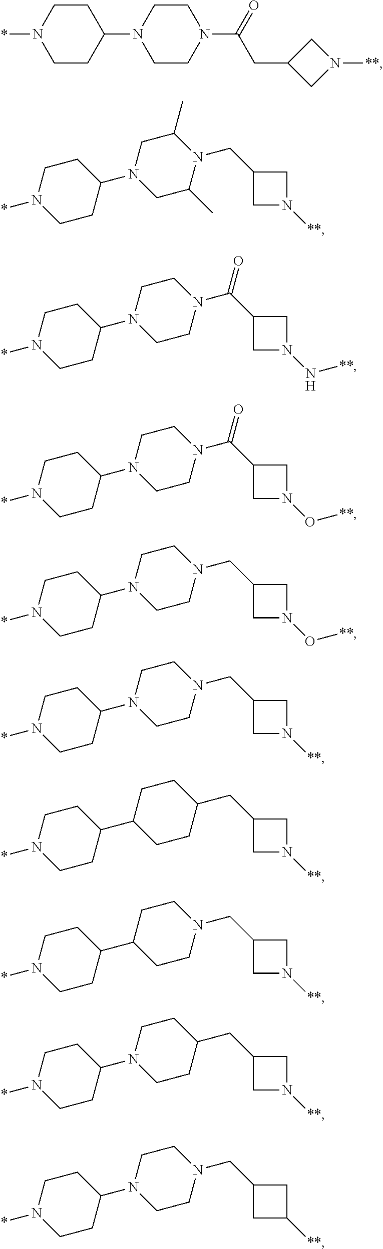

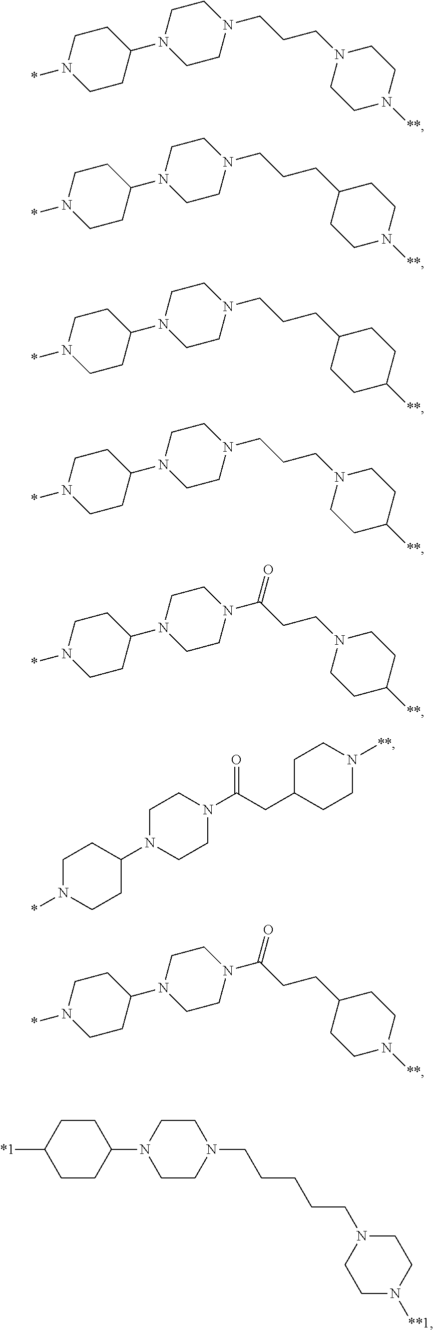

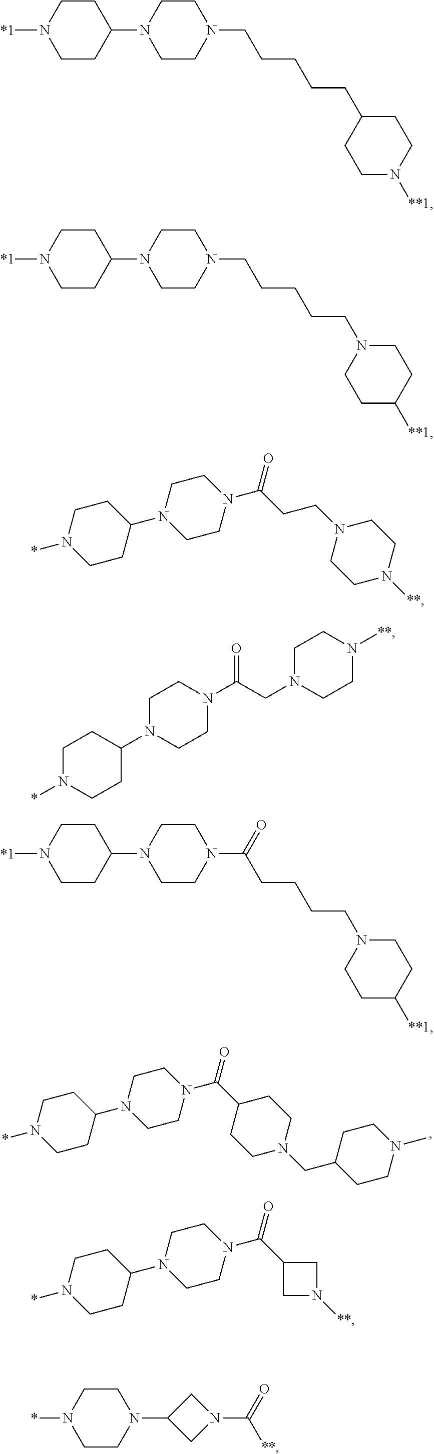

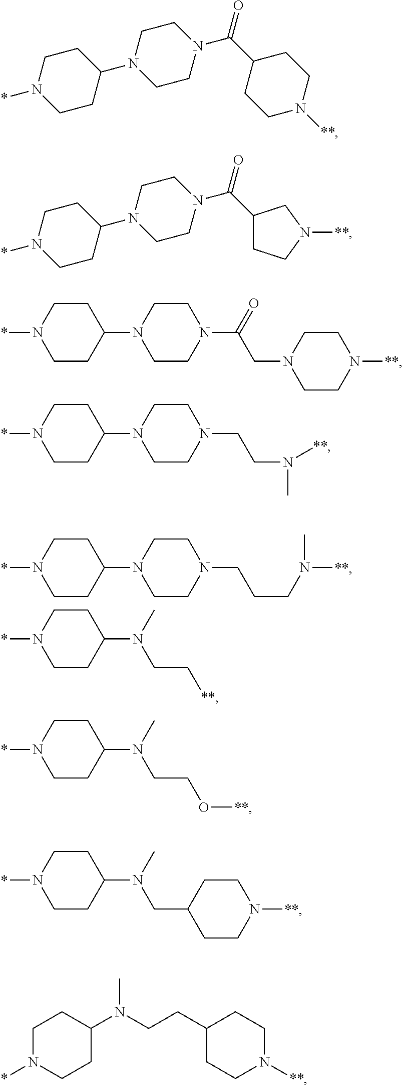

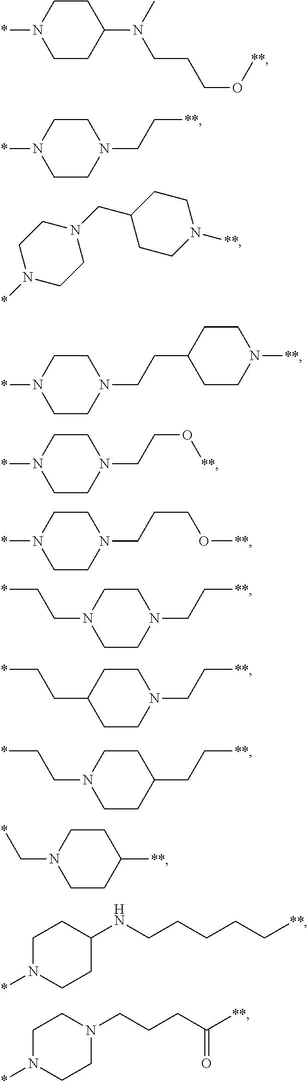

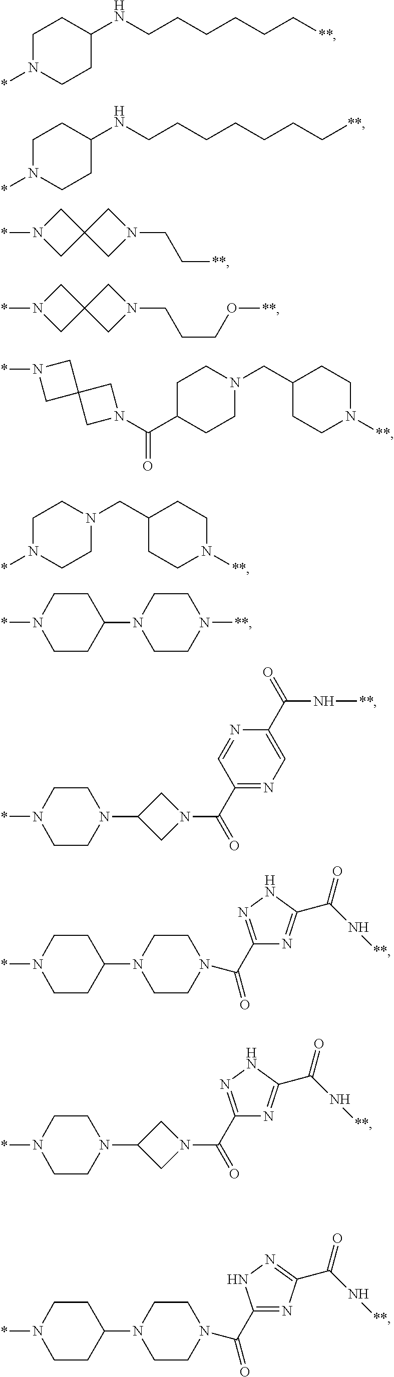

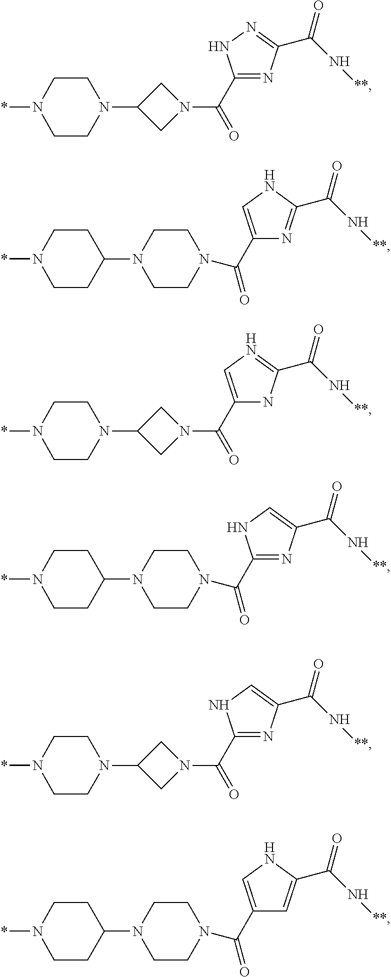

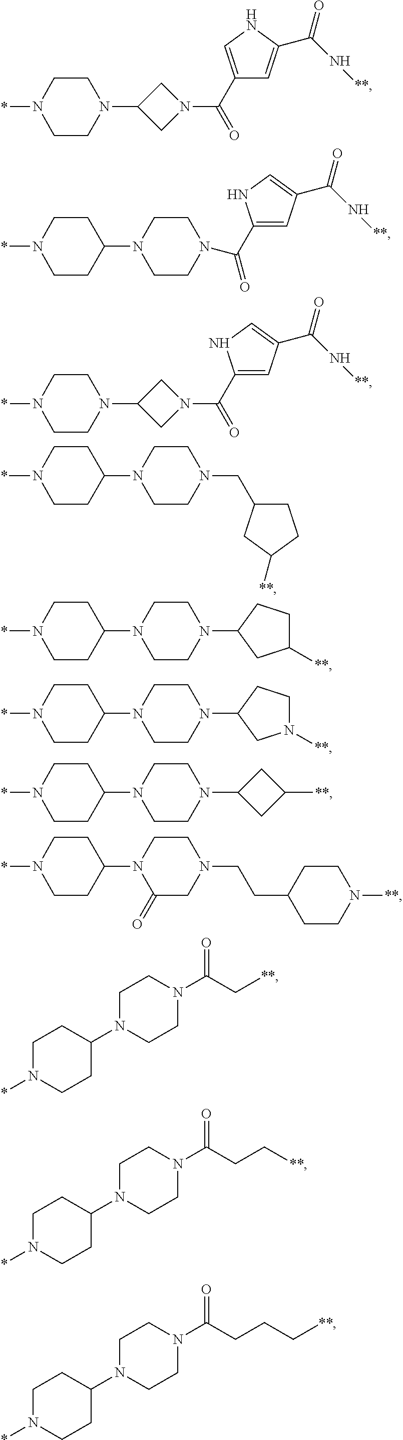

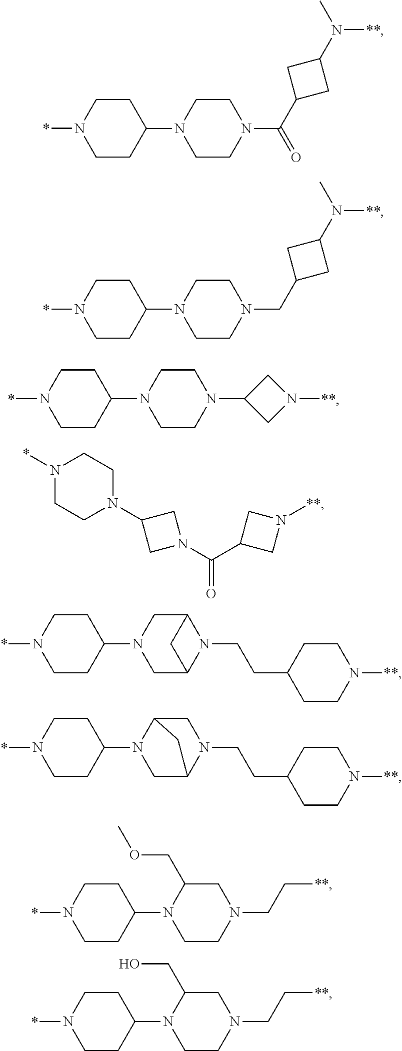

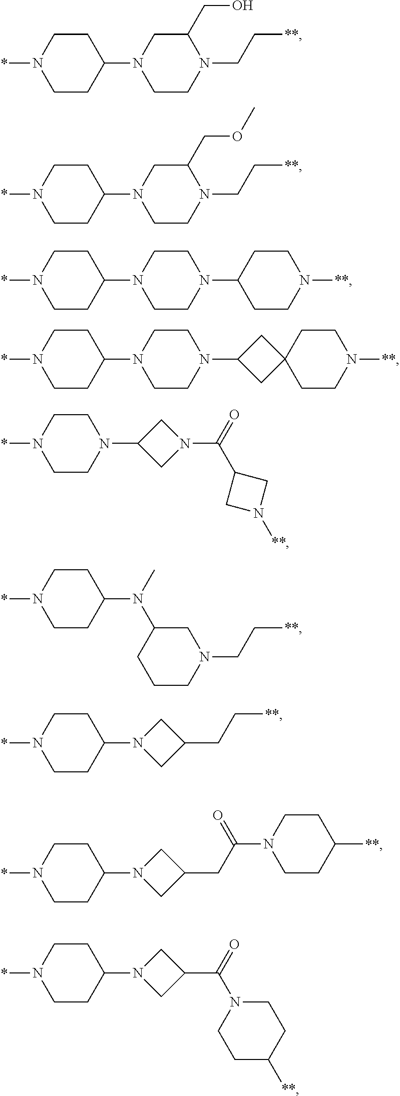

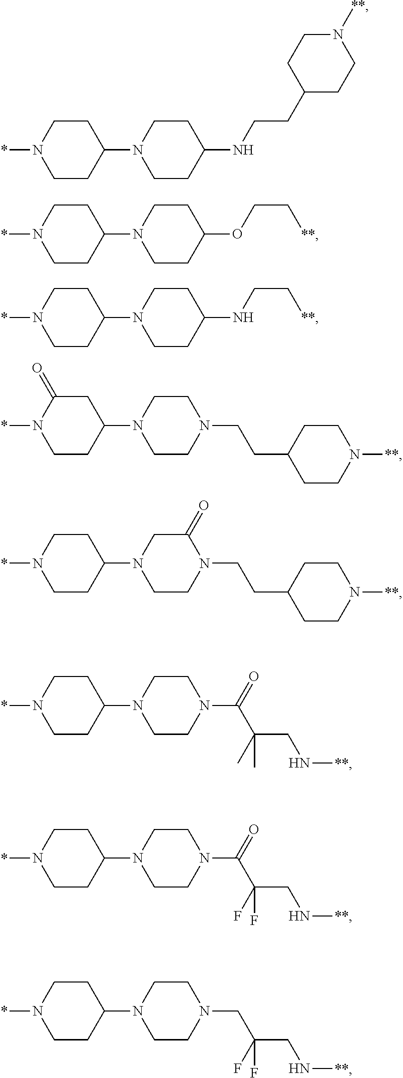

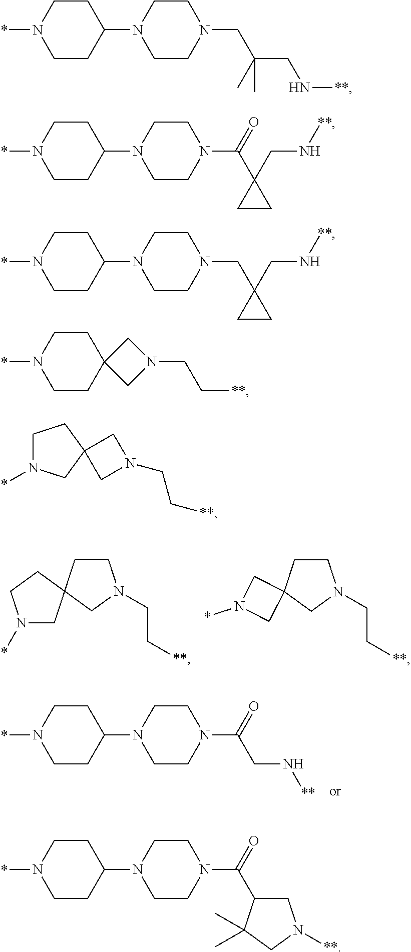

Aspect 18. The compound of any one of aspects 1-17, wherein Warhead is a moiety which binds to a target protein, wherein said target protein is selected from the group consisting of structural proteins, receptors, enzymes, cell surface proteins, proteins pertinent to the integrated function of a cell, including proteins involved in catalytic activity, aromatase activity, motor activity, helicase activity, metabolic processes (anabolism and catabolism), antioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity and translation regulator activity.

Aspect 19. The compound of any one of aspects 1-18, wherein Warhead is a moiety which binds to a target protein, wherein said target protein is selected from the group consisting of ErbB receptors, B7.1 and B7, TINFR1m, TNFR2, NADPH oxidase, Bcl-Bax and other partners in the apotosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases (including Bruton's Tyrosine Kinase), CD23, CD124, tyrosine kinase p561ck, CD4, CD5, IL-2 receptor, IL-1 receptor, TNF-alphaR, ICAM1, Cat+ channels, VCAM, VLA-4 integrin, selectins, CD40/CD40L, newokinins and receptors, inosine monophosphate dehydrogenase, p38 MAP Kinase, RAS-RAF-MEK-ERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, adenosine receptors, adenosine kinase and AMP deaminase, purinergic receptors (P2Y1, P2Y2, P2Y4, P2Y6, P2X 1-7), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase, ecdysone 20-monooxygenase, ion channel of the GABA gated chloride channel, acetylcholinesterase, voltage-sensitive sodium channel protein, calcium release channel, chloride channels, Acetyl-CoA carboxylase, adenylosuccinate synthetase, protoporphyrinogen oxidase, L-1 receptor associated kinase-3 (IRAK-3 or IRAK-M) or enolpyruvyl-shikimate-phosphate synthase.

Aspect 20. The compound of any one of aspects 1-19, wherein Warhead is

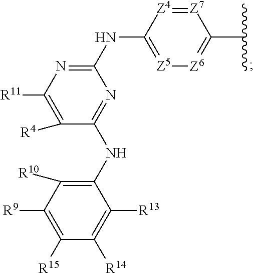

wherein R¹³ is selected from —P(O)R^{13a}R^{13b}, —SO₂R^{13a}, —SO₂—NR^{13a}R^{13b} or —N(R^{13a})—SO₂R^{13b}.
R^{13a} and R^{13b} are each independently selected from hydrogen, —C₁-C₈alkyl or C₃-C₈cycloalkyl, said —C₁-C₈alkyl or C₃-C₈cycloalkyl is optionally substituted with at least one halogen;
R¹⁴ and R¹⁵ are each independently selected from hydrogen, halogen, —C₁-C₈alkyl, —C₃-C₈cycloalkyl, 3- to 8-membered heterocyclyl, C₆-C₁₂aryl, 5- to 12-membered heteroaryl, —CN, —OR^{14a}, —SO₂R^{14a}, —SO₂NR^{14a}R^{14b}, —COR^{14a}, —CO₂R^{14a}, —CONR^{14a}R^{14b}, —NR^{14a}R^{14b}, —NR^{14a}COR^{14b}, —NR^{14a}CO₂R^{14b}, or —NR^{14a}SO₂R^{14b}; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{14d}, or
R¹⁴ and R¹⁵ together with the carbon atoms to which they are attached, form a 5 or 6-membered unsaturated or saturated ring, said ring comprising 0-3 heteroatoms independently selected from nitrogen, oxygen or sulfur; said ring is optionally substituted with at least one substituent R^{14e};
R^{14e}, at each occurrence, is independently hydrogen, halogen, —C₁-C₈alkyl, —C₁-C₈alkoxy, —C₃-C₈cycloalkyl, 3- to 8-membered heterocyclyl, C₆-C₁₂aryl, 5- to 12-membered heteroaryl, oxo (═O), —OR^{14a}, thioxo (═S), —SR^{14a}, —CN, —SO₂R^{14a}, —SO₂NR^{14a}R^{14b}, —COR^{14a}, —CO₂R^{14a}, —CONR^{14a}R^{14b}, —NR^{14a}R^{14b}, —NR^{14a}COR^{14b}, —NR^{14a}CO₂R^{14b} or —NR^{14a}SO₂R^{14b}; each of —C₁-C₈alkyl, —C₁-C₈alkoxy, C₃-C₈cycloalkyl, 3- to 8-membered heterocyclyl, C₆-C₁₂aryl or 5- to 12-membered heteroaryl is optionally substituted with at least one substituent R^{14d};
R^{14a} and R^{14b} are each independently selected from hydrogen, —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl;
R^{14d}, at each occurrence, is independently halogen, —OH, —CN, oxo, —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
R⁴ is selected from hydrogen, halogen, —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl, 5- to 12-membered heteroaryl, —CN, —SO₂R^{4a}, —SO₂NR^{4a}R^{4b}, —COR^{4a}, —CO₂R^{4a}, —CONR^{4a}R^{4b}, —NR^{4a}R^{4b}, —NR^{4a}COR^{4b}, —NR^{4a}CO₂R^{4b} or —NR^{4a}SO₂R^{4b}; each of —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl or 5- to 12-membered heteroaryl is optionally substituted with halogen, —C₁-C₈alkyl, —C₂-C₈alkenyl, —C₂-C₈alkynyl, —C₃-C₈cycloalkyl, 3- to 8-membered heterocyclyl, C₆-C₁₂aryl, 5- to 12-membered heteroaryl, oxo, —CN, —OR^{4c}, —SO₂R^{4c}, —SO₂NR^{4c}R^{4d}, —COR^{4c}, —CO₂R^{4c}, —CONR^{4c}R^{4d}, —NR^{4c}R^{4d}, —NR^{4c} COR^{4d}, —NR^{4c} CO₂R^{4d} or —NR^{4c} SO₂R^{4d};
R^{4a}, R^{4b}, R^{4c} and R^{4d} are each independently hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁹, R¹⁰ and R¹¹ are each independently selected from hydrogen, halogen, —C₁-C₈alkyl, —NR^{9a}R^{9b}, —OR^{9a}, —C₃-C₈cycloalkyl, 3- to 8-membered heterocyclyl, —C₆-C₁₂aryl, 5- to 12-membered heteroaryl, oxo or —CN; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{9c};
R^{9a} and R^{9b} are each independently selected from hydrogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{9d}; or
R^{9c} and R^{9d} are each independently halogen, hydroxy, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl;
Z⁴, Z⁵, Z⁶ and Z⁷ are each independently selected from —CR^{Z4}, or N;
R^{Z4}, at each occurrence, is independently selected from hydrogen, halogen, —C₁-C₈alkyl, —NR^{Z4a}R^{Z4b}, —OR^{Z4a}, SR^{Z4a}, C₃-C₈cycloalkyl, 3- to 8-membered heterocyclyl, C₆-C₁₂aryl, 5- to 12-membered heteroaryl, or CN; each of —C₁-C₈alkyl, C₃-C₈cycloalkyl, 3- to 8-membered heterocyclyl, C₆-C₁₂aryl, or 5- to 12-membered heteroaryl is optionally substituted with at least one R^{Z4}c;
R^{Z4a} and R^{Z4b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{Z4d};
R^{Z4}c and R^{Z4d} are each independently halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl.

Aspect 21. The compound of aspect 20, wherein R¹³ is selected from —P(O)R^{13a}R^{13b} or —N(R^{13a})—SO₂R^{13b}, wherein R^{13a} and R^{13b} are each independently selected from hydrogen, —C₁-C₈alkyl (preferably —CH₃, —C₂H₅, —C₃H₇, —C₄H₉ or —C₅H₁₁; more preferably —CH₃, —CH₂CH₃, —CH₂CH₂CH₃, -iso-C₃H₇, —CH₂CH₂CH₂CH₃, -iso-C₄H₉, -sec-C₄H₉ or -tert-C₄H₉) or C₃-C₈cycloalkyl (preferably cyclopropyl, cyclobutyl or cyclopentyl).

Aspect 22. The compound of aspect 20, wherein R¹³ is selected from —P(O)(CH₃)₂, —NH—SO₂CH₃ or —N(CH₃)—SO₂CH₃.

Aspect 23. The compound of aspect 20, wherein R¹³ is —P(O)(CH₃)₂.

Aspect 24. The compound of aspect 20, wherein R¹⁴ and R¹⁵ are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, —CN, —OR^{14a}, —SO₂R^{14a}, —SO₂NR^{14a}R^{14b}, —COR^{14a}, —CO₂R^{14a}, —CONR^{14a}R^{14b}, —NR^{14a}R^{14b}, —NR^{14a}COR^{14b}, —NR^{14a}CO₂R^{14b}, or —NR^{14a}SO₂R^{14b}; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, C₆-C₁₂aryl or 5- to 12-membered heteroaryl is optionally substituted with at least one substituent R^{14d}, or
R¹⁴ and R¹⁵ together with the carbon atoms to which they are attached, form a 5 or 6-membered unsaturated or saturated ring, said ring comprising 0, 1, 2 or 3 heteroatoms independently selected from nitrogen, oxygen or sulfur; said ring is optionally substituted with at least one substituent R^{14e};
R^{14e}, at each occurrence, is independently —H, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, phenyl, 3- to 8-membered heterocyclyl, 5- to 12-membered heteroaryl, oxo, —CN, CF₃, CHF₂, CH₂F, thioxo, —SCF₃, —SCHF₂, —SCH₂F, —SCH₂CF₃, —SCF₂CH₃, —SCF₂CF₃, —SO₂R^{14a}, —SO₂NR^{14a}R^{14b}, —COR^{14a}, —CO₂R^{14a}, —CONR^{14a}R^{14b}, —NR^{14a}R^{14b}, —NR^{14a}COR^{14b}, —NR^{14a}CO₂R^{14b} or —NR^{14a}SO₂R^{14b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, phenyl, 3- to 8-membered heterocyclyl, 5- to 12-membered heteroaryl is optionally substituted with at least one substituent R^{14d};
R^{14a} and R^{14b} are each independently selected from hydrogen, methyl, ethyl, propyl, butyl, pentyl, hexyl, heptyl, octyl, methoxy, ethoxy, propoxy, butoxy, pentoxy, hexoxy, heptyloxy, octyloxy, C₁-C₈alkoxy-C₁-C₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl;
R^{14d}, at each occurrence, is independently halogen, —OH, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 25. The compound of aspect 20, wherein R¹⁴ and R¹⁵ together with the carbon atoms to which they are attached, form a 5 or 6-membered unsaturated (preferred aromatic) or saturated ring, said ring comprising 1 or 2 nitrogen heteroatoms; said ring is optionally substituted with at least one substituent —H, —F, —Cl, —Br, —I, methyl, ethyl, propyl (n- or iso-), butyl, pentyl, methoxy, ethoxy, propoxy, butoxy, pentoxy, cyclopropyl, cyclobutyl, cyclopentyl, cyclohexyl, cycloheptyl, cyclooctyl, —CH₂OH, —SCH₃, —SC₂H₅, oxo, thioxo, —CF₃, —CHF₂, —CH₂F, —SCF₃, —OMe, —OC₂H₅, —CN, —C(O)CH₃,

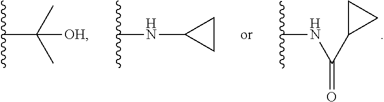

Aspect 26. The compound of aspect 20, wherein R¹⁴ and R¹⁵ together with the carbon atoms to which they are attached, form a 6-membered unsaturated (preferred aromatic), said ring comprising 1 or 2 nitrogen heteroatoms; said ring is optionally substituted with one substituent —H, —F, —Cl, —Br, —I, methyl, ethyl or cyclopropyl.

Aspect 27. The compound of aspect 20, wherein R⁴ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —C₂-C₈alkenyl, —C₂-C₈alkynyl or —C₁-C₈alkoxy; each of methyl, ethyl, propyl, butyl, pentyl, hexyl, heptyl, octyl, cyclopropyl, cyclobutyl, cyclopentyl, cyclohexyl, cycloheptyl, cyclooctyl, —C₂-C₈alkenyl or —C₂-C₈alkynyl is optionally substituted with —F, —Cl, —Br, —I, oxo, or —CN.

Aspect 28. The compound of aspect 20, wherein R⁴ is hydrogen, —F, —Cl, —Br, —I, —CH₃, —CF₃, —CH₂F, or —CHF₂.

Aspect 29. The compound of aspect 20, wherein R⁴ is hydrogen, —F, —Cl, —Br or —I.

Aspect 30. The compound of aspect 20, wherein R⁹, R¹⁰ and R¹¹ are each independently selected from hydrogen, —F, —Cl, —Br, —I, methyl, ethyl, propyl, butyl, pentyl, hexyl, heptyl, octyl, —NR^{9a}R^{9b}, —OR^{9a}, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo, or —CN; each of -methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9c};
R^{9a} and R^{9b} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9d};
R^{9c} and R^{9d} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl.

Aspect 31. The compound of aspect 20, wherein R⁹, R¹⁰ and R¹¹ are each independently selected from hydrogen, —F, —Cl, —Br, —I, methyl, ethyl, propyl, butyl, —NH₂, —NHCH₃, —OH, —OCH₃, —OC₂H₅, cyclopropyl, cyclobutyl, cyclopentyl, cyclohexyl, —CH₂OH, —CH₂OMe, oxo, or —CN.

Aspect 32. The compound of aspect 20, wherein R⁹, R¹⁰ and R¹¹ are each independently selected from hydrogen, —CH₃, —F, —Cl, —Br or —I.

Aspect 33. The compound of aspect 20, wherein Z⁴, Z⁵, Z⁶ and Z⁷ are each independently —CR^{4z};
R^{4Z}, at each occurrence, is independently selected from hydrogen, —F, —Cl, —Br, —I, methyl, ethyl, propyl, butyl, pentyl, hexyl, heptyl, octyl, —NR^{4Za}R^{4Zb}, OR^{4Za}, —SR^{4Za}, cyclopropyl, cyclobutyl, cyclopentyl, cyclohexyl, cycloheptyl, cyclooctyl, 3- to 8-membered heterocyclyl, phenyl, 5- to 12-membered heteroaryl, or CN; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one R^{4Zc};
R^{4Za} and R^{4Zb} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{4Zd};
R^{4Zc} and R^{4Zd} are each independently —F, —Cl, —Br, —I, hydroxy, methyl, ethyl, propyl, butyl, pentyl, hexyl, heptyl, octyl, —C₁-C₈alkoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

Aspect 34. The compound of aspect 20, wherein R^{4z} is selected from H, —CH₃, —C₂H₅, F, —CH₂F, —CHF₂, —CF₃, —OCH₃, —OC₂H₅, —C₃H₇, —OCH₂F, —OCHF₂, —OCH₂CF₃, —OCF₃, —SCF₃, —CF₃, —CH(OH)CH₃,

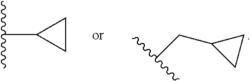

Aspect 35. The compound of any one of aspects 1-19, wherein Warhead is

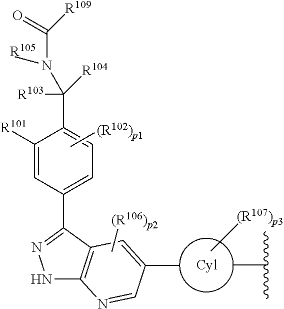

wherein

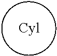

is a 5- or 6-membered aromatic ring comprising 0-3 heteroatoms selected from nitrogen, oxygen and sulfur;
R¹⁰¹, R¹⁰², R¹⁰³, R¹⁰⁴, R¹⁰⁵, R¹⁰⁶ and R¹⁰⁷ are each independently hydrogen, halogen, —C₁₋₈alkyl, —C₁₋₈alkoxy, —C₂₋₈alkenyl, —C₂₋₈alkynyl, cycloalkyl, heterocyclyl, aryl, heteroaryl, —CN, —NO₂, —OR^{10a}, —SO₂R^{10a}-COR^{10a}, —CO₂R^{10a}, —CONR^{10a}R^{10b}, —C(═NR^{10a})NR^{10b}R^{10c}, —NR^{10a}R^{10b}, —NR^{10a}COR^{10b}, —NR^{10a}CONR^{10b}R^{10c}, —NR^{10a}CO₂R^{10b}, —NR^{10a}SONR^{10b}R^{10c}, —NR^{10a}SO₂NR^{10b}R^{10c}, or —NR^{10a}SO₂R^{10b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl;
R¹⁰⁹ is 5- or 6-membered aromatic ring comprising 0-3 heteroatoms selected from nitrogen, oxygen and sulfur; said aromatic ring is optionally substituted with halogen, —C₁₋₈alkyl, —C₂₋₈alkenyl, —C₂₋₈alkynyl, —C₁₋₈alkoxy, cycloalkyl, heterocyclyl, aryl, heteroaryl, —CN, —NO₂, —OR^{10c}, —SO₂R^{10a}, —COR^{10a}, —CO₂R^{10a}, —CONR^{10a}R^{10b}, —C(═NR^{10a})NR^{10b}R^{10c}, —NR^{10a}R^{10b}, —NR^{10a}COR^{10b}, —NR^{10a}CONR^{10b}R^{10c}, —NR^{10a}CO₂R^{10b}, —NR^{10a}SONR^{10b}R^{10c}, —NR^{10a}SO₂NR^{10b}R^{10c}, or —NR^{10a}SO₂R^{10b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl;
p1, p2 and p3 are each independently 0, 1, 2, 3 or 4;
R^{10a}, R^{10b} and R^{10c} are each independently hydrogen, —C₁₋₈alkyl, —C₂₋₈alkenyl, —C₂₋₈alkynyl, cycloalkyl, heterocyclyl, aryl, or heteroaryl.

Aspect 36. The compound of aspect 35, wherein

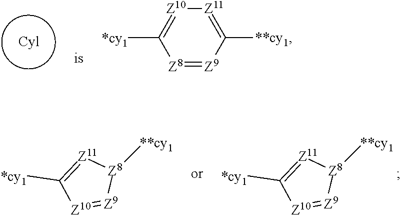

wherein Z⁸, Z⁹, Z¹⁰ and Z¹¹ are each independently selected from CH or N; wherein *cy1 refers to the position attached to the

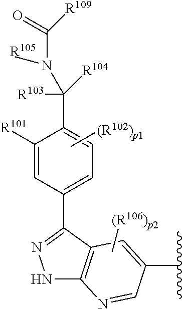

moiety, and **cy1 refers to the position attached to L¹.

Aspect 37. The compound of aspect 35, wherein

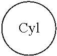

is selected from

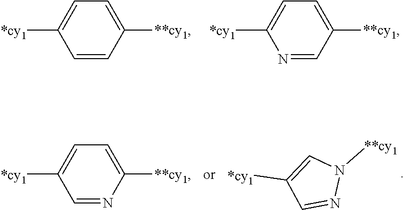

Aspect 38. The compound of aspect 35, wherein p3 is 0, 1, or 2, and each R¹⁰⁷ is independently selected from halogen, —C₁₋₈alkyl, or —C₁₋₈alkoxy, preferably F, Cl, Br, I, CH₃, or —OCH₃.

Aspect 39. The compound of aspect 35, wherein R^{10a} and R^{10b} are independently selected from hydrogen or CH₃; and n1 is 1 or 2.

Aspect 40. The compound of aspect 35, wherein R¹⁰¹ is methyl, —CH₂OH, —OCH₃, —CH₂OCH₃ or halogen; p1 is 0 or 1, and R¹⁰² is halogen.

Aspect 41. The compound of aspect 35, wherein R¹⁰³ and R¹⁰⁵ are hydrogen; and R¹⁰⁴ is selected from hydrogen or methyl.

Aspect 42. The compound of aspect 35, wherein R¹⁰⁹ is

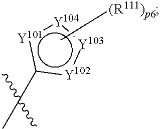

Y¹⁰¹, Y¹⁰², Y¹⁰³ and Y¹⁰⁴ are selected from CH, O, S or N; R¹¹¹ is selected from hydrogen, halogen, —C₁₋₈alkyl, —C₁₋₈alkoxy, —C₂₋₈alkenyl, —C₂₋₈alkynyl, cycloalkyl, heterocyclyl, aryl, heteroaryl, —CN, —NO₂, —OR^{10a}, —SO₂R^{10a}, —COR^{10a}, —CO₂R^{10a}, —CONR^{10a}R^{10b}, —C(═NR^{10a})NR^{10b}R^{10c}, —NR^{10a}R^{10b}, —NR^{10a}COR^{10b}, —NR^{10a}CONR^{10b}R^{10c}, —NR^{10a}CO₂R^{10b}, —NR^{10a}SONR^{10b}R^{10c}, —NR^{10a}SO₂NR^{10b}R^{10c}, or —NR^{10a}SO₂R^{10b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl; R^{10a}, R^{10b}, and R^{10c} are each independently hydrogen, —C₁₋₈alkyl, —C₂₋₈alkenyl, —C₂₋₈alkynyl, cycloalkyl, heterocyclyl, aryl, or heteroaryl; and p6 is 0, 1, 2, 3 or 4.

Aspect 43. The compound of aspect 35, wherein Y¹⁰¹ is CH, S, N or O; Y¹⁰² is CH, O or N; Y¹⁰³ is O, S or N; and Y¹⁰⁴ is S, CH or N.

Aspect 44. The compound of any one of aspects 1-19, wherein Warhead is

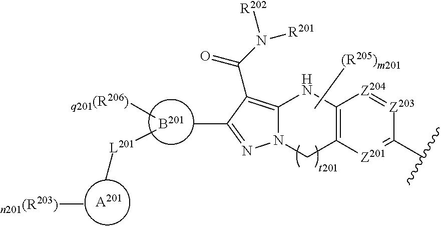

ring A²⁰¹ and B²⁰¹ are each independently an aromatic ring comprising 0-3 heteroatoms selected from nitrogen, sulfur and oxygen as ring member(s);
Z²⁰¹, Z²⁰³ and Z²⁰⁴ are each independently N or CR^{20z};
L²⁰¹ is independently a bond, —C₁₋₈alkylene-, —N(R²⁰⁴)—, —O—, —S—, *^{L201}-C₁₋₈alkylene-O-**^{L201}, *^{L201}-O—C₁₋₈alkylene-**^{L201}, *^{L201}-N(R²⁰⁴)CO-**^{L201}, *^{L201}-CON(R²⁰⁴)-**^{L201}, *^{L201}-N(R²⁰⁴)CO—C₁₋₈alkylene-**^{L201}, *^{L201}-CON(R²⁰⁴)—C₁₋₈alkylene-**^{L201}, *^{L201}-N(R²⁰⁴)—C₁₋₈alkylene-**^{L201}, *^{L201}-C₁₋₈alkylene-N(R²⁰⁴)-**^{L201}, -heterocyclene-, or -heteroarylene-, wherein each of said —C₁₋₈alkylene-, *^{L201}-C₁₋₈alkylene-O-**^{L201}, *^{L201}-O—C₁₋₈alkylene-**^{L201}, *^{L201}-N(R²⁰⁴)CO—C₁₋₈alkylene-**^{L201}, *^{L201}-CON(R²⁰⁴)—C₁₋₈alkylene-**^{L201}, *^{L201}-N(R²⁰⁴)—C₁₋₈alkylene-**^{L201}, *^{L201}-C₁₋₈alkylene-N(R²⁰⁴)-**^{L201}, -heterocyclene- and -heteroarylene- is optionally substituted with at least one substituent R^{20L};
wherein *^{L201} refers to the position attached to ring A, and **^{L201} refers to the position attached to ring B;
m201, n201 and q201 are each independently 0, 1, 2, 3 or 4;
t201 is 0, 1 or 2;
R²⁰¹, R²⁰², and R²⁰⁴ are each independently hydrogen, —C₁₋₈alkyl, —C₂₋₈alkenyl, —C₂₋₈alkynyl, cycloalkyl, heterocyclyl, aryl, or heteroaryl, wherein each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
R^{20L}, R²⁰³, R²⁰⁵ and R²⁰⁶ are each independently hydrogen, halogen, —C₁₋₈alkyl, —C₂₋₈alkenyl, —C₂₋₈alkynyl, cycloalkyl, heterocyclyl, aryl, heteroaryl, —CN, —NO₂, —OR^{20a}, —SO₂R^{20a}, —COR^{20a}, —CO₂R^{20a}, —CONR^{20a}R^{20b}, —C(═NR²a)NR^{20b}R^{20c}, —NR^{20a}R^{20b}, —NR^{20a}COR^{20b}, —NR^{20a}CONR^{20b}R^{20c}, —NR^{20a}CO₂R^{20b}, —NR^{20a}SONR^{20b}R^{20c}, —NR^{20a}SO₂NR^{20b}R^{20c}, or —NR^{20a}SO₂R^{20b}, wherein each of said —C₁₋₈alkyl, —C₂₋₈alkenyl, —C₂₋₈alkynyl, cycloalkyl, heterocyclyl, aryl, or heteroaryl is optionally substituted with at least one halogen, hydroxy, —C₁₋₈alkyl, —C₁₋₈alkyoxy, cycloalkyl, heterocyclyl, aryl, or heteroaryl;
R^{20z} is selected from hydrogen, halogen, —C₁₋₈alkyl, —C₂₋₈alkenyl, —C₂₋₈alkynyl, cycloalkyl, heterocyclyl, aryl, heteroaryl, —CN, —NO₂, —OR^{20a}, —SO₂R^{20a}, —COR^{20a}, —CO₂R^{20a}, —CONR^{20a}R^{20b}, —C(═NR²⁰a)NR^{20b}R^{20c}, —NR^{20a}R^{20b}, —NR^{20a}COR^{20b}, —NR^{20a}CONR^{20b}R^{20c}, —NR^{20a}CO₂R^{20b}, —NR^{20a}SONR^{20b}R^{20c}, —NR^{20a}SO₂NR^{20b}R^{20c}, or —NR^{20a}SO₂R^{20b}, each of said —C₁₋₈alkyl, —C₂₋₈alkenyl, —C₂₋₈alkynyl, cycloalkyl, heterocyclyl, aryl, or heteroaryl is optionally substituted with at least one halogen, hydroxy, —C₁₋₈alkyl, —C₁₋₈alkyoxy, cycloalkyl, heterocyclyl, aryl, or heteroaryl;
R^{20a}, R^{20b}, and R^{20c} are each independently hydrogen, —C₁₋₈alkyl, —C₂₋₈alkenyl, —C₂₋₈alkynyl, cycloalkyl, heterocyclyl, aryl, or heteroaryl;
or two R^{20L}, together with the atom(s) to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{203f}, —SO₂R^{203f}, —SO₂NR^{203f}R^{203g}, —COR^{203f}, —CO₂R^{203f}, —CONR^{203f}R^{203g}, —C(═NR²⁰³f)NR^{203g}R^{203h}, —NR^{203f}R^{203g}, —NR^{203f}COR^{203g}, —NR^{203f}CONR^{203g}R^{203h}, —NR^{203f}CO₂R^{203f}, —NR^{203f}SONR^{203f}R^{203g}, NR^{203f}SO₂NR^{203g}R^{203h} or —NR^{203f}SO₂R^{203g} each of said —C₁₋₈alkyl, —C₂₋₈alkenyl, —C₂₋₈alkynyl, cycloalkyl, heterocyclyl, aryl, or heteroaryl is optionally substituted with at least one substituent selected from halogen, —C₁₋₈alkyl, —OR²⁰³ⁱ, —NR²⁰³ⁱR^{203j}, cycloalkyl, heterocyclyl, aryl, or heteroaryl;
or two R²⁰³, together with the atoms to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{203f}, —SO₂R^{203f}, -SO₂NR^{203f}R^{203g}, —COR^{203f}, —CO₂R^{203f}, —CONR^{203f}R^{203g}, —C(═NR²⁰³f)NR^{203g}R^{203h}, —NR^{203f}R^{203g}, —NR^{203f}COR^{203g}, —NR^{203f}CONR^{203g}R^{203h}, —NR^{203f}CO₂R^{203f}, —NR^{203f}SONR^{203f}R^{203g}, NR^{203f}SO₂NR^{203g}R^{203h}, or —NR^{203f}SO₂R^{203g}, each of said —C₁₋₈alkyl, —C₂₋₈alkenyl, —C₂₋₈alkynyl, cycloalkyl, heterocyclyl, aryl, or heteroaryl is optionally substituted with at least one substituent selected from halogen, —C₁₋₈alkyl, —OR²⁰³ⁱ, —NR²⁰³ⁱR^{203j}, cycloalkyl, heterocyclyl, aryl, or heteroaryl;
or R⁴ and one of R³, together with the atoms to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{203f}, —SO₂R^{203f}, -SO₂NR²⁰³R^{203g}, —COR^{203f}, —CO₂R^{203f}-CONR^{203f}R^{203g}, —C(═NR²⁰³f)NR^{203g}R^{203h}, —NR^{203f}R^{203g}, —NR^{203f}COR^{203g}, —NR^{203f}CONR^{203g}R^{203h}, —NR^{203f}CO₂R^{203f}, —NR^{203f}SONR^{203f}R^{203g}, NR^{203f}SO₂NR^{203g}R^{203h}, or -NR^{203f}SO₂R^{203g}, each of said —C₁₋₈alkyl, —C₂₋₈alkenyl, —C₂₋₈alkynyl, cycloalkyl, heterocyclyl, aryl, or heteroaryl is optionally substituted with at least one substituent selected from halogen, —C₁₋₈alkyl, —OR²⁰³ⁱ, —NR²⁰³ⁱR^{203j}, cycloalkyl, heterocyclyl, aryl, or heteroaryl;
R^{203f}, R^{203g}, R^{203h}, R²⁰³ⁱ, and R^{203j} are each independently hydrogen, —C₁₋₈alkyl, C₁₋₈alkoxy-C₁₋₈alkyl-, —C₂₋₈alkenyl, —C₂₋₈alkynyl, cycloalkyl, heterocyclyl, aryl, or heteroaryl.

Aspect 45. The compound according to any one of Aspects 44, wherein L²⁰¹ is a bond, —CH₂—, —C₂H₄—, —C₃H₆—, —C₄H₈—, —C₅H₁₀—, —O—, —NH—, *^{L201}-NHCH₂-**^{L201}, *^{L201}-NHC₂H₄-**^{L201}, *^{L201}-NHC₃H₆-**^{L201}, *^{L201}-NHC₄H₈-**^{L201}, *^{L201}-NHC₅H₁₀-**^{L201}, *^{L201}-OCH₂-**^{L201}, *^{L201}-OC₂H₄-**^{L201}, *^{L201}-C₃H₆-**^{L201}, *^{L201}-OC₄H₈-**^{L201}, *^{L201}-C₅H₁₀-**^{L201}, *^{L201}-CH₂O-**^{L201}, *^{L201}-C₂H₄O-**^{L201}, *^{L201}-C₃H₆O-**^{L201}, *^{L201}-C₄H₈O-**^{L201}, *^{L201} C₅H₁₀-**^{L201}, *^{L201}-CONH-**^{L201}, *^{L201}-NHCO-**^{L201}, *^{L201}-CONHCH₂-**^{L201}, *^{L201}-CONHC₂H₄-**^{L201}, *^{L201}-CONHC₃H₆-**^{L201}, *^{L201}-CONHC₄H₈-**^{L201}, *^{L201}-CONHC₅H₁₀-**^{L201}, 3- to 8-membered -heterocyclene- or 5- to 6-membered -heteroarylene-; wherein each of said —CH₂—, —C₂H₄—, —C₃H₆—, —C₄H₈—, —C₅H₁₀—, —O—, —NH—, *^{L201}-NHCH₂-**^{L201}, *^{L201}-NHC₂H₄-**^{L201}, *^{L201}-NHC₃H-**^{L201}, *^{L201}NHC₄H₈**^{L201}, *^{L201}-NHC₅H₁₀**^{L201}, *^{L201}-OCH₂**^{L201}, *^{L201}-OC₂H₄-**^{L201}, *^{L201}-OC₃H-**^{L201}, *^{L201}-OC₄H₈-**^{L201}, *^{L201}-OC₅H₁₀-**^{L201}, *^{L201}-CH₂O-**^{L201}, *^{L201}-C₂H₄O**^{L201}, *^{L201}-C₃H₆O-**^{L201}, *^{L201}-C₄H₈**^{L201}, *^{L201}-C₅H₁₀**^{L201}, *^{L201}-CONH-**^{L201}, *^{L201}-NHCO-**^{L201}, *^{L201}-CONHCH₂**^{L201}, *^{L201}-CONHC₂H₄-**^{L201}, *^{L201}-CONHC₃H₆-**^{L201}, *^{L201}-CONHC₄H₈-**^{L201}, *^{L201}-CONHC₅H₁₀-**^{L201}, 3- to 8-membered heterocyclene- and 5- to 6-membered heteroarylene- is optionally substituted with at least one substituent R^{20L}; wherein R^{20L} is defined as above.

Aspect 46. The compound according to Aspect 44, wherein L²⁰¹ is a bond, —O—, *^{L201}-OCH₂-**^{L201}, *^{L201}-CH₂O-**^{L201}, —NH—, *^{L201}-CONH-**^{L201}, *^{L201}-NHCO-**^{L201}, *^{L201}-CONHCH₂-**^{L201}, *^{L201}-CONHCH₂CH₂-**^{L201}, *^{L201}-CONHCH₂CH₂CH₂-**^{L201}, *^{L201}-CONHCH(CH₃)-**^{L201}, *^{L201}-CONHCH(C₂H₅)-**^{L201}, *^{L201}-NHCH₂-**^{L201}, *^{L201}-NHCH₂CH₂-**^{L201}, *^{L201}-NHCH₂CH₂CH₂-**^{L201}, *^{L201}-NHCH(CH₃)-**^{L201} or *L-NHCH(C₂H₅)-**^{L}.

Aspect 47. The compound according to Aspect 44, wherein L²⁰¹ is *^{L201}-N(R²⁰⁴)CO-**^{L201}, R²⁰³ and R²⁰⁴, together with the atoms to which they are attached, form a 5-, 6- or 7-membered ring, said ring comprising 0, 1 or 2 additional heteroatoms independently selected from nitrogen, oxygen or optionally oxidized sulfur as ring member(s), said ring is optionally substituted with at least one substituent independently selected from —F, —Cl, —Br, —I, —CH₃, —C₂H₅, —C₃H₇, —C₄H₉, —C₅H₁₁, cycloalkyl, heterocyclyl, aryl, heteroaryl, or oxo.

Aspect 48. The compound according to Aspect 44, wherein

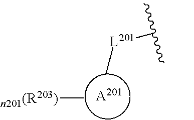

moiety is

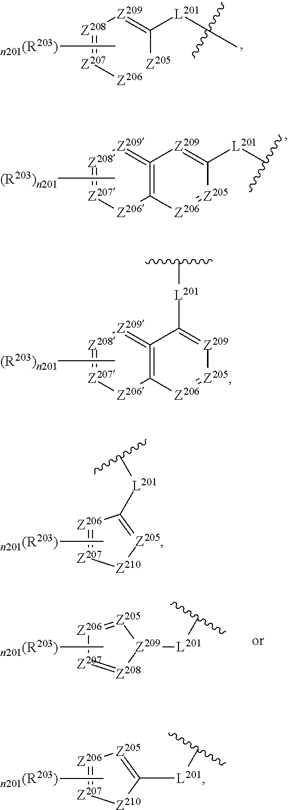

wherein Z²⁰⁵, Z²⁰⁶, Z²⁰⁷, Z²⁰⁸, Z²⁰⁹, Z²⁰⁶′, Z²⁰⁷′, Z²⁰⁸ and Z²⁰⁹′ are each independently N or C(H); Z²¹⁰ is N(H), O or S.

Aspect 49. The compound according to Aspect 44, wherein ring A201 is a 5- to 6-membered aromatic ring comprising 0-3 heteroatoms selected from nitrogen, sulfur and oxygen as ring member(s).

Aspect 50. The compound according to Aspect 44, wherein ring A201 is phenyl, naphthalenyl, quinoxalinyl, pyridinyl, pyridazinyl, pyrimidinyl, imidazolyl, thiazolyl, oxazolyl, oxadiazole, pyridyl, pyrazinyl, pyridazinyl, pyrrolyl, imidazolyl, pyrazolyl, triazolyl, furanyl, pyrimidinyl, pyrazinyl, pyrrolopyridinyl or dihydropyrrolopyrazinyl.

Aspect 51. The compound according to Aspect 44, wherein the

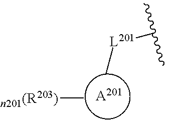

moiety is

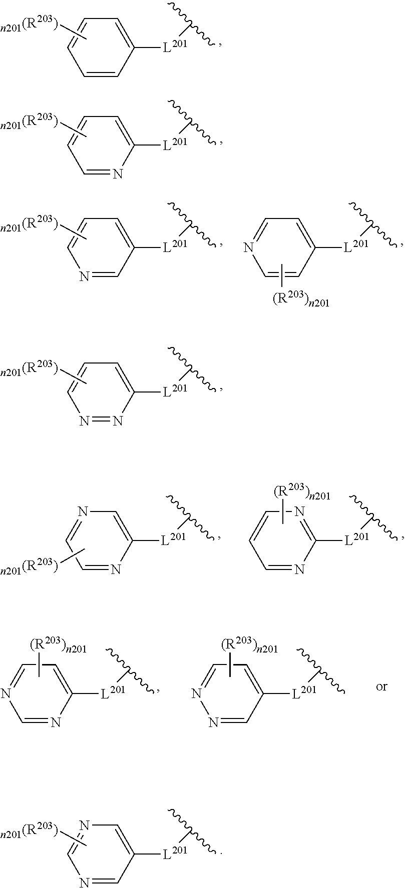

Aspect 52. The compound according to Aspect 44, wherein R²⁰³ is hydrogen, oxo, —F, —Cl, —Br, —I, —CH₃, —C₂H₅, —C₃H₇, —C₄H₉, —C₅H₁₁, —OCH₃, —OC₂H₅, —OC₃H₇, —OC₄H₉, —OC₅H₁₁, cyclopropyl, cyclobutyl, cyclopentyl, tetrahydropyrrolyl or phenyl, wherein each of said —CH₃, —C₂H₅, —C₃H₇, —C₄H₉, —C₅H₁₁, —OCH₃, —OC₂H₅, —OC₃H₇, —OC₄H₉, —OC₅H₁₁, cyclopropyl, cyclobutyl, cyclopentyl, tetrahydropyrrolyl or phenyl, is optionally substituted with at least one —F, —Cl, —Br, —I, —CH₃, —C₂H₅, —C₃H₇, —C₄H₉, —C₅H₁₁, —OCH₃, —OC₂H₅, —OC₃H₇, —OC₄H₉, —OC₅H₁₁, —OH, cyclopropyl, cyclobutyl or cyclopentyl.

Aspect 53. The compound according to Aspect 44, wherein R²⁰³ is hydrogen, oxo, —F, —Cl, —Br, —I, methyl, ethyl, n-propyl, iso-propyl, n-butyl, iso-butyl, sec-butyl, tert-butyl, trifluoromethyl, difluoromethyl, fluoromethyl, —OMe, —OEt, —OPr, —OBu, cyclopropyl, cyclobutyl, tetrahydropyrrolyl or phenyl.

Aspect 54. The compound according to Aspect 44, wherein two R²⁰³, together with the atoms to which they are attached, form a 4-, 5-, 6-, 7- or 8-membered ring, said ring comprising 0, 1 or 2 additional heteroatoms independently selected from nitrogen, oxygen or optionally oxidized sulfur as ring member(s), said ring is optionally substituted with at least one substituent independently selected from —F, —Cl, —Br, —I, —CH₃, —C₂H₅, —C₃H₇, —C₄H₉, —C₅H₁₁, —OCH₃, —OC₂H₅, —OC₃H₇, —OC₄H₉, —OC₅H₁₁, —OH, —CN, cyclopropyl, cyclobutyl or cyclopentyl.

Aspect 55. The compound according to Aspect 44, wherein

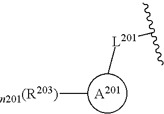

moeity is

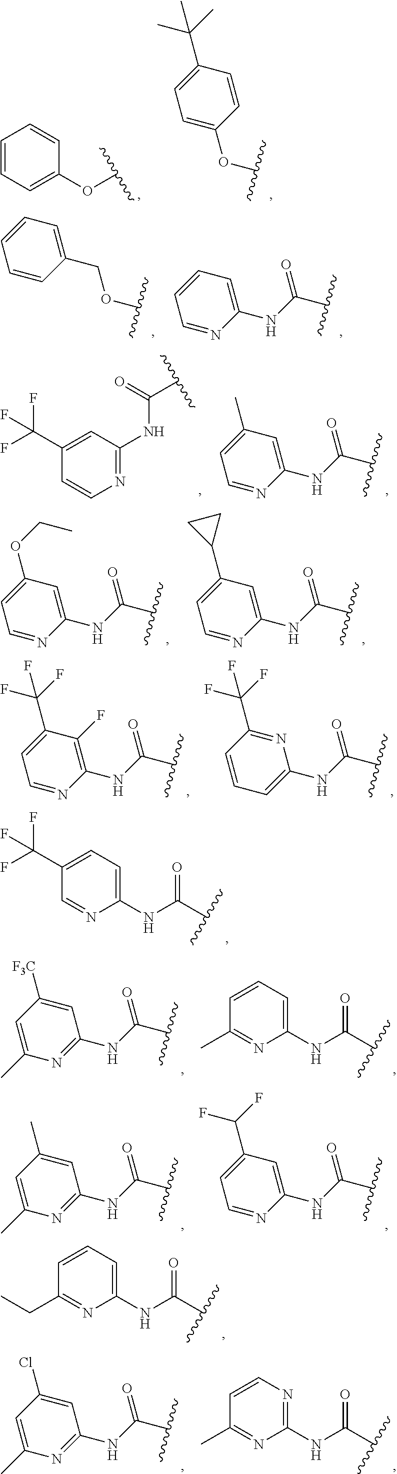

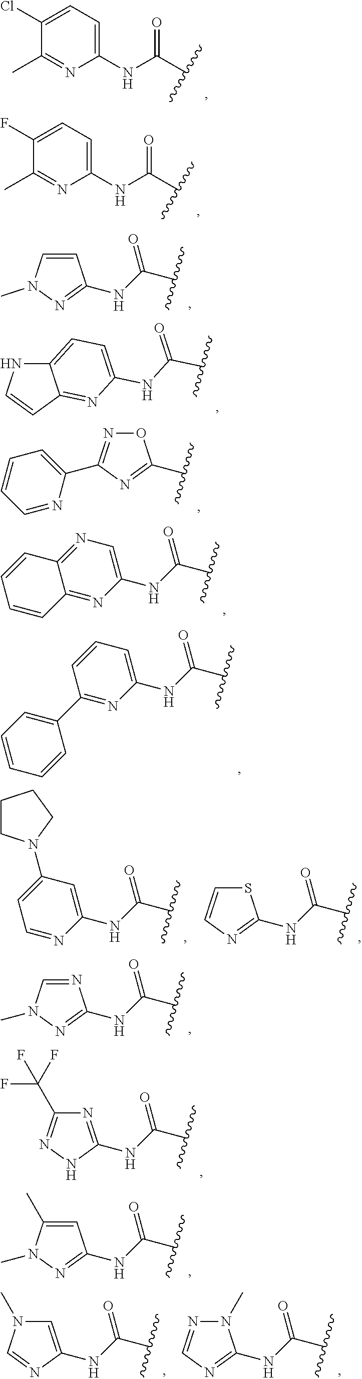

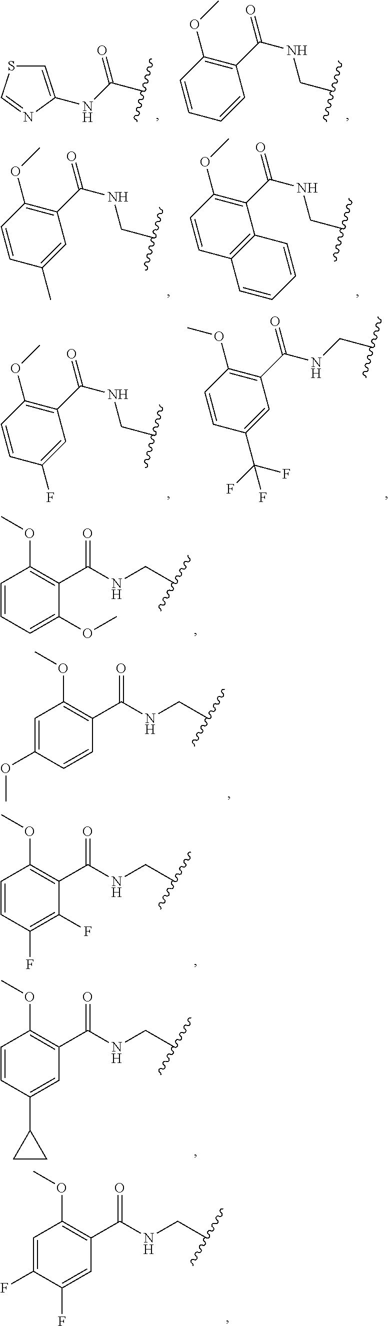

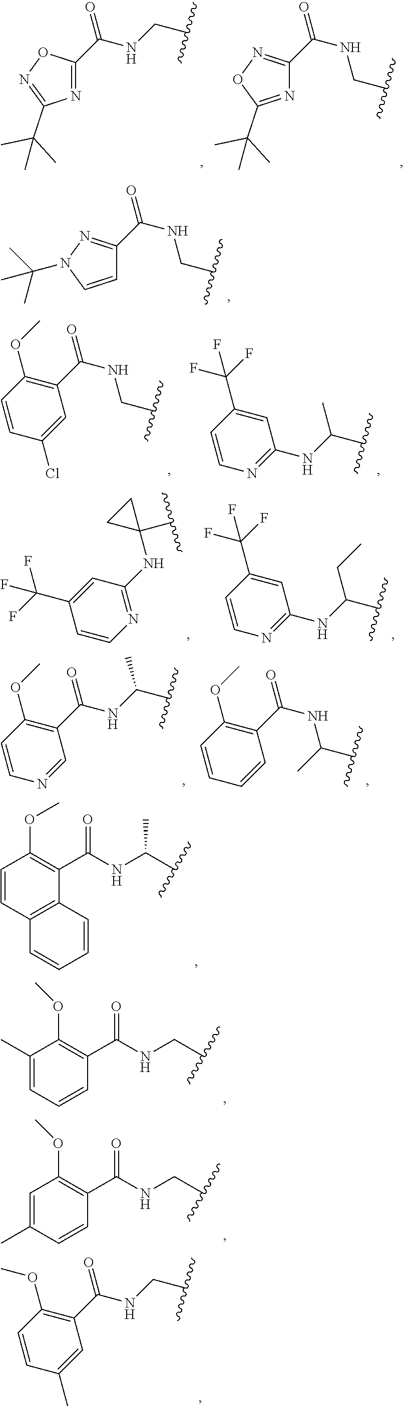

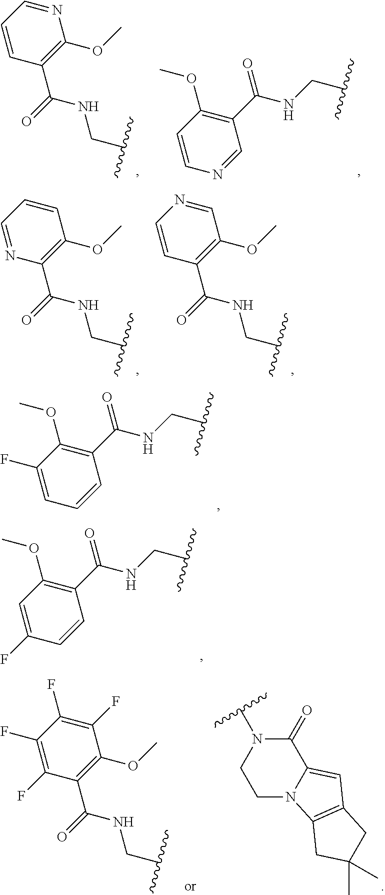

Aspect 56. The compound according to Aspect 44, wherein the

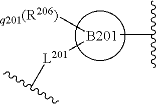

moiety is

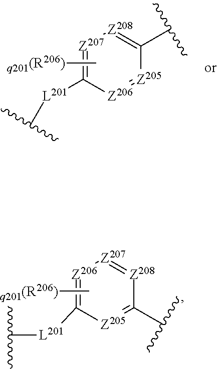

wherein Z²⁰⁵, Z²⁰⁶, Z²⁰⁷, and Z²⁰⁸ are defined as above.

Aspect 57. The compound according to Aspect 44, wherein the

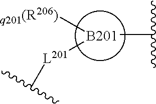

moiety is

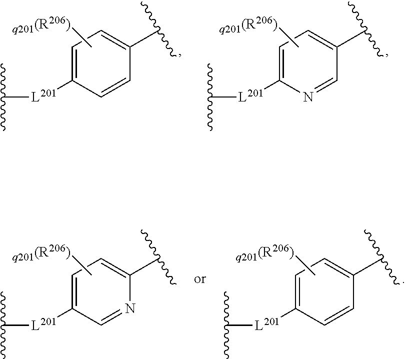

Aspect 58. The compound according to Aspect 44, wherein ring B201 is phenyl, pyridinyl, imidazolyl, thiazolyl, oxazolyl, pyridyl, pyrimidinyl, pyrazinyl, pyridazinyl, pyrrolyl, imidazolyl, pyrazolyl, thiazolyl, oxazolyl, triazolyl, thiophenyl, furanyl, pyrimidinyl or pyrazinyl, each of which is optionally substituted with (R²⁰⁶)_{q201}.

Aspect 59. The compound according to Aspect 44, wherein R²⁰⁶ is hydrogen, —F, —Cl, —Br, —I, —CH₃, —C₂H₅, —C₃H₇, —C₄H₉, —C₅H₁₁, —CN, —OCH₃, —OC₂H₅, —OC₃H₇, —OC₄H₉ or —OC₅H₁₁, wherein each of said —CH₃, —C₂H₅, —C₃H₇, —C₄H₉, —C₅H₁₁, —OCH₃, —OC₂H₅, —OC₃H₇, —OC₄H₉ or —OC₅H₁₁ is optionally substituted with —F, —Cl, —Br, —I, hydroxy, —C₁₋₈alkyoxy, cycloalkyl, heterocyclyl, aryl, or heteroaryl.

Aspect 60. The compound according to Aspect 44, wherein the

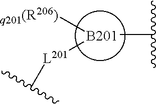

moiety is

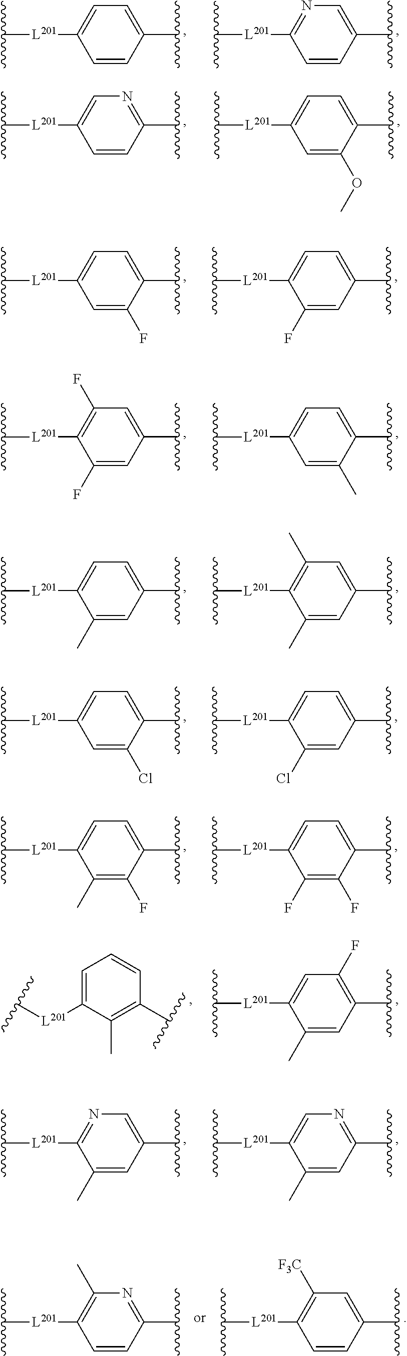

Aspect 61. The compound according to Aspect 44, wherein R²⁰¹ and R²⁰² are each independently hydrogen, —CH₃, —C₂H₅, —C₃H₇, —C₄H₉, —C₅H₁₁, —C₂₋₈alkenyl, —C₂₋₈alkynyl or aryl.

Aspect 62. The compound according to Aspect 44, wherein R²⁰¹ and R²⁰² are both H.

Aspect 63. The compound according to Aspect 44, wherein R²⁰⁵ is independently hydrogen, —F, —Cl, —Br, —I, —CH₃, —C₂H₅, —C₃H₇, —C₄H₉, —C₅H₁₁, —C₂₋₈alkenyl, —C₂₋₈alkynyl or aryl.

Aspect 64. The compound according to Aspect 44, wherein R^{20z} is hydrogen, —F, —Cl, —Br, —I, —CH₃, —C₂H₅, —C₃H₇, —C₄H₉ or —C₅H₁₁.

Aspect 65. The compound according to any one of Aspects 44 to 64, wherein the

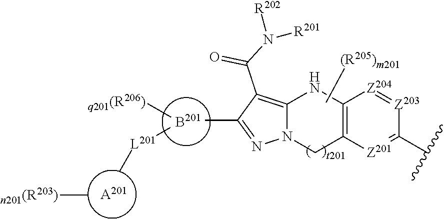

moiety is

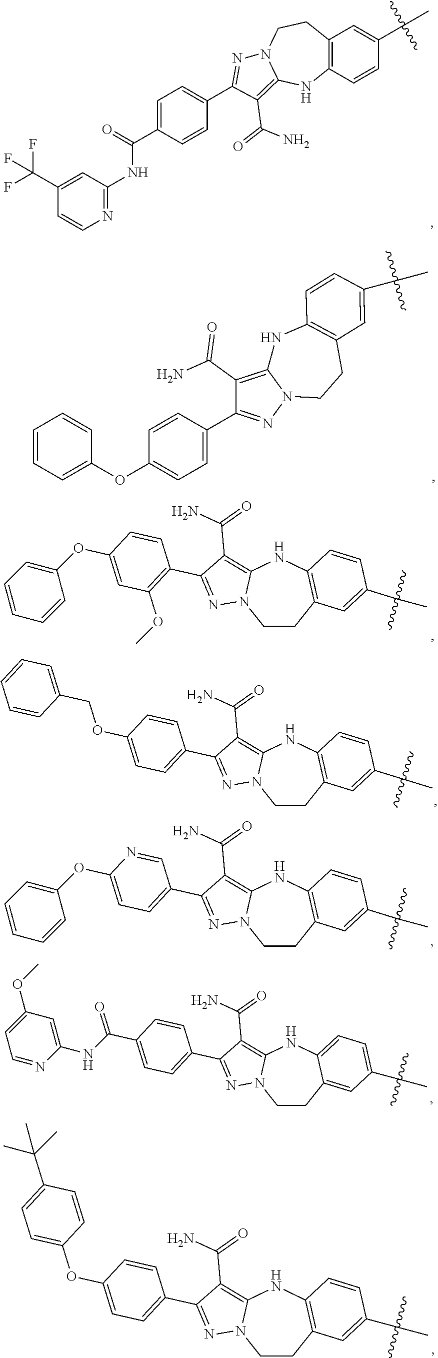

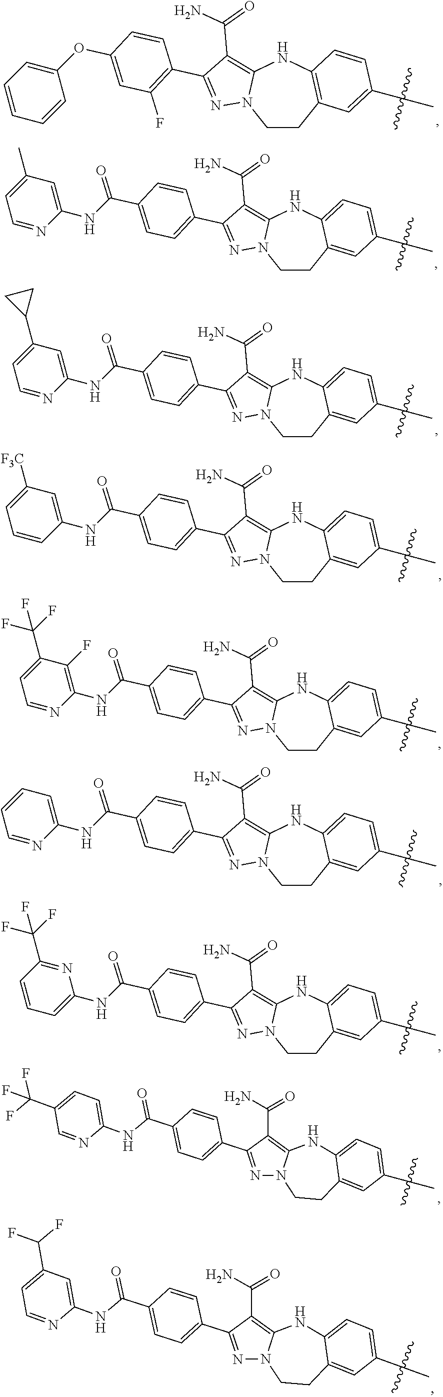

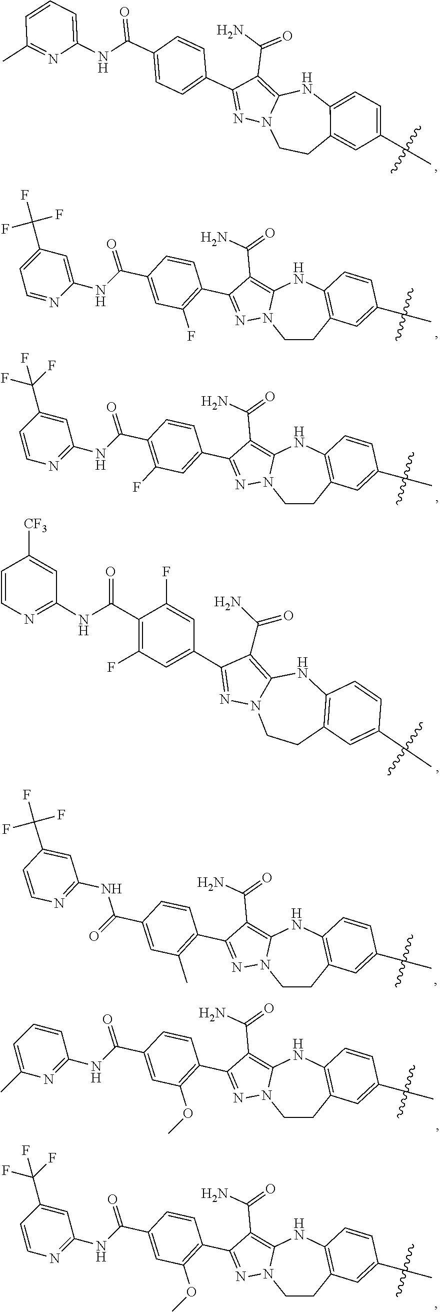

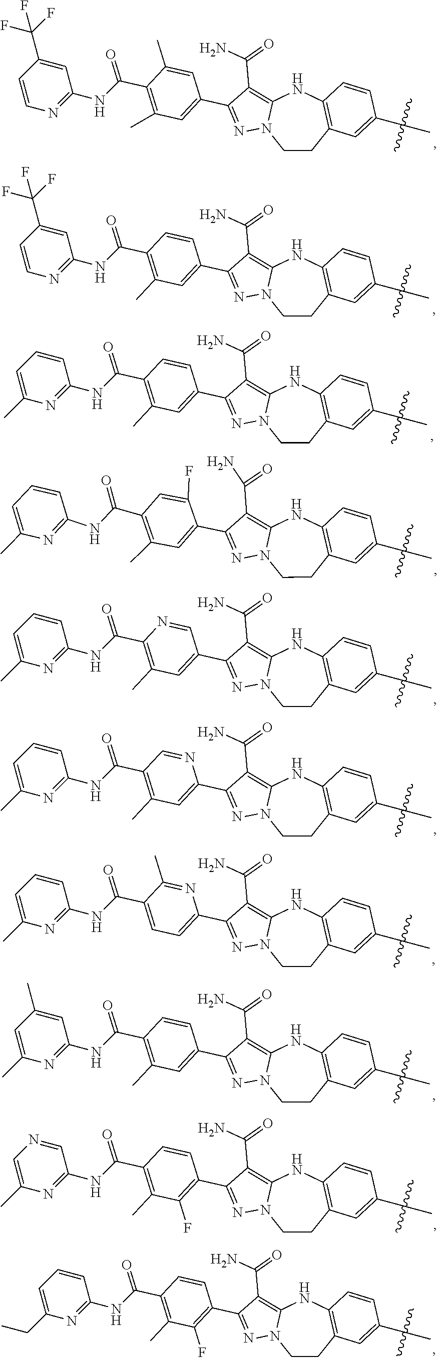

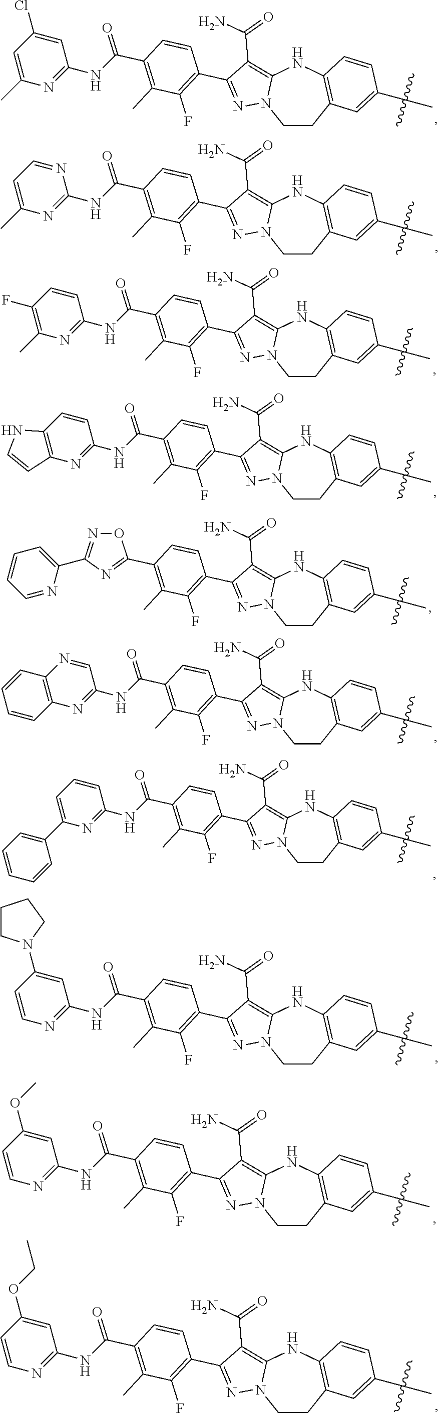

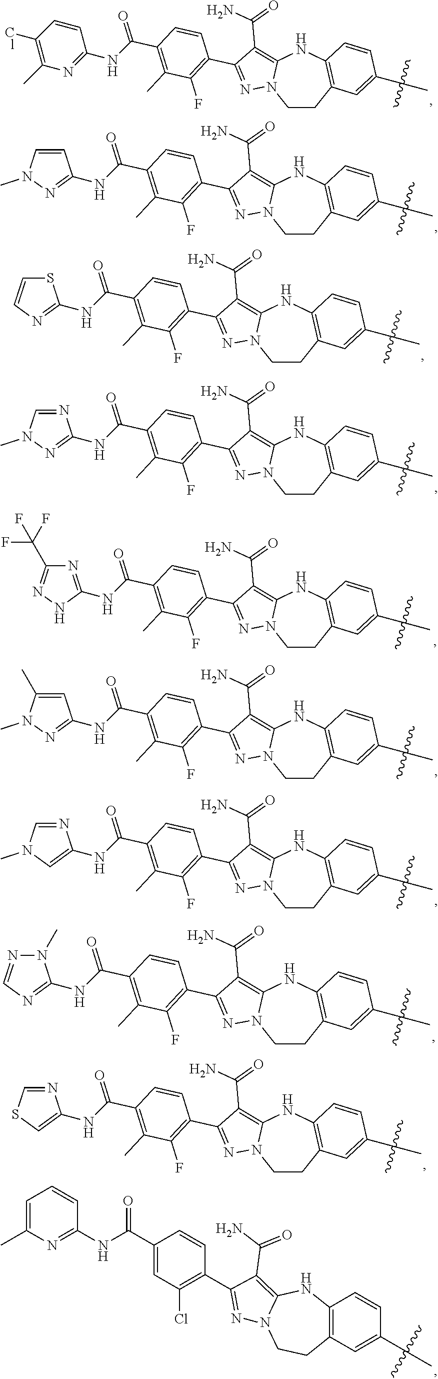

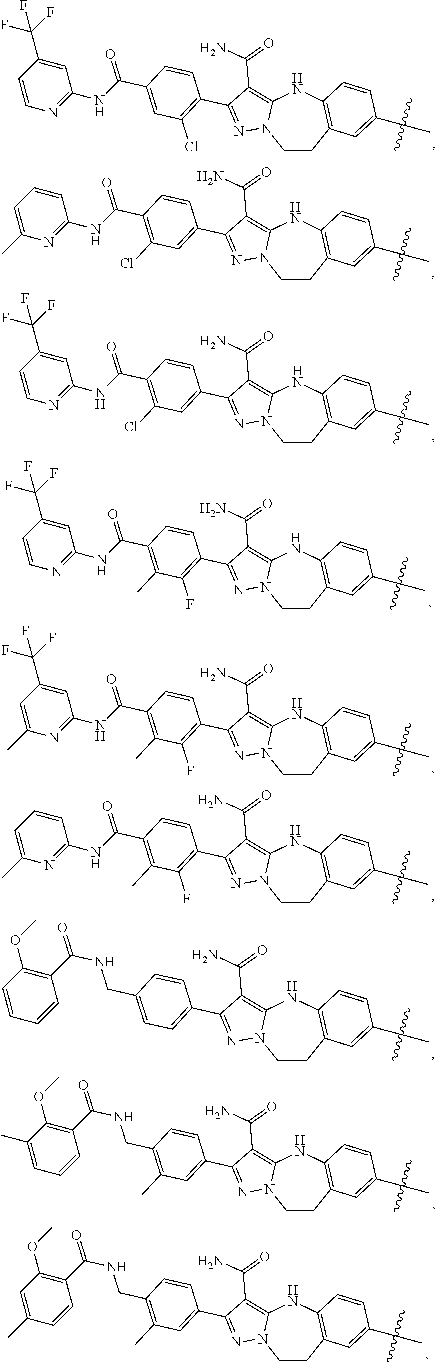

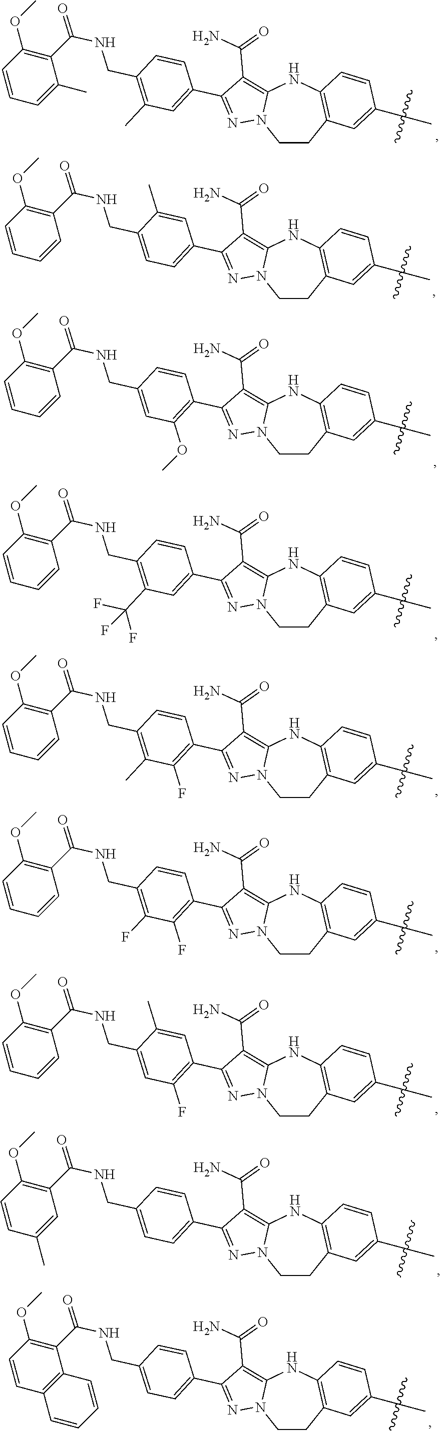

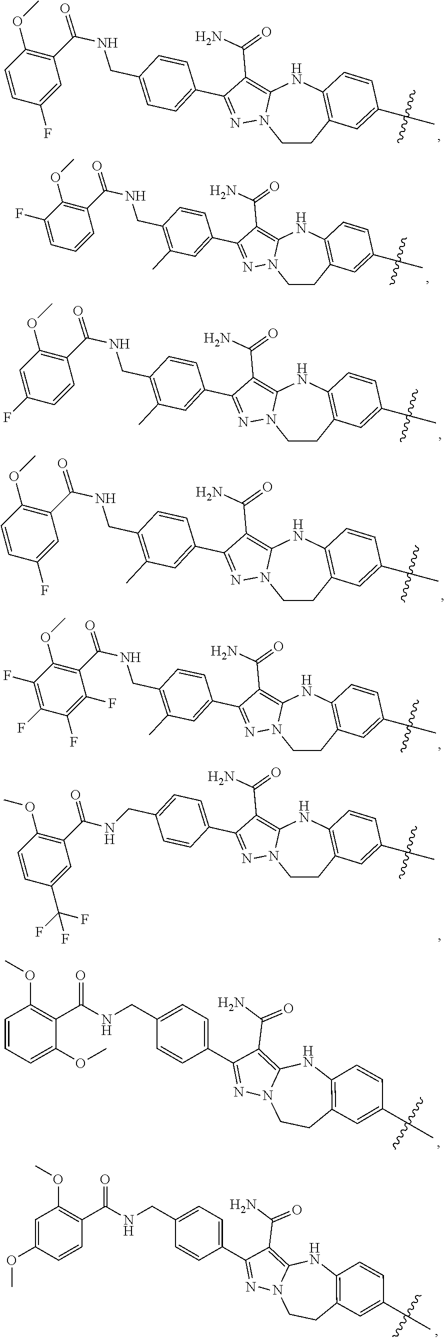

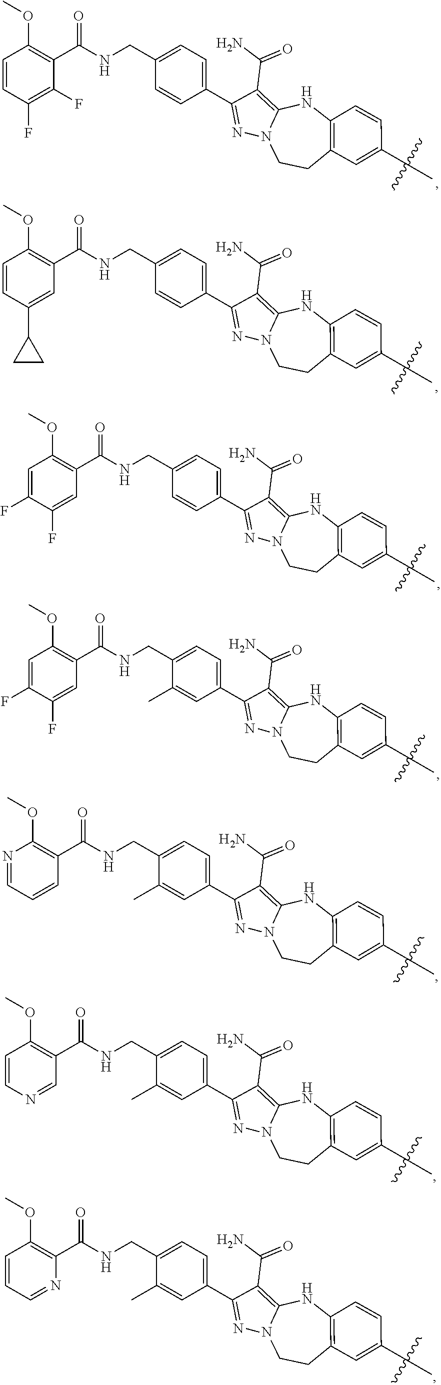

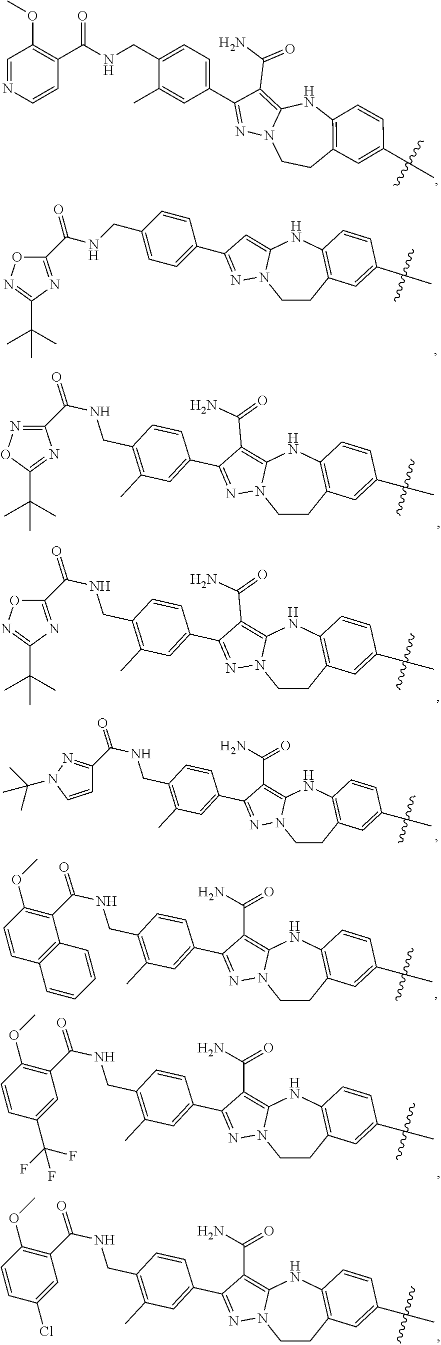

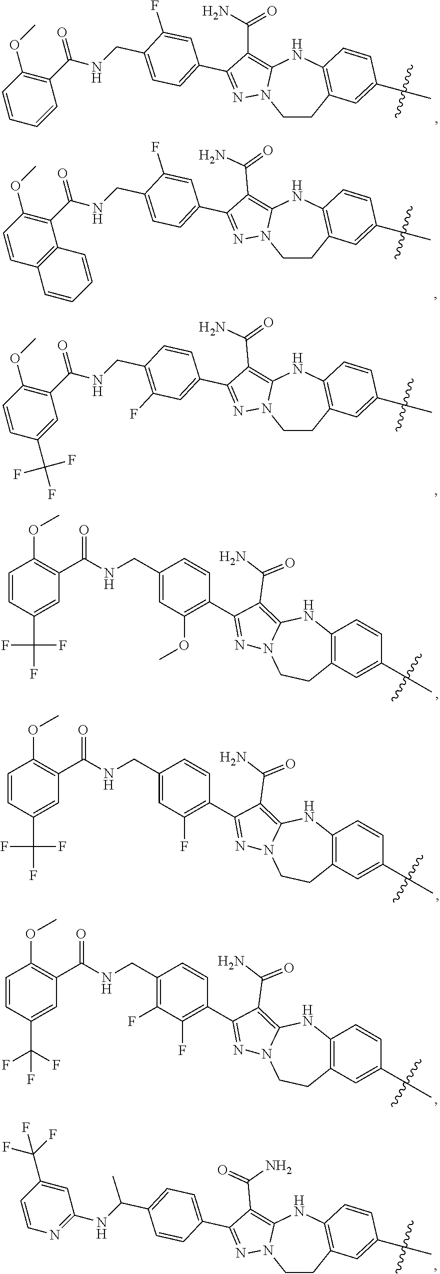

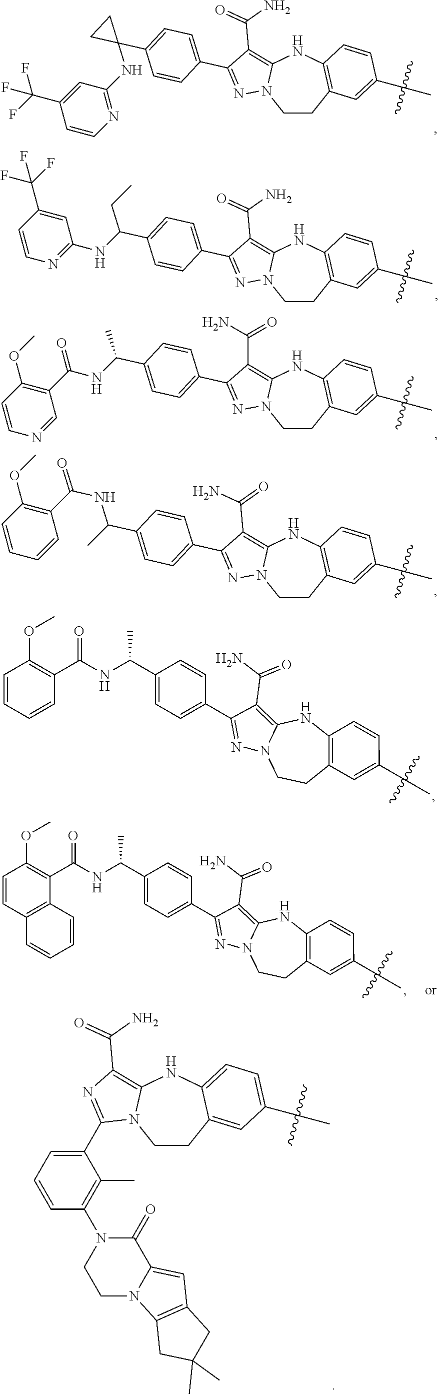

Aspect 66. The compound of any one of aspects 1-19, wherein Warhead is

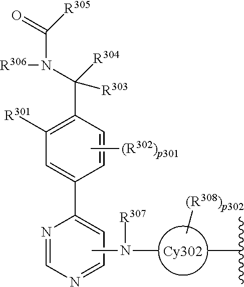

wherein:
Cy302 is a 5- or 6-membered saturated ring or unsaturated ring (preferably aromatic ring) comprising 0-3 heteroatoms selected from nitrogen, oxygen and sulfur as ring member(s);
each of occurrence, R³⁰¹, R³⁰², R³⁰³, R³⁰⁴ and R³⁰⁸ are each independently hydrogen, halogen, —C₁₋₈alkyl, —C₁₋₈alkoxy, —C₂₋₈alkenyl, —C₂₋₈alkynyl, cycloalkyl, heterocyclyl, aryl, heteroaryl, oxo (═O), —CN, —NO₂, —OR^{30c}, —SO₂R^{30c}, —COR^{30c}, —CO₂R^{30c}, —CONR^{30c}R^{30d}, —C(═NR^{30c})NR^{30d}R^{30e}, —NR^{30c}R^{30d}, —NR^{30c}COR^{30d}, —NR^{30c}CONR^{30d}R^{30e}, —NR^{30c}CO₂R^{30d}, —NR^{30c}SONR^{30d}R^{30e}, —NR^{30c}SO₂NR^{30d}R^{30e}, or —NR^{30c}SO₂R^{30d}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl;
R³⁰⁶ and R³⁰⁷ are each independently hydrogen, —C₁₋₈alkyl, —C₂₋₈alkenyl, —C₂₋₈alkynyl, cycloalkyl, heterocyclyl, aryl or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl;
R³⁰⁵ is 5- or 6-membered aromatic ring comprising 0-3 heteroatoms selected from nitrogen, oxygen and sulfur as ring member(s); said aromatic ring is optionally substituted with halogen, —C₁₋₈alkyl, —C₂₋₈alkenyl, —C₂₋₈alkynyl, —C₁₋₈alkoxy, cycloalkyl, heterocyclyl, aryl, heteroaryl, —CN, —NO₂, —OR^{30c}, —SO₂R^{30c}, —COR^{30c}, —CO₂R^{30c}, —CONR^{30c}R^{30d}, —C(═NR^{30c})NR^{30d}R^{30e}, —NR^{30c}R^{30d}, —NR^{30c}COR^{30d}, —NR^{30c}CONR^{30d}R^{30e}, —NR^{30c}CO₂R^{30d}, —NR^{30c}SONR^{30d}R^{30e}, —NR^{30c}SO₂NR^{30d}R^{30e}, or —NR^{30c}SO₂R^{30d}, each of said —C₁₋₈alkyl, —C₂₋₈alkenyl, —C₂₋₈alkynyl, cycloalkyl, heterocyclyl, aryl, or heteroaryl is optionally substituted with —C₁₋₈alkyl, halogen, hydroxy, -haloC₁₋₈alkyl, —C₁₋₈alkyoxy, cycloalkyl, heterocyclyl, aryl, or heteroaryl;
p301 and p302 are each independently 0, 1, 2, 3 or 4;
R^{30c}, R^{30d} and R^{30e} are each independently hydrogen, —C₁₋₈alkyl, —C₂₋₈alkenyl, —C₂₋₈alkynyl, cycloalkyl, heterocyclyl, aryl, or heteroaryl; or
(R^{30c} and R^{30d}) or (R^{30d} and R^{30e}) together with the atom(s) to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂.

Aspect 67. The compound of Aspect 66, wherein
R³⁰¹ is hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN, —NO₂, —OR^{30c}, —SO₂R^{30c}, —COR^{30c}, —CO₂R^{30c}, —CONR^{30c}R^{30d}, —C(═NR^{30c})NR^{30d}R^{30e}, —NR^{30c}R^{30d}, —NR^{30c}COR^{30d}, —NR^{30c}CONR^{30d}R³⁰, —NR^{30c}CO₂R^{30d}, —NR^{30c}SONR^{30d}R^{30e}, —NR^{30c}SO₂NR^{30d}R^{30e}, or —NR^{30c}SO₂R^{30d}, each of said methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with F, Cl, Br, I, hydroxy, -haloC₁₋₈alkyl, - methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, heterocyclyl, aryl, or heteroaryl;
R^{30c}, R^{30d} and R^{30e} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, or heteroaryl; or
(R^{30c} and R^{30d}) or (R^{30d} and R^{30e}) together with the atom(s) to which they are attached, form a 3-, 4-, 5-, 6-, 7- or 8-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂.

Aspect 68. The compound of any aspect of Aspects 66-67, wherein R³⁰¹ is hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN or —NO₂; preferably RI is hydrogen, F, Cl, Br, I, methyl, ethyl, propyl, butyl, cyclopropyl, cyclobutyl.

Aspect 69. The compound of any aspect of Aspects 66-68, wherein
R³⁰² is hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN, —NO₂, —OR^{30c}, —SO₂R^{30c}, —COR^{30c}, —CO₂R^{30c}, —CONR^{30c}R^{30d}, —C(═NR^{30c})NR^{30d}R^{30e}, —NR^{30c}R^{30d}, —NR^{30c}COR^{30d}, —NR^{30c}CONR^{30d}R^{30e}, —NR^{30c}CO₂R^{30d}, —NR^{30c}SONR^{30d}R^{30e}, —NR^{30c}SO₂NR^{30d}R^{300e}, or —NR^{30c}SO₂R^{30d}, each of said methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with F, Cl, Br, I, hydroxy, -haloC₁₋₈alkyl, - methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, heterocyclyl, aryl, or heteroaryl;
R^{30c}, R^{30d} and R^{30e} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, or heteroaryl; or
(R^{30c} and R^{30d}) or (R^{30d} and R^{30e}) together with the atom(s) to which they are attached, form a 3-, 4-, 5-, 6-, 7- or 8-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂.

Aspect 70. The compound of any aspect of Aspects 66-69, wherein R³⁰² is hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN or —NO₂; preferably R³⁰² is hydrogen, F, Cl, Br, I, methyl, ethyl, propyl, butyl, methoxy, ethoxy, propoxy, butoxy, cyclopropyl, cyclobutyl, cyclopentyl, —CN or —NO₂.

Aspect 71. The compound of any aspect of Aspects 66-70, wherein
R³⁰³ and R³⁰⁴ are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN, —NO₂, —OR^{30c}, —SO₂R^{30c}, —COR^{30c}, —CO₂R^{30c}, —CONR^{30c}R^{30d}, —C(═NR³⁰c)NR^{30d}R^{30e}, —NR^{30c}R^{30d}, —NR^{30c}COR^{30d}, —NR^{30c}CONR^{30d}R^{30e}, —NR^{30c}CO₂R^{30d}, —NR^{30c}SONR^{30d}R^{30e}, —NR^{30c}SO₂NR^{30d}R^{30e}, or —NR^{30c}SO₂R^{30d}, each of said methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with F, Cl, Br, I, hydroxy, -haloC₁₋₈alkyl, - methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, heterocyclyl, aryl, or heteroaryl;
R^{30c}, R^{30d} and R^{30e} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, or heteroaryl; or
(R^{30c} and R^{30d}) or (R^{30d} and R^{30e}) together with the atom(s) to which they are attached, form a 3-, 4-, 5-, 6-, 7- or 8-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂.

Aspect 72. The compound of any aspect of Aspects 66-71, wherein R³⁰³ and R³⁰⁴ are each independently hydrogen, F, Cl, Br, I, methyl, ethyl, propyl, butyl, pentyl, hexyl, heptyl, octyl, methoxy, ethoxy, propoxy, cyclopropyl, cyclobutyl, cyclopentyl, cyclohexyl, cycloheptyl, cyclooctyl, heterocyclyl, aryl, heteroaryl or —CN; preferably R³⁰³ and R³⁰⁴ are each independently hydrogen, methyl, ethyl, propyl, butyl, cyclopropyl, cyclobutyl or cyclopentyl.

Aspect 73. The compound of any aspect of Aspects 66-72, wherein the

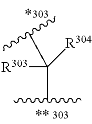

moiety is

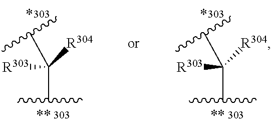

wherein *303 refers to the position attached to the

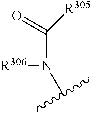

moiety, and **303 refers to the position attached to

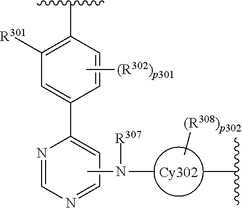

moiety.

Aspect 74. The compound of any aspect of Aspects 66-73, wherein R³⁰⁶ and R³⁰⁷ are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl, each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with F, Cl, Br, I, hydroxy, -haloC₁₋₈alkyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, heterocyclyl, aryl or heteroaryl; preferably, R³⁰⁶ and R³⁰⁷ are each independently H, methyl, ethyl, propyl butyl, pentyl, hexyl, cyclopropyl, cyclobutyl, cyclopentyl or cyclohexyl.

Aspect 75. The compound of any aspect of Aspects 66-74, wherein R³⁰⁵ is

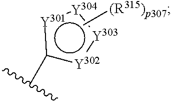

Y³⁰¹, Y³⁰², Y³⁰³ and Y³⁰⁴ are each independently selected from CH, O, S or N; R³¹⁵ is each independently selected from hydrogen, halogen, —C₁₋₈alkyl, —C₁₋₈alkoxy, —C₂₋₈alkenyl, —C₂₋₈alkynyl, cycloalkyl, heterocyclyl, aryl, heteroaryl, —CN, —NO₂, —OR^{30c}, —SO₂R^{30c}, —COR^{30c}, —CO₂R^{30c}, —CONR^{30c}R^{30d}, —C(═NR^{30c})NR^{30d}R^{30e}, —NR^{30c}R^{30d}, —NR^{30c}COR^{30d}, —NR^{30c}CONR^{30d}R^{30e}, —NR^{30c}CO₂R^{30d}, —NR^{30c}SONR^{30d}R^{30e}, —NR^{30c}SO₂NR^{30d}R^{30e}, or —NR^{30c}SO₂R^{30d}, each of said —C₁₋₈alkyl, —C₁₋₈alkoxy, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl; R^{30c}, R^{30d}, and R^{30e} are each independently defined as in aspect 66; and p307 is 0, 1, 2, 3 or 4.

Aspect 76. The compound of Aspect 75, wherein Y³⁰¹ is CH, S, N or O; Y³⁰² is CH, S, O or N; Y³⁰³ is CH, O, S or N; and Y³⁰⁴ is CH, O, S or N.

Aspect 77. The compound of Aspect 75, wherein

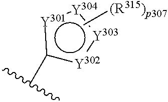

is selected from

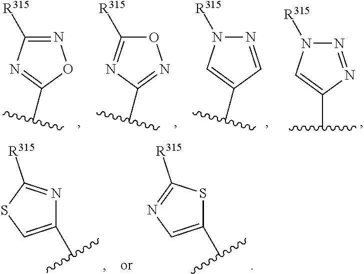

Aspect 78. The compound according to Aspect 75, wherein, R³¹⁵ is selected from —H, —F, —Cl, —Br, —I, —CH₃, —C₂H₅, —C₃H₇, —C₄H₉, —C₅H₁₁, —C₆H₁₃, methoxy, ethoxy, propoxy, butoxy, pentoxy, hexoxy, cyclopropyl, cyclobutyl, cyclopentyl, cyclohexyl, —C₂₋₈alkenyl, —C₂₋₈alkynyl, —CH₂OH, —CH₂CH₂OH, —CH(OH)CH₃, —CH₂CH₂CH₂OH, —CH(OH)CH₂CH₃, —CH₂CH(OH)CH₃, —CH₂OCH₃, —CFH₂, —CF₂H, —CF₃, —CH₂CF₃, —CH₂CH₂CF₃, each of said —CH₃, —C₂H₅, —C₃H₇, —C₄H₉, —C₅H₁₁, —C₆H₁₃, methoxy, ethoxy, propoxy, butoxy, pentoxy, hexoxy, cyclopropyl, cyclobutyl, cyclopentyl, cyclohexyl, —C₂₋₈alkenyl, —C₂₋₈alkynyl is optionally substituted with at least one F, Cl, Br, I, hydroxy, -haloC₁₋₈alkyl, —C₁₋₈alkyoxy, cycloalkyl, heterocyclyl, aryl, or heteroaryl.

Aspect 79. The compound according to Aspect 75, wherein, R³¹⁵ is selected from

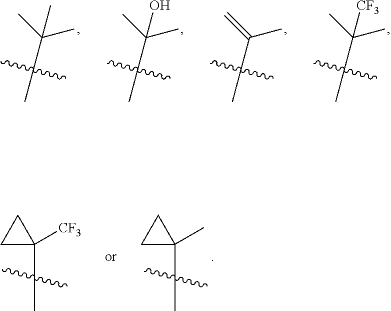

Aspect 80. The compound of any aspect of Aspects 66-79, wherein the

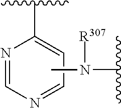

moiety is

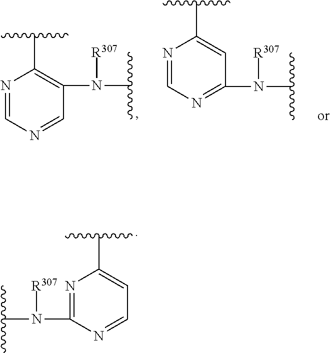

Aspect 81. The compound of any aspect of Aspects 66-80, wherein Cy302 is a 5- or 6-membered aromatic ring comprising 0-3 heteroatoms selected from nitrogen, oxygen and sulfur as ring member(s).

Aspect 82. The compound of any aspect of Aspects 66-81, wherein

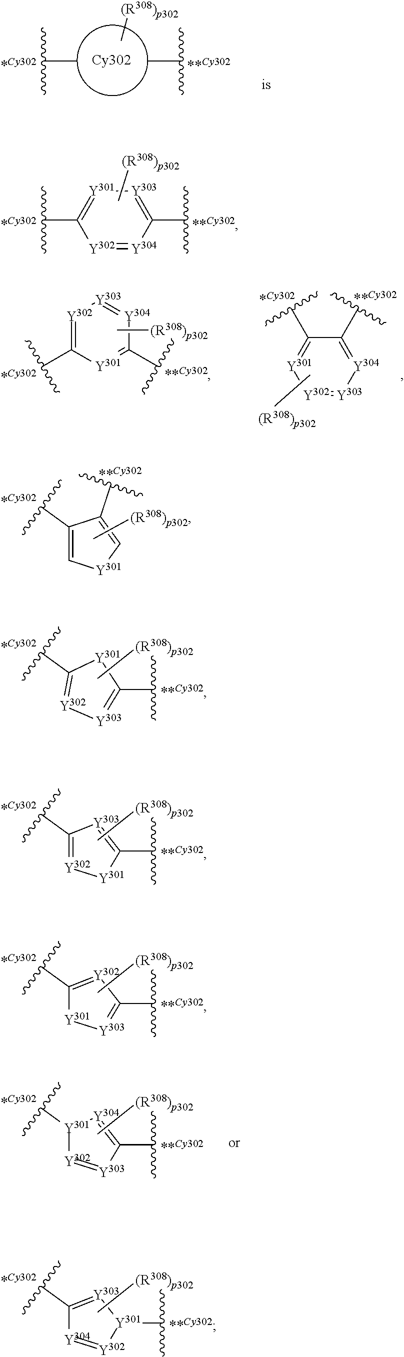

Y³⁰¹, Y³⁰², Y³⁰³ and Y³⁰⁴ are each independently defined as in aspect 66;
wherein *Cy302 refers to the position attached to —N(R³⁰⁷)— in the

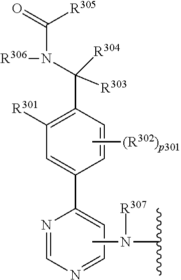

moiety, and **Cy302 refers to the position attached to

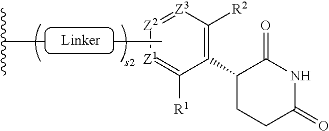

moiety.

Aspect 83. The compound of any aspect of Aspects 66-82, wherein

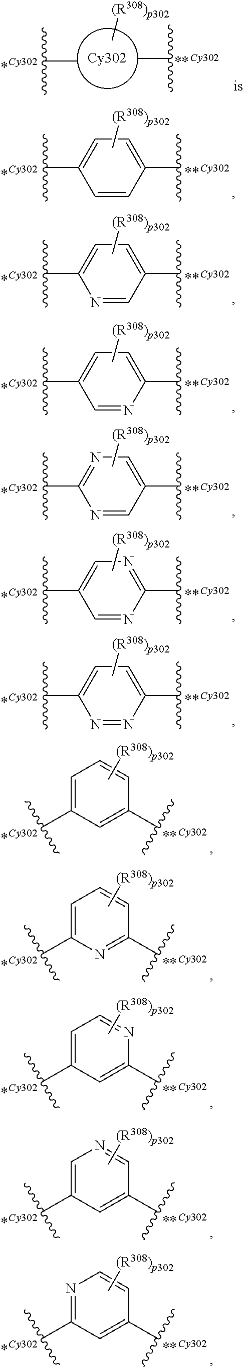

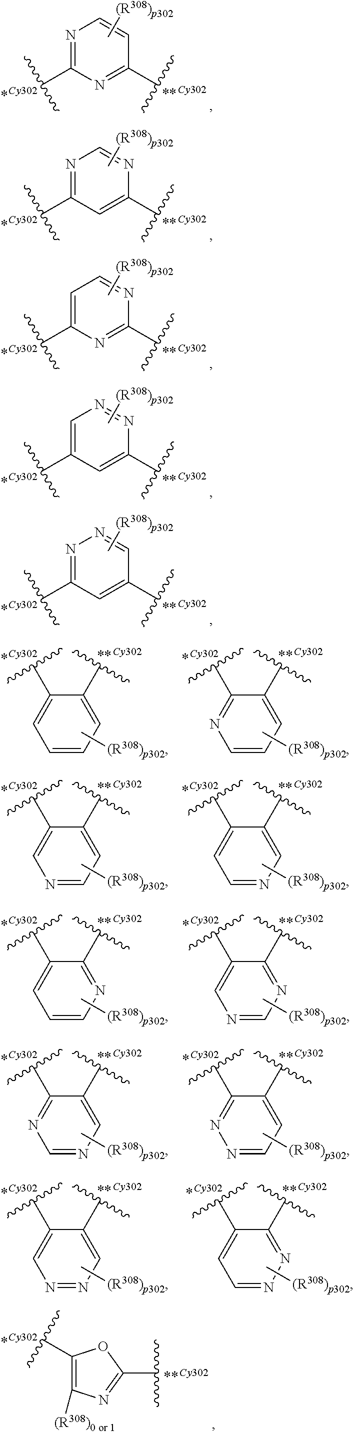

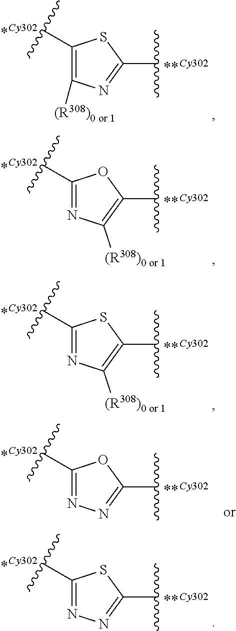

Aspect 84. The compound of any aspect of Aspects 66-83, wherein
R³⁰⁸ is hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN, —NO₂, —OR^{30c}, —SO₂R^{30c}, —COR^{30c}, —CO₂R^{30c}, —CONR^{30c}R^{30d}, —C(═NR^{30c})NR^{30d}R^{30e}, —NR^{30c}R^{30d}, —NR^{30c}COR^{30d}, —NR^{30c}CONR^{30d}R^{30e}, —NR^{30c}CO₂R^{30d}, —NR^{30c}SONR^{30d}R^{30e}, —NR^{30c}SO₂NR^{30d}R^{30e}, or —NR^{30c} SO₂R^{30d}, each of said methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with F, Cl, Br, I, hydroxy, -haloC₁₋₈alkyl, - methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, heterocyclyl, aryl, or heteroaryl;
R^{30c}, R^{30d} and R^{30e} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, or heteroaryl; or
(R^{30c} and R^{30d}) or (R^{30d} and R^{30e}) together with the atom(s) to which they are attached, form a 3-, 4-, 5-, 6-, 7- or 8-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂.

Aspect 85. The compound of any aspect of Aspects 66-84, wherein R³⁰⁸ is hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN or —NO₂; preferably R³⁰⁸ is hydrogen, F, Cl, Br, I, methyl, ethyl, propyl, butyl, methoxy, ethoxy, propoxy, butoxy, cyclopropyl, cyclobutyl, cyclopentyl, —CN or —NO₂.

Aspect 86. The compound of any aspect of Aspects 66-85, wherein

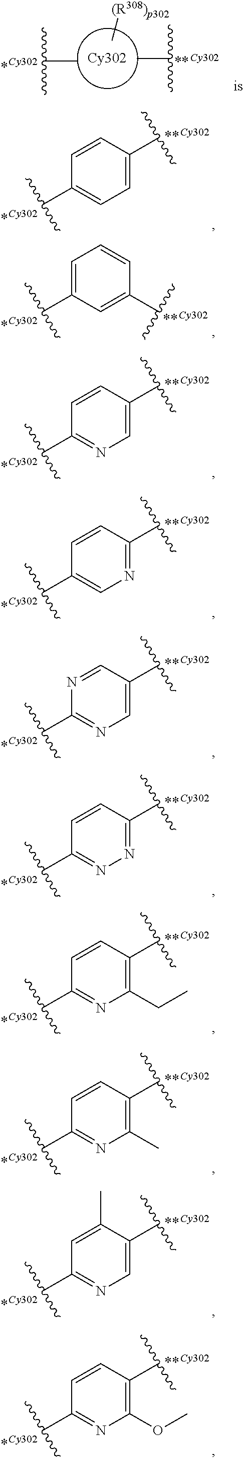

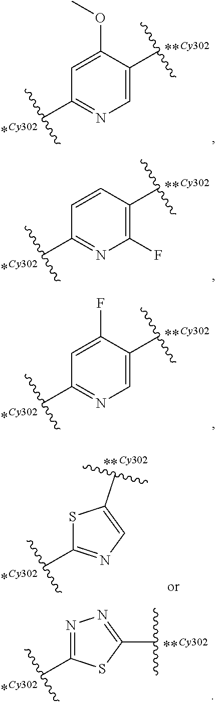

Aspect 87. The compound of any aspect of Aspects 66-85, wherein the

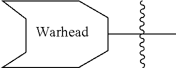

moiety is selected from

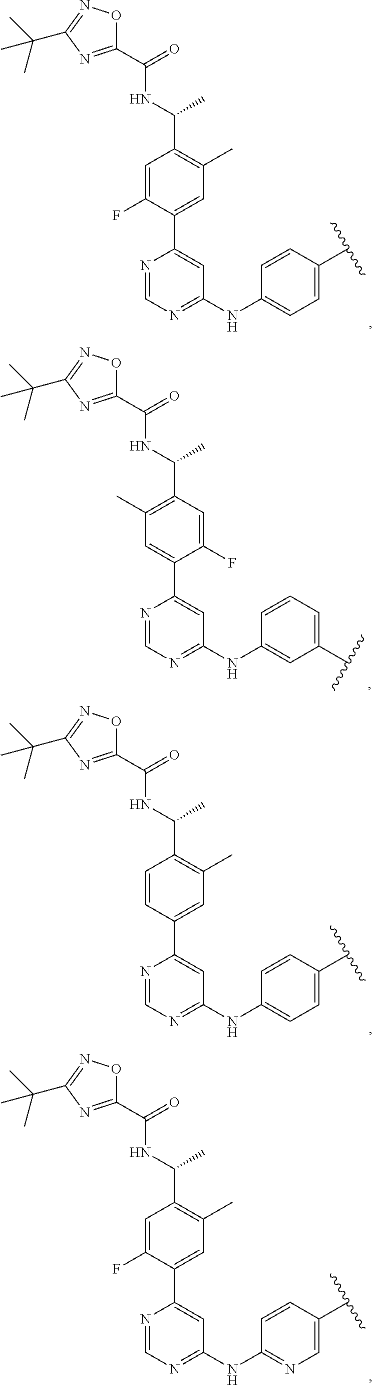 [SMILES: *c1ccc(Nc2cc(-c3cc(C)c([C@@H](C)NC(=O)c4nc(C(C)(C)C)no4)cc3F)ncn2)cc1 ; *c1cccc(Nc2cc(-c3cc(C)c([C@@H](C)NC(=O)c4nc(C(C)(C)C)no4)cc3F)ncn2)c1 ; *c1ccc(Nc2cc(-c3ccc([C@@H](C)NC(=O)c4nc(C(C)(C)C)no4)c(C)c3)ncn2)cc1 ; *c1ccc(Nc2cc(-c3cc(C)c([C@@H](C)NC(=O)c4nc(C(C)(C)C)no4)cc3F)ncn2)nc1]

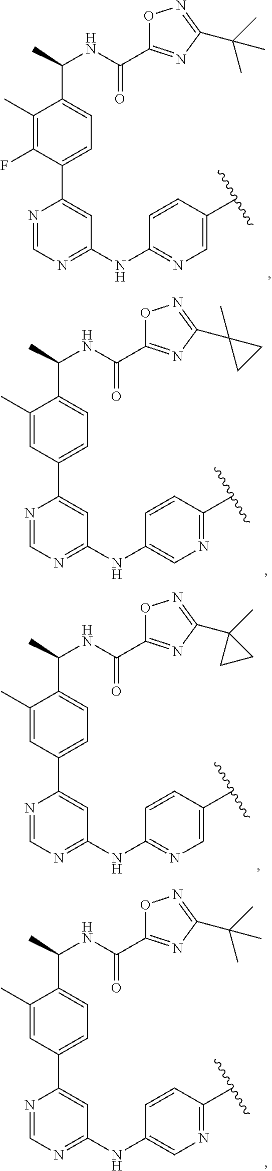

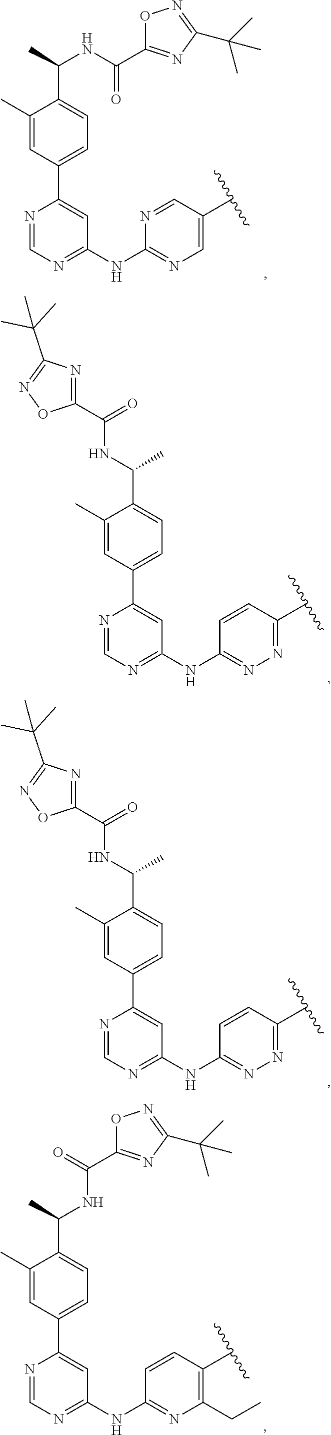

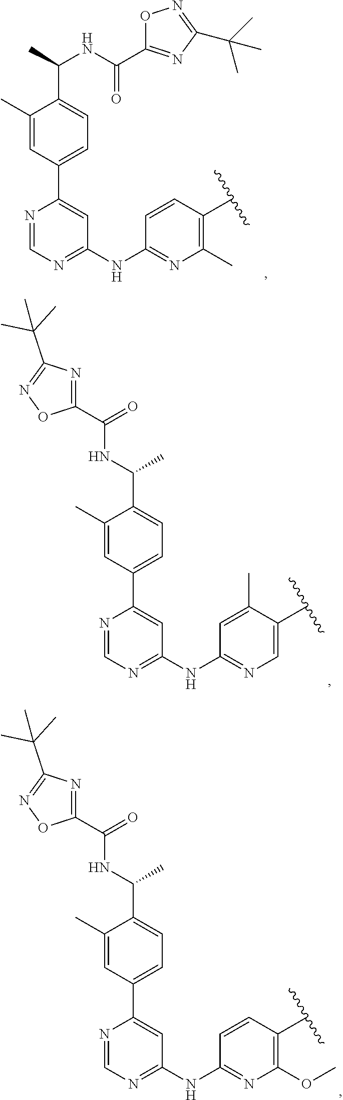

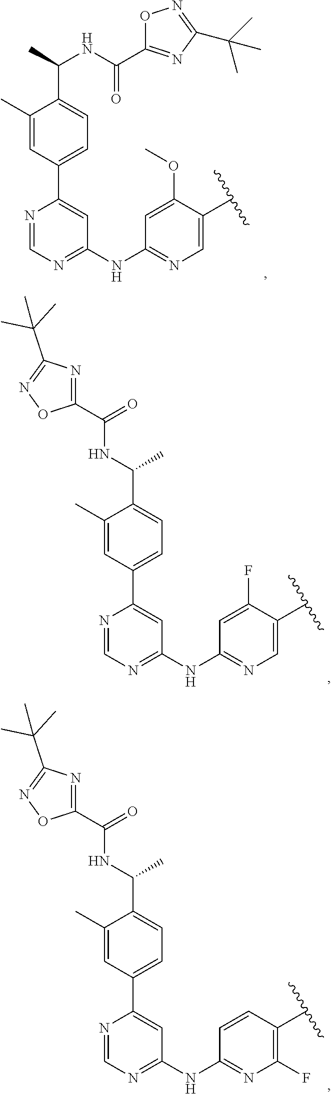

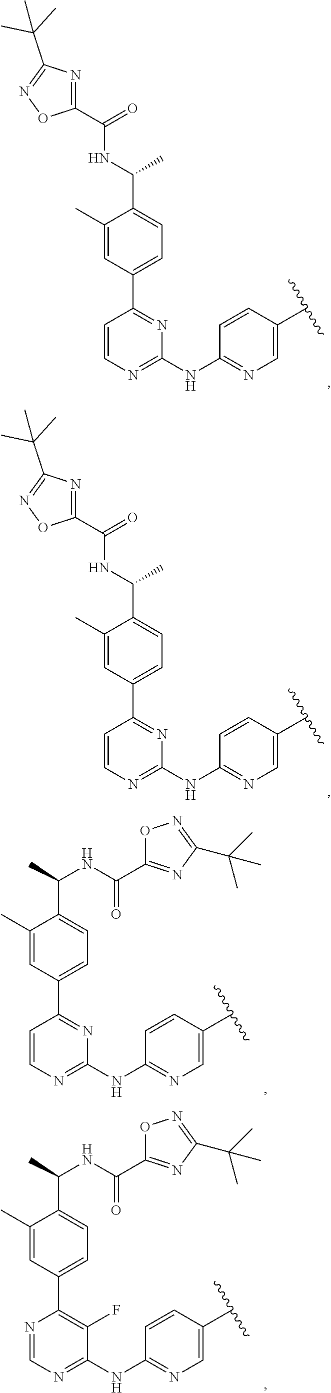

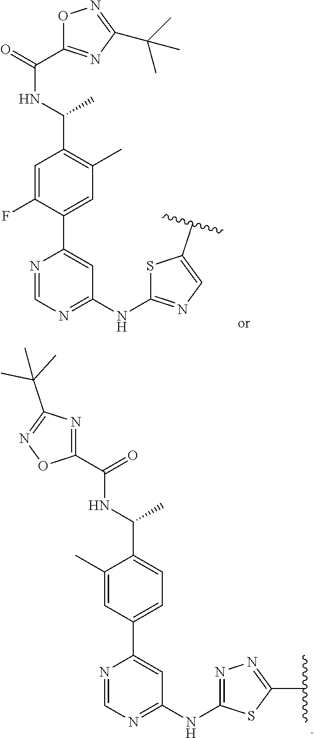

Aspect 88. A pharmaceutical composition comprising a compound of any one of Aspects 1-87 or a pharmaceutically acceptable salt, tautomer or prodrug thereof, together with a pharmaceutically acceptable excipient.

Aspect 89. A method of treating a disease that can be treated by degrading the target protein that the Warhead can combine by using a compound of any one of Aspects 1-87.

Aspect 90. The method of aspect 89, wherein said target protein is selected from the group consisting of structural proteins, receptors, enzymes, cell surface proteins, proteins pertinent to the integrated function of a cell, including proteins involved in catalytic activity, aromatase activity, motor activity, helicase activity, metabolic processes (anabolism and catabolism), antioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity and translation regulator activity.

Aspect 91. The method of aspect 89, wherein Warhead is a moiety which binds to a target protein, wherein said target protein is selected from the group consisting of ErbB receptors, B7.1 and B7, TINFR1m, TNFR2, NADPH oxidase, Bcl-Bax and other partners in the apotosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases (including Bruton's Tyrosine Kinase), CD23, CD124, tyrosine kinase p561ck, CD4, CD5, IL-2 receptor, IL-1 receptor, TNF-alphaR, ICAM1, Cat+ channels, VCAM, VLA-4 integrin, selectins, CD40/CD40L, newokinins and receptors, inosine monophosphate dehydrogenase, p38 MAP Kinase, RAS-RAF-MEK-ERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, adenosine receptors, adenosine kinase and AMP deaminase, purinergic receptors (P2Y1, P2Y2, P2Y4, P2Y6, P2X 1-7), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase, ecdysone 20-monooxygenase, ion channel of the GABA gated chloride channel, acetylcholinesterase, voltage-sensitive sodium channel protein, calcium release channel, chloride channels, Acetyl-CoA carboxylase, adenylosuccinate synthetase, protoporphyrinogen oxidase, L-1 receptor associated kinase-3 (IRAK-3 or IRAK-M) or enolpyruvyl-shikimate-phosphate synthase.

Aspect 92. The method of Aspect 89, wherein the disease is a cancer.

Aspect 93. A compound of Formula (I):

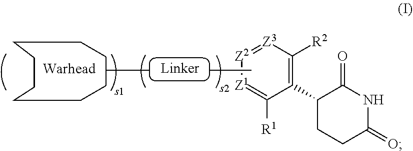

or a pharmaceutically acceptable salt thereof, or a deuterated analog thereof, or a prodrug thereof,
wherein:
Warhead, Linker, s2, Z¹, Z², Z³, R¹ and R² are defined as in any preceding aspects;
s1 is 0.

Aspect 94. A method of binding and altering the specificity of cereblon complex to induce the degradation of a complex-associated protein by using the compound of claim 93, wherein protein is selected from ErbB receptors, B7.1 and B7, TINFR1m, TNFR2, NADPH oxidase, Bcl-Bax and other partners in the apotosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase seine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases (including Bruton's Tyrosine Kinase), CD23, CD124, tyrosine kinase p561ck, CD4, CD5, IL-2 receptor, IL-1 receptor, TNF-alphaR, ICAM1, Cat+ channels, VCAM, VLA-4 integrin, selectins, CD40/CD40L, newokinins and receptors, inosine monophosphate dehydrogenase, p38 MAP Kinase, RAS-RAF-MEK-ERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, adenosine receptors, adenosine kinase and AMP deaminase, purinergic receptors (P2Y1, P2Y2, P2Y4, P2Y6, P2X 1-7), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase, ecdysone 20-monooxygenase, ion channel of the GABA gated chloride channel, acetylcholinesterase, voltage-sensitive sodium channel protein, calcium release channel, chloride channels, Acetyl-CoA carboxylase, adenylosuccinate synthetase, protoporphyrinogen oxidase, L-1 receptor associated kinase-3 (IRAK-3 or IRAK-M), enolpyruvyl-shikimate-phosphate synthase or neo-substrates (like IKZF1, IKZF3, and CK1a).

Aspect 95. A method of treating an CRBN-mediated disorder, disease, or condition in a patient comprising administering to said patient the pharmaceutical composition of claim 93, preferably, the disorder disease, or condition is selected from proliferative disorders, neurological disorders and disorder associated with transplantation.

In one of another aspects, the compound is selected from

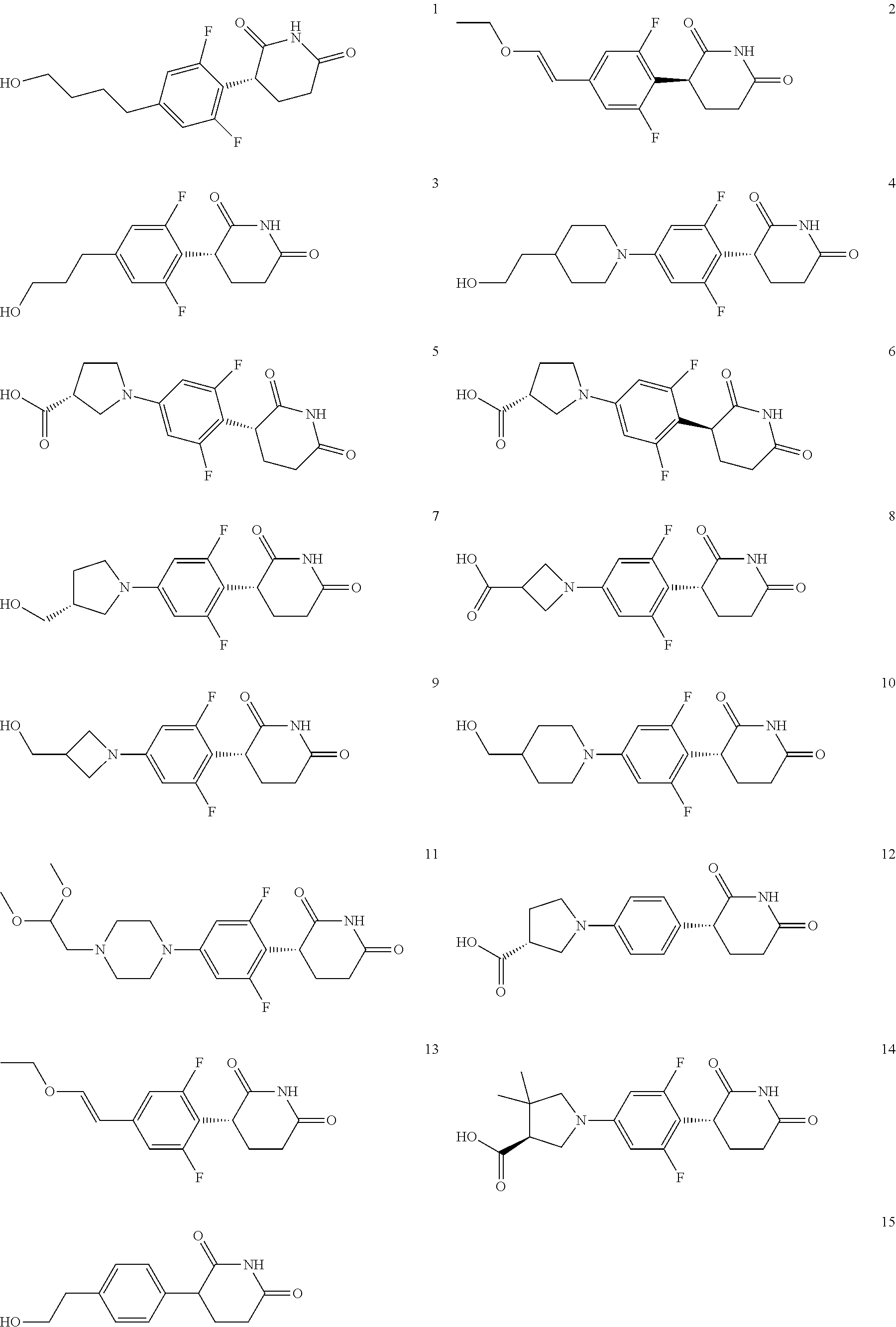

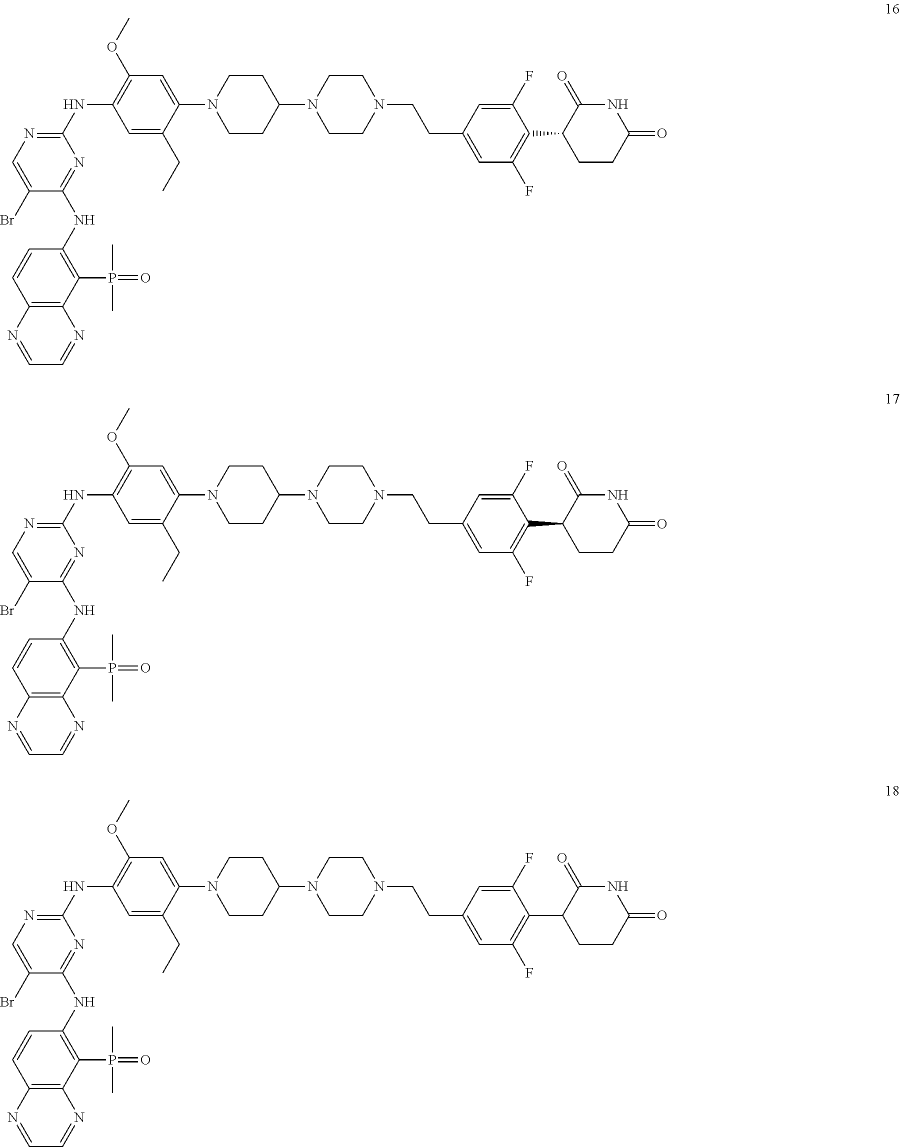 [SMILES: CCc1cc(Nc2ncc(Br)c(Nc3ccc4nccnc4c3P(C)(C)=O)n2)c(OC)cc1N1CCC(N2CCN(CCc3cc(F)c([C@H]4CCC(=O)NC4=O)c(F)c3)CC2)CC1 ; CCc1cc(Nc2ncc(Br)c(Nc3ccc4nccnc4c3P(C)(C)=O)n2)c(OC)cc1N1CCC(N2CCN(CCc3cc(F)c([C@@H]4CCC(=O)NC4=O)c(F)c3)CC2)CC1 ; CCc1cc(Nc2ncc(Br)c(Nc3ccc4nccnc4c3P(C)(C)=O)n2)c(OC)cc1N1CCC(N2CCN(CCc3cc(F)c(C4CCC(=O)NC4=O)c(F)c3)CC2)CC1]

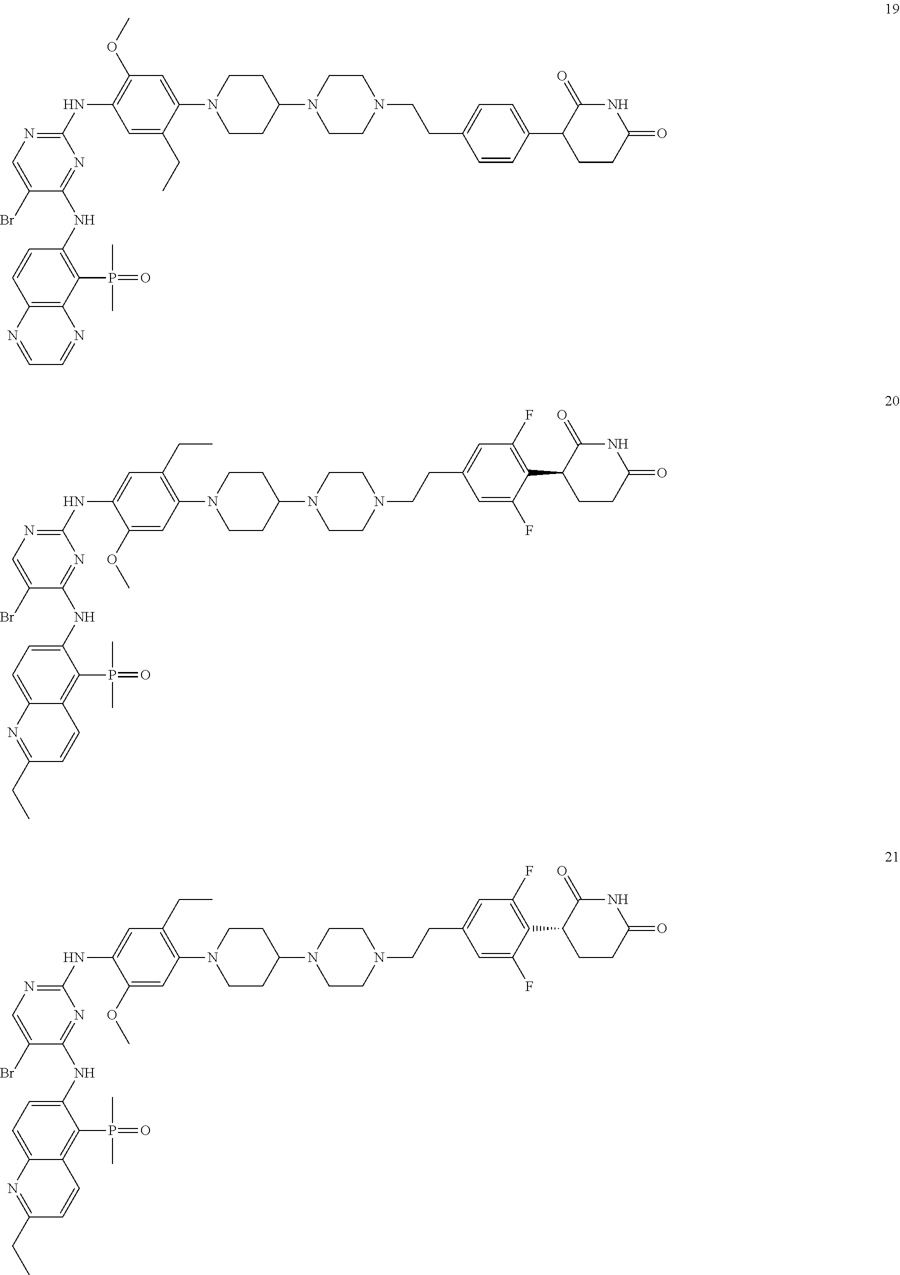 [SMILES: CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)cc3)CC2)CC1 ; CCc1ccc2c(P(C)(C)=O)c(Nc3nc(Nc4cc(CC)c(N5CCC(N6CCN(CCc7cc(F)c([C@@H]8CCC(=O)NC8=O)c(F)c7)CC6)CC5)cc4OC)ncc3Br)ccc2n1 ; CCc1ccc2c(P(C)(C)=O)c(Nc3nc(Nc4cc(CC)c(N5CCC(N6CCN(CCc7cc(F)c([C@H]8CCC(=O)NC8=O)c(F)c7)CC6)CC5)cc4OC)ncc3Br)ccc2n1]

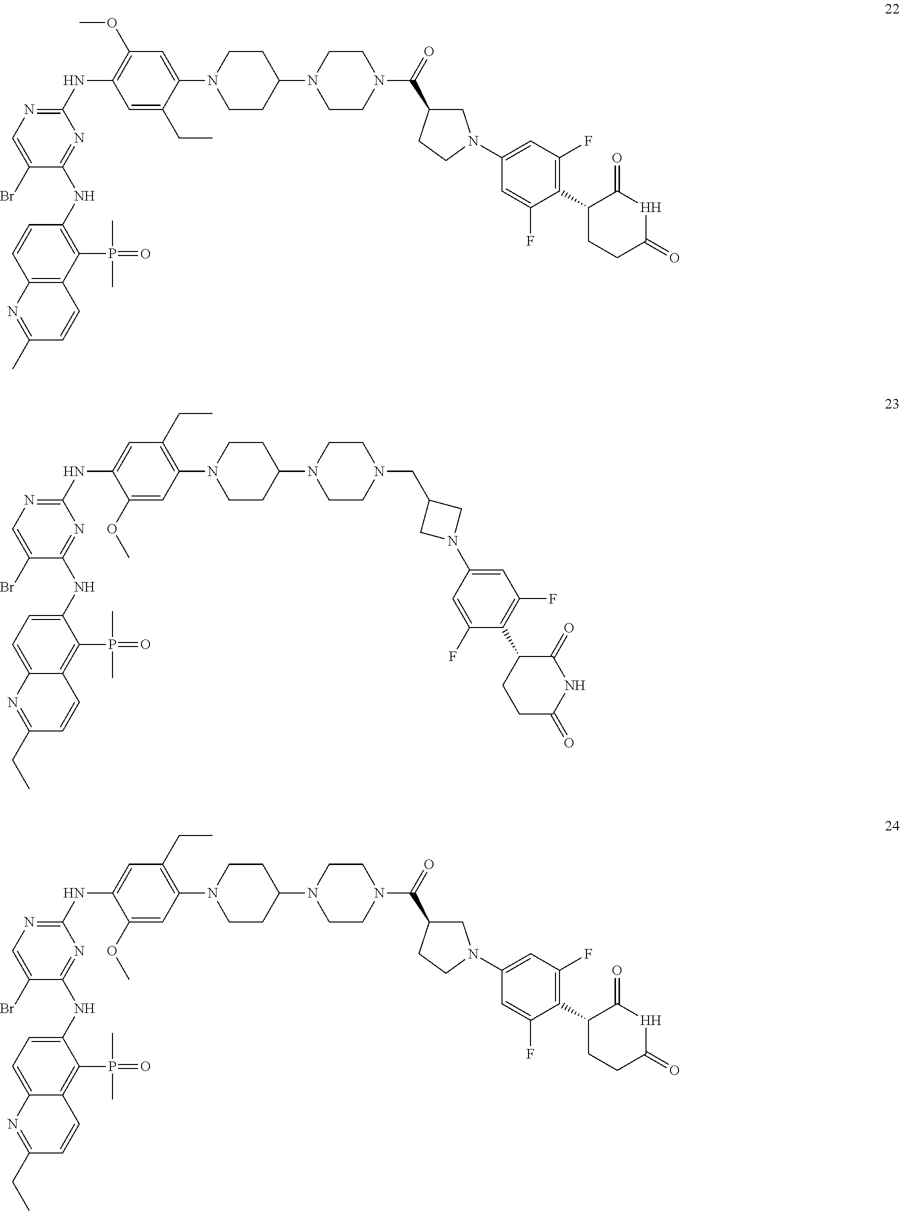

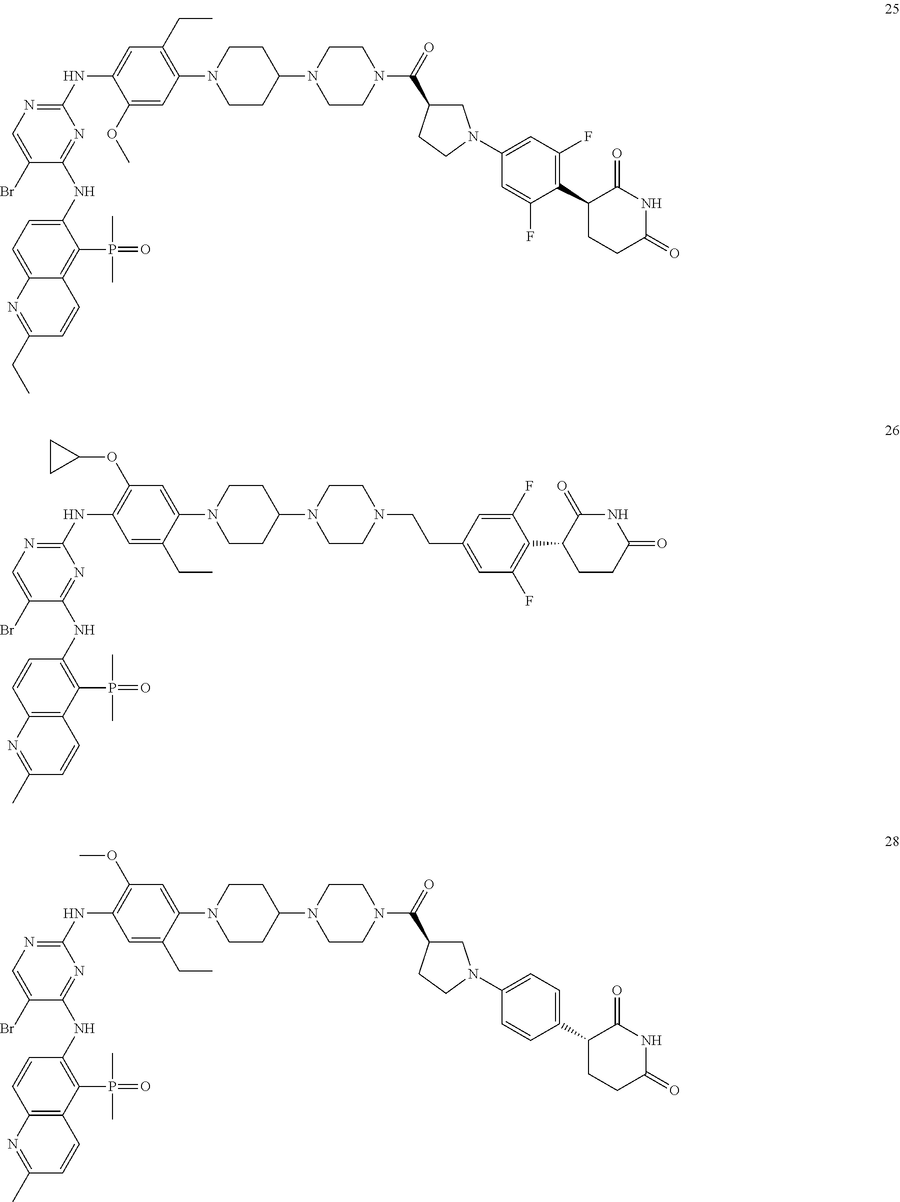 [SMILES: CCc1ccc2c(P(C)(C)=O)c(Nc3nc(Nc4cc(CC)c(N5CCC(N6CCN(C(=O)[C@@H]7CCN(c8cc(F)c([C@@H]9CCC(=O)NC9=O)c(F)c8)C7)CC6)CC5)cc4OC)ncc3Br)ccc2n1 ; CCc1cc(Nc2ncc(Br)c(Nc3ccc4nc(C)ccc4c3P(C)(C)=O)n2)c(OC2CC2)cc1N1CCC(N2CCN(CCc3cc(F)c([C@H]4CCC(=O)NC4=O)c(F)c3)CC2)CC1 ; CCc1cc(Nc2ncc(Br)c(Nc3ccc4nc(C)ccc4c3P(C)(C)=O)n2)c(OC)cc1N1CCC(N2CCN(C(=O)[C@@H]3CCN(c4ccc([C@H]5CCC(=O)NC5=O)cc4)C3)CC2)CC1]

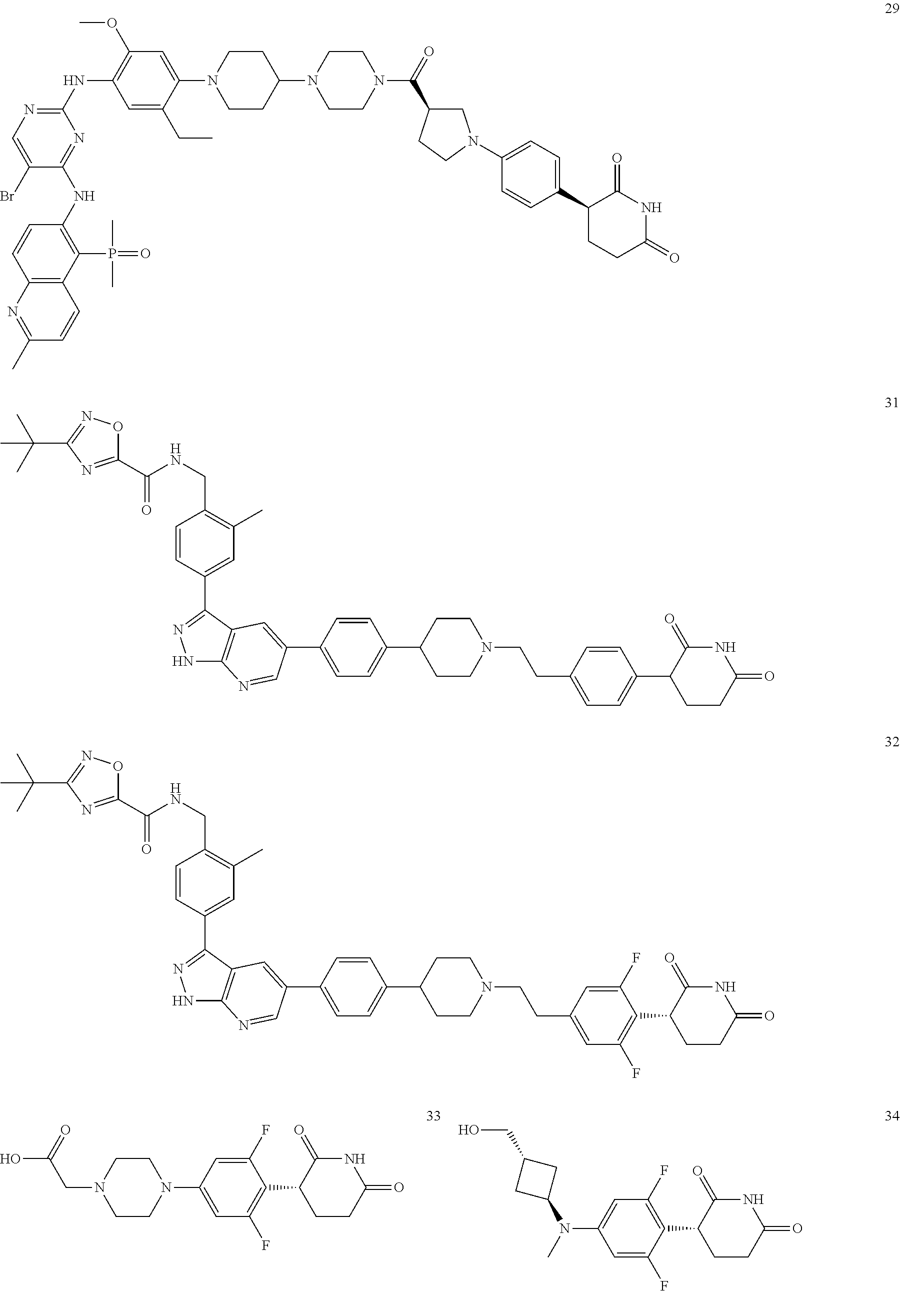

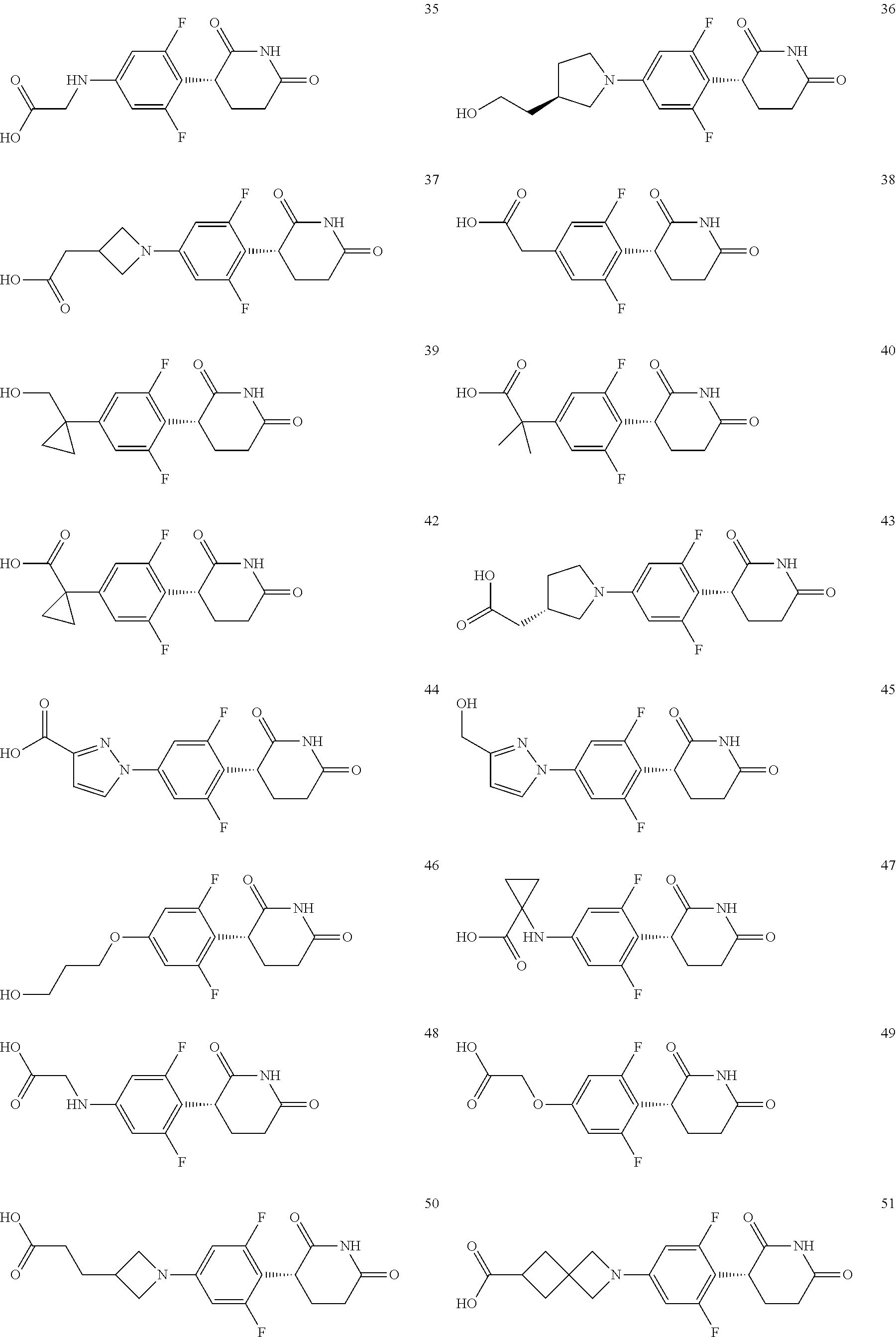

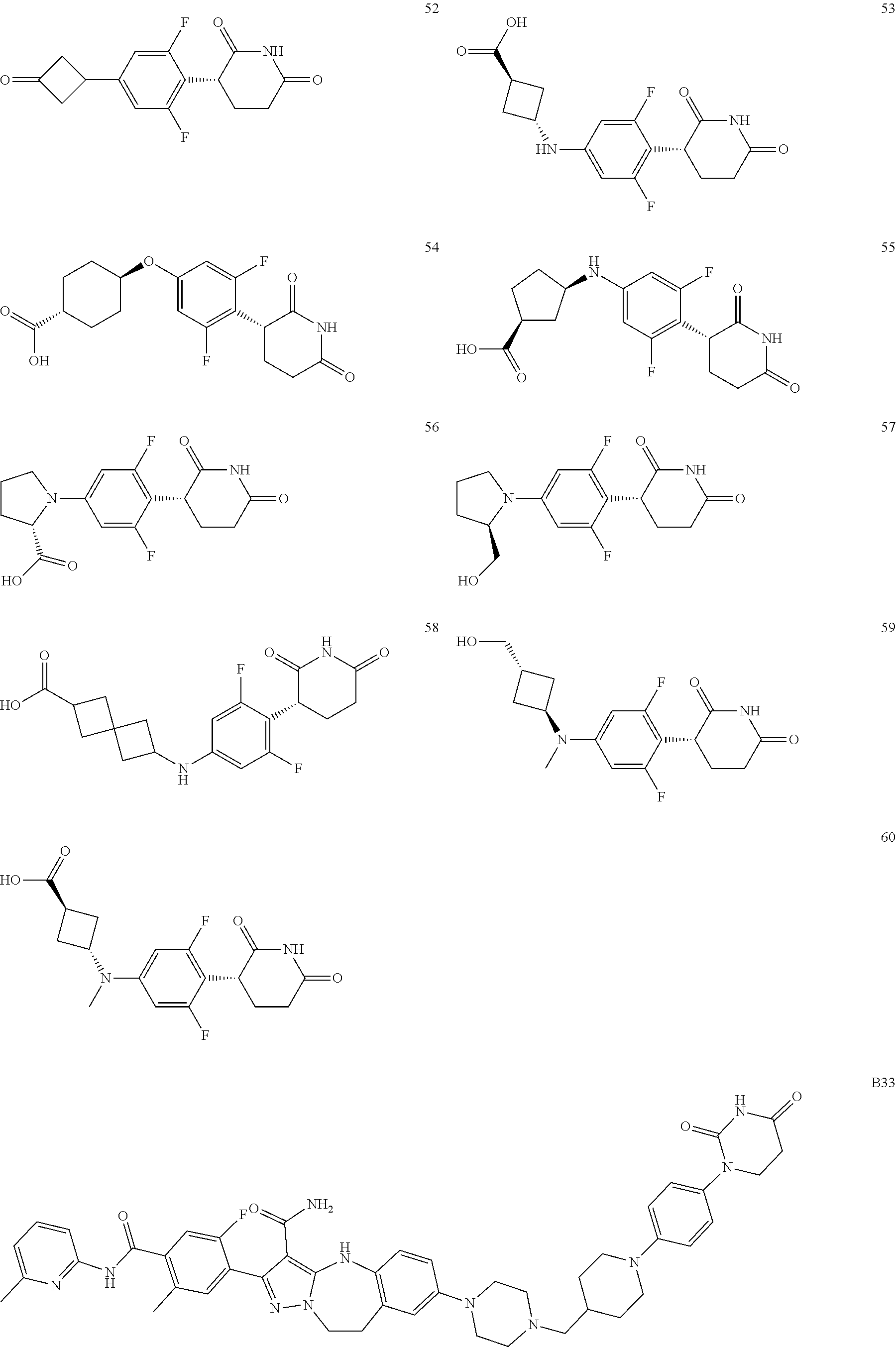

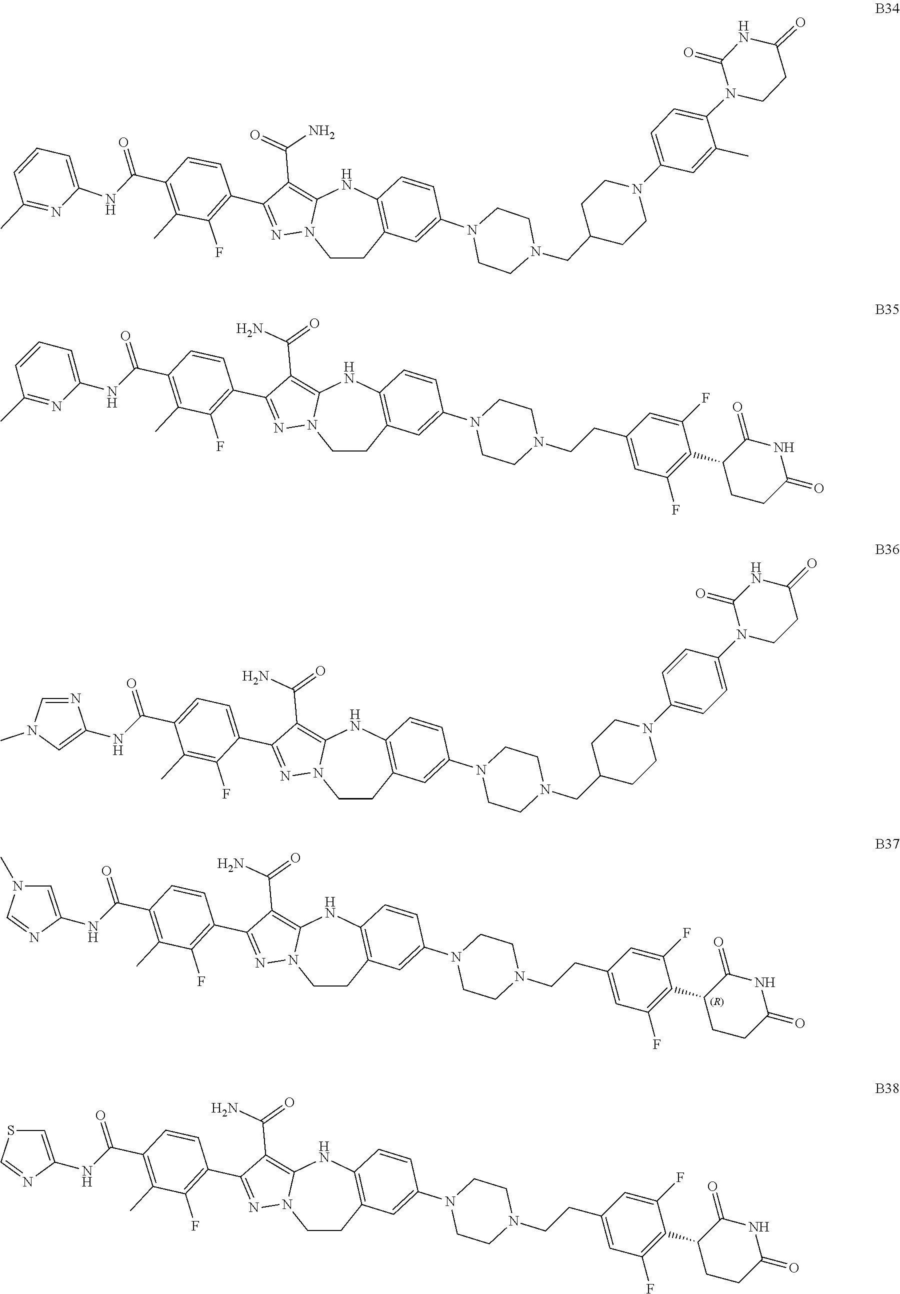

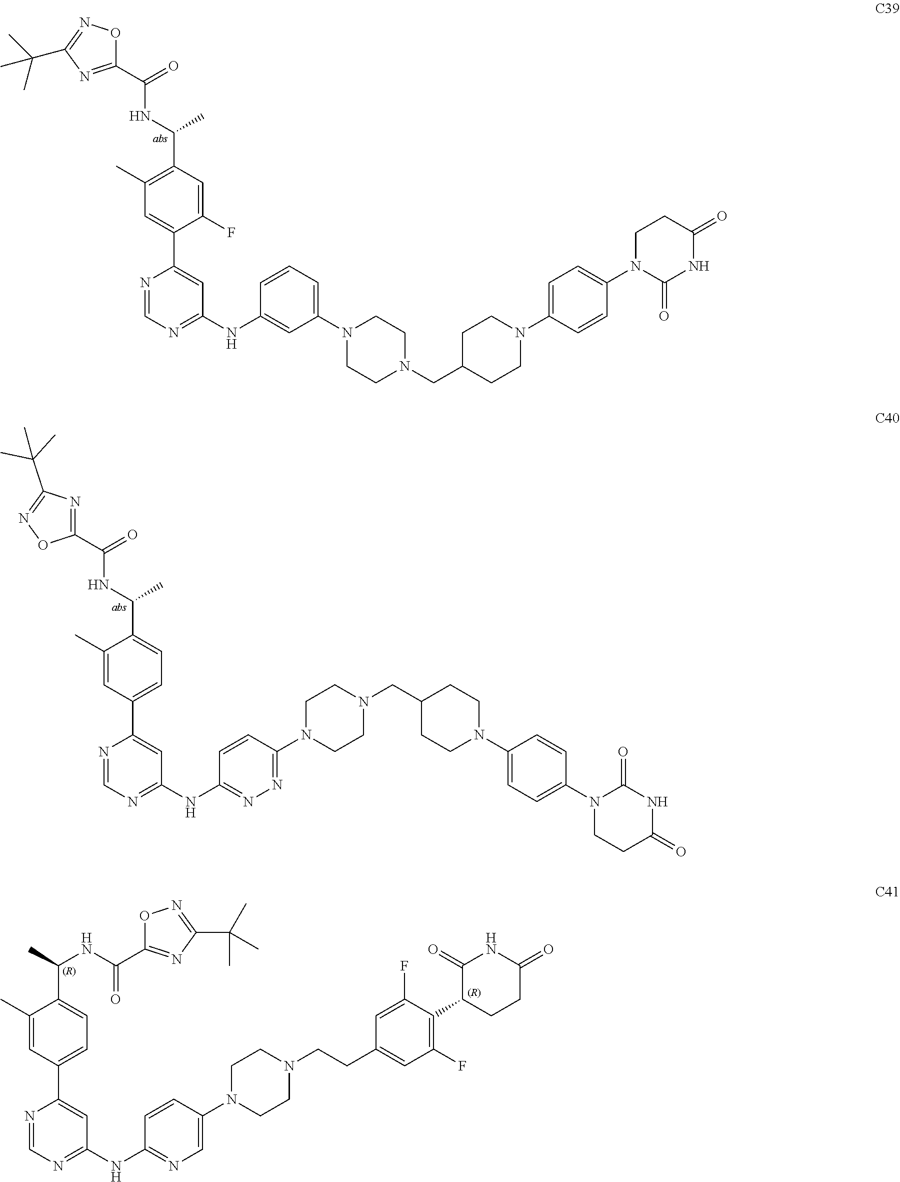

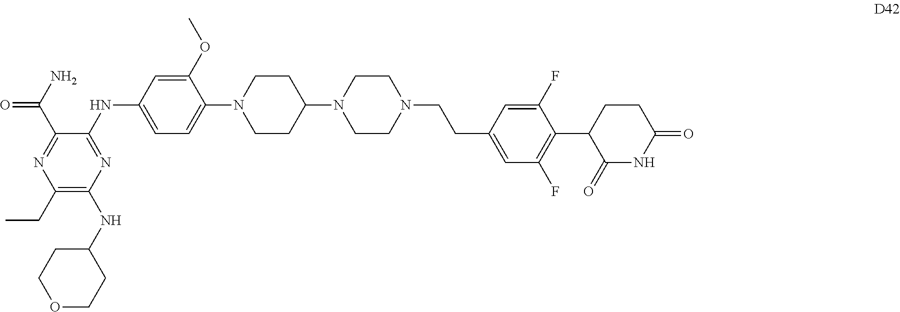 [SMILES: CCc1nc(C(N)=O)c(Nc2ccc(N3CCC(N4CCN(CCc5cc(F)c(C6CCC(=O)NC6=O)c(F)c5)CC4)CC3)c(OC)c2)nc1NC1CCOCC1]

### DETAILED DESCRIPTION OF THE INVENTION

The following terms have the indicated meanings throughout the specification:

Unless specifically defined elsewhere in this document, all other technical and scientific terms used herein have the meaning commonly understood by one of ordinary skill in the art to which this invention belongs.

The following terms have the indicated meanings throughout the specification:

As used herein, including the appended Aspects, the singular forms of words such as “a”, “an”, and “the”, include their corresponding plural references unless the context clearly indicates otherwise.

The term “or” is used to mean, and is used interchangeably with, the term “and/or” unless the context clearly dictates otherwise.

The term “subject” used herein refers to mammal and human, preferably human.

The term “alkyl” includes a hydrocarbon group selected from linear and branched, saturated hydrocarbon groups comprising from 1 to 18, such as from 1 to 12, further such as from 1 to 10, more further such as from 1 to 8, or from 1 to 6, or from 1 to 4, carbon atoms. Examples of alkyl groups comprising from 1 to 6 carbon atoms (i.e., C₁₋₆ alkyl) include, but not limited to, methyl, ethyl, 1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”), 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

The term “propyl” includes 1-propyl or n-propyl (“n-Pr”), 2-propyl or isopropyl (“i-Pr”).

The term “butyl” includes 1-butyl or n-butyl (“n-Bu”), 2-methyl-1-propyl or isobutyl (“i-Bu”), 1-methylpropyl or s-butyl (“s-Bu”), 1,1-dimethylethyl or t-butyl (“t-Bu”).

The term “pentyl” includes 1-pentyl, 2-pentyl, 3-pentyl, 2-methyl-2-butyl, 3-methyl-2-butyl, 3-methyl-1-butyl, 2-methyl-1-butyl.

The term “hexyl” includes 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl.

The term “alkylene” refers to a divalent alkyl group by removing two hydrogen from alkane. Alkylene includes but not limited to methylene, ethylene, propylene, and so on.

The term “halogen” includes fluoro (F), chloro (Cl), bromo (Br) and iodo (I).

The term “alkenyl” includes a hydrocarbon group selected from linear and branched hydrocarbon groups comprising at least one C═C double bond and from 2 to 18, such as from 2 to 8, further such as from 2 to 6, carbon atoms. Examples of the alkenyl group, e.g., C₂₋₆ alkenyl, include, but not limited to ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-dienyl, hex-i-enyl, hex-2-enyl, hex-3-enyl, hex-4-enyl, and hexa-1,3-dienyl groups.

The term “alkenylene” refers to a divalent alkenyl group by removing two hydrogen from alkene. Alkenylene includes but not limited to, vinylidene, butenylene, and so on.

The term “alkynyl” includes a hydrocarbon group selected from linear and branched hydrocarbon group, comprising at least one C≡C triple bond and from 2 to 18, such as 2 to 8, further such as from 2 to 6, carbon atoms. Examples of the alkynyl group, e.g., C₂₋₆ alkynyl, include, but not limited to ethynyl, 1-propynyl, 2-propynyl (propargyl), 1-butynyl, 2-butynyl, and 3-butynyl groups.

The term “alkynylene” refers to a divalent alkynyl group by removing two hydrogen from alkyne. Alkenylene includes but not limited to ethynylene and so on.

The term “cycloalkyl” includes a hydrocarbon group selected from saturated cyclic hydrocarbon groups, comprising monocyclic and polycyclic (e.g., bicyclic and tricyclic) groups including fused, bridged or spiro cycloalkyl.

For example, the cycloalkyl group may comprise from 3 to 12, such as from 3 to 10, further such as 3 to 8, further such as 3 to 6, 3 to 5, or 3 to 4 carbon atoms. Even further for example, the cycloalkyl group may be selected from monocyclic group comprising from 3 to 12, such as from 3 to 10, further such as 3 to 8, 3 to 6 carbon atoms. Examples of the monocyclic cycloalkyl group include cyclopropyl, cyclobutyl, cyclopentyl, cyclohexyl, cycloheptyl, cyclooctyl, cyclononyl, cyclodecyl, cycloundecyl, and cyclododecyl groups. In particular, examples of the saturated monocyclic cycloalkyl group, e.g., C₃₋₈cycloalkyl, include, but not limited to cyclopropyl, cyclobutyl, cyclopentyl, cyclohexyl, cycloheptyl, and cyclooctyl groups. In a preferred embodiment, the cycloalkyl is a monocyclic ring comprising 3 to 6 carbon atoms (abbreviated as C₃₋₆ cycloalkyl), including but not limited to, cyclopropyl, cyclobutyl, cyclopentyl, and cyclohexyl. Examples of the bicyclic cycloalkyl groups include those having from 7 to 12 ring atoms arranged as a fused bicyclic ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. Further Examples of the bicyclic cycloalkyl groups include those arranged as a bicyclic ring selected from [5,6] and [6,6] ring systems.

The term “spiro cycloalkyl” includes a cyclic structure which contains carbon atoms and is formed by at least two rings sharing one atom.

The term “fused cycloalkyl” includes a bicyclic cycloalkyl group as defined herein which is saturated and is formed by two or more rings sharing two adjacent atoms.

The term “bridged cycloalkyl” includes a cyclic structure which contains carbon atoms and is formed by two rings sharing two atoms which are not adjacent to each other. The term “7 to 10 membered bridged cycloalkyl” includes a cyclic structure which contains 7 to 12 carbon atoms and is formed by two rings sharing two atoms which are not adjacent to each other.

Examples of fused cycloalkyl, fused cycloalkenyl, or fused cycloalkynyl include but are not limited to bicyclo[1.1.0]butyl, bicyclo[2.1.0]pentyl, bicyclo[3.1.0]hexyl, bicyclo[4.1.0]heptyl, bicyclo[3.3.0]octyl, bicyclo[4.2.0]octyl, decalin, as well as benzo 3 to 8 membered cycloalkyl, benzo C₄ 6 cycloalkenyl, 2,3-dihydro-1H-indenyl, 1H-indenyl, 1, 2, 3,4-tetralyl, 1,4-dihydronaphthyl, etc. Preferred embodiments are 8 to 9 membered fused rings, which refer to cyclic structures containing 8 to 9 ring atoms within the above examples.

The term “aryl” used alone or in combination with other terms includes a group selected from:
5- and 6-membered carbocyclic aromatic rings, e.g., phenyl;
bicyclic ring systems such as 7 to 12 membered bicyclic ring systems, wherein at least one ring is carbocyclic and aromatic, e.g., naphthyl and indanyl; and,
tricyclic ring systems such as 10 to 15 membered tricyclic ring systems wherein at least one ring is carbocyclic and aromatic, e.g., fluorenyl.

The terms “aromatic hydrocarbon ring” and “aryl” are used interchangeably throughout the disclosure herein. In some embodiments, a monocyclic or bicyclic aromatic hydrocarbon ring has 5 to 10 ring-forming carbon atoms (i.e., C₅₋₁₀ aryl). Examples of a monocyclic or bicyclic aromatic hydrocarbon ring includes, but not limited to, phenyl, naphth-1-yl, naphth-2-yl, anthracenyl, phenanthrenyl, and the like. In some embodiments, the aromatic hydrocarbon ring is a naphthalene ring (naphth-1-yl or naphth-2-yl) or phenyl ring. In some embodiments, the aromatic hydrocarbon ring is a phenyl ring.

Specifically, the term “bicyclic fused aryl” includes a bicyclic aryl ring as defined herein. The typical bicyclic fused aryl is naphthalene.

The term “heteroaryl” includes a group selected from:
5-, 6- or 7-membered aromatic, monocyclic rings comprising at least one heteroatom, for example, from 1 to 4, or, in some embodiments, from 1 to 3, in some embodiments, from 1 to 2, heteroatoms, selected from nitrogen (N), sulfur (S) and oxygen (O), with the remaining ring atoms being carbon;
7- to 12-membered bicyclic rings comprising at least one heteroatom, for example, from 1 to 4, or, in some embodiments, from 1 to 3, or, in other embodiments, 1 or 2, heteroatoms, selected from N, O, and S, with the remaining ring atoms being carbon and wherein at least one ring is aromatic and at least one heteroatom is present in the aromatic ring; and
11- to 14-membered tricyclic rings comprising at least one heteroatom, for example, from 1 to 4, or in some embodiments, from 1 to 3, or, in other embodiments, 1 or 2, heteroatoms, selected from N, O, and S, with the remaining ring atoms being carbon and wherein at least one ring is aromatic and at least one heteroatom is present in an aromatic ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1. When the heteroaryl group contains more than one heteroatom ring member, the heteroatoms may be the same or different. The nitrogen atoms in the ring(s) of the heteroaryl group can be oxidized to form N-oxides.

Specifically, the term “bicyclic fused heteroaryl” includes a 7- to 12-membered, preferably 7- to 10-membered, more preferably 9- or 10-membered fused bicyclic heteroaryl ring as defined herein. Typically, a bicyclic fused heteroaryl is 5-membered/5-membered, 5-membered/6-membered, 6-membered/6-membered, or 6-membered/7-membered bicyclic. The group can be attached to the remainder of the molecule through either ring.

“Heterocyclyl”, “heterocycle” or “heterocyclic” are interchangeable and include a non-aromatic heterocyclyl group comprising one or more heteroatoms selected from nitrogen, oxygen or optionally oxidized sulfur as ring members, with the remaining ring members being carbon, including monocyclic, fused, bridged, and spiro ring, i.e., containing monocyclic heterocyclyl, bridged heterocyclyl, spiro heterocyclyl, and fused heterocyclic groups.

The term H or hydrogen disclosed herein includes Hydrogen and the non-radioisotope deuterium.

The term “at least one substituent” disclosed herein includes, for example, from 1 to 4, such as from 1 to 3, further as 1 or 2, substituents, provided that the theory of valence is met. For example, “at least one substituent F” disclosed herein includes from 1 to 4, such as from 1 to 3, further as 1 or 2, substituents F.

The term “divalent” refers to a linking group capable of forming covalent bonds with two other moieties. For example, “a divalent cycloalkyl group” refers to a cycloalkyl group obtained by removing two hydrogen from the corresponding cycloalkane to form a linking group. the term “divalent aryl group”, “divalent heterocyclyl group” or “divalent heteroaryl group” should be understood in a similar manner.

Compounds disclosed herein may contain an asymmetric center and may thus exist as enantiomers. “Enantiomers” refer to two stereoisomers of a compound which are non-superimposable mirror images of one another. Where the compounds disclosed herein possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds disclosed herein and/or pharmaceutically acceptable salts thereof are intended to be included. Unless specifically mentioned otherwise, reference to one isomer applies to any of the possible isomers. Whenever the isomeric composition is unspecified, all possible isomers are included.

When compounds disclosed herein contain olefinic double bonds, unless specified otherwise, such double bonds are meant to include both E and Z geometric isomers.

When compounds disclosed herein contain a di-substituted cyclic ring system, substituents found on such ring system may adopt cis and trans formations. Cis formation means that both substituents are found on the upper side of the 2 substituent placements on the carbon, while trans would mean that they were on opposing sides. For example, the di-substituted cyclic ring system may be cyclohexyl or cyclobutyl ring.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art could select and apply the techniques most likely to achieve the desired separation.

“Diastereomers” refer to stereoisomers of a compound with two or more chiral centers but which are not mirror images of one another. Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents (Eliel, E. and Wilen, S. Stereochemistry of Organic Compounds. New York: John Wiley & Sons, Inc., 1994; Lochmuller, C. H., et al. “Chromatographic resolution of enantiomers: Selective review.” J. Chromatogr., 113(3) (1975): pp. 283-302). Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions. See: Wainer, Irving W., Ed. Drug Stereochemistry: Analytical Methods and Pharmacology. New York: Marcel Dekker, Inc., 1993.

Some of the compounds disclosed herein may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

“Prodrug” refers to a derivative of an active agent that requires a transformation within the body to release the active agent. In some embodiments, the transformation is an enzymatic transformation.

Prodrugs are frequently, although not necessarily, pharmacologically inactive until converted to the active agent.

“deuterated analog” refers to a derivative of an active agent that an arbitrary hydrogen is substituted with deuterium. In some embodiments, the deuterated site is on the Warhead moiety. In some embodiments, the deuterated site is on the Linker moiety. In some embodiments, the deuterated site is on the Degron moiety.

“Pharmaceutically acceptable salts” refer to those salts which are, within the scope of sound medical judgment, suitable for use in contact with the tissues of humans and lower animals without undue toxicity, irritation, allergic response and the like, and are commensurate with a reasonable benefit/risk ratio. A pharmaceutically acceptable salt may be prepared in situ during the final isolation and purification of the compounds disclosed herein, or separately by reacting the free base function with a suitable organic acid or by reacting the acidic group with a suitable base. The term also includes salts of the stereoisomers (such as enantiomers and/or diastereomers), tautomers and prodrugs of the compound of the invention.

In addition, if a compound disclosed herein is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

The terms “administration”, “administering”, “treating” and “treatment” herein, when applied to an animal, human, experimental subject, cell, tissue, organ, or biological fluid, mean contact of an exogenous pharmaceutical, therapeutic, diagnostic agent, or composition to the animal, human, subject, cell, tissue, organ, or biological fluid. Treatment of a cell encompasses contact of a reagent to the cell, as well as contact of a reagent to a fluid, where the fluid is in contact with the cell. The term “administration” and “treatment” also means in vitro and ex vivo treatments, e.g., of a cell, by a reagent, diagnostic, binding compound, or by another cell. The term “subject” herein includes any organism, preferably an animal, more preferably a mammal (e.g., rat, mouse, dog, cat, and rabbit) and most preferably a human.

The term “effective amount” or “therapeutically effective amount” refers to an amount of the active ingredient, such as compound that, when administered to a subject for treating a disease, or at least one of the clinical symptoms of a disease or disorder, is sufficient to affect such treatment for the disease, disorder, or symptom. The term “therapeutically effective amount” can vary with the compound, the disease, disorder, and/or symptoms of the disease or disorder, severity of the disease, disorder, and/or symptoms of the disease or disorder, the age of the subject to be treated, and/or the weight of the subject to be treated. An appropriate amount in any given instance can be apparent to those skilled in the art or can be determined by routine experiments. In some embodiments, “therapeutically effective amount” is an amount of at least one compound and/or at least one stereoisomer, tautomer or prodrug thereof, and/or at least one pharmaceutically acceptable salt thereof disclosed herein effective to “treat” as defined herein, a disease or disorder in a subject. In the case of combination therapy, the term “therapeutically effective amount” refers to the total amount of the combination objects for the effective treatment of a disease, a disorder or a condition.

The term “disease” refers to any disease, discomfort, illness, symptoms or indications, and can be interchangeable with the term “disorder” or “condition”.

Throughout this specification and the Aspects which follow, unless the context requires otherwise, the term “comprise”, and variations such as “comprises” and “comprising” are intended to specify the presence of the features thereafter, but do not exclude the presence or addition of one or more other features. When used herein the term “comprising” can be substituted with the term “containing”, “including” or sometimes “having”.

Throughout this specification and the Aspects which follow, the term “Cₙ₋ₘ” or “Cₙ-Cₘ” indicates a range which includes the endpoints, wherein n and m are integers and indicate the number of carbons. Examples include C₁₋₈, C₁₋₆, C₁-C₈, C₁-C₆, and the like.

Unless specifically defined elsewhere in this document, all other technical and scientific terms used herein have the meaning commonly understood by one of ordinary skill in the art to which this invention belongs.

### EXAMPLES

The examples below are intended to be purely exemplary and should not be considered to be limiting in any way. Efforts have been made to ensure accuracy with respect to numbers used (for example, amounts, temperature, etc.), but some experimental errors and deviations should be accounted for. Unless indicated otherwise, temperature is in degrees Centigrade. Reagents were purchased from commercial suppliers such as Sigma-Aldrich, Alfa Aesar, or TCI, and were used without further purification unless indicated otherwise. Unless indicated otherwise, the reactions set forth below were performed under a positive pressure of nitrogen or argon or with a drying tube in anhydrous solvents; the reaction flasks were fitted with rubber septa for the introduction of substrates and reagents via syringe; and glassware was oven dried and/or heat dried.

¹H NMR spectra were recorded on an Agilent instrument operating at 400 MHz. ¹HNMR

spectra were obtained using CDCl₃, CD₂Cl₂, CD₃OD, D₂O, d₆-DMSO, d₆-acetone or (CD₃)₂CO as solvent and tetramethylsilane (0.00 ppm) or residual solvent (CDCl₃: 7.25 ppm; CD₃OD: 3.31 ppm; D₂O: 4.79 ppm; d₆-DMSO: 2.50 ppm; d₆-acetone: 2.05; (CD₃)₃CO: 2.05) as the reference standard. When peak multiplicities are reported, the following abbreviations are used: s (singlet), d (doublet), t (triplet), q (quartet), qn (quintuplet), sx (sextuplet), m (multiplet), br (broadened), dd (doublet of doublets), dt (doublet of triplets). Coupling constants, when given, are reported in Hertz (Hz).

LCMS-1: LC-MS spectrometer (Agilent 1260 Infinity) Detector: MWD (190-400 nm), Mass detector: 6120 SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS, LCMS-3: LC-MS spectrometer (Agilent 1260 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.8 mL/min Time (min) A (%) B (%)

| Time (min) | A(%) | B(%) |
|---|---|---|
| 0.00 | 95 | 5 |
| 1.5 | 5 | 95 |
| 2.0 | 5 | 95 |
| 2.1 | 95 | 5 |
| 3.0 | 95 | 5 |

LCMS-2: LC-MS spectrometer (Agilent 1290 Infinity II) Detector: MWD (190-400 nm), Mass detector: G6125C SQ Mobile phase: A: water with 0.1% Formic acid, B: acetonitrile with 0.1% Formic acid Column: Poroshell 120 EC-C18, 4.6×50 mm, 2.7 pm Gradient method: Flow: 1.2 mL/min Time (min) A (%) B (%)

| Time (min) | A(%) | B(%) |
|---|---|---|
| 0.00 | 90 | 10 |
| 1.5 | 5 | 95 |
| 2.0 | 5 | 95 |
| 2.1 | 90 | 10 |
| 3.0 | 90 | 10 |

Preparative HPLC was conducted on a column (150×21.2 mm ID, 5 pm, Gemini NXC 18) at a flow rate of 20 ml/min, injection volume 2 ml , at room temperature and UV Detection at 214 nm and 254 nm. In the following examples, the abbreviations below are used:

| | |
|---|---|
| (BPin)₂ | 4,4,4′,4′,5,5,5′,5′-Octamethyl-2,2′-bi-1,3,2-dioxaborolane |
| Ac₂O | acetic anhydride |
| AcCl | Acetyl chloride |
| ACN or MeCN | Acetonitrile |
| AcOH or HOAc | Acetic acid |
| AcONa or NaOAc | Sodium acetate |
| Aq | Aqueous |
| BINAP | (±)-2,2′-Bis(diphenylphosphino)-1,1′-BINAPhthyl |
| Bn | benzyl |
| BnBr | Benzyl Bromide |
| Boc | t-Butyloxy carbonyl |
| C: 40691-33-6 | dichlorobis(tri-o-tolylphosphine)palladium(II) |
| Cbz | Benzyloxycarbonyl |
| DCM | Dichloromethane |
| Con. | Concentrated |
| DavePhos | 2′-(Dicyclohexylphosphino)-N,N-dimethyl-2-biphenylamine |
| DBU | 1,8-diazabicyclo[5.4.0]undec-7-ene |
| DCE | dichloroethane |
| DHP | 3,4-Dihydro-2H-pyran |
| DIBAL-H | Diisobutylaluminium hydride |
| DIEA or DIPEA | N,N-diisopropylethylamine |
| DMAP | 4-N,N-dimethylaminopyridine |
| DMP | Dess-Martin periodinane |
| DMF | N,N-Dimethylformamide |
| DMSO | Dimethyl sulfoxide |
| Dppf | 1,1″-bis(diphenylphosphino)ferrocene |
| EA or EtOAc | Ethyl acetate |
| EtOH | ethanol |
| FA | Formic acid |
| h or hr | Hour |
| HATU | 2-(7-Azabenzotriazol-1-yl)-N,N,N′,N′-tetramethyluronium |
| | hexafluorophosphate |
| HBTU | O-(7-Benzotriazole-1-yl)-N,N,N′,N′-tetramethyluronium |
| | hexafluorophosphate |
| Hex | Hexane |
| HPLC | High Performance Liquid Chromatography |
| hrs | hours |
| IBX | 2-Iodoxybenzoic acid |
| IPA | 2-propanol |
| i-PrOH | Isopropyl alcohol |
| KHMDS | Potassium bis(trimethylsilyl)amide |
| LiHMDS | Lithium bis(trimethylsilyl)amide |
| KOAc or AcOK | Potassium Acetate |
| MeCN or ACN | Acetonitrile |
| MeOH | Methanol |
| Min | Minutes |
| ms or MS | Mass spectrum |
| MsCl | Methanesulfonyl chloride |
| MsOH | Methanesulfonic acid |
| MTBE | Methyl tert-butyl ether |
| o/n | overnight |
| Pd(dppf)Cl₂ | [1,1′-Bis(diphenylphosphino)ferrocene]dichloropalladium(II) |
| Pd₂(dba)₃ | Tris(dibenzylideneacetone)dipalladium |
| PE | Petroleum ether |
| PhMe | Toluene |
| PMB | 4-Methoxybenzyl |
| PPA | Polyphosphoric acid |
| R.T. or r.t. | Room temperature |
| Rt | Retention time |
| Selectfluor | 1-Chloromethyl-4-fluoro-1,4-diazoniabicyclo[2.2.2]octane |
| | bis(tetrafluoroborate) |
| SEMCl | 2-(Trimethylsilyl)ethoxymethyl chloride |
| STAB | Sodium Triacetoxyborohydride; or sodium triacetoborohydride |
| Sat. | saturated |
| SFC | Supercritical Fluid Chromatography |
| TBAF | Tetra-butyl ammonium fluoride |
| TBDPS | tert-Butyldiphenylsilyl |
| TBS | tert-Butyldimethylsilyl |
| TBSCl | tert-Butyldimethylsilyl chloride |
| Ti(OiPr)₄ | Titanium tetraisopropanolate |
| t-Bu | tert-butyl |
| t-BuOH | tert-Butanol |
| t-BuONa | Sodium tert-butoxide |
| t-BuOK | Potassium tert-butoxide |
| TEA | Triethylamine |
| Tf₂O | Triflic anhydride |
| TFA | Trifluoroacetic acid |
| THF | Tetrahydrofuran |
| TLC | Thin layer chromatography |
| TMSOK | Potassium trimethylsilanolate |
| Ts | para-Toluenesulfonyl |
| TsCl | 4-Toluenesulfonyl chloride |
| TsOH | p-toluenesulfonic acid |
| TsOH, Py | Pyridinium toluene-4-sulphonate |
| Xphos or X-phos | 2-Dicyclohexylphosphino-2′,4′,6′-triisopropylbiphenyl |
| XantPhos | 4,5-Bis(diphenylphosphino)-9,9-dimethylxanthene |

### Examples 2 and 13: (S,E)-3-(4-(2-ethoxyvinyl)-2,6-difluorophenyl)piperidine-2,6-dione (2) and (R,E)-3-(4-(2-ethoxyvinyl)-2,6-difluorophenyl)piperidine-2,6-dione (13)

### Step 1: ethyl 4-(4-bromo-2,6-difluorophenyl)-4-cyanobutanoate

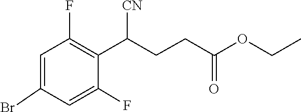 [SMILES: CCOC(=O)CCC(C#N)c1c(F)cc(Br)cc1F]

To a solution of 2-(4-bromo-2,6-difluorophenyl)acetonitrile (10 g, 43.1 mmol) in THF (150 mL) was added LDA (2M, 24 mL, 48 mmol) dropwise in 20 min at −65° C., the reaction solution was stirred for 1 hour at this temperature, then to this was added ethyl 3-bromopropanoate (9.4 g, 51.7 mmol) in THF (30 mL) dropwise in 10 min. The resulting solution was stirred for 30 min at −65° C., then the temperature was allowed to rise to room temperature naturally. The reaction was quenched by the addition of sat. aq. NH₄Cl (50 mL), and the layers were separated. The aqueous layer was extracted with EtOAc (100 mL×3), the organic layers were combined and washed with brine, and dried over anhydrous Na₂SO₄. After filtration, the filtrate was concentrated under reduced pressure to afford the product (13.8 g, 96.5%). [M+H]⁺=332.0.

### Step 2: 4-(4-bromo-2,6-difluorophenyl)-4-cyanobutanoic acid

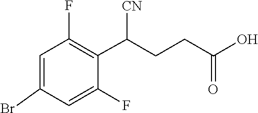 [SMILES: N#CC(CCC(=O)O)c1c(F)cc(Br)cc1F]

To a solution of ethyl 4-(4-bromo-2,6-difluorophenyl)-4-cyanobutanoate (13.5 g, 40.7 mmol) in THF/H₂O (90 mL/30 mL) was added LiOH (2.9 g, 0.122 mol). The reaction mixture was stirred for 12 h at room temperature. The resulting mixture was diluted with water, and the layers were separated. The pH value of the aqueous layer was adjusted to 4-5 with 1 M HCl, and then extracted with EtOAc (50 mL×3). The combined organic layers were washed with brine (50 mL), dried over anhydrous Na₂SO₄. After filtration, the filtrate was concentrated under reduced pressure to afford the product (10.2 g, 82.5%). [M+H]⁺=304.2.

### Step 3: 3-(4-bromo-2,6-difluorophenyl)piperidine-2,6-dione

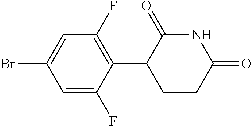 [SMILES: O=C1CCC(c2c(F)cc(Br)cc2F)C(=O)N1]

To a stirred solution of 4-(4-bromo-2,6-difluorophenyl)-4-cyanobutanoic acid (10.2 g, 33.5 mmol) in toluene(100 mL) was added conc.H₂SO₄ (2 mL, 36.9 mmol). The resulting solution was stirred at 100° C. for 3 h. The reaction mixture was concentrated under vacuum, then the mixture was poured into water. The pH value was adjusted to 7-8 with sat. aq. NaHCO₃, and then the mixture was extracted with EtOAc (50 mL×3). The combined organic layers were washed with water (50 mL) and brine (50 mL), dried over anhydrous Na₂SO₄, filtered and concentrated under reduced pressure to afford the product (8.2 g, 80.4%). [M+H]⁺=304.3.

### Step 4: (S,E)-3-(4-(2-ethoxyvinyl)-2,6-difluorophenyl)piperidine-2,6-dione and (R,E)-3-(4-(2-ethoxyvinyl)-2,6-difluorophenyl)piperidine-2,6-dione

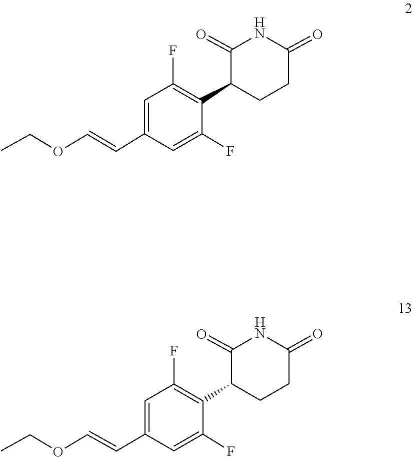 [SMILES: CCO/C=C/c1cc(F)c([C@@H]2CCC(=O)NC2=O)c(F)c1 ; CCO/C=C/c1cc(F)c([C@H]2CCC(=O)NC2=O)c(F)c1]

To a stirred solution of 3-(4-bromo-2,6-difluorophenyl)piperidine-2,6-dione (8.2 g, 27.0 mmol) and (E)-2-(2-ethoxyvinyl)-4,4,5,5-tetramethyl-1,3,2-dioxaborolane (6.4 g, 32.4 mmol) in DMF/H₂O (100 mL/20 mL) were added Pd(dtbpf)Cl₂ (883 mg, 1.35 mmol) and CsF(8.2 g, 54.0 mmol). The resulting mixture was stirred for 2 h at 80° C. under nitrogen atmosphere. The reaction solution was diluted with water (400 mL), and extracted with EtOAc (100 mL×2). The combined organic layers were washed with water (100 mL) and brine (100 mL), dried over anhydrous Na₂SO₄, filtered and concentrated under reduced pressure. The residue was purified by SFC ((IH(3*25 cm, 5 um), 13% EtOH/87% CO₂, 100 bar, 100 ml/min) to afford (S,E)-3-(4-(2-ethoxyvinyl)-2,6-difluorophenyl)piperidine-2,6-dione corresponded to peak B @ 2.049 min/254 nm (3.1 g, 39.0%) and (R,E)-3-(4-(2-ethoxyvinyl)-2,6-difluorophenyl)piperidine-2,6-dione corresponded to peak A @ 1.679 min/254 nm (2.9 g, 36.5%). (S,E)-3-(4-(2-ethoxyvinyl)-2,6-difluorophenyl)piperidine-2,6-dione and (R,E)-3-(4-(2-ethoxyvinyl)-2,6-difluorophenyl)piperidine-2,6-dione had the same ¹H NMR and LCMS data. ¹H NMR (500 MHz, DMSO) δ 10.92 (s, 1H), 7.41 (d, J=12.9 Hz, 1H), 7.06 (d, J=10.7 Hz, 2H), 5.82 (d, J=12.9 Hz, 1H), 4.17-4.13 (m, 1H), 3.92-3.88 (m, 2H), 3.45-3.39 (m, 1H), 2.82-2.76 (m, 1H), 2.12-2.07 (m, 1H), 2.00-1.96 (m, 1H), 1.26 (t, J=7.0 Hz, 3H). [M+H]⁺=295.9.

### Example 5 and 6: (R)-1-(4-((R)-2,6-dioxopiperidin-3-yl)-3,5-difluorophenyl)pyrrolidine-3-carboxylic acid (5) and (R)-1-(4-((S)-2,6-dioxopiperidin-3-yl)-3,5-difluorophenyl)pyrrolidine-3-carboxylic acid (6)

### Step 1: methyl (R)-1-(4-bromo-3,5-difluorophenyl)pyrrolidine-3-carboxylate

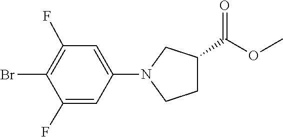 [SMILES: COC(=O)[C@@H]1CCN(c2cc(F)c(Br)c(F)c2)C1]

To the solution of 2-bromo-1,3-difluoro-5-iodobenzene (15 g, 47 mmol), methyl (R)-pyrrolidine-3-carboxylate hydrochloride (8.56 g, 51.7 mmol) and K₃PO₄ (20 g, 94 mmol) in 250 mL DMSO, CuI (893 mg, 4.7 mmol) and L-Proline (1 g, 9.4 mmol) was added. The mixture was stirred at 80° C. for 16 hours. After LCMS showed the reaction was completed, the mixture was diluted with water (500 mL) and extracted with EtOAc (300 mL×3). The combined organic layers were washed with brine and separated. The organic phase was concentrated and purified by silica column chromatography (PE:EA=50:1-30:1) to afford the product (4.9 g, 32.5%). [M+H]⁺=320.1.

### Step 2: methyl (R)-1-(4-(2,6-bis(benzyloxy)pyridin-3-yl)-3,5-difluorophenyl)pyrrolidine-3-carboxylate

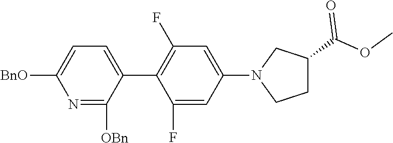 [SMILES: COC(=O)[C@@H]1CCN(c2cc(F)c(-c3ccc(OCc4ccccc4)nc3OCc3ccccc3)c(F)c2)C1]

To the solution of methyl (R)-1-(4-bromo-3,5-difluorophenyl)pyrrolidine-3-carboxylate (4.9 g, 15.3 mmol), 2,6-bis(benzyloxy)-3-(4,4,5,5-tetramethyl-1,3,2-dioxaborolan-2-yl)pyridine (6.7 g, 16 mmol) and CsF (4.6 g, 30.6 mmol) in 150 mL DMF and 15 mL water, Pd(dtbpf)Cl₂ (498 mg, 0.8 mmol) was added. The mixture was stirred at 80° C. for 4 hours. After LCMS showed the reaction was completed, the mixture was washed with water and extracted with EtOAc. The organic layer was washed with brine and separated. The organic phase was concentrated in vacuum and purified by combi-flash (EA:PE=1:10) to afford the product (7.9 g, 97.4%). [M+H]⁺=531.3.

### Step 3: (R)-1-(4-(2,6-bis(benzyloxy)pyridin-3-yl)-3,5-difluorophenyl)pyrrolidine-3-carboxylic acid

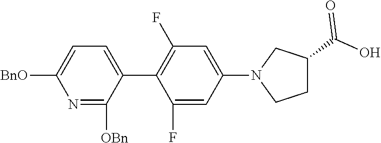 [SMILES: O=C(O)[C@@H]1CCN(c2cc(F)c(-c3ccc(OCc4ccccc4)nc3OCc3ccccc3)c(F)c2)C1]

To the solution of methyl (R)-1-(4-(2,6-bis(benzyloxy)pyridin-3-yl)-3,5-difluorophenyl)pyrrolidine-3-carboxylate (7.9 g, 14.9 mmol) in 100 mL THF and 20 mL water, LiOH (394 mg, 16.4 mmol) in 10 mL water was added dropwise at room temperature. The mixture was stirred at room temperature for 15 minutes. After TLC showed the reaction was completed, the mixture was concentrated in vacuum at room temperature. The residue was diluted with water and adjust to pH<5 with 1 N HCl aqueous. The liquid was extracted with EtOAc and separated. The organic phase was dried with Na₂SO₄ and concentrated in vacuum to afford the product (7.4 g, 96.0%). [M+H]⁺=517.2.

### Step 4: (R,)-1-(4-((R)-2,6-dioxopiperidin-3-yl)-3,5-difluorophenyl)pyrrolidine-3-carboxylic acid (5) and (R)-1-(4-((S)-2,6-dioxopiperidin-3-yl)-3,5-difluorophenyl)pyrrolidine-3-carboxylic acid (6)

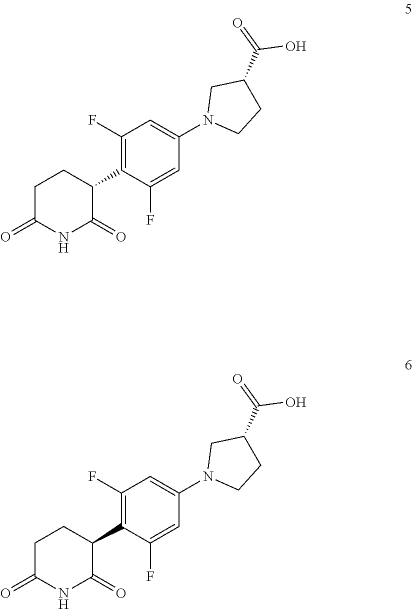 [SMILES: O=C1CC[C@H](c2c(F)cc(N3CC[C@@H](C(=O)O)C3)cc2F)C(=O)N1 ; O=C1CC[C@@H](c2c(F)cc(N3CC[C@@H](C(=O)O)C3)cc2F)C(=O)N1]

To the solution of (R)-1-(4-(2,6-bis(benzyloxy)pyridin-3-yl)-3,5-difluorophenyl)pyrrolidine-3-carboxylic acid (7.4 g, 14.3 mmol) in 50 mL DCM and 250 mL iPrOH, Pd/C (7.4 g, 10%) was added. The mixture was stirred at 40° C. for 16 hours under hydrogen atmosphere (balloon). After LCMS showed the reaction was completed, the mixture was cooled to room temperature and filtered by celite directly. The filtrate was concentrated in vacuum to afford the crude product which was purified by SFC ((IH(3*25 cm, 5 um), 13% EtOH/87% CO₂, 100 bar, 100 ml/min). The product (R)-1-(4-((R)-2,6-dioxopiperidin-3-yl)-3,5-difluorophenyl)pyrrolidine-3-carboxylic acid corresponded to peak A @ 0.655 min/254 nm (1.6 g, 34%) ¹H NMR (500 MHz, DMSO) δ 12.52 (s, 1H), 10.84 (s, 1H), 6.23 (d, J=12.1 Hz, 2H), 4.02 (dd, J=12.6, 5.0 Hz, 1H), 3.47-3.36 (m, 2H), 3.32-3.22 (m, 2H), 3.21-3.14 (m, 1H), 2.83-2.72 (m, 1H), 2.49 (m, 1H), 2.26-2.03 (m, 3H), 1.94 (m, 1H). [M+H]⁺=338.9. and (R)-1-(4-((S)-2,6-dioxopiperidin-3-yl)-3,5-difluorophenyl)pyrrolidine-3-carboxylic acid corresponded to peak B @ 1.811 min/254 nm (1.5 g, 32%). ¹H NMR (500 MHz, DMSO) δ 12.53 (s, 1H), 10.84 (s, 1H), 6.23 (d, J=12.1 Hz, 2H), 4.02 (dd, J=12.6, 5.0 Hz, 1H), 3.49-3.36 (m, 2H), 3.32-3.21 (m, 2H), 3.21-3.14 (m, 1H), 2.77 (m, 1H), 2.49 (m, 1H), 2.25-2.03 (m, 3H), 1.99-1.88 (m, 1H). [M+H]⁺=339.0.

### Example 7: (R)-3-(2,6-difluoro-4-((R)-3-(hydroxymethyl)pyrrolidin-1-yl)phenyl)piperidine-2,6-dione

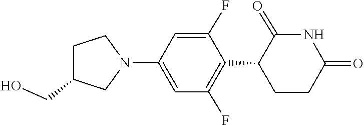 [SMILES: O=C1CC[C@H](c2c(F)cc(N3CC[C@@H](CO)C3)cc2F)C(=O)N1]

The title compound was prepared in a procedure similar to Example 9. (R)-3-(2,6-difluoro-4-((R)-3-(hydroxymethyl)pyrrolidin-1-yl)phenyl)piperidine-2,6-dione was purified by SFC ((IH(3*25 cm, 5 um), 13% EtOH/87% CO₂, 100 bar, 100 ml/min) and corresponded to peak A @ 2.028 min/254 nm (230 mg, 31%). ¹H NMR (500 MHz, DMSO) δ 10.83 (s, 1H), 6.17 (d, J=12.2 Hz, 2H), 4.71 (t, J=5.2 Hz, 1H), 4.01 (dd, J=12.6, 5.0 Hz, 1H), 3.46-3.35 (m, 2H), 3.31-3.16 (m, 3H), 2.99 (dd, J=9.7, 6.3 Hz, 1H), 2.83-2.72 (m, 1H), 2.48 (m, 1H), 2.46-2.37 (m, 1H), 2.14-1.90 (m, 3H), 1.74 (ddd, J=14.9, 7.4 Hz, 1H). [M+H]⁺=325.0.

### Example 9: 3-(2,6-difluoro-4-(3-(hydroxymethyl)azetidin-1-yl)phenyl)piperidine-2,6-dione

### Step 1: (1-(4-(2,6-bis(benzyloxy)pyridin-3-yl)-3,5-difluorophenyl)azetidin-3-yl)methanol

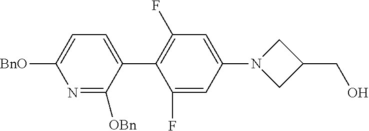 [SMILES: OCC1CN(c2cc(F)c(-c3ccc(OCc4ccccc4)nc3OCc3ccccc3)c(F)c2)C1]

To a stirred mixture of methyl 1-(4-(2,6-bis(benzyloxy)pyridin-3-yl)-3,5-difluorophenyl)azetidine-3-carboxylate (1.7 g, 3.29 mmol) in THF (20 mL) was added LiAlH₄ (1 M in THF, 4.27 mL, 4.27 mmol) dropwise at 0° C. Then the mixture was stirred for 2 hours, the reaction was quenched with water (10 mL) at 0° C. The resulting mixture was extracted with EtOAc (3×50 mL). The combined organic layers were washed with brine, dried over anhydrous Na₂SO₄. After filtration, the filtrate was concentrated under reduced pressure to afford the product (1.4 g, 87%) [M+H]⁺=489.2.

### Step 2: 3-(2,6-difluoro-4-(3-(hydroxymethyl)azetidin-1-yl)phenyl)piperidine-2,6-dione

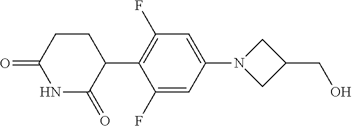 [SMILES: O=C1CCC(c2c(F)cc(N3CC(CO)C3)cc2F)C(=O)N1]

To a solution of (1-(4-(2,6-bis(benzyloxy)pyridin-3-yl)-3,5-difluorophenyl)azetidin-3-yl)methanol (1.40 g, 2.87 mmol) in iPrOH (20 mL) and DCM (20 mL) was added Pd/C (1.0 g, 10% wt) which was stirred at room temperature under hydrogen atmosphere for 48 hours. The resulting mixture was filtered, and the solid was washed with MeOH (20 mL). The filtrate was concentrated under reduced pressure to afford the product (450 mg, 51%). ¹H NMR (500 MHz, DMSO) δ 10.85 (s, 1H), 6.08 (d, J=11.2 Hz, 2H), 4.77 (t, J=5.2 Hz, 1H), 4.02 (dd, J=12.6, 4.9 Hz, 1H), 3.84 (t, J=7.7 Hz, 2H), 3.56 (t, J=5.8 Hz, 4H), 2.77 (ddd, J=18.2, 13.0, 5.3 Hz, 2H), 2.50 (m, 1H), 2.14-2.01 (m, 1H), 1.98-1.88 (m, 1H). [M+H]⁺=311.0.

### Example 12: (3R)-1-(4-(2,6-dioxopiperidin-3-yl)phenyl)pyrrolidine-3-carboxylic acid

### Step 1: 26-bis(benzyloxy)-3-(4-bromophenyl)pyridine

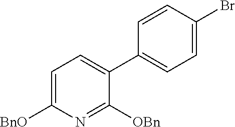 [SMILES: Brc1ccc(-c2ccc(OCc3ccccc3)nc2OCc2ccccc2)cc1]

To a stirred mixture of 2,6-bis(benzyloxy)-3-(4,4,5,5-tetramethyl-1,3,2-dioxaborolan-2-yl)pyridine (The intermediate can be prepared according to the way described in WO2017197046) (8.3 g, 20 mmol) and 4-bromoiodobenzene (5.6 g, 20 mmol) in 1,4-dioxane (100 mL) and H₂O (10 mL) was added K₂CO₃ (5.5 g, 40 mmol) and Pd(dppf)Cl₂ (1.4 g, 2 mmol) at room temperature under nitrogen atmosphere. The resulting mixture was stirred for 16 h at 80° C. under nitrogen atmosphere. The reaction mixture was allowed to cool down to room temperature. The resulting mixture was extracted with EtOAc (3×500 mL). The combined organic layers were washed with brine (500 mL), dried over anhydrous Na₂SO₄, filtered and concentrated under reduced pressure. The residue was purified by silica gel column chromatography (EA:PE=1:10) to afford the product (4.5 g, 50%). [M+H]⁺=446.2.

### Step 2: methyl (R)-1-(4-(2,6-bis(benzyloxy)pyridin-3-yl)phenyl)pyrrolidine-3-carboxylate

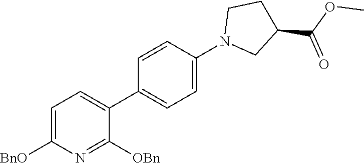 [SMILES: COC(=O)[C@@H]1CCN(c2ccc(-c3ccc(OCc4ccccc4)nc3OCc3ccccc3)cc2)C1]

To the solution of 2,6-bis(benzyloxy)-3-(4-bromophenyl)pyridine (4.5 g, 10 mmol), methyl (R)-pyrrolidine-3-carboxylate hydrochloride (2.5 g, 15 mmol) and Cs₂CO₃ (15 g, 45 mmol) in 50 mL 1,4-dioxane, was added Pd₂(dba)₃ (915 mg, 1 mmol) and Xantphos (1.16 g, 2 mmol). The mixture was stirred at 80° C. for 16 hours under nitrogen atmosphere. After LCMS shown the reaction was completed, the mixture was concentrated under reduced pressure and purified by silica column chromatography (EA:PE=1:10) to afford the product (2.1 g, 42.5%). [M+H]⁺=494.9.

### Step 3: (R)-1-(4-(2,6-bis(benzyloxy)pyridin-3-yl)phenyl)pyrrolidine-3-carboxylic acid

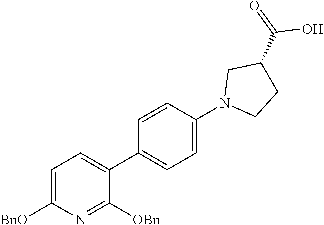 [SMILES: O=C(O)[C@@H]1CCN(c2ccc(-c3ccc(OCc4ccccc4)nc3OCc3ccccc3)cc2)C1]

To the solution of methyl (R)-1-(4-(2,6-bis(benzyloxy)pyridin-3-yl)phenyl)pyrrolidine-3-carboxylate (2.1 g, 4.25 mmol) in 30 mL THF and 10 mL water, was added LiOH H₂O (178 mg, 4.25 mmol) in 2 mL water dropwise at room temperature. The mixture was stirred at room temperature for 15 minutes. Once the reaction was completed determined by TLC, the mixture was concentrated under reduced pressure. The residue was diluted with water and adjust to pH<5 with 1 N HCl aqueous. The liquid was extracted with EtOAc (100 mL×3). The combined organic layers were washed with brine (60 mL×3), dried over anhydrous Na₂SO₄, filtered and concentrated in vacuum to afford the product (2 g, 98%). [M+H]⁺=481.6.

### Step 4: (3R)-1-(4-(2,6-dioxopiperidin-3-yl)phenyl)pyrrolidine-3-carboxylic acid

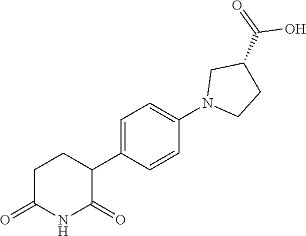 [SMILES: O=C1CCC(c2ccc(N3CC[C@@H](C(=O)O)C3)cc2)C(=O)N1]

To the solution of (R)-1-(4-(2,6-bis(benzyloxy)pyridin-3-yl)phenyl)pyrrolidine-3-carboxylic acid (2 g, 4.17 mmol) in 5 mL DCM and 100 mL iPrOH, Pd/C (2 g, 10%) was added. The mixture was stirred at 45° C. f or 16 hours under hydrogen atmosphere (balloon). Once the reaction has completed determined by LCMS, the mixture was cooled to room temperature and filtered through celite directly. The solid was dispensed in DCM (5 mL) and MeOH (50 mL), which was sonicated for 5 min. The mixture was then filtered through celite and the combined filtrate was concentrated in vacuum to afford the product (1.2 g, 95%). ¹H NMR (500 MHz, DMSO) δ 12.49 (s, 1H), 10.74 (s, 1H), 7.00 (d, J=8.4 Hz, 2H), 6.51 (d, J=8.5 Hz, 2H), 3.68 (dd, J=10.7, 4.9 Hz, 1H), 3.40 (m, 2H), 3.31-3.20 (m, 2H), 3.19-3.12 (m, 1H), 2.61 (ddd, J=16.6, 10.9, 5.3 Hz, 1H), 2.44 (m, 1H), 2.25-2.06 (m, 3H), 2.04-1.93 (m, 1H). [M+H]⁺=303.1.

### Example 14: (S)-1-(4-((R)-2,6-dioxopiperidin-3-yl)-3,5-difluorophenyl)-4,4-dimethylpyrrolidine-3-carboxylic acid

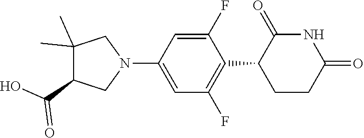 [SMILES: CC1(C)CN(c2cc(F)c([C@H]3CCC(=O)NC3=O)c(F)c2)C[C@H]1C(=O)O]

The title compound was prepared in a procedure similar to Example 12. (S)-1-(4-((R)-2,6-dioxopiperidin-3-yl)-3,5-difluorophenyl)-4,4-dimethylpyrrolidine-3-carboxylic acid was purified by SFC (IH(3*25 cm, 5 um), 13% EtOH/87% CO₂, 100 bar, 100 ml/min) and corresponded to peak A @ 1.165 min/254 nm (670 mg, 26%). ¹H NMR (500 MHz, DMSO) δ 12.47 (s, 1H), 10.84 (s, 1H), 6.17 (d, J=12.1 Hz, 2H), 4.01 (m, 1H), 3.48 (m, 2H), 3.16 (d, J=9.5 Hz, 1H), 3.08 (d, J=9.4 Hz, 1H), 2.89 (t, J=8.0 Hz, 1H), 2.83-2.72 (m, 1H), 2.54 (s, 1H), 2.08 (m, 1H), 1.98-1.86 (m, 1H), 1.23 (s, 3H), 1.00 (s, 3H). [M+H]⁺=367.1.

### Example 15: 3-(4-(2-hydroxyethyl)phenyl)piperidine-2,6-dione

### Step 1: 2-(4-(4,4,5,5-tetramethyl-1,3,2-dioxaborolan-2-yl)phenyl)ethan-1-ol

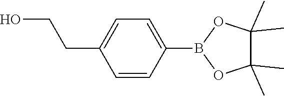 [SMILES: CC1(C)OB(c2ccc(CCO)cc2)OC1(C)C]

2-(4-bromophenyl)ethan-1-ol (20 g, 100 mmol), 4,4,4′,4′,5,5,5′,5′-octamethyl-2,2′-bi-1,3,2-dioxaborolane (38.1 g, 150 mmol), Pd(dppf)Cl₂ (7.3 g, 10 mmol), KOAc (19.6 g, 200 mmol) were placed in 1,4-dioxane (400 mL). The resulting mixture was then heated to 100° C. and stirred for 2 h. The mixture was cooled to room temperature. After filtration, the filtrate was concentrated in vacuum to afford the crude product (28 g, crude) which was used directly without further purification. [M+H]⁺=249.2.

### Step 2: 2-(4-(2,6-bis(benzyloxy)pyridin-3-yl)phenyl)ethan-1-ol

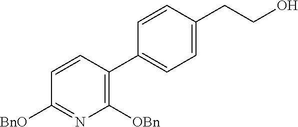 [SMILES: OCCc1ccc(-c2ccc(OCc3ccccc3)nc2OCc2ccccc2)cc1]

A mixture of 2-(4-(4,4,5,5-tetramethyl-1,3,2-dioxaborolan-2-yl)phenyl)ethan-1-ol (28 g, crude), Pd(dppf)Cl₂ (7.3 g, 10 mmol), 2,6-bis(benzyloxy)-3-bromopyridine (36.9 g, 100 mmol) and Cs₂CO₃ (65.2 g, 200 mmol) in 1,4-dioxane (300 mL) and water (30 mL) was stirred at 100° C. overnight under nitrogen atmosphere. The reaction was cooled down to room temperature, the solids were filtered out, the filtrate was concentrated and purified with silica gel column (eluted with EtOAc/Hexane=1:2) to give the crude product which was used directly in the next step. [M+H]⁺=412.2.

### Step 3: 3-(4-(2-hydroxyethyl)phenyl)piperidine-2,6-dione

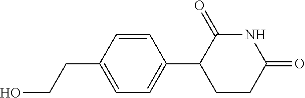 [SMILES: O=C1CCC(c2ccc(CCO)cc2)C(=O)N1]

2-(4-(2,6-bis(benzyloxy)pyridin-3-yl)phenyl)ethan-1-ol (the crude from last step) was dissolved in MeOH (500 mL), Pd/C (10%, w/w, 5 g) was added to the solution in one portion. The resulting mixture was stirred under hydrogen atmosphere (1 atm) overnight. The solids were filtered out, the filtrate was concentrated in vacuum to give a crude product. The crude was triturated with MTBE (50 mL) to give desire product (13.5 g, 57.9% yield over 3 steps). ¹H NMR (500 MHz, DMSO) δ 10.81 (s, 1H), 7.17 (d, J=8.0 Hz, 2H), 7.11 (d, J=8.1 Hz, 2H), 4.63 (t, J=5.2 Hz, 1H), 3.80 (dd, J=11.4, 4.9 Hz, 1H), 3.59 (dd, J=12.3, 7.0 Hz, 2H), 2.76-2.58 (m, 3H), 2.49-2.44 (m, 1H), 2.23-2.09 (m, 1H), 2.02 (dt, J=8.3, 4.8 Hz, 1H). [M+H]⁺=234.1.

### Example 16: (R)-3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

### Step 1: tert-butyl 4-(1-(2-bromo-5-methoxy-4-nitrophenyl)piperidin-4-yl)piperazine-1-carboxylate

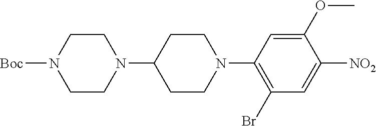 [SMILES: COc1cc(N2CCC(N3CCN(C(=O)OC(C)(C)C)CC3)CC2)c(Br)cc1[N+](=O)[O-]]

A mixture of 1-bromo-2-fluoro-4-methoxy-5-nitrobenzene (4 g, 16 mmol), tert-butyl 4-(piperidin-4-yl)piperazine-1-carboxylate (6.4 g, 24 mmol), K₂CO₃ (4.4 g, 32 mmol) in DMF (50 mL) was stirred in a flask at 80° C. overnight. The reaction mixture was allowed to cool down to room temperature. The resulting mixture was diluted with water and extracted with EtOAc (3×500 mL). The combined organic layers were washed with brine (500 mL), dried over anhydrous Na₂SO₄. After filtration, the filtrate was concentrated under reduced pressure. The residue was purified by silica gel column chromatography, eluted with PE/EtOAc (1:1) to afford the product (7 g, 90%). [M+H]⁺=499.0.

### Step 2: tert-butyl 4-(1-(5-methoxy-4-nitro-2-vinylphenyl)piperidin-4-yl)piperazine-1-carboxylate

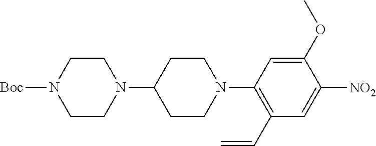 [SMILES: C=Cc1cc([N+](=O)[O-])c(OC)cc1N1CCC(N2CCN(C(=O)OC(C)(C)C)CC2)CC1]

A mixture of tert-butyl 4-(1-(2-bromo-5-methoxy-4-nitrophenyl)piperidin-4-yl)piperazine-1-carboxylate (7 g, 14 mmol), 4,4,5,5-tetramethyl-2-vinyl-1,3,2-dioxaborolane (4.3 g, 28 mmol), Pd(dppf)Cl₂ (1.1 g, 1.4 mmol) and K₃PO₄ (8.9 g, 42 mmol) in DMF (160 mL) and water (20 mL) was stirred in a flask at 90° C. under nitrogen atmosphere for 16 hrs. The reaction mixture was allowed to cool down to room temperature. The resulting mixture was extracted with EtOAc (3×1000 mL). The combined organic layers were washed with brine (500 mL), dried over anhydrous Na₂SO₄. After filtration, the filtrate was concentrated under reduced pressure. The residue was purified by silica gel column chromatography, eluted with PE/EtOAc (1:1) to afford the product (5 g, 80%). [M+H]⁺=447.0.

### Step 3: tert-butyl 4-(1-(4-amino-2-ethyl-5-methoxyphenyl)piperidin-4-yl)piperazine-1-carboxylate

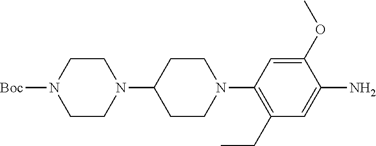 [SMILES: CCc1cc(N)c(OC)cc1N1CCC(N2CCN(C(=O)OC(C)(C)C)CC2)CC1]

To a stirred solution of tert-butyl 4-(1-(5-methoxy-4-nitro-2-vinylphenyl)piperidin-4-yl)piperazine-1-carboxylate (5 g, 11.2 mmol) in MeOH (100 mL) and DCM (20 mL) was added Pd/C (wet, 10%) (1 g) under nitrogen atmosphere. The resulting mixture was stirred for 16 hrs at room temperature under hydrogen atmosphere. The resulting mixture was filtered, the solid was washed with DCM/CH₃OH (10:1, 200 mL). The filtrate was concentrated under reduced pressure to afford the product (4.0 g, 85.3%). [M+H]⁺=419.1.

### Step 4: (6-aminoquinoxalin-5-yl)dimethylphosphine oxide

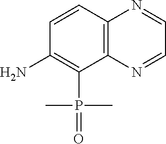

A mixture of 5-bromoquinoxalin-6-amine (10 g, 44.8 mmol), dimethylphosphine oxide (10.5 g, 134.5 mmol), Pd(OAc)₂ (1.0 g, 4.5 mmol) Xanphos (5.2 g, 9 mmol) and K₃PO₄ (28 g, 134 mmol) in DMF (250 mL) and water (50 mL) was stirred in a flask at 130° C. under nitrogen atmosphere for 16 hrs. The reaction mixture was allowed to cool down to room temperature. The resulting mixture was extracted with DCM (3×1000 mL). The combined organic layers were washed with brine (1000 mL), dried over anhydrous Na₂SO₄. After filtration, the filtrate was concentrated under reduced pressure. The residue was purified by silica gel column chromatography, eluted with DCM/MeOH (10:1) to afford the product (6 g, 60%), [M+H]⁺=222.0.

### Step 5: (6-((5-bromo-2-chloropyrimidin-4-yl)amino)quinoxalin-5-yl)dimethylphosphine oxide

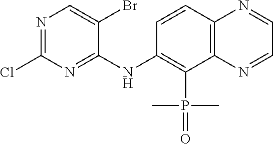 [SMILES: CP(C)(=O)c1c(Nc2nc(Cl)ncc2Br)ccc2nccnc12]

A mixture of (6-aminoquinoxalin-5-yl)dimethylphosphine oxide (6 g, 27.3 mmol), 5-bromo-2,4-dichloropyrimidine (12.3 g, 54.6 mmol) in THF (200 mL) was stirred in a flask at 0° C. under nitrogen atmosphere, before 54 mL KHMDS (1M in THF) was added. The reaction mixture was allowed to warm up to room temperature for 2 hours. The reaction was quenched with water and the mixture was extracted with DCM, washed three times with saturated brine, dried over anhydrous Na₂SO₄. After filtration, the filtrate was concentrated under reduced pressure. The residue was purified by silica gel column chromatography, eluted with DCM/MeOH (10:1) to afford the product (4 g, 35%). [M+H]⁺=412.0.

### Step 6: (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide

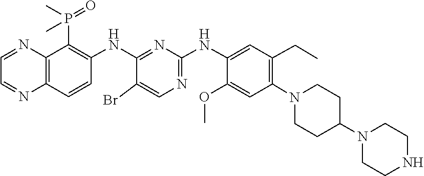 [SMILES: CCc1cc(Nc2ncc(Br)c(Nc3ccc4nccnc4c3P(C)(C)=O)n2)c(OC)cc1N1CCC(N2CCNCC2)CC1]

A mixture of(6-((5-bromo-2-chloropyrimidin-4-yl)amino)quinoxalin-5-yl)dimethylphosphine oxide (2 g, 4.8 mmol), tert-butyl 4-(1-(4-amino-2-ethyl-5-methoxyphenyl)piperidin-4-yl)piperazine-1-carboxylate (2.6 g, 6.3 mmol) and MsOH (184 mg, 1.92 mmol) in t-BuOH (20 mL) was stirred in a flask at 90° C. under nitrogen atmosphere for overnight. The reaction mixture was concentrated under reduced pressure. The residue was purified by silica gel column chromatography (DCM:MeOH=10:1) to afford the product (2 g, 60%). [M+H]⁺=694.0.

### Step 7: (R)-2-(4-(2,6-dioxopiperidin-3-yl)-3,5-difluorophenyl)acetaldehyde

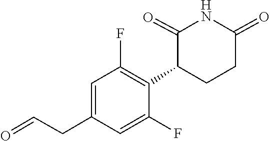 [SMILES: O=CCc1cc(F)c([C@H]2CCC(=O)NC2=O)c(F)c1]

(R,E)-3-(4-(2-ethoxyvinyl)-2,6-difluorophenyl)piperidine-2,6-dione (3.1 g, 10.4 mmol) was dissolved in FA(50 mL). The resulting solution was stirred for 2 h at room temperature. The reaction solution was evaporated to dryness to afford the product (2.6 g, 91.8%). [M+H]⁺=268.1.

### Step 8: (R)-3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

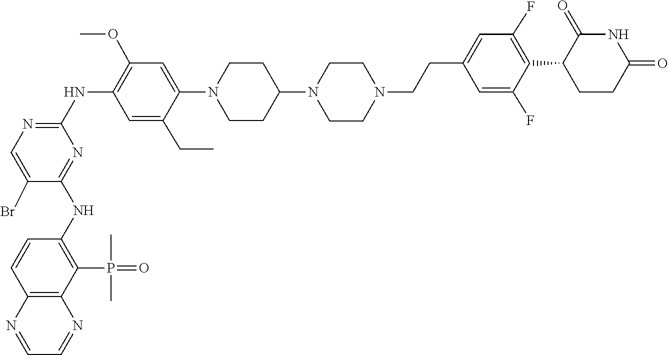 [SMILES: CCc1cc(Nc2ncc(Br)c(Nc3ccc4nccnc4c3P(C)(C)=O)n2)c(OC)cc1N1CCC(N2CCN(CCc3cc(F)c([C@H]4CCC(=O)NC4=O)c(F)c3)CC2)CC1]

The title compound (34 mg, 25%) was prepared in a manner similar to that in Example 21 step 9 from (6-((5-bromo-2-((5-ethyl-2-methoxy-4-(4-(piperazin-1-yl)piperidin-1-yl)phenyl)amino)pyrimidin-4-yl)amino)quinoxalin-5-yl)dimethylphosphine oxide and (R)-2-(4-(2,6-dioxopiperidin-3-yl)-3,5-difluorophenyl)acetaldehyde. ¹H NMR (500 MHz, DMSO) δ 12.65 (s, 1H), 10.96 (s, 1H), 8.86 (dt, J=22.8, 11.4 Hz, 3H), 8.28 (d, J=9.1 Hz, 2H), 7.90 (d, J=8.9 Hz, 1H), 7.37 (s, 1H), 7.03 (d, J=10.0 Hz, 2H), 6.81 (s, 1H), 4.20 (dd, J=12.8, 5.0 Hz, 1H), 3.77 (s, 3H), 3.01 (d, J=11.5 Hz, 2H), 2.76 (m, 6H), 2.54 (d, J=1.8 Hz, 6H), 2.48 (s, 5H), 2.36 (s, 2H), 2.13 (d, J=9.6 Hz, 1H), 2.02 (d, J=14.4 Hz, 7H), 1.87 (d, J=11.4 Hz, 2H), 1.58 (d, J=8.8 Hz, 2H), 0.93 (t, J=7.2 Hz, 3H); [M+H]⁺=945.6.

### Example 17: (S)-3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

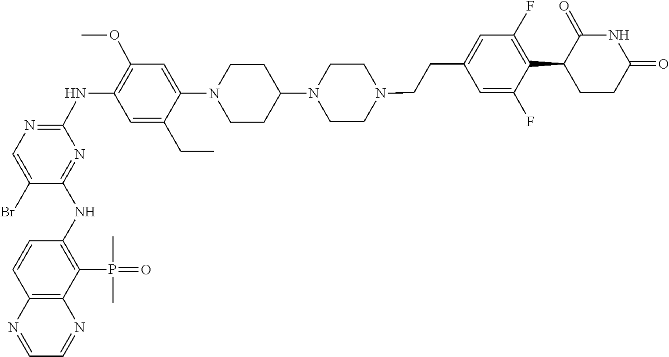 [SMILES: CCc1cc(Nc2ncc(Br)c(Nc3ccc4nccnc4c3P(C)(C)=O)n2)c(OC)cc1N1CCC(N2CCN(CCc3cc(F)c([C@@H]4CCC(=O)NC4=O)c(F)c3)CC2)CC1]

The title compound was synthesized in a manner similar to that in Example 16. ¹H NMR (500 MHz, DMSO) δ 12.65 (s, 1H), 10.96 (s, 1H), 8.86 (dt, J=22.8, 11.4 Hz, 3H), 8.28 (d, J=9.1 Hz, 2H), 7.90 (d, J=8.9 Hz, 1H), 7.37 (s, 1H), 7.03 (d, J=10.0 Hz, 2H), 6.81 (s, 1H), 4.20 (dd, J=12.8, 5.0 Hz, 1H), 3.77 (s, 3H), 3.01 (d, J=11.5 Hz, 2H), 2.76 (m, 6H), 2.54 (d, J=1.8 Hz, 6H), 2.48 (s, 5H), 2.36 (s, 2H), 2.13 (d, J=9.6 Hz, 1H), 2.02 (d, J=14.4 Hz, 7H), 1.87 (d, J=11.4 Hz, 2H), 1.58 (d, J=8.8 Hz, 2H), 0.93 (t, J=7.2 Hz, 3H); [M+H]⁺=945.6.

### Example 18: 3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

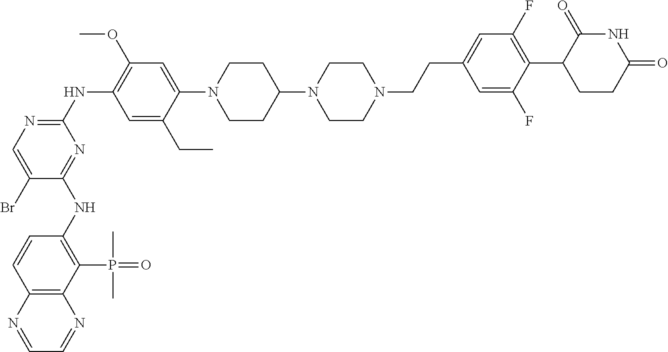 [SMILES: CCc1cc(Nc2ncc(Br)c(Nc3ccc4nccnc4c3P(C)(C)=O)n2)c(OC)cc1N1CCC(N2CCN(CCc3cc(F)c(C4CCC(=O)NC4=O)c(F)c3)CC2)CC1]

The title compound was synthesized in a manner similar to that in Example 16. ¹H NMR (500 MHz, DMSO) δ 12.65 (s, 1H), 10.96 (s, 1H), 8.86 (dt, J=22.8, 11.4 Hz, 3H), 8.28 (d, J=9.1 Hz, 2H), 7.90 (d, J=8.9 Hz, 1H), 7.37 (s, 1H), 7.03 (d, J=10.0 Hz, 2H), 6.81 (s, 1H), 4.20 (dd, J=12.8, 5.0 Hz, 1H), 3.77 (s, 3H), 3.01 (d, J=11.5 Hz, 2H), 2.76 (m, 6H), 2.54 (d, J=1.8 Hz, 6H), 2.48 (s, 5H), 2.36 (s, 2H), 2.13 (d, J=9.6 Hz, 1H), 2.02 (d, J=14.4 Hz, 7H), 1.87 (d, J=11.4 Hz, 2H), 1.58 (d, J=8.8 Hz, 2H), 0.93 (t, J=7.2 Hz, 3H); [M+H]⁺=945.6.

### Example 19:3-(4-(2-(4-(1-(4-((5-bromo-4-((5-(dimethylphosphoryl)quinoxalin-6-yl)amino)pyrimidin-2-yl)amino)-2-ethyl-5-methoxyphenyl)piperidin-4-yl)piperazin-1-yl)ethyl)phenyl)piperidine-2,6-dione

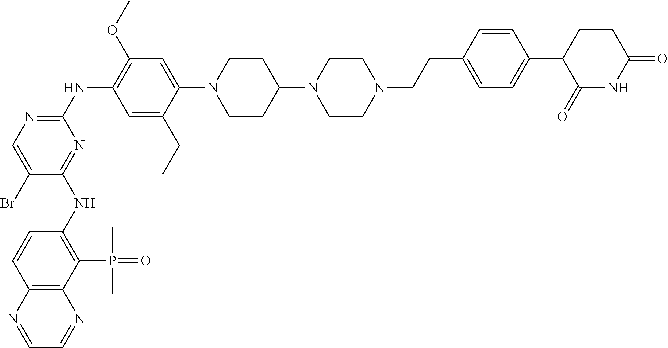 [SMILES: CCc1cc(Nc2ncc(Br)c(Nc3ccc4nccnc4c3P(C)(C)=O)n2)c(OC)cc1N1CCC(N2CCN(CCc3ccc(C4CCC(=O)NC4=O)cc3)CC2)CC1]

The title compound was synthesized in a manner similar to that in Example 16. ¹H NMR (500 MHz, DMSO) δ 12.65 (s, 1H), 10.83 (s, 1H), 8.87 (d, J=4.2 Hz, 3H), 8.28 (d, J=8.8 Hz, 2H), 7.92 (s, 2H), 7.16 (d, J=19.2 Hz, 4H), 6.81 (s, 1H), 3.77 (s, 4H), 3.00-3.02 (m, 4H), 2.71-2.75 (m, 6H), 2.26-2.40 (m, 12H), 2.02 (m, 7H), 1.85-1.87 (m, 2H), 1.58 (d, J=11.1 Hz, 2H), 0.93 (s, 3H); [M+H]⁺=909.3.

### Example 20: (S)-3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

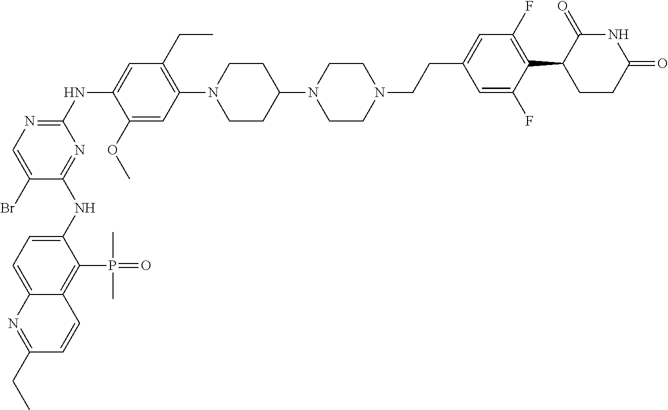 [SMILES: CCc1ccc2c(P(C)(C)=O)c(Nc3nc(Nc4cc(CC)c(N5CCC(N6CCN(CCc7cc(F)c([C@@H]8CCC(=O)NC8=O)c(F)c7)CC6)CC5)cc4OC)ncc3Br)ccc2n1]

A mixture of (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide (500 mg, 0.694 mmol) and (S)-2-(4-(2,6-dioxopiperidin-3-yl)-3,5-difluorophenyl)acetaldehyde (222.49 mg, 0.832 mmol, the compound was obtained through the similar way with “(R)-2-(4-(2,6-dioxopiperidin-3-yl)-3,5-difluorophenyl)acetaldehyde”) in dichloromethane (8 mL) was stirred in a flask at room temperature for 2 hour. To the mixture was added sodium triacetoxyborohydride (146.34 mg, 0.694 mmol) and the reaction was stirred at room temperature for another 2 h. The resulting mixture was diluted with H₂O (60 mL) and the layers were separated. The aqueous layer was extracted with DCM (3×30 mL). The combined organic layers were washed with brine (50 mL×3), dried over anhydrous Na₂SO₄, filtered and concentrated under reduced pressure. The residue was purified by silica gel column chromatography to afford the crude product (600 mg), which was purified with HPLC chromatography (0.1% FA in water:acetonitrile=90:10˜50:50 gradient elution) to give the product (475 mg, 70%). ¹H NMR (500 MHz, DMSO) δ 11.73 (s, 1H), 10.88 (s, 1H), 8.49 (d, J=8.8 Hz, 1H), 8.20 (s, 1H), 8.15 (d, J=12.4 Hz, 1H), 7.94 (s, 1H), 7.80 (d, J=9.4 Hz, 1H), 7.37 (d, J=8.9 Hz, 1H), 7.26 (s, 1H), 6.96 (d, J=10.0 Hz, 2H), 6.68 (s, 1H), 4.13 (dd, J=12.6, 5.0 Hz, 1H), 3.69 (s, 3H), 2.86 (dd, J=15.2, 7.6 Hz, 4H), 2.79-2.65 (m, 4H), 2.59 (t, J=11.3 Hz, 3H), 2.47 (s, 4H), 2.41-2.31 (m, 4H), 2.22 (d, J=4.7 Hz, 3H), 2.05 (s, 2H), 1.92 (d, J=13.3 Hz, 7H), 1.77 (d, J=10.2 Hz, 2H), 1.47 (d, J=8.8 Hz, 2H), 1.25 (t, J=7.6 Hz, 3H), 0.70 (s, 3H). [M+H]⁺=972.7

### Example 21: (R)-3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

### Step 1: 6-nitroquinolin-2-ol

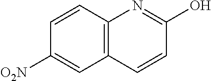 [SMILES: O=[N+]([O-])c1ccc2nc(O)ccc2c1]

To a solution of quinolin-2-ol (6 g, 41.3 mmol) in conc. H₂SO₄ (98%) (50 mL) was added dropwise a solution of conc. HNO₃ (63%) (3.12 g, 49.6 mmol) with stirring at 0° C. Then the mixture was stirred at rt for 1 h. The mixture was diluted with water (200 mL) at 0° C., the reaction mixture was filtered and the solid was washed with H₂O(500 ml ), dried in vacuum to afford 6-nitroquinolin-2-ol (5.5 g 69.9%) [M+H]⁺=191.1.

### Step 2: 2-chloro-6-nitroquinoline

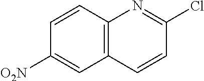 [SMILES: O=[N+]([O-])c1ccc2nc(Cl)ccc2c1]

A solution of 6-nitroquinolin-2-ol (5.5 g, 28.78 mmol) in POCl₃ (50 mL) was stirred at 100° C. for 2 hrs. Then the mixture was cooled to rt, then concentrated in vacuo. The residue was purified by Combi-Flash (silica column, 40 g, DCM:MeOH=15:1) to give 2-chloro-6-nitroquinoline (5 g 82.9%) [M+H]⁺=209.1.

### Step 3: 6-nitro-2-vinylquinoline

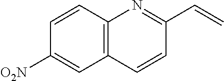

To a suspension of 2-chloro-6-nitroquinoline (5 g, 23.9 mmol) and 4,4,5,5-tetramethyl-2-vinyl-1,3,2-dioxaborolane (7.37 g, 47.84 mmol) in dioxane (40 mL) and water (10 mL) was added K₂CO₃ (9.91 g, 71.77 mmol) and Pd(dppf)Cl₂ (1.74 g, 2.39 mmol) under N₂ The mixture was warmed to 100° C. and stirred for 16 hrs. Then the mixture was cooled to rt and filtered. The filtrate was concentrated in vacuo. The residue was purified by Combi-Flash (silica column, 40 g, DCM:MeOH=15:1) to give 6-nitro-2-vinylquinoline (4.5 g, 93.9%). [M+H]⁺=201.1.

### Step 4: 2-ethylquinolin-6-amine

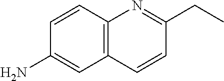

To a suspension of 6-nitro-2-vinylquinoline (4.5 g, 22.38 mmol) in MeOH (20 mL) was added Pd/C (1.5 g). The mixture was stirred at rt for 16 hrs under hydrogen atmosphere. Then the mixture was filtered and the solid was washed with MeOH. The filtrate was concentrated in vacuo to afford 2-ethylquinolin-6-amine (3.84 g, 99.2%). [M+H]⁺=173.1.

### Step 5: 2-ethyl-5-iodoquinolin-6-amine

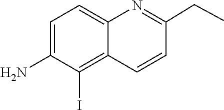

The title compound (4.5 g, 75.3%) was prepared in a manner similar to that in Example 22 step 1 from 2-ethylquinolin-6-amine and IC. [M+H]⁺=299.1.

### Step 6: (6-amino-2-ethylquinolin-5-yl)dimethylphosphine oxide

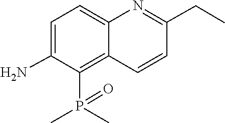 [SMILES: CCc1ccc2c(P(C)(C)=O)c(N)ccc2n1]

The title compound (3.5 g, 93.5%). was prepared in a manner similar to that in Example 22 step 2 from 2-ethyl-5-iodoquinolin-6-amine and dimethylphosphineoxide. [M+H]⁺=249.1.

### Step 7: (6-((5-bromo-2-chloropyrimidin-4-yl)amino)-2-ethylquinolin-5-yl)dimethylphosphine oxide

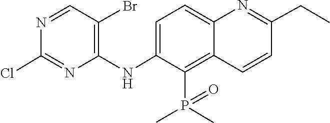 [SMILES: CCc1ccc2c(P(C)(C)=O)c(Nc3nc(Cl)ncc3Br)ccc2n1]

The title compound (2.5 g, 40.38%). was prepared in a manner similar to that in Example 22 step 3 from (6-amino-2-ethylquinolin-5-yl)dimethylphosphine oxide and 5-bromo-2,4-dichloropyrimidine. [M+H]⁺=439.6.

### Step 8: (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide

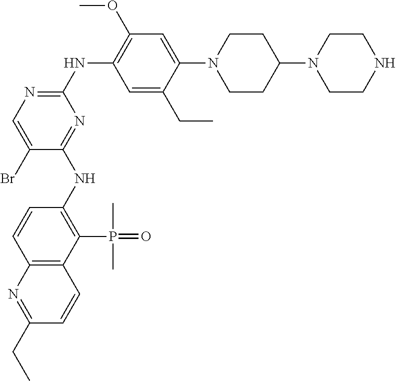 [SMILES: CCc1ccc2c(P(C)(C)=O)c(Nc3nc(Nc4cc(CC)c(N5CCC(N6CCNCC6)CC5)cc4OC)ncc3Br)ccc2n1]

The title compound (2.0 g, 48.78%) was prepared in a manner similar to that in Example 22 step 4 from (6-((5-bromo-2-chloropyrimidin-4-yl)amino)-2-ethylquinolin-5-yl)dimethylphosphine oxide and tert-butyl 4-(1-(4-amino-5-ethoxy-2-ethylphenyl)piperidin-4-yl)piperazine-1-carboxylate. [M+H]⁺=721.5.

### Step 9: (R)-3-(4-(2-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazin-1-yl)ethyl)-2,6-difluorophenyl)piperidine-2,6-dione

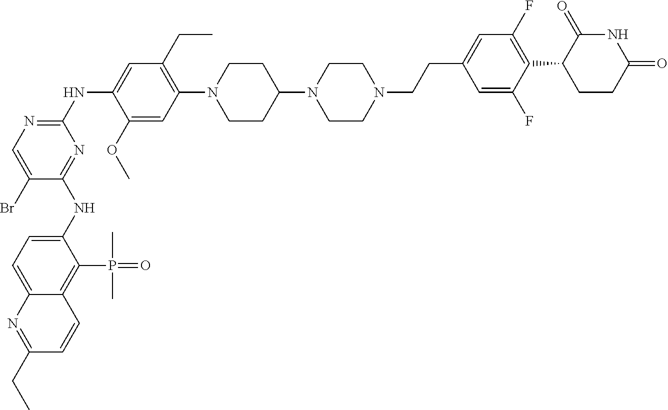 [SMILES: CCc1ccc2c(P(C)(C)=O)c(Nc3nc(Nc4cc(CC)c(N5CCC(N6CCN(CCc7cc(F)c([C@H]8CCC(=O)NC8=O)c(F)c7)CC6)CC5)cc4OC)ncc3Br)ccc2n1]

A mixture of (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide (500 mg, 0.694 mmol) and (R)-2-(4-(2,6-dioxopiperidin-3-yl)-3,5-difluorophenyl)acetaldehyde (222.49 mg, 0.832 mmol) in dichloromethane (8 mL) was stirred in a flask at room temperature for 2 hour. To the mixture was added sodium triacetoxyborohydride (146.34 mg, 0.694 mmol) and the reaction was stirred at room temperature for another 2 h. The resulting mixture was diluted with H₂O (60 mL) and the layers were separated. The aqueous layer was extracted with DCM (3×30 mL). The combined organic layers were washed with brine (50 mL×3), dried over anhydrous Na₂SO₄, filtered and concentrated under reduced pressure. The residue was purified with HPLC chromatography (0.1% FA in water:acetonitrile=90:10˜50:50 gradient elution) to give the product (480 mg, 71.2%). ¹H NMR (500 MHz, DMSO) δ 11.73 (s, 1H), 10.88 (s, 1H), 8.49 (d, J=8.8 Hz, 1H), 8.20 (s, 1H), 8.15 (d, J=12.4 Hz, 1H), 7.94 (s, 1H), 7.80 (d, J=9.4 Hz, 1H), 7.37 (d, J=8.9 Hz, 1H), 7.26 (s, 1H), 6.96 (d, J=10.0 Hz, 2H), 6.68 (s, 1H), 4.13 (dd, J=12.6, 5.0 Hz, 1H), 3.69 (s, 3H), 2.86 (dd, J=15.2, 7.6 Hz, 4H), 2.79-2.65 (m, 4H), 2.59 (t, J=11.3 Hz, 3H), 2.47 (s, 4H), 2.41-2.31 (m, 4H), 2.22 (d, J=4.7 Hz, 3H), 2.05 (s, 2H), 1.92 (d, J=13.3 Hz, 7H), 1.77 (d, J=10.2 Hz, 2H), 1.47 (d, J=8.8 Hz, 2H), 1.25 (t, J=7.6 Hz, 3H), 0.70 (s, 3H). [M+H]⁺=972.7

### Example 22: (R)-3-(4-((R)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

### Step 1: 5-iodo-2-methylquinolin-6-amine

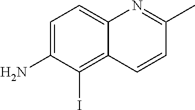

To a solution of 2-methylquinolin-6-amine (5 g, 31.62 mmol) in AcOH(50 mL) was added dropwise a solution of ICI (8.85 g, 37.95 mmol) in AcOH(10 mL). The mixture was stirred at 5° C.˜10° C. Then the mixture was stirred at rt for 1 h. The reaction solution was concentrated to dryness and the mixture was diluted with water (200 mL), neutralized with solid K₂CO₃. The mixture was extracted with DCM (3×150 mL). The combined organic phase was washed with brine (2×100 mL), dried over Na₂SO₄, filtered, and concentrated in vacuum. The residue was purified by column chromatography (DCM:MeOH=20:1) to afford product (7.1 g, 79.06%). [M+H]⁺=285.2.

### Step 2: (6-amino-2-methylquinolin-5-yl)dimethylphosphine oxide

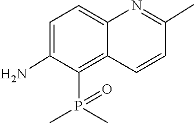 [SMILES: Cc1ccc2c(P(C)(C)=O)c(N)ccc2n1]

To a solution of 5-iodo-2-methylquinolin-6-amine (7.1 g, 24.99 mmol) and dimethylphosphine oxide (2.93 g, 37.49 mmol) in dioxane (100 mL) was added Pd(OAc)₂ (0.55 g, 2.45 mmol), Xantphos (2.89 g, 4.99 mmol), and K₃PO₄ (10.61 g, 49.98 mmol) under nitrogen atmosphere. The mixture was degassed under vacuum and purged with nitrogen several times. The mixture was stirred under nitrogen balloon at 100° C. for 6 h. The reaction mixture was extracted with DCM (3×50 mL). The combined organic phase was washed with brine (100 mL), dried over Na₂SO₄ and concentrated in vacuum. The residue was purified by column chromatography (DCM:MeOH=15:1) to afford the product (4.5 g, 76.9%). [M+H]⁺=235.2.

### Step 3: (6-((5-bromo-2-chloropyrimidin-4-yl)amino)-2-methylquinolin-5-yl)dimethylphosphine oxide

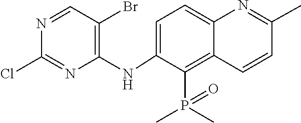 [SMILES: Cc1ccc2c(P(C)(C)=O)c(Nc3nc(Cl)ncc3Br)ccc2n1]

A solution of (6-amino-2-methylquinolin-5-yl)dimethylphosphine oxide (4.5 g, 19.21 mmol), 5-bromo-2,4-dichloropyrimidine (13.13 g, 57.64 mmol) and DIEA (7.45 g, 57.64 mmol) in n-BuOH (100 mL) was stirred at 120° C. for 12 h. The reaction solution was concentrated to dryness, then the crude product was purified by re-crystallization from EA:PE=5:1 (50 mL). The mixture was filtered and the filter cake was washed with DCM (3×50 mL). The combined organic phase was washed with brine (2×100 mL), dried over Na₂SO₄ and concentrated in vacuum to afford product (5.5 g, 67.3%). [M+H]⁺=425.2.

### Step 4: (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide

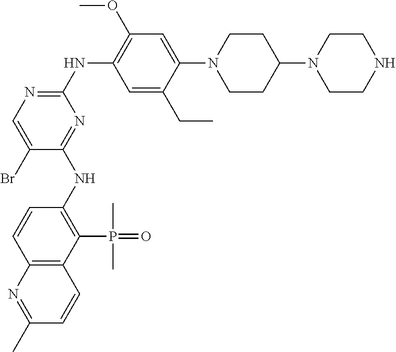 [SMILES: CCc1cc(Nc2ncc(Br)c(Nc3ccc4nc(C)ccc4c3P(C)(C)=O)n2)c(OC)cc1N1CCC(N2CCNCC2)CC1]

A solution of (6-((5-bromo-2-chloropyrimidin-4-yl)amino)-2-methylquinolin-5-yl)dimethylphosphine oxide (5.5 g, 12.91 mmol), TsOH (6.67 g, 38.73 mmol) and tert-butyl 4-(1-(4-amino-2-ethyl-5-methoxyphenyl)piperidin-4-yl)piperazine-1-carboxylate (5.67 g, 13.56 mmol) in n-BuOH (80 mL) was stirred at 100° C. for 12 h. The reaction mixture was adjusted to pH=8 with 1M NaOH, and then extracted with DCM (3×80 mL). The combined organic phase was washed with brine (2×100 mL), dried over Na₂SO₄ and concentrated in vacuum, The residue was purified by column chromatograph (DCM:MeOH=8:1) to afford the product (5.2 g, 57.01%). [M+H]⁺=707.3.

### Step 5: (R)-3-(4-((R)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

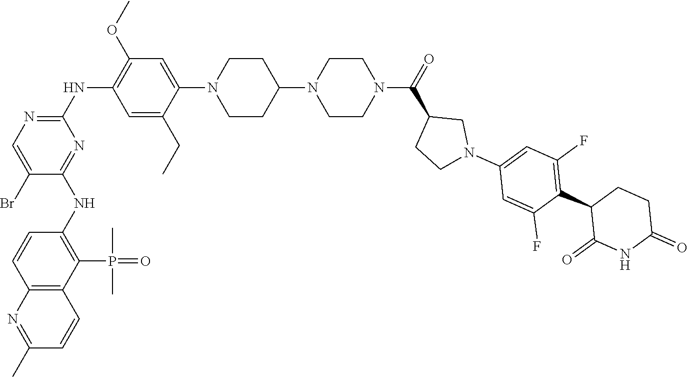 [SMILES: CCc1cc(Nc2ncc(Br)c(Nc3ccc4nc(C)ccc4c3P(C)(C)=O)n2)c(OC)cc1N1CCC(N2CCN(C(=O)[C@@H]3CCN(c4cc(F)c([C@H]5CCC(=O)NC5=O)c(F)c4)C3)CC2)CC1]

To a solution of (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide (50 mg, 0.07 mmol), (R)-1-(4-((R)-2,6-dioxopiperidin-3-yl)-3,5-difluorophenyl)pyrrolidine-3-carboxylic acid (24 mg, 0.07 mmol) and DIEA (26 mg, 0.2 mmol) in 10 mL DCM, 50% w.t. T₃P in EtOAc solution (64 mg, 0.1 mmol) was added. The mixture was stirred at 25° C. for 16 hours. After LCMS showed the reaction was completed, the mixture was quenched with 10 mL water. The organic phase was concentrated in vacuum and purified by prep-HPLC with C-18 column chromatography (0.1% FA in water:acetonitrile=90:10˜60:40 gradient elution) to afford the desired product (21.69 mg, 30.1% yield). ¹H NMR (500 MHz, DMSO) δ 11.76 (s, 1H), 10.84 (s, 1H), 8.56 (d, J=8.9 Hz, 1H), 8.30 (d, J=7.1 Hz, 1H), 8.21 (s, 1H), 7.98 (s, 1H), 7.87 (d, J=9.2 Hz, 1H), 7.46-7.33 (m, 2H), 6.74 (s, 1H), 6.23 (d, J=12.1 Hz, 2H), 4.02 (dd, J=12.6, 5.1 Hz, 1H), 3.76 (s, 3H), 3.59-3.42 (m, 6H), 3.36-3.22 (m, 4H), 2.95 (d, J=10.7 Hz, 2H), 2.83-2.73 (m, 1H), 2.70-2.62 (m, 5H), 2.56 (d, J=15.8 Hz, 2H), 2.45-2.35 (m, 3H), 2.30 (d, J=7.1 Hz, 2H), 2.21-2.03 (m, 3H), 2.03-1.91 (m, 7H), 1.84 (d, J=10.2 Hz, 2H), 1.65-1.50 (m, 2H), 0.92-0.63 (m, 3H). [M+H]⁺=1027.6.

### Example 24: (R)-3-(4-((R)-3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

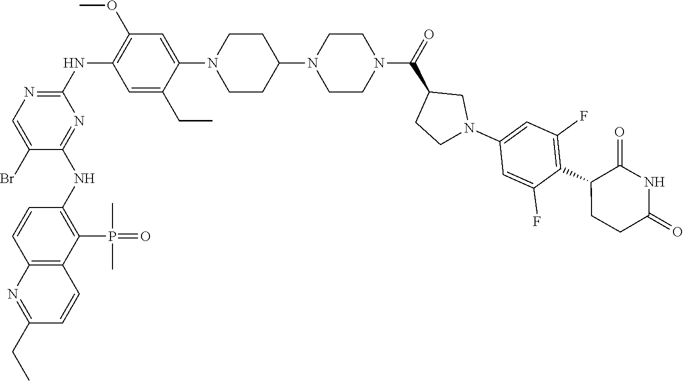 [SMILES: CCc1ccc2c(P(C)(C)=O)c(Nc3nc(Nc4cc(CC)c(N5CCC(N6CCN(C(=O)[C@@H]7CCN(c8cc(F)c([C@H]9CCC(=O)NC9=O)c(F)c8)C7)CC6)CC5)cc4OC)ncc3Br)ccc2n1]

A mixture of (6-((5-bromo-2-((5-ethyl-2-methoxy-4-(4-(piperazin-1-yl)piperidin-1-yl)phenyl)amino)pyrimidin-4-yl)amino)-2-ethylquinolin-5-yl)dimethylphosphine oxide (1.15 g, 1.60 mmol), (R)-1-(4-((R)-2,6-dioxopiperidin-3-yl)-3,5-difluorophenyl)pyrrolidine-3-carboxylic acid (595 mg, 1.76 mmol), DIEA (411 mg, 3.19 mmol) and T₃P (763 mg, 2.4 mmol) in dichloromethane (8 mL) was stirred in a flask at room temperature for 0.5 h. Then the mixture was evaporated in vacuum to afford the crude product, which was purified with prep-HPLC chromatography (0.10% FA in water:acetonitrile=90:10˜50:50 gradient elution) to give the product (800 mg, 48.3%). ¹H NMR (500 MHz, DMSO) δ 11.80 (s, 1H), 10.84 (s, 1H), 8.56 (d, J=8.9 Hz, 1H), 8.27 (s, 1H), 8.21 (s, 1H), 8.00 (s, 1H), 7.88 (d, J=9.1 Hz, 1H), 7.44 (d, J=8.9 Hz, 1H), 7.34 (s, 1H), 6.75 (s, 1H), 6.23 (d, J=12.2 Hz, 2H), 4.02 (dd, J=12.3, 4.7 Hz, 1H), 3.76 (s, 3H), 3.65-3.41 (m, 7H), 3.31-3.23 (m, 4H), 3.01-2.88 (m, 4H), 2.84-2.74 (m, 1H), 2.68 (t, J=11.1 Hz, 2H), 2.57 (m, 3H), 2.38 (m, 1H), 2.30 (m, 2H), 2.19-2.06 (m, 3H), 1.98 (d, J=13.3 Hz, 6H), 1.96-1.91 (m, 1H), 1.84 (d, J=10.1 Hz, 2H), 1.57 (d, J=9.9 Hz, 2H), 1.32 (t, J=7.5 Hz, 3H), 0.77 (s, 3H). [M+H]⁺=1041.7.

### Example 25: (S)-3-(4-((R)-3-(4-(1-(4-((5-bromo-4-((5-(dimethylphosphoryl)-2-ethylquinolin-6-yl)amino)pyrimidin-2-yl)amino)-2-ethyl-5-methoxyphenyl)piperidin-4-yl)piperazine-1-carbonyl)pyrrolidin-1-yl)-2,6-difluorophenyl)piperidine-2,6-dione

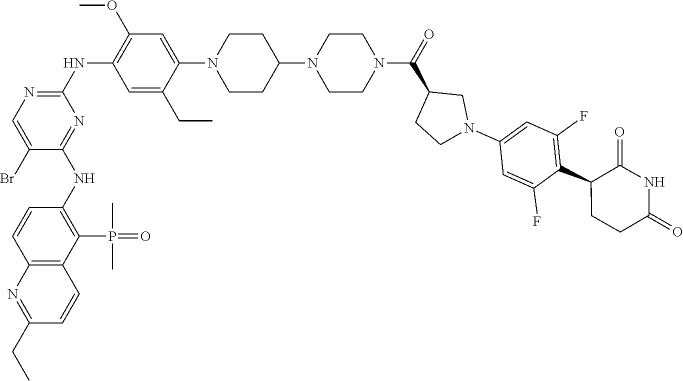 [SMILES: CCc1ccc2c(P(C)(C)=O)c(Nc3nc(Nc4cc(CC)c(N5CCC(N6CCN(C(=O)[C@@H]7CCN(c8cc(F)c([C@@H]9CCC(=O)NC9=O)c(F)c8)C7)CC6)CC5)cc4OC)ncc3Br)ccc2n1]

The title compound was prepared in the procedure similar to Example 24. ¹H NMR (500 MHz, DMSO) δ 11.80 (s, 1H), 10.84 (s, 1H), 8.56 (d, J=8.9 Hz, 1H), 8.27 (s, 1H), 8.21 (s, 1H), 8.00 (s, 1H), 7.88 (d, J=9.1 Hz, 1H), 7.44 (d, J=8.9 Hz, 1H), 7.34 (s, 1H), 6.75 (s, 1H), 6.23 (d, J=12.2 Hz, 2H), 4.02 (dd, J=12.3, 4.7 Hz, 1H), 3.76 (s, 3H), 3.65-3.41 (m, 7H), 3.31-3.23 (m, 4H), 3.01-2.88 (m, 4H), 2.84-2.74 (m, 1H), 2.68 (t, J=11.1 Hz, 2H), 2.57 (m, 3H), 2.38 (m, 1H), 2.30 (m, 2H), 2.19-2.06 (m, 3H), 1.98 (d, J=13.3 Hz, 6H), 1.96-1.91 (m, 1H), 1.84 (d, J=10.1 Hz, 2H), 1.57 (d, J=9.9 Hz, 2H), 1.32 (t, J=7.5 Hz, 3H), 0.77 (s, 3H). [M+H]⁺=1041.7.

### Example 26: (R)-3-(4-(2-(4-(1-(4-((5-bromo-4-((5-(dimethylphosphoryl)-2-methylquinolin-6-yl)amino)pyrimidin-2-yl)amino)-5-cyclopropoxy-2-ethylphenyl)piperidin-4-yl)piperazin-1-yl)ethyl)-2,6-difluorophenyl)piperidine-2,6-dione

### Step 1: 1-bromo-2-chloro-4-cyclopropoxy-5-nitrobenzene

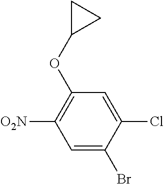 [SMILES: O=[N+]([O-])c1cc(Br)c(Cl)cc1OC1CC1]

To a solution of 1-bromo-2-chloro-4-fluoro-5-nitrobenzene (4 g, 15.7 mmol) in DMSO (50 mL) was added cyclopropanol (912 mg, 15.7 mmol) and K₂CO₃ (4.34 g, 31.4 mmol) at 20° C. Then the mixture was warmed to 70° C. and stirred for 16 hrs. Then the mixture was diluted with EA (200 mL), washed with water (100 mL×2) and brine (100 mL×2). Then the organic layer was combined and dried over anhydrous sodium sulfate, filtered and concentrated in vacuo. The residue was purified by Combi-Flash (silica column, 80 g, PE:EA=10:1) to give the product (3.5 g, 76.2%).

### Step 2: tert-butyl 4-(1-(2-bromo-5-cyclopropoxy-4-nitrophenyl)piperidin-4-yl)piperazine-1-carboxylate

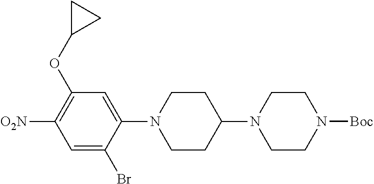 [SMILES: CC(C)(C)OC(=O)N1CCN(C2CCN(c3cc(OC4CC4)c([N+](=O)[O-])cc3Br)CC2)CC1]

To a solution of 1-bromo-2-chloro-4-cyclopropoxy-5-nitrobenzene (3.5 g, 12.0 mmol) in MeCN (50 mL) was added tert-butyl 4-(piperidin-4-yl)piperazine-1-carboxylate (3.56 g, 13.2 mmol) and K₂CO₃ (3.31 g, 24.0 mmol) at 25° C. Then the mixture was stirred at 80° C. for 16 hrs. Then the mixture was cooled to rt and filtered. The solid was washed with EA. Then the filtrate was concentrated in vacuo. The residue was purified by Combi-Flash (silica column, 80 g, DCM: MeOH=30:1) to give tert-butyl 4-(1-(2-bromo-5-cyclopropoxy-4-nitrophenyl)piperidin-4-yl)piperazine-1-carboxylate (4 g, 63.3%). [M+H]⁺=525.3.

### Step 3: tert-butyl 4-(1-(5-cyclopropoxy-4-nitro-2-vinylphenyl)piperidin-4-yl)piperazine-1-carboxylate

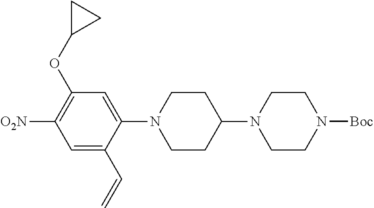 [SMILES: C=Cc1cc([N+](=O)[O-])c(OC2CC2)cc1N1CCC(N2CCN(C(=O)OC(C)(C)C)CC2)CC1]

To a suspension of tert-butyl 4-(1-(2-bromo-5-cyclopropoxy-4-nitrophenyl)piperidin-4-yl)piperazine-1-carboxylate (2 g, 3.8 mmol) and 4,4,5,5-tetramethyl-2-vinyl-1,3,2-dioxaborolane (879 mg, 5.7 mmol) in dioxane (16 mL) and water (4 mL) was added K₂CO₃ (1.57 g, 11.4 mmol) and Pd(dppf)Cl₂ (139 mg, 0.19 mmol) under nitrogen atmosphere. The mixture was warmed to 100° C. and stirred for 16 hrs. Then the mixture was cooled to rt and filtered. The filtrate was concentrated in vacuo. The residue was purified by Combi-Flash (silica column, 40 g, DCM: MeOH=15:1) to give tert-butyl 4-(1-(5-cyclopropoxy-4-nitro-2-vinylphenyl)piperidin-4-yl)piperazine-1-carboxylate (1.4 g, 77.9%). [M+H]⁺=473.3.

### Step 4: tert-butyl 4-(1-(4-amino-5-cyclopropoxy-2-ethylphenyl)piperidin-4-yl)piperazine-1-carboxylate

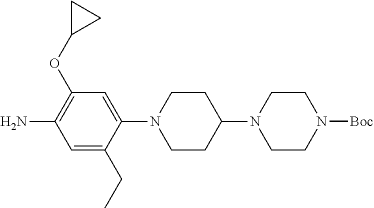 [SMILES: CCc1cc(N)c(OC2CC2)cc1N1CCC(N2CCN(C(=O)OC(C)(C)C)CC2)CC1]

To a suspension of tert-butyl 4-(1-(5-cyclopropoxy-4-nitro-2-vinylphenyl)piperidin-4-yl)piperazine-1-carboxylate (1.4 g, 3.0 mmol) in MeOH (20 mL) was added Pd/C (1 g). The mixture was stirred at rt for 16 hrs under hydrogen atmosphere. Then the mixture was filtered and the solid was washed with MeOH. The filtrate was concentrated in vacuo to afford tert-butyl 4-(1-(4-amino-5-cyclopropoxy-2-ethylphenyl)piperidin-4-yl)piperazine-1-carboxylate (1.2 g, 90.0%). [M+H]⁺=445.3.

### Step 5: (6-((5-bromo-2-((2-cyclopropoxy-5-ethyl-4-(4-(piperazin-1-yl)piperidin-1-yl)phenyl)amino)pyrimidin-4-yl)amino)-2-methylquinolin-5-yl)dimethylphosphine oxide

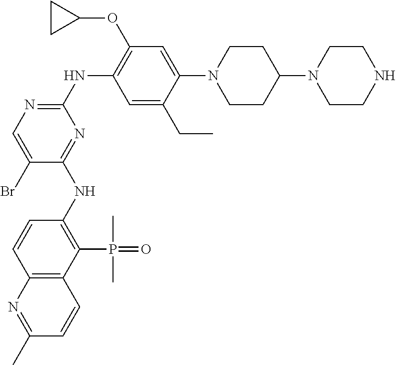 [SMILES: CCc1cc(Nc2ncc(Br)c(Nc3ccc4nc(C)ccc4c3P(C)(C)=O)n2)c(OC2CC2)cc1N1CCC(N2CCNCC2)CC1]

To a solution of (6-((5-bromo-2-chloropyrimidin-4-yl)amino)-2-methylquinolin-5-yl)dimethylphosphine oxide (553 mg, 1.3 mmol) in n-BuOH (10 mL) was added tert-butyl 4-(1-(4-amino-5-cyclopropoxy-2-ethylphenyl)piperidin-4-yl)piperazine-1-carboxylate (600 mg, 1.3 mmol) at 20° C. 4-Methylbenzenesulfonic acid (783 mg, 4.6 mmol) was added to the reaction mixture at 20° C. Then the mixture was stirred at 100° C. for 13 hrs. The mixture was diluted with water (100 mL), adjusted to pH=8 with 5N NaOH solution and then extracted with DCM (150 mL×3). The combined organic layers were washed with brine (150 mL×3), dried over Na₂SO₄, filtered and concentrated in vacuum. The residue was purified by column chromatography (DCM/MeOH (0.5% NH4OH)=10/1 to 5/1). (6-((5-bromo-2-((2-cyclopropoxy-5-ethyl-4-(4-(piperazin-1-yl)piperidin-1-yl)phenyl)amino)pyrimidin-4-yl)amino)-2-methylquinolin-5-yl)dimethylphosphine oxide (500 mg, 52%) was obtained. [M+H]⁺=733.2.

### Step 6: (R)-3-(4-(2-(4-(1-(4-((5-bromo-4-((5-(dimethylphosphoryl)-2-methylquinolin-6-yl)amino)pyrimidin-2-yl)amino)-5-cyclopropoxy-2-ethylphenyl)piperidin-4-yl)piperazin-1-yl)ethyl)-2,6-difluorophenyl)piperidine-2,6-dione

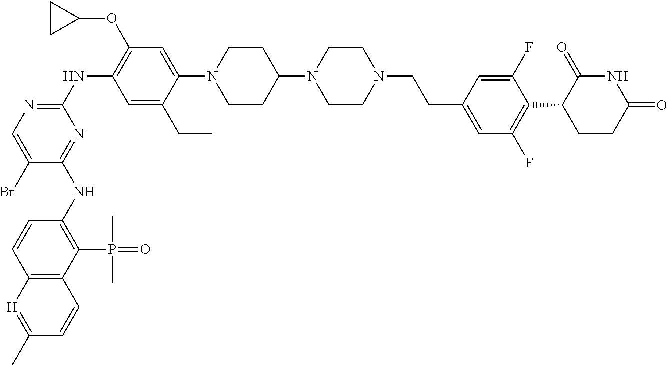

The title compound (32 mg, 48%) was prepared in a manner similar to that in Example 21 step 9 from (6-((5-bromo-2-((2-cyclopropoxy-5-ethyl-4-(4-(piperazin-1-yl)piperidin-1-yl)phenyl)amino)pyrimidin-4-yl)amino)-2-methylquinolin-5-yl)dimethylphosphine oxide and (R)-2-(4-(2,6-dioxopiperidin-3-yl)-3,5-difluorophenyl)acetaldehyde. ¹H NMR (500 MHz, DMSO) δ 11.74 (s, 1H), 10.95 (s, 1H), 8.58 (d, J=8.5 Hz, 1H), 8.27 (d, J=7.5 Hz, 1H), 8.21 (s, 1H), 7.85-7.84 (m, 2H), 7.43 (d, J=8.5 Hz, 1H), 7.39 (s, 1H), 7.04 (d, J=10.0 Hz, 2H), 6.98 (s, 1H), 4.20 (dd, J=12.5, 5.0 Hz, 1H), 3.81 (dq, J=9.0, 3.0 Hz, 1H), 2.98 (d, J=10.5 Hz, 2H), 2.85-2.76 (m, 4H), 2.75-2.51 (m, 14H), 2.17 (m, 5H), 1.96 (m, 9H), 1.69-1.53 (m, 2H), 0.75 (t, J=7.5, 3H), 0.70 (m, 2H), 0.61-0.56 (m, 2H). [M+H]⁺=984.3.

### Example 28 and 29: (R)-3-(4-((R)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)pyrrolidin-1-yl)phenyl)piperidine-2,6-dione (28) and (S)-3-(4-((R)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)pyrrolidin-1-yl)phenyl)piperidine-2,6-dione (29)

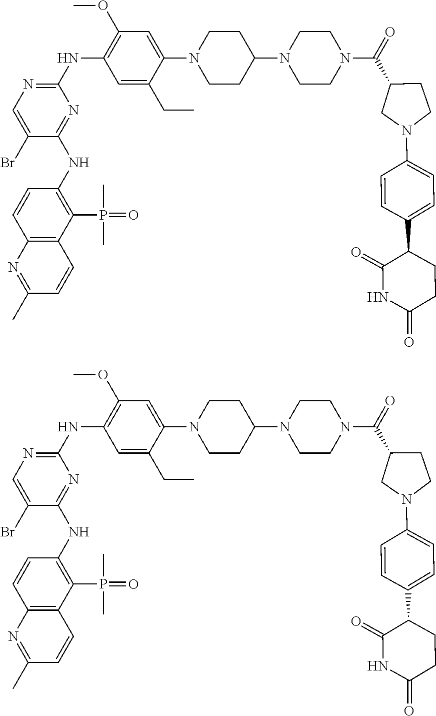 [SMILES: CCc1cc(Nc2ncc(Br)c(Nc3ccc4nc(C)ccc4c3P(C)(C)=O)n2)c(OC)cc1N1CCC(N2CCN(C(=O)[C@@H]3CCN(c4ccc([C@H]5CCC(=O)NC5=O)cc4)C3)CC2)CC1 ; CCc1cc(Nc2ncc(Br)c(Nc3ccc4nc(C)ccc4c3P(C)(C)=O)n2)c(OC)cc1N1CCC(N2CCN(C(=O)[C@@H]3CCN(c4ccc([C@@H]5CCC(=O)NC5=O)cc4)C3)CC2)CC1]

To the solution of (6-((5-bromo-2-((5-ethyl-2-methoxy-4-(4-(piperazin-1-yl)piperidin-1-yl)phenyl)amino)pyrimidin-4-yl)amino)-2-methylquinolin-5-yl)dimethylphosphine oxide (140 mg, 0.2 mmol), (3R)-1-(4-(2,6-dioxopiperidin-3-yl)phenyl)pyrrolidine-3-carboxylic acid (60 mg, 0.2 mmol) and DIEA (51 mg, 0.4 mmol) in 5 mL anhydrous DCM, T₃P (190 mg, 0.3 mmol, 50% w.t. EtOAc solution) was added. The mixture was stirred at room temperature for 30 minutes. Once the reaction has completed determined by LCMS, the mixture was diluted with 10 mL water. The mixture was extracted with DCM (10 mL×3). The combined organic layers were washed with brine (10 mL×3), dried over anhydrous Na₂SO₄, filtered and concentrated in vacuum. Purification by prep-TLC (DCM:MeOH=15:1) afforded the mixture of two diastereomers, which could be separated by chiral-HPLC (IF(2*25 cm, 5 um), 60% MtBE/40% MeOH:DCM=1:1/0.1% DEA, 80 bar, 20 ml/min). The product (R)-3-(4-((R)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)pyrrolidin-1-yl)phenyl)piperidine-2,6-dione (28) corresponded to peak A @ 1.192 min/254 nm) (2.7 mg, 1%) ¹H NMR (500 MHz, DMSO) δ 11.78 (s, 1H), 10.75 (s, 1H), 8.56 (d, J=8.8 Hz, 1H), 8.31 (d, J=5.2 Hz, 1H), 8.21 (s, 1H), 7.98 (s, 1H), 7.87 (d, J=9.2 Hz, 1H), 7.42 (d, J=8.9 Hz, 1H), 7.39 (s, 1H), 7.00 (d, J=8.3 Hz, 2H), 6.74 (s, 1H), 6.51 (d, J=8.4 Hz, 2H), 3.76 (s, 3H), 3.69 (dd, J=10.6, 4.8 Hz, 1H), 3.59-3.42 (m, 6H), 3.32-3.21 (m, 3H), 2.95 (d, J=10.2 Hz, 2H), 2.70-2.60 (m, 5H), 2.60-2.53 (m, 3H), 2.49-2.41 (m, 3H), 2.37 (m, 1H), 2.30 (d, J=6.5 Hz, 2H), 2.20-2.06 (m, 3H), 2.00 (m, 7H), 1.83 (d, J=10.6 Hz, 2H), 1.57 (m, 2H), 0.77 (s, 3H). [M+H]+=991.7. and (S)-3-(4-((R)-3-(4-(1-(4-((5-bromo-4-((5-(dimethylphosphoryl)-2-methylquinolin-6-yl)amino)pyrimidin-2-yl)amino)-2-ethyl-5-methoxyphenyl)piperidin-4-yl)piperazine-1-carbonyl)pyrrolidin-1-yl)phenyl)piperidine-2,6-dione (29) corresponded to peak B @ 2.190 min/254 nm (2.8 mg, 1%). ¹H NMR (500 MHz, DMSO) δ 11.78 (s, 1H), 10.75 (s, 1H), 8.56 (d, J=8.8 Hz, 1H), 8.31 (d, J=5.2 Hz, 1H), 8.21 (s, 1H), 7.98 (s, 1H), 7.87 (d, J=9.2 Hz, 1H), 7.42 (d, J=8.9 Hz, 1H), 7.39 (s, 1H), 7.00 (d, J=8.3 Hz, 2H), 6.74 (s, 1H), 6.51 (d, J=8.4 Hz, 2H), 3.76 (s, 3H), 3.69 (dd, J=10.6, 4.8 Hz, 1H), 3.57-3.40 (m, 6H), 3.30-3.19 (m, 3H), 2.95 (d, J=10.2 Hz, 2H), 2.72-2.63 (m, 5H), 2.60-2.53 (m, 3H), 2.49-2.41 (m, 3H), 2.37 (m, 1H), 2.30 (d, J=6.4 Hz, 2H), 2.15-2.06 (m, 3H), 2.00 (m, 7H), 1.83 (d, J=10.6 Hz, 2H), 1.57 (m, 2H), 0.77 (s, 3H). [M+H]⁺=991.7.

### Example 31: 3-(tert-butyl)-N-(4-(5-(4-(1-(4-(2,6-dioxopiperidin-3-yl)phenethyl)piperidin-4-yl)phenyl)-1H-pyrazolo[3,4-b]pyridin-3-yl)-2-methylbenzyl)-1,2,4-oxadiazole-5-carboxamide

### Step 1: (2-methyl-4-(4,4,5,5-tetramethyl-1,3,2-dioxaborolan-2-yl)phenyl)methanamine

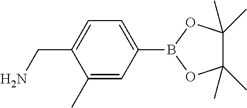 [SMILES: Cc1cc(B2OC(C)(C)C(C)(C)O2)ccc1CN]

A solution of tert-butyl (2-methyl-4-(4,4,5,5-tetramethyl-1,3,2-dioxaborolan-2-yl)benzyl)carbamate (1.05 g, 3.0 mmol) in HCl/dioxane (10 mL) was stirred at room temperature for 2 hours. After the reaction was completed, the solvent was removed under reduced pressure to give the desired product (850 mg, 99%). [M+H]⁺=248.1.

### Step 2: 5-(tert-butyl)-N-(2-methyl-4-(4,4,5,5-tetramethyl-1,3,2-dioxaborolan-2-yl)benzyl)-1,2,4-oxadiazole-3-carboxamide

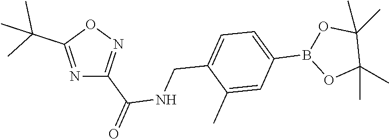 [SMILES: Cc1cc(B2OC(C)(C)C(C)(C)O2)ccc1CNC(=O)c1noc(C(C)(C)C)n1]

To a solution of (2-methyl-4-(4,4,5,5-tetramethyl-1,3,2-dioxaborolan-2-yl)phenyl)methanamine (850 mg, 3.0 mmol), sodium 5-(tert-butyl)-1,2,4-oxadiazole-3-carboxylate (860 mg, 4.5 mmol) and DIEA (1.2 g, 9.0 mmol) in DMF (10 mL) was added PyBOP (2.1 g, 4.5 mmol). The resulting mixture was stirred at room temperature for 1 h. The reaction was quenched with water and extracted with EtOAc. The combined organic layers were washed with brine, dried over anhydrous Na₂SO₄, and evaporated in vacuum to afford the crude product, which was further purified with silica gel column chromatography (PE: EtOAc=10: 1-2:1 gradient elution) to give the product (1.0 g, 73%). [M+H]⁺=400.2.

### Step 3: tert-butyl 5-bromo-3-(4-((5-(tert-butyl)-1,2,4-oxadiazole-3-carboxamido)methyl)-3-methylphenyl)-1H-pyrazolo[3,4-b]pyridine-1-carboxylate

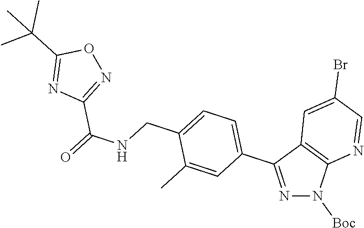 [SMILES: Cc1cc(-c2nn(C(=O)OC(C)(C)C)c3ncc(Br)cc23)ccc1CNC(=O)c1noc(C(C)(C)C)n1]

A mixture of tert-butyl 5-bromo-3-iodo-1H-pyrazolo[3,4-b]pyridine-1-carboxylate (550 mg, 1.3 mmol), 5-(tert-butyl)-N-(2-methyl-4-(4,4,5,5-tetramethyl-1,3,2-dioxaborolan-2-yl)benzyl)-1,2,4-oxadiazole-3-carboxamide (522 mg, 1.3 mmol), Pd(dppf)Cl₂ (95 mg, 0.13 mmol) and Cs₂CO₃ (638 mg, 1.95 mmol) in dioxane (15 mL)/H₂O (3 mL) was stirred in a round bottom flask at 80° C. for 1 h under nitrogen atmosphere. The solvent was removed under reduced pressure and the crude product was purified with silica gel column chromatography (PE: EtOAc=10: 1-4:1 gradient elution) to give the product (390 mg, 53%). [M+H]⁺=569.1.

### Step 4: tert-butyl 5-(4-(1-(tert-butoxycarbonyl)piperidin-4-yl)phenyl)-3-(4-((5-(tert-butyl)-1,2,4-oxadiazole-3-carboxamido)methyl)-3-methylphenyl)-1H-pyrazolo[3,4-b]pyridine-1-carboxylate

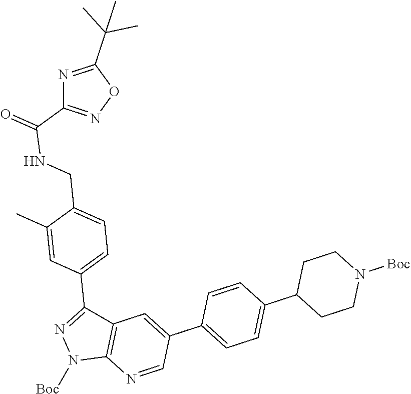 [SMILES: Cc1cc(-c2nn(C(=O)OC(C)(C)C)c3ncc(-c4ccc(C5CCN(C(=O)OC(C)(C)C)CC5)cc4)cc23)ccc1CNC(=O)c1noc(C(C)(C)C)n1]

A mixture of tert-butyl 5-bromo-3-(4-((5-(tert-butyl)-1,2,4-oxadiazole-3-carboxamido)methyl)-3-methylphenyl)-1H-pyrazolo[3,4-b]pyridine-1-carboxylate (390 mg, 0.68 mmol), tert-butyl 4-(4-(4,4,5,5-tetramethyl-1,3,2-dioxaborolan-2-yl)phenyl)piperidine-1-carboxylate (265 mg, 0.68 mmol), Pd(dppf)Cl₂ (50 mg, 0.068 mmol) and Cs₂CO₃ (335 mg, 1.03 mmol) in dioxane (15 mL)/H₂O (3 mL) was stirred in a round bottom flask at 90° C. for 3 h under N₂. The solvent was removed under reduced pressure and the crude product was purified with silica gel column chromatography (DCM: MeOH=100: 1-10:1 gradient elution) to give the product (270 mg, 53%). [M+H]⁺=750.5.

### Step 5: 5-(tert-butyl)-N-(2-methyl-4-(5-(4-(piperidin-4-yl)phenyl)-1H-pyrazolo[3,4-b]pyridin-3-yl)benzyl)-1,2,4-oxadiazole-3-carboxamide

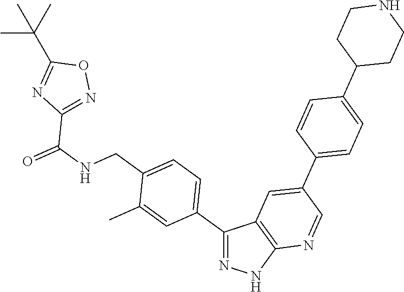 [SMILES: Cc1cc(-c2n[nH]c3ncc(-c4ccc(C5CCNCC5)cc4)cc23)ccc1CNC(=O)c1noc(C(C)(C)C)n1]

A solution of tert-butyl 5-(4-(1-(tert-butoxycarbonyl)piperidin-4-yl)phenyl)-3-(4-((5-(tert-butyl)-1,2,4-oxadiazole-3-carboxamido)methyl)-3-methylphenyl)-1H-pyrazolo[3,4-b]pyridine-1-carboxylate (270 mg, 0.36 mmol) in HCl/dioxane (10 mL) was stirred at room temperature for 2 hours. After the reaction was completed, the solvent was removed under reduced pressure to give the desired product (220 mg, 99%). [M+H]⁺=550.3.

### Step 6: 2-(4-(2,6-dioxopiperidin-3-yl)phenyl)acetaldehyde

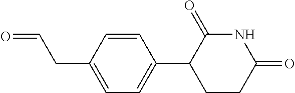 [SMILES: O=CCc1ccc(C2CCC(=O)NC2=O)cc1]

A mixture of 3-(4-(2-hydroxyethyl)phenyl)piperidine-2,6-dione (235 mg, 1 mmol) and IBX (420 mg, 1.5 mmol) in DMSO (10 mL) was stirred in a flask at 25° C. for 2 hrs. The reaction was quenched with water and extracted with EtOAc. The combined organic layers were washed with brine, dried over anhydrous Na₂SO₄, and evaporated in vacuum to afford the product (200 mg, 86%). [M+H]⁺=232.1.

### Step 7: 3-(tert-butyl)-N-(4-(5-(4-(1-(4-(2,6-dioxopiperidin-3-yl)phenethyl)piperidin-4-yl)phenyl)-1H-pyrazolo[34-b]pyridin-3-yl)-2-methylbenzyl)-1,2,4-oxadiazole-5-carboxamide

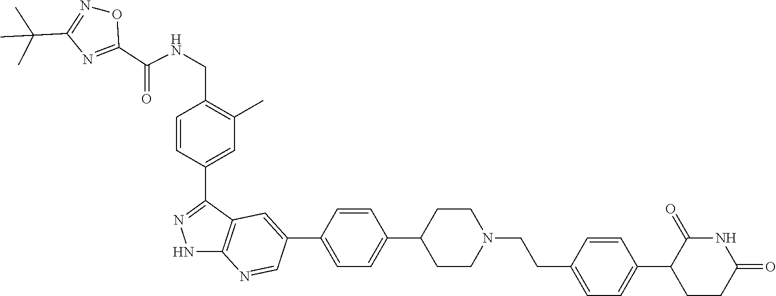 [SMILES: Cc1cc(-c2n[nH]c3ncc(-c4ccc(C5CCN(CCc6ccc(C7CCC(=O)NC7=O)cc6)CC5)cc4)cc23)ccc1CNC(=O)c1nc(C(C)(C)C)no1]

The title compound (12 mg, 25%) was prepared in a manner similar to that in Example 21 step 9 from 3-(tert-butyl)-N-(2-methyl-4-(5-(4-(piperazin-1-yl)phenyl)-1H-pyrazolo[3,4-b]pyridin-3-yl)benzyl)-1,2,4-oxadiazole-5-carboxamide and 2-(4-(2,6-dioxopiperidin-3-yl)phenyl)acetaldehyde. ¹H NMR (500 MHz, DMSO) δ 13.84 (s, 1H), 10.82 (s, 1H), 9.85 (t, J=6.0 Hz, 1H), 8.84 (d, J=2.0 Hz, 1H), 8.66 (d, J=1.9 Hz, 1H), 7.97-7.87 (m, 2H), 7.74 (d, J=8.2 Hz, 2H), 7.41 (m, 3H), 7.21 (d, J=8.1 Hz, 2H), 7.14 (d, J=8.1 Hz, 2H), 4.53 (d, J=5.9 Hz, 2H), 3.82 (dd, J=11.4, 4.9 Hz, 1H), 3.10 (d, J=11.2 Hz, 2H), 2.81-2.72 (m, 2H), 2.70-2.55 (m, 3H), 2.46 (m, 4H), 2.16 (m, 3H), 2.07-1.98 (m, 1H), 1.81 (m, 2H), 1.77-1.66 (m, 2H), 1.37 (s, 9H). [M+H]⁺=766.3.

### Example 32: (R)-3-(tert-butyl)-N-(4-(5-(4-(1-(4-(2,6-dioxopiperidin-3-yl)-3,5-difluorophenethyl)piperidin-4-yl)phenyl)-1H-pyrazolo[3,4-b]pyridin-3-yl)-2-methylbenzyl)-1,2,4-oxadiazole-5-carboxamide

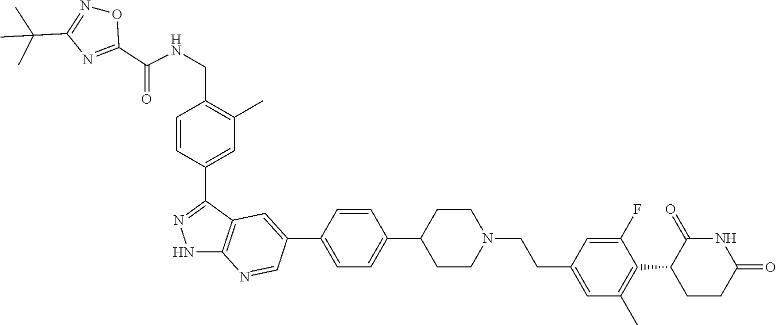 [SMILES: Cc1cc(-c2n[nH]c3ncc(-c4ccc(C5CCN(CCc6cc(C)c([C@H]7CCC(=O)NC7=O)c(F)c6)CC5)cc4)cc23)ccc1CNC(=O)c1nc(C(C)(C)C)no1]

The title compound was prepared in the procedure similar to that in Example 31. ¹H NMR (500 MHz, DMSO) δ 13.84 (s, 1H), 10.95 (s, 1H), 9.85 (t, J=6.0 Hz, 1H), 8.84 (d, J=2.0 Hz, 1H), 8.65 (d, J=2.0 Hz, 1H), 7.95-7.84 (m, 2H), 7.74 (d, J=8.2 Hz, 2H), 7.41 (dd, J=14.9, 8.1 Hz, 3H), 7.05 (d, J=10.0 Hz, 2H), 4.53 (d, J=5.9 Hz, 2H), 4.21 (dd, J=12.8, 5.0 Hz, 1H), 3.08 (d, J=10.1 Hz, 2H), 2.87-2.76 (m, 3H), 2.59 (m, 3H), 2.45 (s, 3H), 2.13 (m, 3H), 2.05-1.96 (m, 1H), 1.80 (m, 2H), 1.75-1.64 (m, 2H), 1.37 (s, 9H). [M+H]⁺=802.2.

### Example B33: 7-(4-((1-(4-(2,4-dioxotetrahydropyrimidin-1(2H)-yl)phenyl)piperidin-4-yl)methyl)piperazin-1-yl)-2-(2-fluoro-5-methyl-4-((6-methylpyridin-2-yl)carbamoyl)phenyl)-9,10-dihydro-4H-benzo[d]pyrazolo[1,5-a][1,3]diazepine-3-carboxamide

### Step 1: 5-amino-3-bromo-1-(2,5-dibromophenethyl)-1H-pyrazole-4-carbonitrile

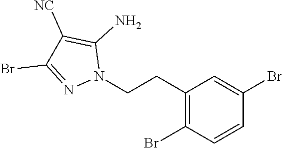 [SMILES: N#Cc1c(Br)nn(CCc2cc(Br)ccc2Br)c1N]

A mixture of 5-amino-3-bromo-1H-pyrazole-4-carbonitrile (20.0 g, 107 mmol), 2,5-dibromophenethyl 4-methylbenzenesulfonate (60.1 g, 139.2 mmol) and K₂CO₃ (29.6 g, 214 mmol) in DMF (300.0 mL) was stirred at 80° C. overnight. The mixture was treated with water (500 mL), extracted with dichloromethane (3×30 mL), and washed with brine (500 mL). The organic phase was dried over anhydrous sodium sulfate, filtered and concentrated under reduced pressure to afford the crude product, which was purified with silica gel column chromatography (DCM: MeOH=100:0˜93:7 gradient elution) to give the product (8.0 g, 16.8%). [M+H]⁺=448.0.

### Step 2: 2,7-dibromo-9,10-dihydro-4H-benzo[d]pyrazolo[1,5-a][1,3]diazepine-3-carbonitrile

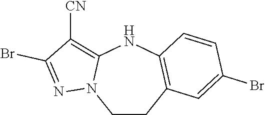 [SMILES: N#Cc1c(Br)nn2c1Nc1ccc(Br)cc1CC2]

A mixture of 5-amino-3-bromo-1-(2,5-dibromophenethyl)-1H-pyrazole-4-carbonitrile (8.0 g, 18 mmol), (1S,2S)—N1,N2-dimethylcyclohexane-1,2-diamine (383.4 mg, 2.7 mmol), copper(I) iodide (513 mg, 2.7 mmol), potassium carbonate (7.45 g, 54 mmol) in DMF (100 mL) was stirred at 95° C. overnight under nitrogen. The mixture was treated with water (300 mL) and filtered to give the crude product, which was purified with silica gel column chromatography (PE: EA=80:20˜50:50 gradient elution) to give the product (2.2 g, 33.3%). ¹H NMR (400 MHz, DMSO) δ 10.00 (s, 1H), 7.44 (s, 1H), 7.39 (d, J=8.5 Hz, 1H), 7.23 (d, J=8.4 Hz, 1H), 4.33 (s, 2H), 3.13 (s, 2H); [M+H]⁺=367.0.

### Step 3: benzyl 4-(2-bromo-3-cyano-9,10-dihydro-4H-benzo[d]pyrazolo[1,5-a][1,3]diazepin-7-yl)piperazine-1-carboxylate

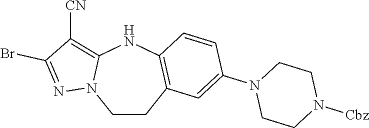 [SMILES: N#Cc1c(Br)nn2c1Nc1ccc(N3CCN(C(=O)OCc4ccccc4)CC3)cc1CC2]

A mixture of 2,7-dibromo-9,10-dihydro-4H-benzo[d]pyrazolo[1,5-a][1,3]diazepine-3-carbonitrile (1.6 g, 4.37 mmol), tert-butyl piperazine-1-carboxylate (1.44 g, 6.56 mmol), BrettPhos Pd G4 (349 mg, 0.437 mmol), BrettPhos (235 mg, 0.437 mmol) and LiHMDS (11 mL, 22 mmol, 2.0 M) in THF (10.0 mL) was stirred at 75° C. for 6 hours in sealed tube. Then the mixture was evaporated in vacuum to afford the crude product, which was purified with silica gel column chromatography (PE: EA=80:20˜50:50 gradient elution) to give the product (350 mg, 15.9%). ¹H NMR (400 MHz, DMSO) δ 9.64 (s, 1H), 7.35 (d, J=15.9 Hz, 5H), 7.15 (d, J=8.8 Hz, 1H), 6.84 (d, J=13.2 Hz, 2H), 5.11 (s, 2H), 4.28 (s, 2H), 3.53 (s, 4H), 3.06 (s, 6H); [M+H]⁺=507.0.

### Step 4: 4-bromo-5-fluoro-2-methyl-N-(6-methylpyridin-2-yl)benzamide

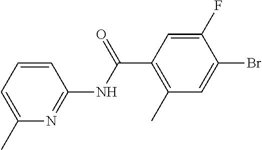 [SMILES: Cc1cccc(NC(=O)c2cc(F)c(Br)cc2C)n1]

A mixture of methyl 4-bromo-5-fluoro-2-methylbenzoate (1 g, 0.00406 mol) and 6-methylpyridin-2-amine (0.44 g, 0.00406 mol) in THF (30 mL) was stirred at 0° C.˜5° C. for 5 mins, then LiHMDS (6.2 mL, 0.00812 mol, 1.3 M) was added dropwise. The mixture was stirred at room temperature overnight. Then the mixture was quenched by NH₄Cl solution, extracted with EA (3×50 mL) and washed with brine (100 mL). The organic phase was dried over anhydrous Na₂SO₄, filtered, and concentrated under reduced pressure to afford the product (1.67 g, crude). [M+H]⁺=323.0.

### Step 5: 5-fluoro-2-methyl-N-(6-methylpyridin-2-yl)-4-(4,4,5,5-tetramethyl-1,3,2-dioxaborolan-2-yl)benzamide

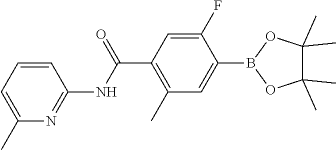 [SMILES: Cc1cccc(NC(=O)c2cc(F)c(B3OC(C)(C)C(C)(C)O3)cc2C)n1]

A mixture of 4-bromo-5-fluoro-2-methyl-N-(6-methylpyridin-2-yl)benzamide (1.5 g, 0.00467 mol), 4,4,4′,4′,5,5,5′,5′-octamethyl-2,2′-bi(1,3,2-dioxaborolane) (1.78 g, 0.00700 mol), Pd(dppf)Cl₂ (170 mg, 0.000233 mol) and KOAc (915 mg, 0.00934 mol) in dioxane (30.0 mL) was stirred at 93° C. overnight under N₂. The mixture was cooled down to room temperature, and concentrated under reduced pressure. The residue was purified by silica gel column chromatography PE: EA=100%:0%˜50%:50% to afford the product (530 mg, 31%). [M+H]⁺=371.0.

### Step 6: benzyl 4-(3-cyano-2-(2-fluoro-5-methyl-4-((6-methylpyridin-2-yl)carbamoyl)phenyl)-9,10-dihydro-4H-benzo[d]pyrazolo[1,5-a][1,3]diazepin-7-yl)piperazine-1-carboxylate

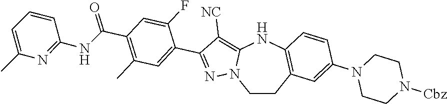 [SMILES: Cc1cccc(NC(=O)c2cc(F)c(-c3nn4c(c3C#N)Nc3ccc(N5CCN(C(=O)OCc6ccccc6)CC5)cc3CC4)cc2C)n1]

A mixture of benzyl 4-(2-bromo-3-cyano-9,10-dihydro-4H-benzo[d]pyrazolo[1,5-a][1,3]diazepin-7-yl)piperazine-1-carboxylate (725 mg, 1.43 mmol), 5-fluoro-2-methyl-N-(6-methylpyridin-2-yl)-4-(4,4,5,5-tetramethyl-1,3,2-dioxaborolan-2-yl)benzamide (530 mg, 1.43 mmol), Na₂CO₃ (303 mg, 1.86 mmol) and Pd(PPh₃)₄ (165 mg, 0.143 mmol) in dioxane/water (20 mL/4 mL) was stirred at 95° C. overnight under N₂. The mixture was cooled down to room temperature, and concentrated under reduced pressure. The residue was purified by silica gel column chromatography PE: EA=100%:0%˜0%:100% to afford the desired product (347 mg, 36%). [M+H]⁺=671.0.

### Step 7: 2-(2-fluoro-5-methyl-4-((6-methylpyridin-2-yl)carbamoyl)phenyl)-7-(piperazin-1-yl)-9,10-dihydro-4H-benzo[d]pyrazolo[1,5-a][1,3]diazepine-3-carboxamide

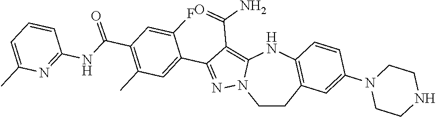 [SMILES: Cc1cccc(NC(=O)c2cc(F)c(-c3nn4c(c3C(N)=O)Nc3ccc(N5CCNCC5)cc3CC4)cc2C)n1]

A mixture of benzyl 4-(3-cyano-2-(2-fluoro-5-methyl-4-((6-methylpyridin-2-yl)carbamoyl)phenyl)-9,10-dihydro-4H-benzo[d]pyrazolo[1,5-a][1,3]diazepin-7-yl)piperazine-1-carboxylate (347 mg, 0.517 mmol) in methanesulfonic acid (10.0 mL) was stirred at 100° C. for 30 mins. The mixture was then cooled, basified with aqueous sodium hydroxide solution to pH 12, extracted with dichloromethane (3×30 mL) and washed with water (3×30 mL). The organic phase was dried over anhydrous sodium sulfate, filtered, and concentrated under reduced pressure to afford the product (225 mg, crude).

### Step 8: 7-(4-((1-(4-(2,4-dioxotetrahydropyrimidin-1(2H)-yl)phenyl)piperidin-4-yl)methyl)piperazin-1-yl)-2-(2-fluoro-5-methyl-4-((6-methylpyridin-2-yl)carbamoyl)phenyl)-9,10-dihydro-4H-benzo[d]pyrazolo[1,5-a][1,3]diazepine-3-carboxamide

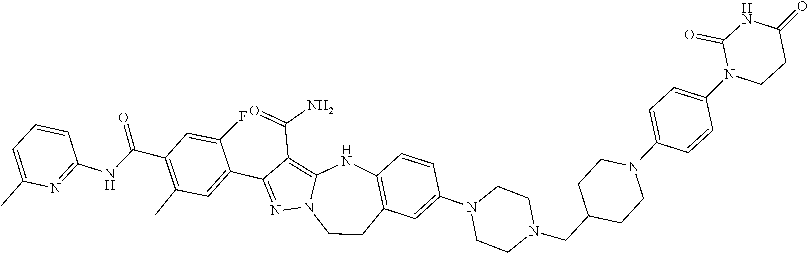 [SMILES: Cc1cccc(NC(=O)c2cc(F)c(-c3nn4c(c3C(N)=O)Nc3ccc(N5CCN(CC6CCN(c7ccc(N8CCC(=O)NC8=O)cc7)CC6)CC5)cc3CC4)cc2C)n1]

A mixture of 2-(2-fluoro-5-methyl-4-((6-methylpyridin-2-yl)carbamoyl)phenyl)-7-(piperazin-1-yl)-9,10-dihydro-4H-benzo[d]pyrazolo[1,5-a][1,3]diazepine-3-carboxamide (100 mg, crude), 1-(4-(2,4-dioxotetrahydropyrimidin-1(2H)-yl)phenyl)piperidine-4-carbaldehyde (70 mg, 0.234 mmol) in dichloromethane (5 mL) and MeOH (5 mL) was stirred at room temperature for 5 mins. Then HOAc (0.06 mL) was added. The mixture was stirred at room temperature overnight. Then NaBH(OAc)₃ (191 mg, 0.90 mmol) was added and stirred at room temperature for 2 hours. The reaction mixture was concentrated in vacuum, and the residue was purified by silica gel column chromatography (DCM: MeOH=100%: 0% 90%: 10% gradient elution) to give crude product, which was further purified by prep-TLC (DCM: MeOH=10:1) to afford desired product (9.28 mg, 6%). ¹H NMR (400 MHz, DMSO) δ 10.82 (s, 1H), 10.27 (s, 1H), 9.70 (s, 1H), 8.02 (d, J=8.0 Hz, 1H), 7.74 (t, J=8.0 Hz, 1H), 7.41 (dd, J=20.0, 8.0 Hz, 2H), 7.13 (d, J=8.0 Hz, 2H), 7.04 (d, J=8.0 Hz, 1H), 6.93 (d, J=8.0 Hz, 2H), 6.90-6.86 (m, 2H), 6.83 (d, J=8.0 Hz, 1H), 4.40-4.32 (m, 2H), 3.73-3.64 (m, 4H), 3.20-3.13 (m, 2H), 3.09 (s, 4H), 2.71-2.62 (m, 4H), 2.53-2.51 (m, 6H), 2.42 (s, 3H), 2.37 (s, 3H), 2.22 (d, J=8.0 Hz, 2H), 1.81 (d, J=12.0 Hz, 2H), 1.72 (s, 1H), 1.28-1.16 (m, 2H); [M+H]⁺=840.5.

### Example B34: 7-(4-((1-(4-(2,4-dioxotetrahydropyrimidin-1(2H)-yl)-3-methylphenyl)piperidin-4-yl)methyl)piperazin-1-yl)-2-(2-fluoro-3-methyl-4-((6-methylpyridin-2-yl)carbamoyl)phenyl)-9,10-dihydro-4H-benzo[d]pyrazolo[1,5-a][1,3]diazepine-3-carboxamide

### Step 1: 1-(4-(2,4-dioxotetrahydropyrimidin-1(2H)-yl)-3-methylphenyl)piperidine-4-carbaldehyde

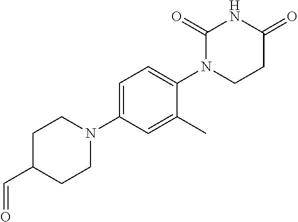 [SMILES: Cc1cc(N2CCC(C=O)CC2)ccc1N1CCC(=O)NC1=O]

To a mixture of 1-(4-(4-(hydroxymethyl)piperidin-1-yl)-2-methylphenyl)dihydropyrimidine-2,4(1H,3H)-dione (95.1 mg, 0.30 mmol) in DMSO (5 mL) was added IBX (109 mg, 0.39 mmol). The reaction was stirred at rt for 16 hours. Then the mixture was washed by water and extracted with dichloromethane (3×60 mL). The organic phase was combined and washed by brine. The mixture was dried over anhydrous sodium sulfate, filtered, and concentrated under reduced pressure to afford the crude product, which was purified with silica gel column chromatography (PE: EA=80:20˜30:70 gradient elution) to give the product (40 mg, 42%). [M+H]⁺=315.2.

### Step 2: 7-(4-((1-(4-(2,4-dioxotetrahydropyrimidin-1(2H)-yl)-3-methylphenyl)piperidin-4-yl)methyl)piperazin-1-yl)-2-(2-fluoro-3-methyl-4-((6-methylpyridin-2-yl)carbamoyl)phenyl)-9,10-dihydro-4H-benzo[d]pyrazolo[1,5-a][1,3]diazepine-3-carboxamide

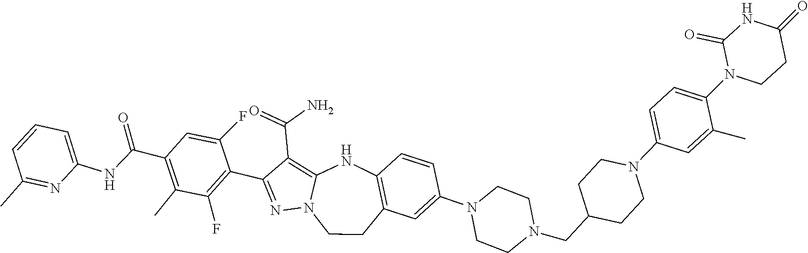 [SMILES: Cc1cccc(NC(=O)c2cc(F)c(-c3nn4c(c3C(N)=O)Nc3ccc(N5CCN(CC6CCN(c7ccc(N8CCC(=O)NC8=O)c(C)c7)CC6)CC5)cc3CC4)c(F)c2C)n1]

A mixture of 1-(4-(2,4-dioxotetrahydropyrimidin-1(2H)-yl)-3-methylphenyl)piperidine-4-carbaldehyde (40 mg, 0.13 mmol) and 2-(2-fluoro-3-methyl-4-((6-methylpyridin-2-yl)carbamoyl)phenyl)-7-(piperazin-1-yl)-9,10-dihydro-4H-benzo[d]pyrazolo[1,5-a][1,3]diazepine-3-carboxamide (the compound was synthesized through the similar way of the intermediate in B33) (70 mg, 0.13 mmol) in MeOH (2.0 mL), DCM (6.0 mL) and acetic acid (0.1 mL) was stirred at room temperature for 16 hours, and then NaBH(OAc)₃ (110 mg, 0.52 mmol) was added and stirred at room temperature for 1 hour. The mixture was treated with water (30 mL), extracted with dichloromethane (3×30 mL), and washed with brine (30 mL). The organic phase was dried over anhydrous sodium sulfate, filtered, and concentrated under reduced pressure to afford the crude product, which was purified with silica gel column chromatography (DCM: MeOH=100:0˜90:10 gradient elution) to give the product (14 mg, 16%). ¹H NMR (400 MHz, DMSO) δ 10.85 (s, 1H), 10.30 (s, 1H), 9.72 (s, 1H), 8.02 (d, J=8.5 Hz, 1H), 7.75 (t, J=7.9 Hz, 1H), 7.36 (q, J=7.9 Hz, 2H), 7.12-7.00 (m, 4H), 6.91-6.80 (m, 3H), 4.41-4.33 (m, 2H), 3.71 (t, J=6.7 Hz, 2H), 3.20-3.00 (m, 9H), 2.64 (dt, J=23.7, 9.2 Hz, 6H), 2.42 (s, 3H), 2.33-2.20 (m, 8H), 1.88-1.79 (m, 2H), 1.77-1.63 (m, 1H), 1.36-1.19 (m, 4H); [M+H]⁺=854.7.

### Example B35: (R)-7-(4-(4-(2,6-dioxopiperidin-3-yl)-3,5-difluorophenethyl)piperazin-1-yl)-2-(2-fluoro-3-methyl-4-((6-methylpyridin-2-yl)carbamoyl)phenyl)-9,10-dihydro-4H-benzo[d]pyrazolo[1,5-a][1,3]diazepine-3-carboxamide

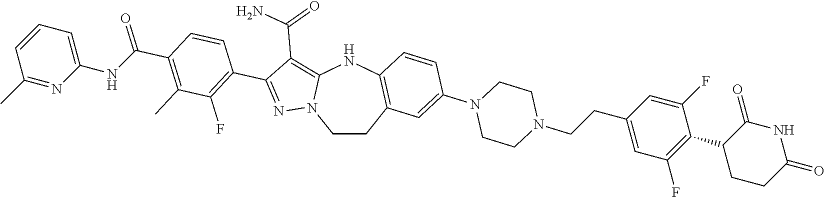

The titled compound was synthesized in the procedures similar to Example B34. ¹H NMR (500 MHz, DMSO) δ 10.98 (s, 1H), 10.82 (s, 1H), 9.79 (s, 1H), 8.02 (d, J=8.1 Hz, 1H), 7.75 (t, J=7.9 Hz, 1H), 7.40-7.32 (m, 2H), 7.14 (d, J=9.8 Hz, 2H), 7.05 (d, J=7.5 Hz, 1H), 6.98-6.91 (m, 3H), 4.41-4.36 (m, 2H), 4.24 (dd, J=12.6, 5.0 Hz, 1H), 3.84-3.77 (m, 2H), 3.69-3.62 (m, 2H), 3.57-2.48 (m, 2H), 3.25-3.15 (m, 4H), 3.11-3.06 (m, 2H), 3.02-2.94 (m, 2H), 2.88-2.77 (m, 1H), 2.58-2.52 (m, 1H), 2.43 (s, 3H), 2.31 (d, J=1.7 Hz, 3H), 2.18-2.10 (m, 1H), 2.04-1.97 (m, 1H); [M+H]⁺=806.6.

### Example B36: 7-(4-((1-(4-(2,4-dioxotetrahydropyrimidin-1(2H)-yl)phenyl)piperidin-4-yl)methyl)piperazin-1-yl)-2-(2-fluoro-3-methyl-4-((1-methyl-1H-imidazol-4-yl)carbamoyl)phenyl)-9,10-dihydro-4H-benzo[d]pyrazolo[1,5-a][1,3]diazepine-3-carboxamide

### Step 1: 4-bromo-2-fluoro-3-methylaniline

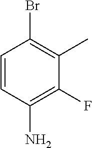

To a solution of 2-fluoro-3-methylaniline (11.5 g, 92 mmol) dissolved in ACN (170 mL) was added NBS (19.7 g, 110.4 mmol) at 0° C. The mixture was stirred at room temperature for 3 h. The mixture was washed by water and extracted with DCM. The organic layers were combined and dried over Na₂SO₄ and filtered. The filtrate was concentrated in vacuum. The residue was purified by silica gel column chromatography (PE: EtOAc=9:1) to afford the product (14.6 g, 78%). [M+H]⁺=204.1.

### Step 2: methyl 4-amino-3-fluoro-2-methylbenzoate

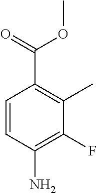

To a solution of 4-bromo-2-fluoro-3-methylaniline (10.2 g, 50 mmol), Pd(dppf)Cl₂ (1.83 g, 2.50 mmol) dissolved in MeOH (140 mL) was dropwise added Et₃N (10.1 g, 100 mmol). The mixture was stirred at 90° C. for 16 h under CO atmosphere. The mixture was concentrated in vacuum. The mixture was washed by water and extracted with EtOAc. The organic layers were combined and dried over Na₂SO₄ and filtered. The filtrate was concentrated in vacuum. The residue was purified by silica gel column chromatography (PE: EtOAc=4:1) to afford the product (2.53 g, 28%). [M+H]⁺=184.1.

### Step 3: methyl 3-fluoro-2-methyl-4-(4,4,5,5-tetramethyl-1,3,2-dioxaborolan-2-yl)benzoate

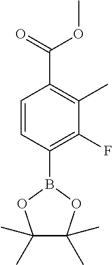 [SMILES: COC(=O)c1ccc(B2OC(C)(C)C(C)(C)O2)c(F)c1C]

To a solution of methyl 4-amino-3-fluoro-2-methylbenzoate (2.53 g, 13.8 mmol), tert-butyl nitrite (2.84 g, 27.6 mmol) and BPO (334 mg, 1.38 mmol) dissolved in ACN (70 mL) was added (Bpin)₂ (3.86 g, 15.2 mmol). The mixture was stirred at room temperature for 16 h. The mixture was concentrated in vacuum. The mixture was washed by water and extracted with EtOAc. The organic layers were combined, dried over Na₂SO₄ and filtered. The filtrate was concentrated in vacuum. The residue was purified by silica gel column chromatography (PE: EtOAc=12:1) to afford the product (1.83 g, 45%). ¹H NMR (500 MHz, CDCl₃) δ 7.67-7.53 (m, 2H), 3.90 (s, 3H), 2.47 (d, J=2.1 Hz, 3H), 1.37 (s, 12H).

### Step 4: benzyl 4-(3-cyano-2-(2-fluoro-4-(methoxycarbonyl)-3-methylphenyl)-9,10-dihydro-4H-benzo[d]pyrazolo[1,5-a][1,3]diazepin-7-yl)piperazine-1-carboxylate

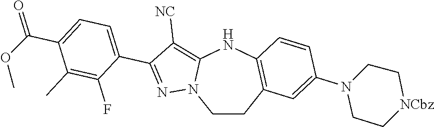 [SMILES: COC(=O)c1ccc(-c2nn3c(c2C#N)Nc2ccc(N4CCN(C(=O)OCc5ccccc5)CC4)cc2CC3)c(F)c1C]

A mixture of benzyl 4-(2-bromo-3-cyano-9,10-dihydro-4H-benzo[d]pyrazolo[1,5-a][1,3]diazepin-7-yl)piperazine-1-carboxylate (0.84 g, 1.66 mmol), methyl 3-fluoro-2-methyl-4-(4,4,5,5-tetramethyl-1,3,2-dioxaborolan-2-yl)benzoate (0.636 g, 2.16 mmol), Pd(dppf)Cl₂ (0.124 g, 0.17 mmol) and Na₂CO₃ (0.352 g, 3.32 mmol) in dioxane (25 mL) and H₂O (3 mL) was stirred at 95° C. for 16 hours under N₂. Then the mixture was washed by water and extracted with dichloromethane (3×60 mL). The organic phase was dried over anhydrous sodium sulfate, filtered, and concentrated under reduced pressure to afford the crude product, which was purified with silica gel column chromatography (PE: EA=80:20˜50:50 gradient elution) to give the product (345 mg, 35%). [M+H]⁺=595.3.

### Step 5: benzyl 4-(3-cyano-2-(2-fluoro-3-methyl-4-((1-methyl-1H-imidazol-4-yl)carbamoyl)phenyl)-9,10-dihydro-4H-benzo[d]pyrazolo[1,5-a][1,3]diazepin-7-yl)piperazine-1-carboxylate

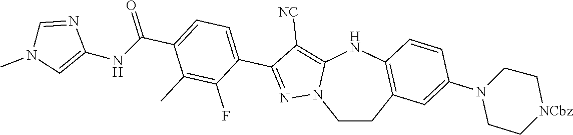 [SMILES: Cc1c(C(=O)Nc2cn(C)cn2)ccc(-c2nn3c(c2C#N)Nc2ccc(N4CCN(C(=O)OCc5ccccc5)CC4)cc2CC3)c1F]

To a mixture of benzyl 4-(3-cyano-2-(2-fluoro-4-(methoxycarbonyl)-3-methylphenyl)-9,10-dihydro-4H-benzo[d]pyrazolo[1,5-a][1,3]diazepin-7-yl)piperazine-1-carboxylate (0.345 g, 0.581 mmol) and 1-methyl-1H-imidazol-4-amine hydrochloride (0.077 g, 0.581 mmol) in THF (8.0 mL) was added LiHMDS (1.74 mL, 1 M) dropwise. The reaction was stirred at 60° C. for 2 hours and then cooled. The mixture was washed by water and extracted with dichloromethane (3×30 mL). The organic phase was dried over anhydrous sodium sulfate, filtered, and concentrated under reduced pressure to afford the product (365 mg, 95%). [M+H]⁺=660.2.

### Step 6: 2-(2-fluoro-3-methyl-4-((1-methyl-1H-imidazol-4-yl)carbamoyl)phenyl)-7-(piperazin-1-yl)-9,10-dihydro-4H-benzo[d]pyrazolo[1,5-a][1,3]diazepine-3-carboxamide

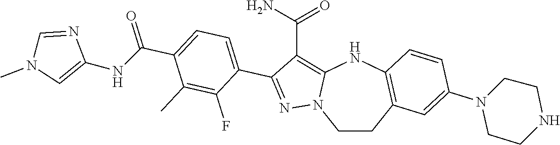 [SMILES: Cc1c(C(=O)Nc2cn(C)cn2)ccc(-c2nn3c(c2C(N)=O)Nc2ccc(N4CCNCC4)cc2CC3)c1F]

A mixture of benzyl 4-(3-cyano-2-(2-fluoro-3-methyl-4-((1-methyl-1H-imidazol-4-yl)carbamoyl)phenyl)-9,10-dihydro-4H-benzo[d]pyrazolo[1,5-a][1,3]diazepin-7-yl)piperazine-1-carboxylate (0.36 g, 0.55 mmol) in methanesulfonic acid (10.0 mL) was stirred at 100° C. for 1 hour. The mixture was then cooled, acidified with aqueous sodium hydroxide solution to pH 12 and extracted with dichloromethane (3×30 mL) and water (3×30 mL). The organic phase was dried over anhydrous sodium sulfate, filtered, and concentrated under reduced pressure to afford the product (290 mg, 97%). [M+H]⁺=544.1.

### Step 7: 7-(4-((1-(4-(2,4-dioxotetrahydropyrimidin-1(2H)-yl)phenyl)piperidin-4-yl)methyl)piperazin-1-yl)-2-(2-fluoro-3-methyl-4-((1-methyl-1H-imidazol-4-yl)carbamoyl)phenyl)-9,10-dihydro-4H-benzo[d]pyrazolo[1,5-a][1,3]diazepine-3-carboxamide

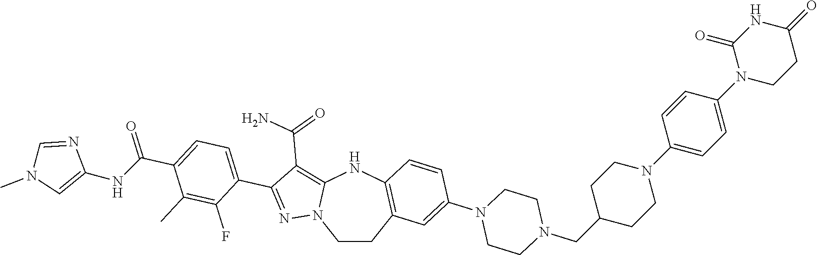 [SMILES: Cc1c(C(=O)Nc2cn(C)cn2)ccc(-c2nn3c(c2C(N)=O)Nc2ccc(N4CCN(CC5CCN(c6ccc(N7CCC(=O)NC7=O)cc6)CC5)CC4)cc2CC3)c1F]

A mixture of 2-(2-fluoro-3-methyl-4-((1-methyl-1H-imidazol-4-yl)carbamoyl)phenyl)-7-(piperazin-1-yl)-9,10-dihydro-4H-benzo[d]pyrazolo[1,5-a][1,3]diazepine-3-carboxamide (140 mg, 0.26 mmol) and 1-(4-(2,4-dioxotetrahydropyrimidin-1(2H)-yl)phenyl)piperidine-4-carbaldehyde (78 mg, 0.26 mmol) in MeOH (2.0 mL), DCM (6.0 mL) and acetic acid (0.1 mL) was stirred at room temperature for 16 hours, and then NaBH(OAc)₃ (220 mg, 1.04 mmol) was added and stirred at room temperature for 1 hour. The mixture was treated with water (30 mL), extracted with dichloromethane (3×30 mL), and washed with brine (30 mL). The organic phase was dried over anhydrous sodium sulfate, filtered, and concentrated under reduced pressure to afford the crude product, which was purified with silica gel column chromatography (DCM: MeOH=100: 0-85:15 gradient elution) to give the product (120 mg, 56%). ¹H NMR (500 MHz, DMSO) δ 10.76 (s, 1H), 10.25 (s, 1H), 9.69 (s, 1H), 7.43 (s, 1H), 7.38 (s, 1H), 7.33 (s, 2H), 7.13 (d, J=8.8 Hz, 2H), 6.93 (d, J=8.9 Hz, 2H), 6.88 (d, J=8.5 Hz, 2H), 6.83 (d, J=8.4 Hz, 1H), 4.39-4.33 (m, 2H), 3.75-3.61 (m, 8H), 3.33 (d, J=1.5 Hz, 3H), 3.19-3.14 (m, 2H), 3.11-3.03 (m, 4H), 2.69-2.62 (m, 5H), 2.28 (d, J=1.6 Hz, 3H), 2.25-2.19 (m, 2H), 1.84-1.78 (m, 2H), 1.76-1.66 (m, 1H), 1.30-1.16 (m, 3H); [M+H]⁺=829.6.

### Example B37: (R)-7-(4-(4-(2,6-dioxopiperidin-3-yl)-3,5-difluorophenethyl)piperazin-1-yl)-2-(2-fluoro-3-methyl-4-((1-methyl-1H-imidazol-4-yl)carbamoyl)phenyl)-9,10-dihydro-4H-benzo[d]pyrazolo[1,5-a][1,3]diazepine-3-carboxamide

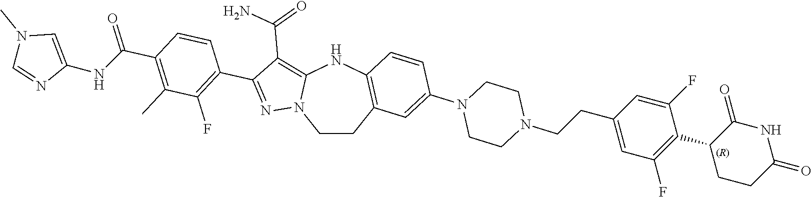

The titled compound was synthesized in the procedures similar to Example B36. ¹H NMR (500 MHz, DMSO) δ 10.95 (s, 1H), 10.75 (s, 1H), 9.69 (s, 1H), 7.43 (s, 1H), 7.38 (d, J=1.1 Hz, 1H), 7.35-7.31 (m, 2H), 7.06 (d, J=10.0 Hz, 2H), 6.88 (d, J=8.4 Hz, 2H), 6.86-6.82 (m, 1H), 4.40-4.32 (m, 2H), 4.20 (dd, J=12.5, 4.9 Hz, 1H), 3.66 (s, 3H), 3.19-3.14 (m, 2H), 3.12-3.06 (m, 4H), 2.85-2.77 (m, 3H), 2.65-2.53 (m, 7H), 2.28 (d, J=2.0 Hz, 3H), 2.18-2.08 (m, 1H), 2.04-1.97 (m, 1H); [M+H]⁺=795.5.

### Example B38: (R)-7-(4-(4-(2,6-dioxopiperidin-3-yl)-3,5-difluorophenethyl)piperazin-1-yl)-2-(2-fluoro-3-methyl-4-(thiazol-4-ylcarbamoyl)phenyl)-9,10-dihydro-4H-benzo[d]pyrazolo[1,5-a][1,3]diazepine-3-carboxamide

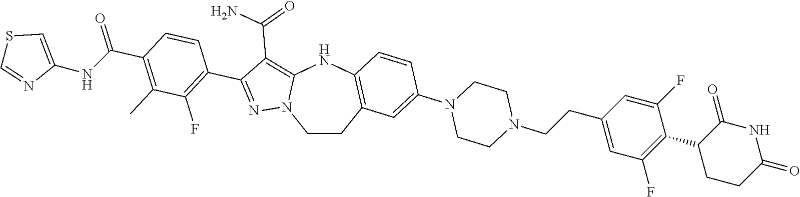

The titled compound was synthesized in the procedures similar to Example B36. ¹H NMR (500 MHz, DMSO) δ 11.52 (s, 1H), 10.98 (s, 1H), 9.76 (s, 1H), 9.03 (d, J=2.1 Hz, 1H), 7.84 (d, J=2.1 Hz, 1H), 7.44-7.35 (m, 2H), 7.13 (d, J=9.8 Hz, 2H), 6.99-6.90 (m, 3H), 4.43-4.35 (m, 2H), 4.24 (dd, J=12.7, 5.0 Hz, 1H), 3.85-3.75 (m, 2H), 3.69-3.61 (m, 2H), 3.51-3.44 (m, 2H), 3.26-3.14 (m, 4H), 3.09-3.06 (m, 2H), 3.04-2.94 (m, 2H), 2.87-2.77 (m, 1H), 2.58-2.52 (m, 1H), 2.32 (d, J=1.7 Hz, 3H), 2.18-2.10 (m, 1H), 2.04-1.97 (m, 1H); [M+H]⁺=797.8.

### Example C39: (R)-3-(tert-butyl)-N-(1-(4-(6-((3-(4-((1-(4-(2,4-dioxotetrahydropyrimidin-1(21)-yl)phenyl)piperidin-4-yl)methyl)piperazin-1-yl)phenyl)amino)pyrimidin-4-yl)-5-fluoro-2-methylphenyl)ethyl)-1,2,4-oxadiazole-5-carboxamide

### Step 1: (R)-3-(tert-butyl)-N-(1-(5-fluoro-2-methyl-4-(4,4,5,5-tetramethyl-1,3,2-dioxaborolan-2-yl)phenyl)ethyl)-1,2,4-oxadiazole-5-carboxamide

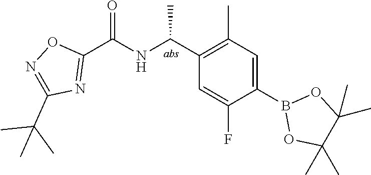 [SMILES: Cc1cc(B2OC(C)(C)C(C)(C)O2)c(F)cc1[C@@H](C)NC(=O)c1nc(C(C)(C)C)no1]

A mixture of (R)-1-(5-fluoro-2-methyl-4-(4,4,5,5-tetramethyl-1,3,2-dioxaborolan-2-yl)phenyl)ethan-1-amine hydrochloride (2 g, 6.3 mmol), ethyl 3-(tert-butyl)-1,2,4-oxadiazole-5-carboxylate (2.5 g, 12.6 mmol) and K₂CO₃ (2.6 g, 18.9 mmol) in EtOH (50 mL) was stirred at 80° C. for 16 h. The mixture was cooled and concentrated under reduced pressure. The residue was purified by silica gel column chromatography, eluted with hexane/EtOAc (1:1) to afford the product (660 mg, 24%). [M+H]⁺=432.3.

### Step 2: tert-butyl 4-(3-((6-chloropyrimidin-4-yl)amino)phenyl)piperazine-1-carboxylate

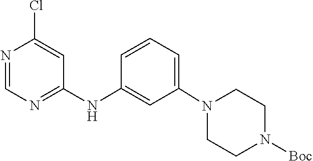 [SMILES: CC(C)(C)OC(=O)N1CCN(c2cccc(Nc3cc(Cl)ncn3)c2)CC1]

To a stirred mixture of tert-butyl 4-(3-aminophenyl)piperazine-1-carboxylate (1.5 g, 5.41 mmol) and 4,6-dichloropyrimidine (0.926 g, 5.95 mmol) in ethanol (30 mL) was added DIEA (2.1 g, 16.23 mmol) at room temperature. The resulting mixture was stirred for 6 h at 78° C. under nitrogen atmosphere and concentrated under reduced pressure. The residue was purified by silica gel column chromatography, eluted with 5%-50% EtOAc in petroleum ether to afford the product (1.4 g, 65%). [M+H]⁺=390.1.

### Step 3: tert-butyl (R)-4-(3-((6-(4-(1-(3-(tert-butyl)-1,2,4-oxadiazole-5-carboxamido)ethyl)-2-fluoro-5-methylphenyl)pyrimidin-4-yl)amino)phenyl)piperazine-1-carboxylate

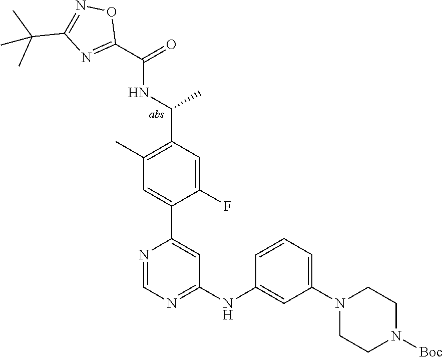 [SMILES: Cc1cc(-c2cc(Nc3cccc(N4CCN(C(=O)OC(C)(C)C)CC4)c3)ncn2)c(F)cc1[C@@H](C)NC(=O)c1nc(C(C)(C)C)no1]

To a stirred mixture of tert-butyl 4-(3-((6-chloropyrimidin-4-yl)amino)phenyl)piperazine-1-carboxylate (296 mg, 0.759 mmol) and (R)-3-(tert-butyl)-N-(1-(5-fluoro-2-methyl-4-(4,4,5,5-tetramethyl-1,3,2-dioxaborolan-2-yl)phenyl)ethyl)-1,2,4-oxadiazole-5-carboxamide (360 mg, 0.835 mmol) in dioxane (12 mL) and H₂O (3 mL) were added K₂CO₃ (210 mg, 1.52 mmol) and Pd(dppf)Cl₂ (31 mg, 0.038 mmol) at room temperature under nitrogen atmosphere. The resulting mixture was stirred for 16 h at 89° C. under nitrogen atmosphere. The mixture was allowed to cool down to room temperature and concentrated under reduced pressure. The residue was purified by silica gel column chromatography, eluted with 10%-90% EtOAc in petroleum ether to afford the product (290 mg, 58%). [M+H]⁺=659.3.

### Step 4: (R)-3-(tert-butyl)-N-(1-(5-fluoro-2-methyl-4-(6-((3-(piperazin-1-yl)phenyl)amino)pyrimidin-4-yl)phenyl)ethyl)-1,2,4-oxadiazole-5-carboxamide

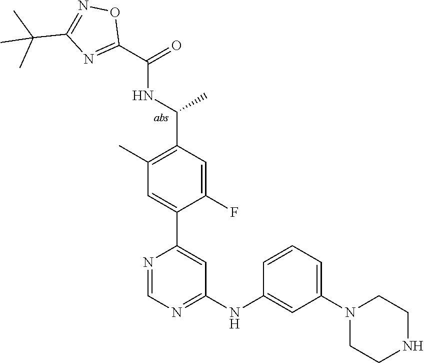 [SMILES: Cc1cc(-c2cc(Nc3cccc(N4CCNCC4)c3)ncn2)c(F)cc1[C@@H](C)NC(=O)c1nc(C(C)(C)C)no1]

To a stirred solution of tert-butyl (R)-4-(3-((6-(4-(1-(3-(tert-butyl)-1,2,4-oxadiazole-5-carboxamido)ethyl)-2-fluoro-5-methylphenyl)pyrimidin-4-yl)amino)phenyl)piperazine-1-carboxylate (290 mg, 0.44 mmol) in DCM (5 mL) was added TFA (5 mL) at room temperature. The resulting solution was stirred for 2 h at room temperature and concentrated under vacuum. The residue (234 mg, crude) was used directly for next step without any further purification. [M+H]⁺=559.3.

### Step 5: (R)-3-(tert-butyl)-N-(1-(4-(6-((3-(4-((1-(4-(2,4-dioxotetrahydropyrimidin-1(2H)-yl)phenyl)piperidin-4-yl)methyl)piperazin-1-yl)phenyl)amino)pyrimidin-4-yl)-5-fluoro-2-methylphenyl)ethyl)-1,2,4-oxadiazole-5-carboxamide

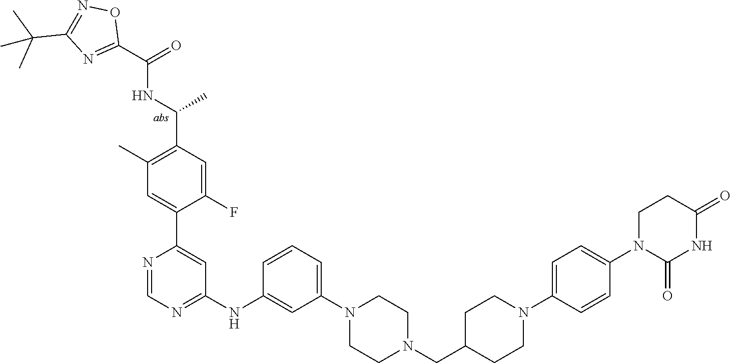 [SMILES: Cc1cc(-c2cc(Nc3cccc(N4CCN(CC5CCN(c6ccc(N7CCC(=O)NC7=O)cc6)CC5)CC4)c3)ncn2)c(F)cc1[C@@H](C)NC(=O)c1nc(C(C)(C)C)no1]

A mixture of (R)-3-(tert-butyl)-N-(1-(5-fluoro-2-methyl-4-(6-((3-(piperazin-1-yl)phenyl)amino)pyrimidin-4-yl)phenyl)ethyl)-1,2,4-oxadiazole-5-carboxamide (0.234 g, 0.418 mmol) and 1-(4-(2,4-dioxotetrahydropyrimidin-1(2H)-yl)phenyl)piperidine-4-carbaldehyde (0.189 g, 0.628 mmol) in 1,2-dichloroethane (8 mL) and HOAc (25 mg) was stirred in a round bottom flask at room temperature for 0.5 hour. To the mixture was added NaBH(OAc)₃ (0.222 g, 1.04 mmol) and stirred at room temperature for 12 hours. Then the mixture was evaporated in vacuum to afford the crude product, which was purified by pre-HPLC to afford the product (0.097 g, 27%). ¹H NMR (500 MHz, DMSO) δ 10.25 (s, 1H), 9.85 (d, J=7.9 Hz, 1H), 9.62 (s, 1H), 8.70 (s, 1H), 7.91 (d, J=8.1 Hz, 1H), 7.43 (d, J=13.1 Hz, 1H), 7.25 (d, J=11.5 Hz, 2H), 7.19-7.08 (m, 4H), 6.93 (d, J=9.1 Hz, 2H), 6.65 (d, J=7.1 Hz, 1H), 5.30 (t, J=7.1 Hz, 1H), 3.69 (t, J=6.7 Hz, 4H), 3.19-3.10 (m, 4H), 2.69-2.65 (m, 4H), 2.39 (s, 4H), 2.22 (d, J=7.1 Hz, 2H), 1.87-1.65 (m, 4H), 1.50 (d, J=7.0 Hz, 3H), 1.37 (s, 9H), 1.29-1.15 (m, 4H), 0.87-0.71 (m, 1H); [M+H]⁺=844.6.

### Example C40: (R)-3-(tert-butyl)-N-(1-(4-(2-((6-(4-((1-(4-(2,4-dioxotetrahydropyrimidin-1(21)-yl)phenyl)piperidin-4-yl)methyl)piperazin-1-yl)pyridazin-3-yl)amino)pyrimidin-4-yl)-2-methylphenyl)ethyl)-1,2,4-oxadiazole-5-carboxamide

### Step 1: tert-butyl 4-(6-chloropyridazin-3-yl)piperazine-1-carboxylate

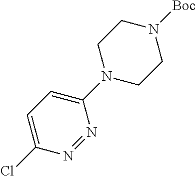 [SMILES: CC(C)(C)OC(=O)N1CCN(c2ccc(Cl)nn2)CC1]

To a stirred solution of 3,6-dichloropyridazine (5 g, 33.56 mmol) and tert-butyl piperazine-1-carboxylate (9.4 g, 50.35 mmol) in DMF (100 mL) was added TEA (10.2 g, 100.69 mmol) dropwise at room temperature. The resulting mixture was stirred for 16 h at 80° C. and diluted with water (500 mL). The resulting mixture was extracted with EtOAc (3×300 mL). The combined organic layers were washed with brine (2×200 mL) and dried over anhydrous Na₂SO₄. After filtration, the filtrate was concentrated under reduced pressure. The residue was purified by silica gel column chromatography to give the titled product (8.1 g, 81%). [M+H]⁺=299.1.

### Step 2: tert-butyl 4-(6-((diphenylmethylene)amino)pyridazin-3-yl)piperazine-1-carboxylate

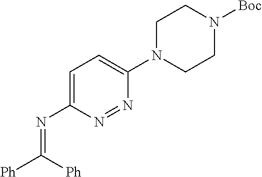 [SMILES: CC(C)(C)OC(=O)N1CCN(c2ccc(N=C(c3ccccc3)c3ccccc3)nn2)CC1]

To a stirred mixture of tert-butyl 4-(6-chloropyridazin-3-yl)piperazine-1-carboxylate (2 g, 6.69 mmol) and diphenylmethanimine (1.8 g, 10.04 mmol) in toluene (40.00 mL) were added Pd₂(dba)₃ (0.31 g, 0.34 mmol), BINAP (0.42 g, 0.67 mmol) and Cs₂CO₃ (4.36 g, 13.39 mmol) in portions at room temperature. The resulting mixture was stirred for 16 h at 100° C. under nitrogen atmosphere and concentrated under vacuum. The residue was purified by silica gel column chromatography to give the titled product (1.4 g, 47%). [M+H]⁺=444.2.

### Step 3: tert-butyl 4-(6-aminopyridazin-3-yl)piperazine-1-carboxylate

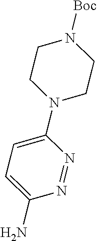 [SMILES: CC(C)(C)OC(=O)N1CCN(c2ccc(N)nn2)CC1]

To a stirred mixture of tert-butyl 4-(6-((diphenylmethylene)amino)pyridazin-3-yl)piperazine-1-carboxylate (1.35 g, 3.04 mmol) and citric acid (13.5 mL, 70.27 mmol) in THF (20 mL) was added H₂O (13.5 mL) dropwise at room temperature. The resulting mixture was stirred overnight at room temperature and concentrated under vacuum. The residue was purified by silica gel column chromatography to give the titled product (710 mg, 83%). [M+H]⁺=280.2.

### Step 4: tert-butyl (R)-4-(6-((6-(4-(1-(3-(tert-butyl)-1,2,4-oxadiazole-5-carboxamido)ethyl)-3-methylphenyl)pyrimidin-4-yl)amino)pyridazin-3-yl)piperazine-1-carboxylate

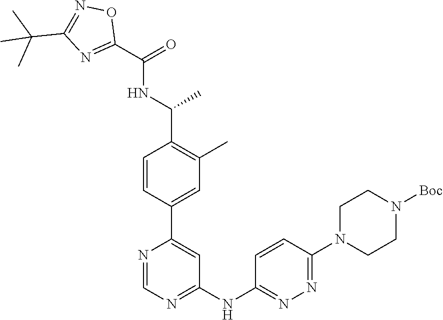 [SMILES: Cc1cc(-c2cc(Nc3ccc(N4CCN(C(=O)OC(C)(C)C)CC4)nn3)ncn2)ccc1[C@@H](C)NC(=O)c1nc(C(C)(C)C)no1]

To a stirred mixture of tert-butyl 4-(6-aminopyridazin-3-yl)piperazine-1-carboxylate (500 mg, 1.79 mmol) and (R)-3-(tert-butyl)-N-(1-(4-(6-chloropyrimidin-4-yl)-2-methylphenyl)ethyl)-1,2,4-oxadiazole-5-carboxamide (the compound was obtained through the similar way of example C39) (858.90 mg, 2.15 mmol) in dioxane (10 mL) were added XPhos (170.66 mg, 0.36 mmol), XPhos Pd G₃ (151.51 mg, 0.18 mmol) and Cs₂CO₃ (1.17 g, 3.58 mmol) in portions at room temperature. The resulting mixture was stirred for 16 h at 100° C. under nitrogen atmosphere and concentrated under vacuum. The residue was purified by silica gel column chromatography to give the titled product (314 mg, 27%). [M+H]⁺=643.2.

### Step 5: (R)-3-(tert-butyl)-N-(1-(2-methyl-4-(6-((6-(piperazin-1-yl)pyridazin-3-yl)amino)pyrimidin-4-yl)phenyl)ethyl)-1,2,4-oxadiazole-5-carboxamide

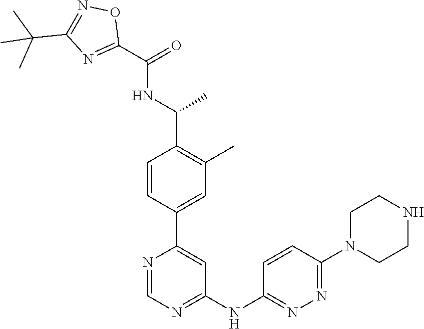 [SMILES: Cc1cc(-c2cc(Nc3ccc(N4CCNCC4)nn3)ncn2)ccc1[C@@H](C)NC(=O)c1nc(C(C)(C)C)no1]

A solution of tert-butyl (R)-4-(6-((6-(4-(1-(3-(tert-butyl)-1,2,4-oxadiazole-5-carboxamido)ethyl)-3-methylphenyl)pyrimidin-4-yl)amino)pyridazin-3-yl)piperazine-1-carboxylate (314 mg, 0.49 mmol) and HCl in 1,4-dioxane (6 mL) in DCM (6 mL) was stirred for 1 h at room temperature. The resulting mixture was concentrated under vacuum. The residue was triturated with Et₂O to afford the titled product (254 mg, 89%). [M+H]⁺=543.3.

### Step 6: (R)-3-(tert-butyl)-N-(1-(4-(2-((6-(4-((1-(4-(2,4-dioxotetrahydropyrimidin-1(2H)-yl)phenyl)piperidin-4-yl)methyl)piperazin-1-yl)pyridazin-3-yl)amino)pyrimidin-4-yl)-2-methylphenyl)ethyl)-1,2,4-oxadiazole-5-carboxamide

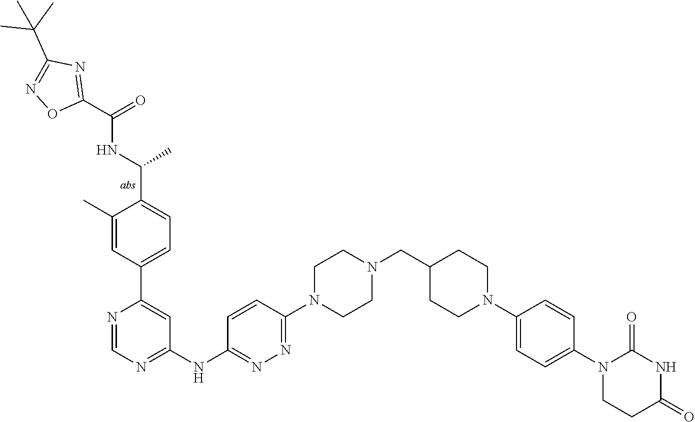 [SMILES: Cc1cc(-c2cc(Nc3ccc(N4CCN(CC5CCN(c6ccc(N7CCC(=O)NC7=O)cc6)CC5)CC4)nn3)ncn2)ccc1[C@@H](C)NC(=O)c1nc(C(C)(C)C)no1]

A mixture of (R)-3-(tert-butyl)-N-(1-(2-methyl-4-(6-((6-(piperazin-1-yl)pyridazin-3-yl)amino)pyrimidin-4-yl)phenyl)ethyl)-1,2,4-oxadiazole-5-carboxamide (100 mg, 0.17 mmol), 1-(4-(2,4-dioxotetrahydropyrimidin-1(2H)-yl)phenyl)piperidine-4-carbaldehyde (60 mg, 0.2 mmol), NaBH(OAc)₃ (106 mg, 0.5 mmol) and NaOAc (82 mg, 1.0 mmol) in DCE was stirred at room temperature for 16 h. The mixture was concentrated and purified by silica gel column chromatography to give the titled product (50 mg, 36%). ¹H NMR (500 MHz, DMSO) δ 10.24 (d, J=5.3 Hz, 2H), 9.89 (d, J=7.8 Hz, 1H), 8.72 (s, 1H), 8.05-7.79 (m, 4H), 7.62 (d, J=8.6 Hz, 1H), 7.40 (d, J=9.9 Hz, 1H), 7.13 (d, J=8.7 Hz, 2H), 6.93 (d, J=8.9 Hz, 2H), 5.39-5.29 (m, 1H), 3.74-3.64 (m, 4H), 3.52 (s, 4H), 2.71-2.61 (m, 4H), 2.51-2.44 (m, 7H), 2.23 (d, J=7.1 Hz, 2H), 1.82 (d, J=11.7 Hz, 2H), 1.78-1.68 (m, 1H), 1.51 (d, J=6.9 Hz, 3H), 1.36 (s, 9H), 1.30-1.26 (m, 2H); [M+H]⁺=828.6.

### Example C41: 3-(tert-butyl)-N—((R)-1-(4-(6-((5-(4-(4-((R)-2,6-dioxopiperidin-3-yl)-3,5-difluorophenethyl)piperazin-1-yl)pyridin-2-yl)amino)pyrimidin-4-yl)-2-methylphenyl)ethyl)-1,2,4-oxadiazole-5-carboxamide

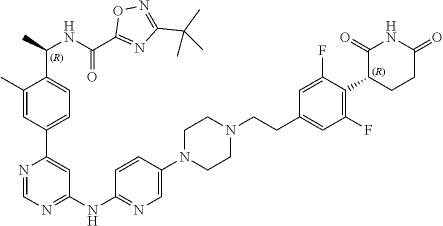

The titled compound was synthesized in the procedures similar to Example C40. ¹H NMR (500 MHz, DMSO) δ 10.95 (s, 1H), 9.99 (s, 1H), 9.89 (d, J=5.0 Hz, 1H), 8.70 (s, 1H), 8.05-8.03 (m, 2H), 7.84-7.83 (m, 2H), 7.69-7.60 (m, 2H), 7.46 (dd, J=10.0 Hz, 5.0 Hz, 1H), 7.06 (d, J=10.0 Hz, 2H), 5.35-5.31 (m, 1H), 4.22-4.18 (m, 1H), 3.14 (s, 4H), 2.83-2.77 (m, 3H), 2.62-2.60 (m, 5H), 2.47 (s, 3H), 2.15-2.11 (m, 1H), 2.01-1.99 (m, 1H), 1.51 (d, J=5.0 Hz, 3H), 1.38-1.34 (m, 9H), 1.23-1.18 (m, 2H); [M+H]⁺=793.7.

### Example 42: 3-((4-(4-(4-(4-(2,6-dioxopiperidin-3-yl)-3,5-difluorophenethyl)piperazin-1-yl)piperidin-1-yl)-3-methoxyphenyl)amino)-6-ethyl-5-((tetrahydro-2H-pyran-4-yl)amino)pyrazine-2-carboxamide

### Step 1: ethyl 2-(3,5-difluoro-4-nitrophenyl)acetate

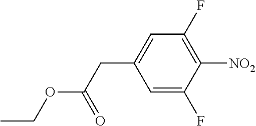 [SMILES: CCOC(=O)Cc1cc(F)c([N+](=O)[O-])c(F)c1]

A solution of 1,3-difluoro-2-nitrobenzene (50.0 g, 314.4 mmol) in NMP (300 mL) were cooled to −20° C. under N₂ atmosphere. Then a mixture of ethyl 2-chloroacetate (65.5 g, 534.7 mmol) and t-BuOK (121.0 g, 1.08 mol) in NMP (50 mL) was added slowly at −10° C. to −20° C. over 2 h. After being stirred for 2 h, the reaction was quenched by pouring into IM HCl (200 mL) and ice-water. The mixture was extracted with EA (300 mL×3). The combined organic layer was washed by brine, dried with Na₂SO₄. The solution was concentrated in vacuum and the residue was purified by silica gel column chromatography (PE/EA=200/1 to 100/1) to provide product (13.7 g, 18%). ¹H NMR (400 MHz, CDCl₃) & 7.06 (d, J=8.4 Hz, 2H), 4.20 (q, J=7.2 Hz, 2H), 3.65 (s, 2H), 1.28 (t, J=7.2 Hz, 3H).

### Step 2: ethyl 2-(4-amino-3,5-difluorophenyl)acetate

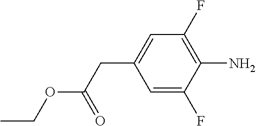

To a solution of ethyl 2-(3,5-difluoro-4-nitrophenyl)acetate (13.7 g, 56 mmol) in MeOH (150 mL) was added 10% Pd/C (1.5 g) at r.t. The mixture was stirred at r.t under H₂ atmosphere for 5 h. Filtrated on vacuum to remove Pd/C and concentrated in vacuum to provide the product (12.2 g), which was used in next step without further purification. ¹H NMR (400 MHz, DMSO d₆) δ_{H} 6.82 (d, J=8.0 Hz, 2H), 5.69 (s, 2H), 4.06 (q, J=7.2 Hz, 2H), 3.52 (s, 2H), 1.17 (t, J=7.2 Hz, 3H). [M+H]⁺=216.4.

### Step 3: ethyl 2-(3,5-difluoro-4-iodophenyl)acetate

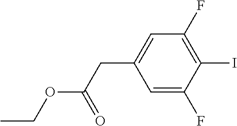

A solution of ethyl 2-(4-amino-3,5-difluorophenyl)acetate (12.2 g, 56 mmol) in MeCN (150 mL) was cooled to 0° C. under N₂ atmosphere and CuI (21.2 g, 112 mmol) was added. After stirring for 10 min, tert-butylnitrite (11.5 g, 112 mmol) was added dropwise over 30 min. Then the mixture was stirred at r.t for overnight. The reaction was quenched by pouring into water and extracted with EA (300 mL×3). All organic layers were combined and washed by brine, dried with Na₂SO₄. The solution was concentrated in vacuum and the residue was purified by silica gel column chromatography (PE/EA=500/1 to 100/1) to provide the product (8.8 g, 48%). [M+H]⁺=326.5.

### Step 4: ethyl 2-(4-(2,6-bis(benzyloxy)pyridin-3-yl)-3,5-difluorophenyl)acetate

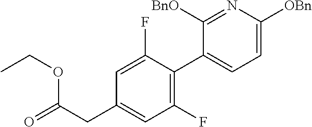 [SMILES: CCOC(=O)Cc1cc(F)c(-c2ccc(OCc3ccccc3)nc2OCc2ccccc2)c(F)c1]

To a solution of ethyl 2-(3,5-difluoro-4-iodophenyl)acetate (8.8 g, 27.0 mmol) in a mixed solvent of 1,4-dioxane/H₂O (100 mL/20 mL) were added K₂CO₃ (9.3 g, 67.4 mmol), 2,6-bis(benzyloxy)-3-(4,4,5,5-tetramethyl-1,3,2-dioxaborolan-2-yl)pyridine (14.6 g, 35.0 mol) and Pd(dppf)Cl₂ (2.9 g, 4.0 mmol) under N₂ atmosphere. The resulting solution was stirred for 6 h at 100° C. The mixture was diluted with water (300 mL) and extracted with EA (300 mL×3). All organic layers were combined and washed with brine (300 mL), dried over Na₂SO₄. The solution was concentrated in vacuum and the residue was purified by silica gel column chromatography (PE/EA=200/1) to give the product (8.2 g, 62%). ¹H NMR (400 MHz, CDCl₃) δ_{H} 7.49 (d, J=8.0 Hz, 1H), 7.40-7.24 (m, 10H), 6.90 (d, J=8.0 Hz, 2H), 6.47 (d, J=8.0 Hz, 1H), 5.38 (s, 2H), 5.33 (s, 2H), 4.19 (q, J=7.2 Hz, 2H), 3.61 (s, 2H), 1.28 (t, J=7.2 Hz, 3H).

### Step 5: 2-(4-(2,6-bis(benzyloxy)pyridin-3-yl)-3,5-difluorophenyl)ethanol

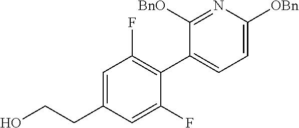 [SMILES: OCCc1cc(F)c(-c2ccc(OCc3ccccc3)nc2OCc2ccccc2)c(F)c1]

A solution of ethyl 2-(4-(2,6-bis(benzyloxy)pyridin-3-yl)-3,5-difluorophenyl)acetate (8.2 g, 16.7 mol) in THF (100 mL) was cooled to 0° C. under N₂ atmosphere and 1.5 M DIBAL-H (45 mL, 67.5 mol) in THF was added dropwise over 30 min. Then the mixture was stirred at r.t for 2 h. The reaction was quenched by pouring into water and extracted with EA (300 mL×3). All organic layers were combined and washed by brine, dried with Na₂SO₄. The solution was concentrated in vacuum and the residue was purified by column chromatography (PE/EA=10/1 to 3/1) to provide the product (6.6 g, 88%). ¹H NMR (400 MHz, CDCl₃) δ_{H} 7.49 (d, J=8.0 Hz, 1H), 7.42-7.25 (m, 9H), 6.84 (d, J=8.0 Hz, 2H), 6.47 (d, J=8.0 Hz, 1H), 5.38 (s, 2H), 5.33 (s, 2H), 3.90 (m, 2H), 2.87 (t, J=6.4 Hz, 2H). [M+H]⁺=448.3.

### Step 6: 3-(2,6-difluoro-4-(2-hydroxyethyl)phenyl)piperidine-2,6-dione

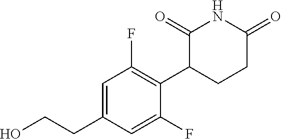 [SMILES: O=C1CCC(c2c(F)cc(CCO)cc2F)C(=O)N1]

To a solution of 2-(4-(2,6-bis(benzyloxy)pyridin-3-yl)-3,5-difluorophenyl)ethanol (6.6 g, 14.7 mmol) in DCM (150 mL) was added TFA (50 mL). After stirred overnight, the mixture was concentrated in vacuo. The residue was dissolved in MeOH (200 mL) and 10% Pd/C (1.0 g) was added. The resulted mixture was stirred for 2 days at r.t under H₂ atmosphere. The mixture was filtered and the filtrate was concentrated to give a residue which was purified by reversed flash C18 chromatography (ACN/water=0% to 30%) to give the title compound (2.1 g, 53%). [M+H]⁺=270.1.

### Step 7: 4-(2,6-dioxopiperidin-3-yl)-3,5-difluorophenethyl methanesulfonate

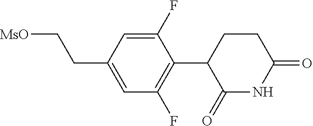 [SMILES: CS(=O)(=O)OCCc1cc(F)c(C2CCC(=O)NC2=O)c(F)c1]

To the solution of 3-(2,6-difluoro-4-(2-hydroxyethyl)phenyl)piperidine-2,6-dione (1 g, 3.71 mmol) and TEA (1.13 g, 11.14 mmol) in 20 mL DCM, MsCl (510 mg, 4.46 mmol) was added dropwise at 0° C. The mixture was stirred at room temperature for 4 hours. The mixture was quenched with NaHCO₃ aqueous and extracted by DCM. The organic layer was separated and concentrated. The residue was purified by silica column chromatography (MeOH:DCM=0-6%) to afford the product (1.1 g, 85.3% yield). [M+H]⁺=348.1.

### Step 8: 3-((4-(4-(4-(4-(2,6-dioxopiperidin-3-yl)-3,5-difluorophenethyl)piperazin-1-yl)piperidin-1-yl)-3-methoxyphenyl)amino)-6-ethyl-5-((tetrahydro-2H-pyran-4-yl)amino)pyrazine-2-carboxamide

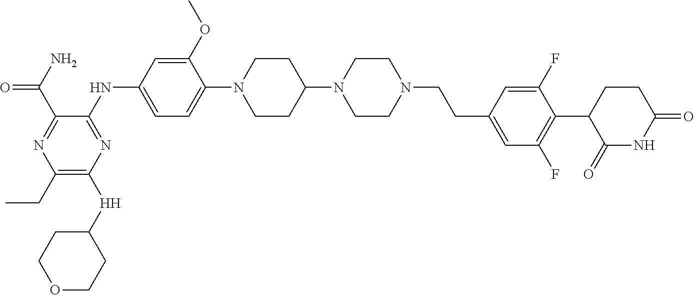

To the solution of 6-ethyl-3-((3-methoxy-4-(4-(piperazin-1-yl)piperidin-1-yl)phenyl)amino)-5-((tetrahydro-2H-pyran-4-yl)amino)pyrazine-2-carboxamide (50 mg, 0.09 mmol) and 4-(2,6-dioxopiperidin-3-yl)-3,5-difluorophenethyl methanesulfonate (48 mg, 0.14 mmol) in 3 mL ACN and 0.5 mL DMSO, KI (46 mg, 0.28 mmol) and DIEA (60 mg, 0.46 mmol) was added. The mixture was stirred at 85° C. for 16 hours. After LCMS shown the reaction was completed, the mixture was concentrated in vacuum. The residue was washed with water and extracted by DCM. The organic layer was separated and concentrated. The residue was purified by prep-TLC (DCM:MeOH=10:1) to afford the crude. The crude product was purified by prep-HPLC to afford the product (8.1 mg, 11% yield). ¹H NMR (500 MHz, DMSO-d₆) δ 11.02 (s, 1H), 10.97 (s, 1H), 7.54 (d, J=2.7 Hz, 1H), 7.28-7.22 (m, 2H), 7.03 (d, J=10.2 Hz, 3H), 6.81 (t, J=8.6 Hz, 2H), 4.20 (dd, J=12.7, 5.0 Hz, 1H), 4.16-4.06 (m, 1H), 3.92 (d, J=6.5 Hz, 2H), 3.81 (s, 3H), 3.41-3.31 (m, 9H), 2.86-2.71 (m, 3H), 2.62-2.52 (m, 8H), 2.47 (s, 2H), 2.26 (t, J=11.1 Hz, 1H), 2.12 (dt, J=13.0, 9.2 Hz, 1H), 2.03-1.96 (m, 1H), 1.89-1.78 (m, 4H), 1.70-1.48 (m, 4H), 1.19 (t, J=7.4 Hz, 3H); [M+H]⁺=790.7.

### Test Example 1

### Cell Degradation

### Cell Line Generation

H1975-clone #28(Del19/T790M/C797S) was stably expressed in H1975 cell lines (from ATCC) by lentivirus-mediated over-expression. The EGFR over-expressed cells then underwent gene knockout, in which the EGFR targeting sgRNA was designed to only target the endogenous EGFR copies and preserve the exogenous EGFR copies. Followed by the gene knockout, the edited H1975 cells were seeded in 96 well plates at the concentration of 1 cell/cell, cultured for about 2 weeks to allow single clones formation. The formed clones were screened by DNA sequencing and whole exon sequencing analysis for the desired edition. H1975-clone #28 was finally confirmed as homozygous Del19/T790M/C797S EGFR clone

### Cell Treatment

1a). BaF3 WT, BaF3-LTC(L858R/T790M/C797S), BaF3-DTC(Del19/T790M/C797S) cells are seeded at 20000 cells/well (LTC & DTC) in cell culture medium [RPMI1640(Gibco, phenol red free, Cat #11835-030), 10% heat-inactive FBS, 1% PS(Gibco, Cat #10378)] in Corning 96 well plate (Cat #3799).

1b). On day 1, H1975-clone #28(Del19/T790M/C797S) 10000 cells/well correspondingly in cell culture medium [RPMI1640(Gibco, Cat #72400-047), 10% heat-inactive FBS, 1% PS(Gibco, Cat #10378)] in Corning 96 well plate (Cat #3599).

BaF3-LTC(L858R/T790M/C797S) and BaF3-DTC(Del19/T790M/C797S) cells are treated with compounds diluted in 0.2% DMSO cell culture medium and incubate for 16 h, 37° C., 5% CO₂, H1975- #28 cells are treated with compounds diluted in 0.2% DMSO cell culture medium on day 2, incubate for 16 h, 37° C., 5% CO₂. the final concentration of compounds in all assay is start with 10 uM, 5-fold dilution, total 8 doses were included.

1c). TMD-8 cells are seeded at 20000 cells/well at a volume of 15 μl/well in cell culture medium [RPMI1640(Gibco, phenol red free, Cat #11835-030), 10% heat-inactive FBS, 1% PS(Gibco, Cat #10378)] in Corning 96 well plate (Cat #3799). TMD-8 cells are treated with compounds diluted in 0.2% DMSO, dilution is done according to the following protocol: (1) make 500× stock solution in DMSO from 1 mM by 6-fold dilution, total 8 doses were included; (2) make 2× solution in cell culture medium by transferring 0.5 μl 500× stock solution into 125 μl medium; (3) 15 μl of 2× solution is added to cells and incubate for 6 h.

### HTRF Assay (EGFR Degradation)

After 16 h treatment, add HTRF lysis buffer to each well; seal the plate and incubate 1 hour at room temperature on a plate shaker; Once the cells are lysed, 16 μL of cell lysate are transferred to a PE 384-well HTRF detection plate; 4 μL of pre-mixed HTRF antibodies are added to each well; Cover the plate with a plate sealer, spin 1000 rpm for 1 min, Incubate overnight at room temperature; Read on BMG PheraStar with HTRF protocol (337 nm-665 nm-620 nm).

The inhibition (degradation) percentage of the compound was calculated by the following equation: Inhibition percentage of Compound=100−100×(Signal−low control)/(High control−low control), wherein signal=each test compound group
Low control=only lysis buffer without cells, indicating that EGFR is completely degraded;
High control=Cell group with added DMSO and without compound, indicating microplate readings without EGFR degradation;
Dmax is the maximum percentage of inhibition (degradation).

HTFR assay (BTK degradation)

After 6 h treatment, add 10 μl 4xlysis buffer to each well; seal the plate and incubate 30 min at room temperature on a plate shaker; Once the cells are lysed, 16 μL of cell lysate are transferred to a PE 384-well HTRF detection plate; 4 μL of pre-mixed HTRF antibodies are added to each well; Cover the plate with a plate sealer, spin 1000 rpm for 1 min, Incubate overnight at room temperature; Read on BMG PheraStar with HTRF protocol (337 nm-665 nm-620 nm).

The inhibition (degradation) percentage of the compound was calculated by the following equation: Inhibition percentage of Compound=100−100×(Signal−low control)/(High control−low control), wherein signal=each test compound group
Low control=only lysis buffer without cells, indicating that BTK is completely degraded;
High control=Cell group with added DMSO and without compound, indicating microplate readings without BTK degradation;
Dmax is the maximum percentage of inhibition (degradation).

The IC₅₀ (DC₅₀) value of a compound can be obtained by fitting the following equation

Y=Bottom+(TOP−Bottom)/(1+((IC₅₀/X)\hat{ }hillslope))

Wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the ability the inhibitory ability of the compound is; Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 1
Degradation (BaF3) result for Example 16 to Example 19

| | |
|---|---|
| DTC | LTC |

| Example | DC₅₀(nM) | Dmax (%) | DC₅₀(nM) | Dmax (%) |
|---|---|---|---|---|
| 16 | 0.61 | 88.65 | 6.10 | 91.47 |
| 17 | 6.75 | 64.81 | 45.13 | 84.63 |
| 18 | 3.74 | 95.55 | 8.82 | 91.88 |
| 19 | 6.87 | 92.16 | 18.23 | 89.29 |

TABLE 2
Degradation (H1975 # 28 DTC and BaF3-
LTC) result for Example 20 to Example 29

| | H1975 # 28 DTC | | BaF3-LTC | |
|---|---|---|---|---|
| Example | DC₅₀(nM) | Dmax (%) | DC₅₀(nM) | Dmax (%) |
| 20 | >10000 | 19.09 | 89.98 | 69.05 |
| 21 | 0.78 | 89.6 | 6.77 | 95.43 |
| 22 | 1.17 | 84.8 | 5.23 | 95.42 |
| 24 | 1.37 | 82.72 | 8.99 | 98.64 |
| 25 | >10000 | −8.4 | 359.8 | 57.2 |
| 26 | 0.851 | 86.08 | 7.54 | 86.94 |
| 28 | 0.595 | 62.25 | 7.8 | 86.46 |
| 29 | >10000 | −4.12 | 85.37 | 77.12 |

TABLE 3
Degradation (TMD-8) result for Example 31 and 32

| Example | DC₅₀(nM) | Dmax (%) |
|---|---|---|
| 31 | >2000.0 | 11.99 |
| 32 | 5.33 | 97.38 |

### Test Example 2

### HTRF (Homogeneous Time-Resolved Fluorescence) Assay

Biochemical potency of compound was determined by using CRBN& DDB1 protein (His Tag). Compounds were tested for blocking the binding of CRBN&DDB1 protein (CRBN, aa 40-442, DDB1, 1-1140, Viva Biotech) with biotin labeled thalidomide in an assay based on the time-resolved fluorescence-resonance energy transfer (TR-FRET) methodology. The assay was carried out in 384-well low volume black plates in a reaction mixture containing CRBN& DDB1 protein, 30 nM biotin labeled thalidomide and 0-10 μM compound in buffer containing 50 mM HEPES pH7.5, 50 mM NaCl, 0.01% BSA, 1 mM DTT and 0.015% Brij-35. The protein was preincubated with compound for 60 minutes at room temperature and biotin labeled thalidomide was added to plate. After further incubation at room temperature for 60 minutes detection reagents Mab Anti-6His Eu cryptate Gold (Cisbio, Cat #61HI2KLB) and Streptavidin-XL665 (Cisbio, Cat #610SAXLG) were added to plate. Plates were sealed and incubated at room temperature for 1 hour, and the TR-FRET signals (ex337 nm, em665 nm/620 nm) were recorded on a PHERAstar FSX plate reader (BMG Labtech). The inhibition percentage of CRBN& DDB1 protein interaction with biotin labeled thalidomide in presence of increasing concentrations of compounds was calculated based on the ratio of fluorescence at 665 nm to that at 620 nm. IC₅₀ was derived from fitting the dose-response % inhibition data to the four-parameter logistic model by Dotmatics.

Four-parameter logistic equation: Y=Bottom+(Top−Bottom)/(1+10\hat{ }((LogIC₅₀−X) *HillSlope)). While X is Log of compound concentration. Y is % inhibition at X. Bottom is the bottom of the curve effect. Top is the top of the curve effect. HillSlope is the hill slope factor.

TABLE 4
HTRF assay result for Example 2 to Example 15

| Example | IC₅₀(nM) |
|---|---|
| 2 | 406 |
| 5 | 26 |
| 6 | 1070 |
| 7 | 22 |
| 9 | 64 |
| 12 | 178 |
| 13 | 27 |
| 14 | 24 |
| 15 | 239 |

### Test Example 3

### Cell Degradation

### Cell Treatment

TMD-8 cells were seeded at 20000 cells/well at a volume of 15 l/well in cell culture medium [RPMI1640(Gibco, phenol red free, Cat #11835-030), 10% heat-inactive FBS, 1% PS(Gibco, Cat #10378)] in Corning 96 well plate (Cat #3799). TMD-8 cells were treated with compounds diluted in 0.2% DMSO, dilution was done according to the following protocol: (1) making 500× stock solution in DMSO from 1 mM by 6-fold dilution, total 8 doses were included; (2) making 2× solution in cell culture medium by transferring 0.5 μl 500× stock solution into 125 d medium; (3) adding 15 μl of 2× solution to cells for incubation of 6 h.

### HTFR Assay

After 6 h treatment, 10 μl 4xlysis buffer was added to each well; the plate was sealed and incubated for 30 min at room temperature on a plate shaker; Once the cells was lysed, 16 μL of cell lysate were transferred to a PE 384-well HTRF detection plate; 4 μL of pre-mixed HTRF antibodies were added to each well; the plate was covered with a plate sealer, and then spinned at 1000 rpm for 1 min, then incubated overnight at room temperature; the results were read on BMG PheraStar with HTRF protocol (337 nm-665 nm-620 nm).

The inhibition (degradation) percentage of the compound was calculated by the following equation: Inhibition percentage of Compound=100−100× (Signal−low control)/(High control−low control), wherein signal=each test compound group
Low control=only lysis buffer without cells, indicating that BTK is completely degraded;
High control=Cell group with added DMSO and without compound, indicating microplate readings without BTK degradation;
Dmax is the maximum percentage of inhibition (degradation).

The IC₅₀ (DC₅₀) value of a compound can be obtained by fitting the following equation

Y=Bottom+(TOP−Bottom)/(1+((IC₅₀/X)\hat{ }hillslope))

wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the ability the inhibitory ability of the compound is; Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

### HEK-293 Cell Treatment

HEK-293 cells were seeded at 2000 cells/well at a volume of 50ul/well in cell culture medium [DMEM (Gibco, Cat #11965-092), 10% heat-inactive FBS(Gibco, Cat #10099), 1% PS(Gibco, Cat #10378)] in Corning 96 well plate (Cat #3903), and then incubated overnight. HEK-293 cells were treated with compounds diluted in 0.2% DMSO, dilution was done according to the following protocol: (1) making 500× stock solution in DMSO from 5 mM by 4-fold dilution, total 8 doses were included; (2) making 2× solution in cell culture medium by transferring 0.5ul 500× stock solution into 125ul medium; (3) adding 50ul of 2× solution to cells for incubation of 72 h.

### Cytotoxicity Detection

25 μl of the CellTiter-Glo®Reagent [(Promega) - Cat No. G7572] was added to each well in the 96-well plate. The contents were mixed for 2 minutes on an orbital shaker to induce cell lysis. The plate was then allowed to incubate at room temperature for 10 minutes to stabilize luminescent signal. Luminescence was recorded on BMG PheraStar with luminescence protocol.

### IC50 Calculation

The inhibition percentage of the compound was calculated by the following equation: Inhibition percentage of Compound=100−100×(Signal−low control)/(High control−low control), wherein signal=each test compound group
Low control=only medium group (without cells), indicating that cells proliferation is completely inhibited;
High control=Cell group with added DMSO and without compound, indicating cells proliferation with no inhibition;
Imax is the maximum percentage of inhibition.

The IC50 value of a compound can be obtained by fitting the following equation

Y=Bottom+(TOP−Bottom)/(1+((IC50/X)\hat{ }hillslope))

Wherein, X and Y are known values, and IC50, Hillslope, Top and Bottom are the parameters obtained by fitting with software. Y is the inhibition percentage (calculated from the equation), X is the concentration of the compound; IC50 is the concentration of the compound when the 50% inhibition is reached. The smaller the IC50 value is, the stronger the inhibitory ability of the compound is. Vice versa, the higher the IC50 value is, the weaker the ability the inhibitory ability of the compound is; Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 5
Degradation and HEK293 result for Example B35 to Example C41

| Example | IC50 of HEK293 (nM) | DC50 (nM) |
|---|---|---|
| B35 | >10000.0 | 2.32 |
| B37 | >10000.0 | 0.414 |
| B38 | 26.78 | 1.7 |
| C39 | >10000.0 | 242.2 |
| C41 | >10000.0 | 2.73 |

### Test Example 4

### Cell Treatment

THP-1 cells are seeded at 100000 cells/well at a volume of 15 l/well in cell culture medium [RPMI1640(Gibco, phenol red free, Cat #11835-030), 10% heat-inactive FBS, 1% PS(Gibco, Cat #10378)] in Corning 96 well plate (Cat #3799). THP-1 cells are treated with compounds diluted in 0.2% DMSO, dilution is done according to the following protocol: (1) make 500× stock solution in DMSO from 5 mM by 5-fold dilution, total 8 doses were included; (2) make 2× solution in cell culture medium by transferring 0.5 μl 500× stock solution into 125 μl medium; (3) 15 μl of 2× solution is added to cells and incubate for 6 h.

### HTFR Assay

After 6 h treatment, add 10 μl 4xlysis buffer to each well; seal the plate and incubate 1 hour at room temperature on a plate shaker; Once the cells are lysed, 16 μL of cell lysate are transferred to a PE 384-well HTRF detection plate(for triple mutant cells, the lysate were diluted by the qual volume 1xlysis buffer before transfer); 4 μL of pre-mixed HTRF antibodies are added to each well; Cover the plate with a plate sealer, spin 1000 rpm for 1 min, Incubate overnight at room temperature; Read on BMG PheraStar with HTRF protocol (337 nm-665 nm-620 nm).

The inhibition (degradation) percentage of the compound was calculated by the following equation: Inhibition percentage of Compound=100−100×(Signal−low control)/(High control−low control), wherein signal=each test compound group
Low control=only lysis buffer without cells, indicating that IRAKM is completely degraded;
High control=Cell group with added DMSO and without compound, indicating microplate readings without IRAKM degradation;
Dmax is the maximum percentage of inhibition (degradation).

The IC₅₀ (DC₅₀) value of a compound can be obtained by fitting the following equation

Y=Bottom+(TOP−Bottom)/(1+((IC₅₀/X)\hat{ }hillslope))

Wherein, X and Y are known values, and IC₅₀, Hillslope, Top and Bottom are the parameters obtained by fitting with software. Y is the inhibition percentage (calculated from the equation), X is the concentration of the compound; IC₅₀ is the concentration of the compound when the 50% inhibition is reached. The smaller the IC₅₀ value is, the stronger the inhibitory ability of the compound is. Vice versa, the higher the IC₅₀ value is, the weaker the ability the inhibitory ability of the compound is; Hillslope represents the slope of the fitted curve, generally around 1*; Bottom represents the minimum value of the curve obtained by data fitting, which is generally 0%±20%; Top represents the maximum value of the curve obtained by data fitting, which is generally 100%±20%. The experimental data were fitted by calculating and analyzing with Dotmatics data analysis software.

TABLE 6
Degradation result for Example D42

| Example | DC₅₀ (nM) | Dmax |
|---|---|---|
| D42 | 2.8 | 88 |

The foregoing examples and description of certain embodiments should be taken as illustrating, rather than as limiting the present invention as defined by the claims. As will be readily appreciated, numerous variations and combinations of the features set forth above can be utilized without departing from the present invention as set forth in the claims. All such variations are intended to be included within the scope of the present invention. All references cited are incorporated herein by reference in their entireties.

It is to be understood that, if any prior art publication is referred to herein, such reference does not constitute an admission that the publication forms a part of the common general knowledge in the art in any country.

What is claimed is:

1. A compound of Formula (I): 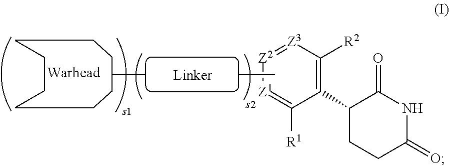
or a pharmaceutically acceptable salt thereof, or a deuterated analog thereof, or a prodrug thereof, wherein:
Warhead is a targeting moiety that binds to a target protein; wherein the target protein is a mediator of a disease in a subject;
Linker is a divalent chemical group that connects the Warhead moiety and the 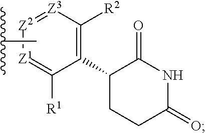
moiety;
s1 is 0 or 1;
s2 is 0 or 1;
Z¹, Z² and Z³ are each independently N or CR^{z}, provided that Z¹, Z² and Z³ are not N at the same time;
R^{z}, at each occurrence, is independently selected from hydrogen, halogen, —C₁₋₈alkyl, —NR^{Za}R^{Zb}, —OR^{Za}, —SR^{Za}, C₃-C₈cycloalkyl, 3- to 8-membered heterocyclyl or CN; each of —C₁₋₈alkyl, C₃-C₈cycloalkyl, 3- to 8-membered heterocyclyl is optionally substituted with at least one R^{Zc};
the 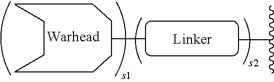
moiety is linked to the 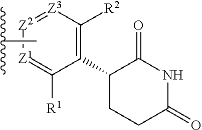
moiety via any one of Z¹, Z² or Z³ which is CR^{z} and R^{z} is hydrogen;
R^{Za} and R^{Zb} are each independently selected from hydrogen, —C₁-C₈alkyl, C₃-C₈cycloalkyl, 3- to 8-membered heterocyclyl, C₆-C₁₂aryl, or 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl is optionally substituted with at least one substituent R^{Zd};
R^{Zc} and R^{Zd} are each independently halogen, hydroxy, —C₁-C₈alkyl, C₃-C₈cycloalkyl, —C₁₋₈alkoxy, 3- to 8-membered heterocyclyl, C₆-C₁₂aryl, or 5- to 12-membered heteroaryl;
R¹ and R² are each independently selected from halogen, —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl, 5- to 12-membered heteroaryl, —CN, —SO₂R^{1a}, —SO₂NR^{1a}R^{1b}, —COR^{1a}, —CO₂R^{1a}, —CONR^{1a}R^{1b}, —NR^{1a}R^{1b}, —NR^{1a}COR^{1b}, —NR^{1a}CO₂R^{1b}, or —NR^{1a}SO₂R^{1b}; each of —C₁₋₈alkyl, —C₂₋₈alkenyl, —C₂₋₈alkynyl, —C₁₋₈alkoxy, —C₃-C₈cycloalkyl, 3- to 8-membered heterocyclyl, —C₆-C₁₂aryl or 5- to 12-membered heteroaryl is optionally substituted with halogen, —C₁₋₈alkyl, —C₂₋₈alkenyl, —C₂₋₈alkynyl, —C₃-C₈cycloalkyl, 3- to 8-membered heterocyclyl, C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}OR^{1d}, —NR^{1c}CO₂R^{1d}, or —NR^{1c}SO₂R^{1d};
R^{1a}, R^{1b}, R^{1c} and R^{1d} are each independently hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, or 5- to 12-membered heteroaryl.

2. The compound of claim 1, wherein at most one of Z¹, Z² and Z³ is N.

3. The compound of any one of claims 1-2, wherein Z¹, Z² and Z³ are each independently CR^{z}.

4. The compound of any one of claims 1-3, wherein R^{Z}, at each occurrence, is independently selected from hydrogen, —F, —Cl, —Br, —I, methyl, ethyl, propyl, butyl, pentyl, hexyl, heptyl, octyl, —NR^{Za}R^{Zb}, —OR^{Za}, —SR^{Za}, cyclopropyl, cyclobutyl, cyclopentyl, cyclohexyl, cycloheptyl, cyclooctyl, 3- to 8-membered heterocyclyl, phenyl, 5- to 12-membered heteroaryl, or CN; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one R^{Zc};
R^{Za} and R^{Zb} are each independently selected from hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{Zd};
R^{Zc} and R^{Zd} are each independently —F, —Cl, —Br, —I, hydroxy, methyl, ethyl, propyl, butyl, pentyl, hexyl, heptyl, octyl, —C₁₋₈alkoxy, —C₂₋₈alkenyl, —C₂₋₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

5. The compound of any one of claims 1-4, wherein R^{z} is selected from H, —CH₃, —C₂H₅, F, —CH₂F, —CHF₂, —CF₃, —OCH₃, —OC₂H₅, —C₃H₇, —OCH₂F, —OCHF₂, —OCH₂CF₃, —OCF₃, —SCF₃, —CF₃ or —CH(OH)CH₃.

6. The compound of any one of claims 1-5, wherein R¹ and R² are each independently selected from F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, —CN, —SO₂R^{1a}, —SO₂NR^{1a}R^{1b}, —COR^{1a}, —CO₂R^{1a}, —CONR^{1a}R^{1b}, —NR^{1a}R^{1b}, —NR^{1a}COR^{1b}, —NR^{1a}CO₂R^{1b}, or —NR^{1a}SO₂R^{1b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl is optionally substituted with F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, 5- to 12-membered heteroaryl, oxo, —CN, —OR^{1c}, —SO₂R^{1c}, —SO₂NR^{1c}R^{1d}, —COR^{1c}, —CO₂R^{1c}, —CONR^{1c}R^{1d}, —NR^{1c}R^{1d}, —NR^{1c}COR^{1d}, —NR^{1c}CO₂R^{1d}, or —NR^{1c}SO₂R^{1d};
R^{1a}, R^{1b}, R^{1c} and R^{1d} are each independently hydrogen, methyl, ethyl, propyl, butyl, pentyl, hexyl, heptyl, octyl, octyloxy, cyclopropyl, cyclobutyl, cyclopentyl, cyclohexyl, cycloheptyl, cyclooctyl, —C₂₋₈alkenyl, —C₂₋₈alkynyl, 3- to 8-membered heterocyclyl, —C₆-C₁₂aryl, or 5- to 12-membered heteroaryl.

7. The compound of any one of claims 1-6, wherein R¹ and R² are each independently selected from F, Cl, Br, I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, —CN, —CH₂F, —CHF₂, —CF₃, —OCH₂F, —OCHF₂, —OCH₂CF₃, —OCF₃, —SCF₃, or phenyl.

8. The compound of any one of claims 1-7, wherein the compound is Formula (II) 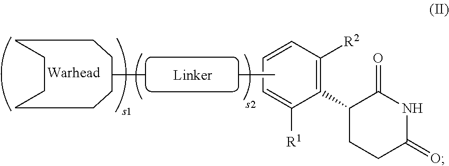
wherein Warhead and Linker are defined as claim 1.

9. The compound of any one of claims 1-8, wherein Linker is 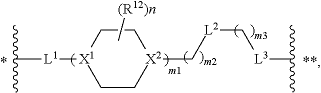
wherein * refers to the position attached to the 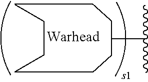
moiety, and ** refers to the position attached to the 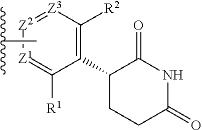
moiety;
L¹ is selected from a single bond, —O—, —SO₂—, —C(O)—, —NR^{L1a}—, —C₃-C₈cycloalkylene-, *^{L1}-O—C₁₋₈alkylene-**^{L1}, *^{L1}-C₁₋₈alkylene-O-**^{L1}, *^{L1}-SO₂—C₁₋₈alkylene-**^{L1}, *^{L1}-C₁₋₈alkylene-SO₂-**^{L1}, *^{L1}-CO—C₁₋₈alkylene-**^{L1}, *^{L1}-C₁₋₈alkylene-CO-**^{L1}, *^{L1}-NR^{L1a}—C₁₋₈alkylene-**^{L1}, *^{L1}-C₁₋₈alkylene-NR^{L1a}-**^{L1}, *^{L1}-NR^{L1a}C(O)-**^{L1}, *^{L1}-C(O)NR^{L1a}-**^{L1}, —C₁₋₈alkylene, —C₂₋₈alkenylene, —C₂₋₈alkynylene, —[O(CR^{L1a}R^{L1b})ₘ₄]ₘ₅—, 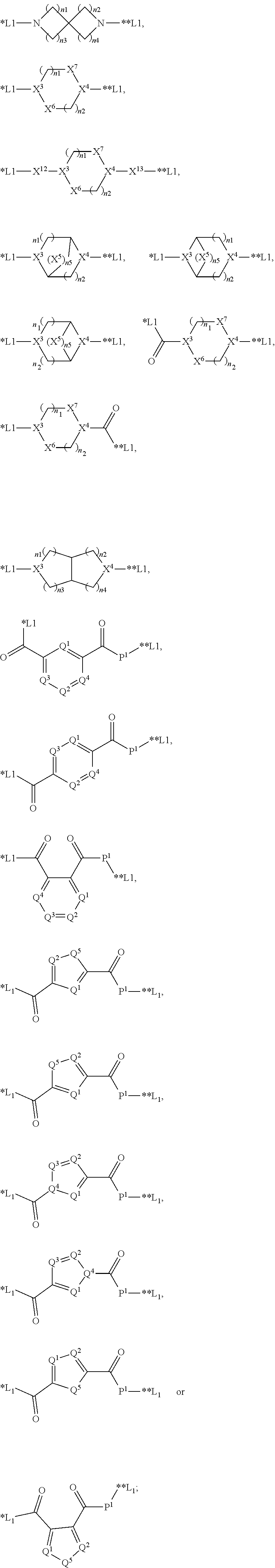
wherein each of said —C₃-C₈cycloalkylene-, *^{L1}-C₁₋₈alkylene**^{L1}, *^{L1}-C₁₋₈alkylene-O-**^{L1}, *^{L1}-SO₂—C₁₋₈alkylene-**^{L1}, *^{L1}-C₁₋₈-alkylene-SO₂-**^{L1}, *^{L1}-CO—C₁₋₈alkylene-**^{L1}, *^{L1}-C₁₋₈alkylene-CO-**^{L1}, *^{L1}-NR^{L1a}—C₁₋₈alkylene-**^{L1}, *^{L1}-C₁₋₈alkylene-NR^{L1a}-**^{L1}, —C₁₋₈alkylene-, —C₂₋₈alkenylene-, —C₂₋₈alkynylene-, 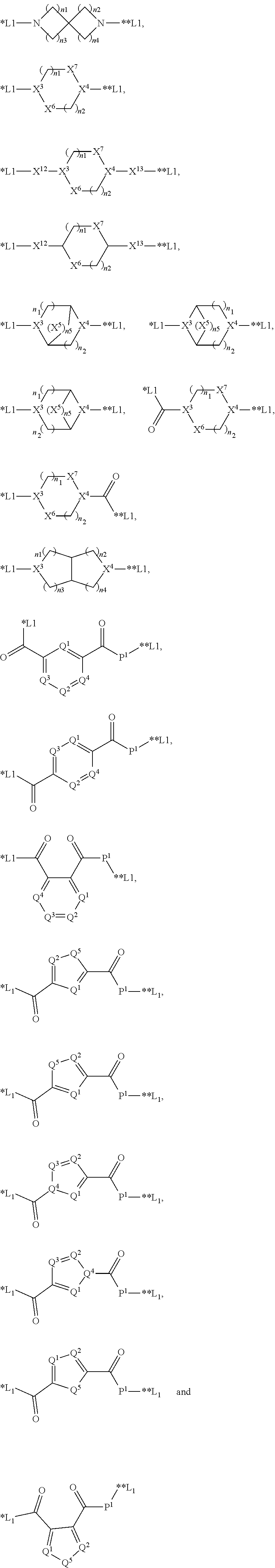
is optionally substituted with at least one R^{L1c};
wherein *^{L1} refers to the position attached to the 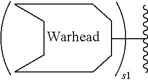
moiety, and **^{L1} refers to the position attached to the 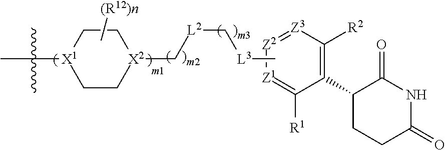
moiety;
R^{L1a} and R^{L1b} are each independently selected from hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl, 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{L1d};
each of said R^{L1c} and R^{L1d} are independently oxo, halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl; or
two R^{L1c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, or —C₁₋₈alkyl;
L² is selected from a single bond, —O—, —SO₂—, —CO—, —NR^{L2a}—, —C₃-C₈cycloalkylene-, *^{L2}-O—C₁₋₈alkylene-**^{L2}, *^{L2}-C₁₋₈alkylene-O-**^{L2}, *^{L2}-SO₂—C₁₋₈alkylene-**^{L2}, *^{L2}-C₁₋₈alkylene-SO₂-**^{L2}, *^{L2}-CO—C₁₋₈alkylene-**^{L2}, *^{L2}-C₁₋₈alkylene-CO-**^{L2}, *^{L2}-NR^{L2a}—C₁₋₈-alkylene-**^{L2}, *^{L2}-C₁₋₈alkylene-NR^{L2a}-**^{L2}, *^{L2}-NR^{L2a}C(O)-**^{L2}, *^{L2}-C(O)NR^{L2a}-**^{L2}, —C₁₋₈alkylene-, —C₂₋₈alkenylene, —C₂₋₈-alkynylene-, —[O(CR^{L2a}R^{L2b})m₄]ₘ₅-, 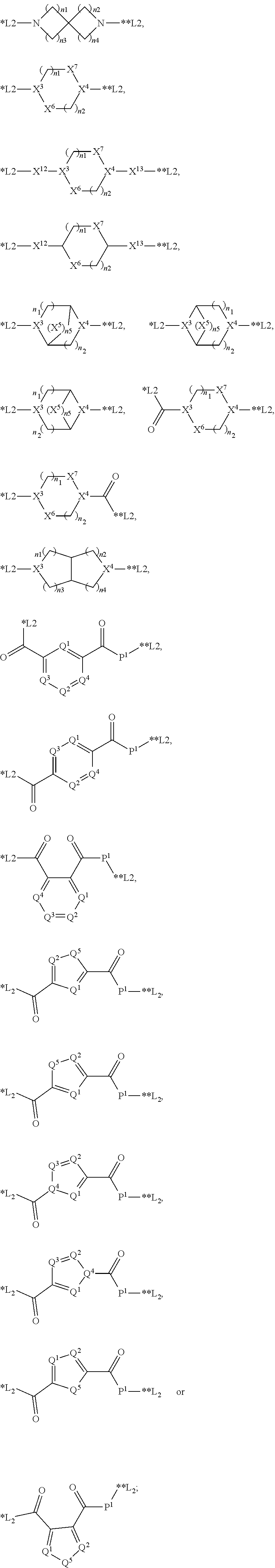
wherein each of said —C₃-C₈cycloalkylene-, *^{L2}-O—C₁₋₈alkylene-**^{L2}, *^{L2}-C₁₋₈alkylene-O-**^{L2}, *^{L2}-SO₂—C₁₋₈alkylene-**^{L2}, *^{L2}-C₁₋₈alkylene-SO₂-**^{L2}, *^{L2}-CO—C₁₋₈alkylene-**^{L2}, *^{L2}-C₁₋₈alkylene-CO-**^{L2}, *^{L2}-NR^{L2a}—C₁₋₈alkylene-**^{L2}, *^{L2}-C₁₋₈alkylene-NR^{L2a}-**^{L2}, —C₁₋₈alkylene-, —C₂₋₈alkenylene-, —C₂₋₈alkynylene-, 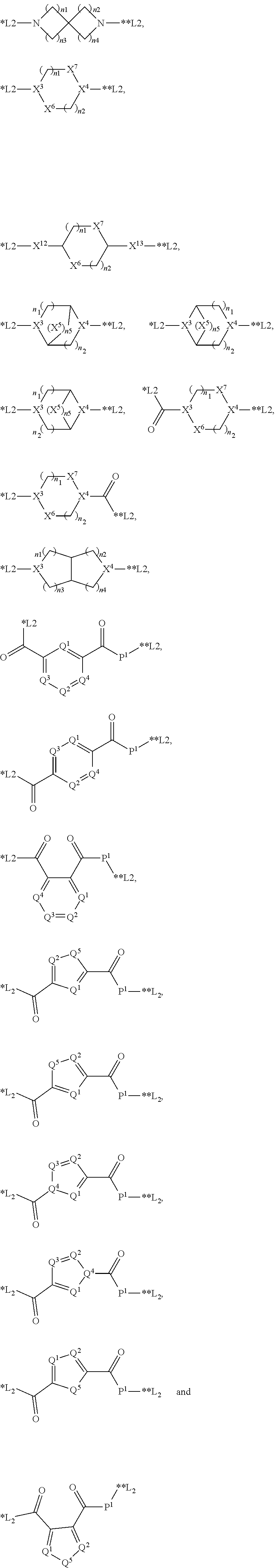
is optionally substituted with at least one substiutent R^{L2c};
wherein *^{L2} refers to the position attached to 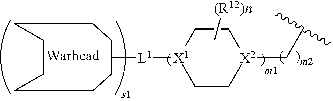
moiety, and **^{L2} refers to the position attached to the 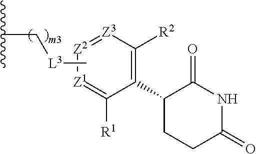
moiety;
R^{L2a} and R^{L2b} are each independently selected from hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C-C2aryl or 5- to 12-membered heteroaryl is optionally substituted with at least one substituent R^{L2d};
each of said R^{L2c} and R^{L2d} are independently oxo, halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl; or
two R^{L2c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁₋₈alkyl;
L³ is selected from a single bond, —O—, —SO₂—, —CO—, —NR^{L3a}—, —C₃-C₈cycloalkylene-, *^{L3}-OC₁₋₈alkylene-**^{L3}, *^{L3}-C₁₋₈alkylene-O-**^{L3}, *^{L3}-SO₂—C₁₋₈alkylene-**^{L3}, *^{L3}-C₁₋₈alkylene-SO₂-**^{L3}, *^{L3}-CO—C₁₋₈alkylene-**^{L3}, *^{L3}-C₁₋₈alkylene-CO-**^{L3}, *^{L3}-NR^{L3a}—C₁₋₈alkylene-**^{L3}, *^{L3}-C₁₋₈alkylene-NR^{L3a}-**^{L3}, *^{L3}-NR^{L3a}C(O)-**^{L3}, *^{L3}-C(O)NR^{L3a}-**^{L3}, —C₁₋₈ alkylene- —C₂₋₈alkenylene-, —C₂₋₈alkynylene-, —[O(CR^{L3a}R^{L3b})ₘ₄]ₘ₅—, 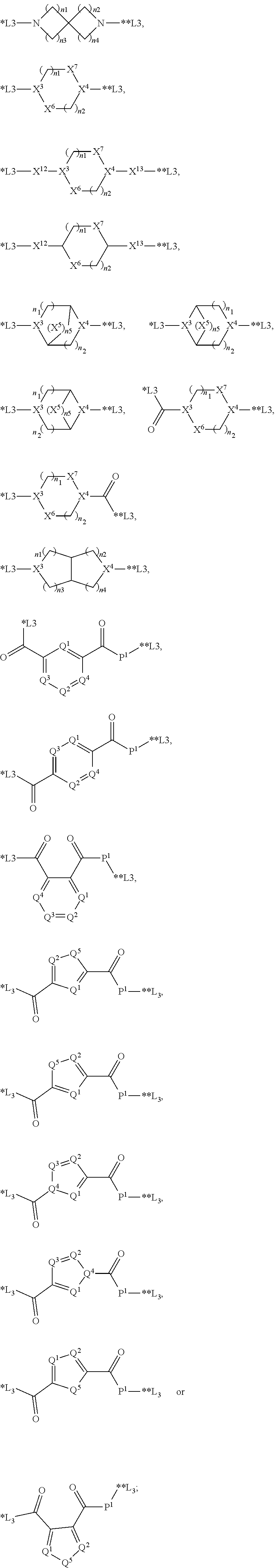
wherein each of said —C₃-C₅cycloalkylene-, *^{L3}-O—C₁₋₈alkylene-**^{L3}, *^{L3}-C₁₋₈alkylene-O-**^{L3}, *^{L3}-SO₂—C₁₋₈alkylene-**^{L3}, *^{L3}-C₁₋₈alkylene-SO₂-**^{L3}, *^{L3}-CO—C₁₋₈alkylene-**^{L3}, *^{L3}-C₁₋₈alkylene-CO-**^{L3}, *^{L3}-NR^{L3a}C₁₋₈alkylene-**^{L3}, *^{L3}-C₁₋₈alkylene-NR^{L3a}-**^{L3}-C₁₋₈alkylene-, —C₂₋₈alkenylene-, —C₂₋₈alkynylene-, 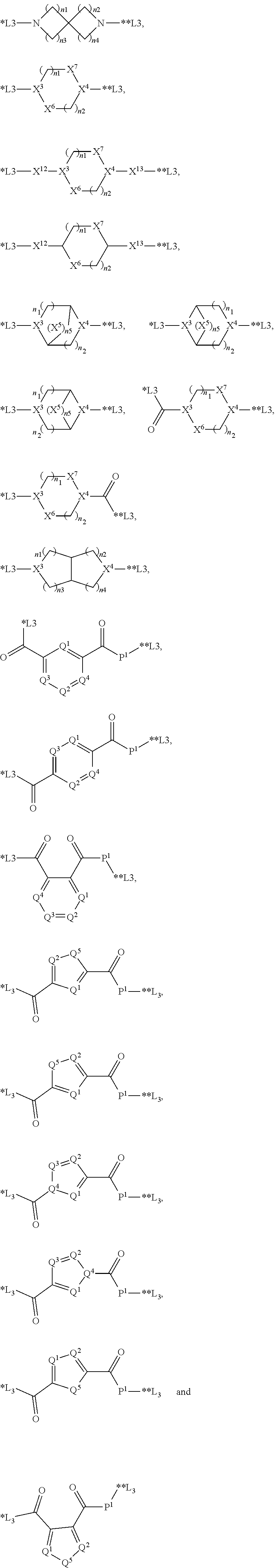
is optionally substituted with at least one substituent R^{L3c};
wherein *^{L3} refers to the position attached to 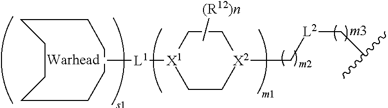
moiety, and **^{L3} refers to the position attached to the 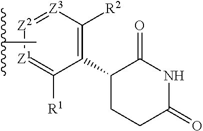
moiety;
R^{L3a} and R^{L3b} are each independently selected from hydrogen, —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{L3d};
each of said R^{L3c} and R^{L3d} are independently oxo, halogen, hydroxy, —C₁₋₈alkyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl; or
two R^{L3c} together with the atoms to which they are attached, form a 3- to 12-membered ring, said ring comprising 0-3 heteroatoms independently selected from nitrogen, oxygen or sulfur; said ring is optionally substituted with at least one substituent halogen, hydroxy, or —C₁₋₈alkyl;
R¹² is independently selected from hydrogen, halogen, —C₁₋₈alkyl, —NR^{12a}R^{12b}, —OR^{12a}, —C₃-C₈cycloalkyl, 3- to 8-membered heterocyclyl, —C₆-C₁₂aryl, 5- to 12-membered heteroaryl, oxo, or —CN;
each of —C₁₋₈alkyl, C₃-C₈cycloalkyl, 3- to 8-membered heterocyclyl, C₆-C₁₂aryl, or 5- to 12-membered heteroaryl is optionally substituted with at least one substituent R^{12c}; or
two R¹² together with the carbon atoms to which they are attached, form a 3- to 12-membered ring, said ring comprising 0-3 heteroatoms independently selected from nitrogen, oxygen or sulfur; said ring is optionally substituted with at least one substituent R^{12c};
R^{12a} and R^{12b} are each independently selected from hydrogen, —C₁₋₈alkyl, —C₂₋₈alkenyl, —C₂₋₈alkynyl, —C₃-C₈cycloalkyl, 3- to 8-membered heterocyclyl, —C₆-C₁₂aryl or 5- to 12-membered heteroaryl, each of said —C₁₋₈alkyl, —C₂₋₈alkenyl, —C₂₋₈alkynyl, C₃-C₈cycloalkyl, 3- to 8-membered heterocyclyl, C₆-C₁₂aryl or 5- to 12-membered heteroaryl is optionally substituted with at least one substituent R^{12d}; or
R^{12c} and R^{12d} are each independently halogen, hydroxy, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl or 5- to 12-membered heteroaryl;
X¹, X², X³ and X⁴ are each independently selected from —CR^{a}, or N;
X⁵, X⁶ and X⁷ are each independently selected from —NR^{a}—, —O—, —S— and —CR^{a}R^{b}—;
X¹² and X¹³ are each independently selected from —C(O)—, —NR^{a}— and —O—;
Q¹, Q², Q³ and Q⁴ are each independently selected from CR^{a} or N;
Q⁵ is each independently selected from —O—, —NR^{a}—, —CR^{a}—, —CR^{a}R^{b}—, —S— or —C(O)—;
P¹ is a single bond, —O—, —NR^{a}—, —CR^{a}R^{b}—, —S—, —SO— or —SO₂—;
at each occurrence, R^{a} and Rb are each independently selected from hydrogen, hydroxy, halogen, CN, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl or 5- to 12-membered heteroaryl, each of said —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl or 5- to 12-membered heteroaryl is optionally substituted with at least one substituent halogen, hydroxy, halogen, —C₁₋₈alkyl, —C₁₋₈alkoxy, —C₂₋₈alkenyl, —C₂₋₈alkynyl, —C₃-C₈cycloalkyl, 3- to 8-membered heterocyclyl, —C₆-C₁₂aryl or 5- to 12-membered heteroaryl; or
Ra and Rb together with the carbon atoms to which they are attached, form a 3- to 12-membered ring, said ring comprising 0-3 heteroatoms independently selected from nitrogen, oxygen or sulfur; said ring is optionally substituted with at least one substituent halogen, hydroxy, —C₁₋₈alkyl, —C₂₋₈alkenyl, —C₂₋₈alkynyl, —C₁₋₈alkoxy, —C₂₋₈alkenyl, —C₂₋₈alkynyl, C₃-C₈cycloalkyl, 3- to 8-membered heterocyclyl, C₆-C₁₂aryl or 5- to 12-membered heteroaryl;
m₁ is 0 or 1;
m₂ and m3 is 0, 1, 2, 3, 4, 5, 6, 7 or 8;
m₄ and m₅ are each independently 0, 1, 2 or 3;
n, n₁, n₂, n₃, n₄ and n₅ are each independently 0, 1, 2 or 3.

10. The compound of claim 9, wherein L¹ is selected from a single bond, —C₁₋₈alkylene- preferably —CH₂—, —C₂H₄—, —C₃H₆—), —CO—, —O—, —N(CH₃)—, —NH—,

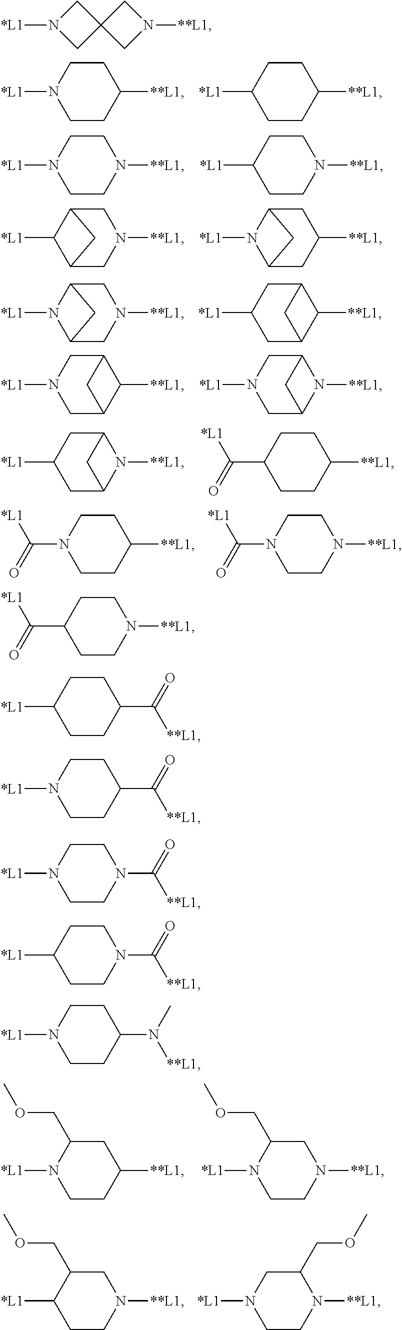

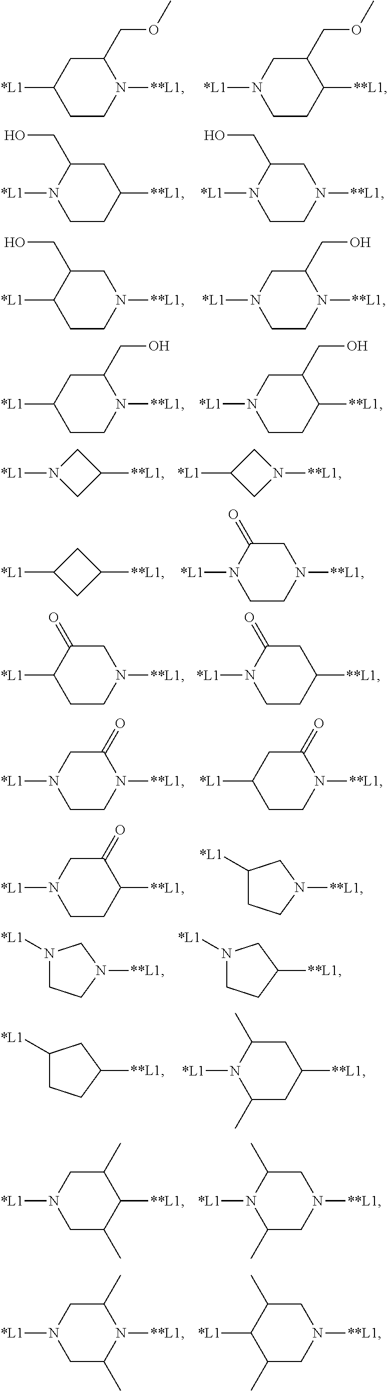

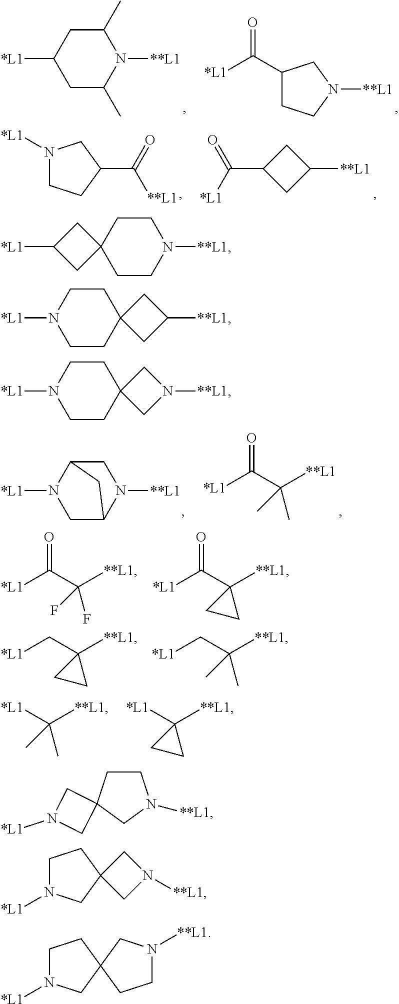

11. The compound of any one of claims 9-10, wherein X¹ and X² are each independently selected from —CR^{a} or N;
R^{a} is selected from hydrogen, —F, —Cl, —Br, —I, CN, methyl, ethyl, methoxy, ethoxy, or cyclopropyl, wherein each of said methyl, ethyl, methoxy, ethoxy, and cyclopropyl is optionally substituted with at least one substituent —F, —Cl, —Br, —I, hydroxy, methyl, ethyl (preferably, X¹ and X² are each independently selected from CH, C(F), C(CH₃) or N);
m₁=1 or 0;
R¹² is hydrogen, oxo, methoxymethyl, hydroxymethyl, —CN or —CH₃.

12. The compound of any one of claims 9-11, wherein m₁ is 1; preferably, 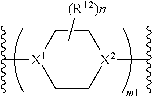
moiety is 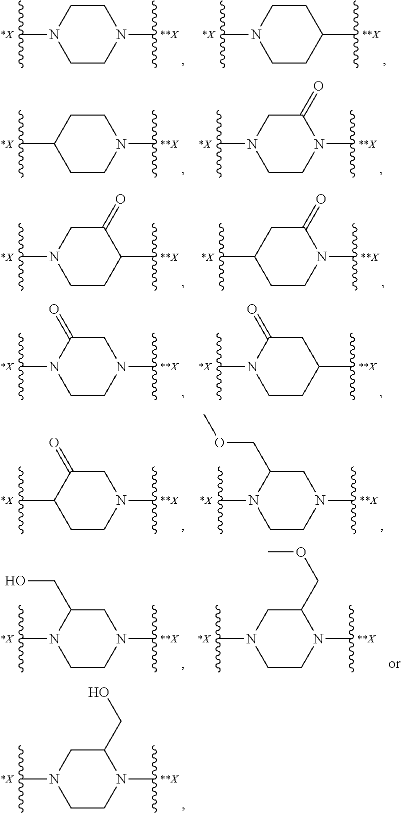
wherein *^{X} refers to the position attached to 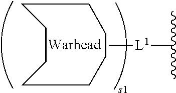
moeity, and **^{X} refers to the position attached to the 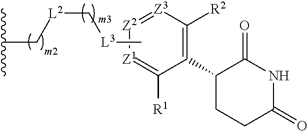
moiety.

13. The compound of any one of claims 9-12, wherein m₁ is 1, 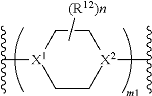
moiety is

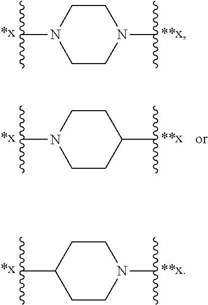 [SMILES: *N1CCN(*)CC1 ; *C1CCN(*)CC1 ; *C1CCN(*)CC1]

14. The compound of any one of claims 9-13, wherein L² is selected from a single bond, —C₁₋₈alkylene-(preferably —CH₂—, —C₂H₄—, —C₃H₆—), —CO—, —O—, —N(CH₃)—, —NH—,

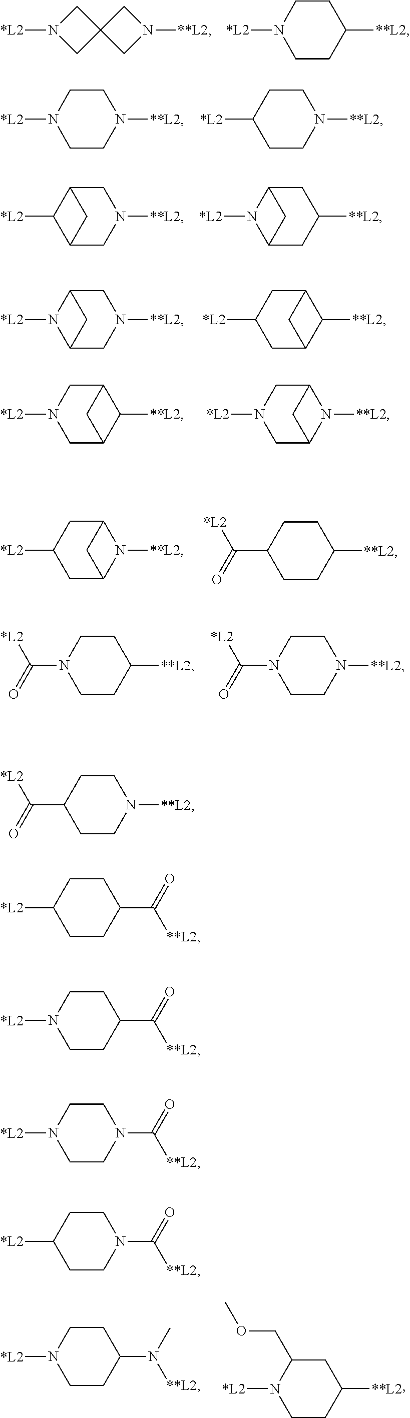

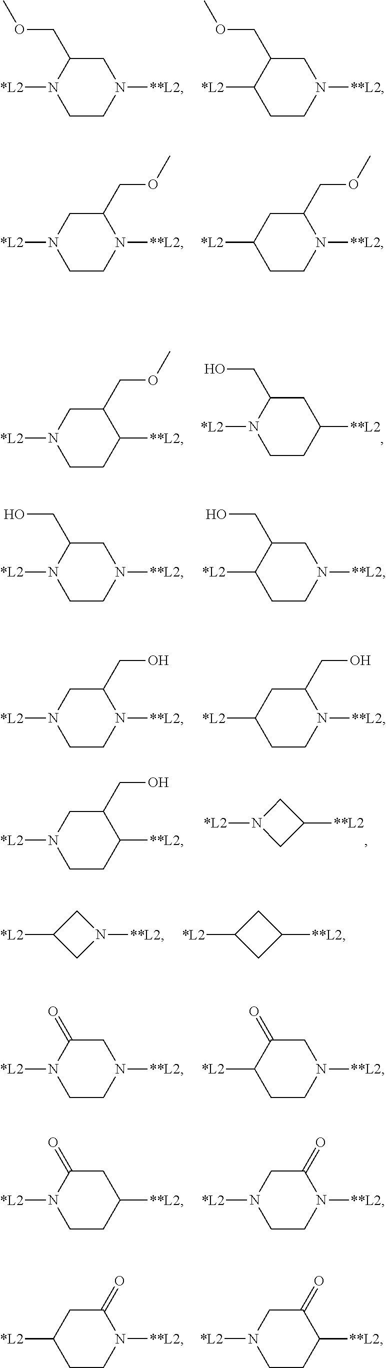

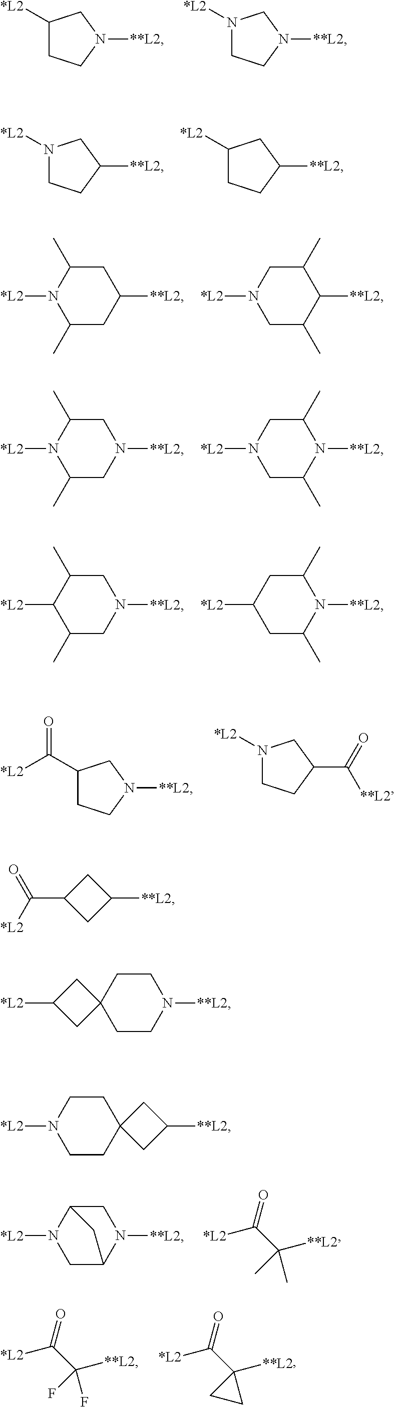

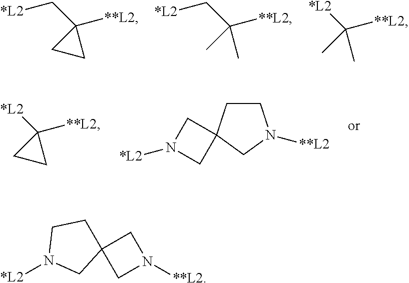

15. The compound of any one of claims 9-14, wherein L³ is selected from single bond, —C₁₋₈alkylene-(preferably —CH₂—, —C₂H₄—, —C₃H₆—), —CO—, —O—, —N(CH₃)—, —NH—,

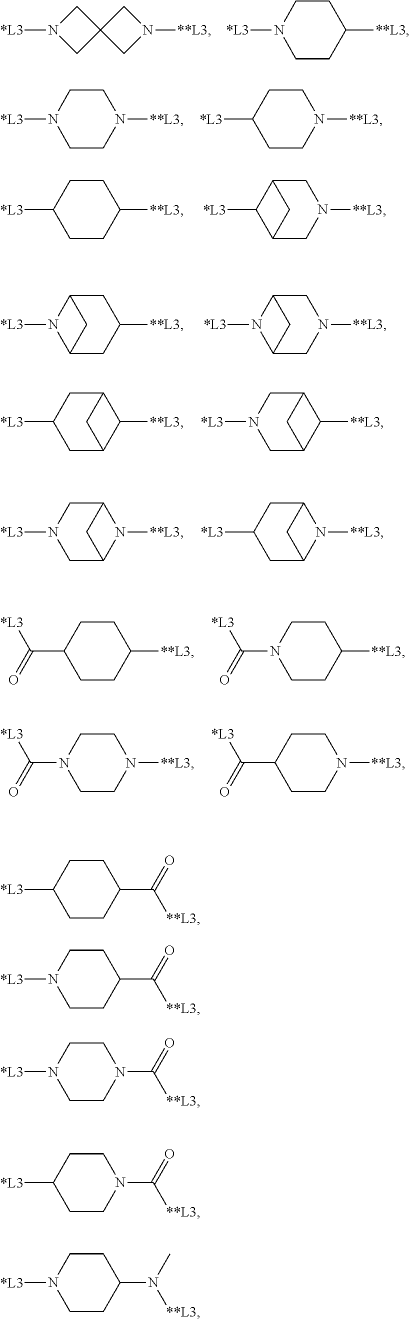

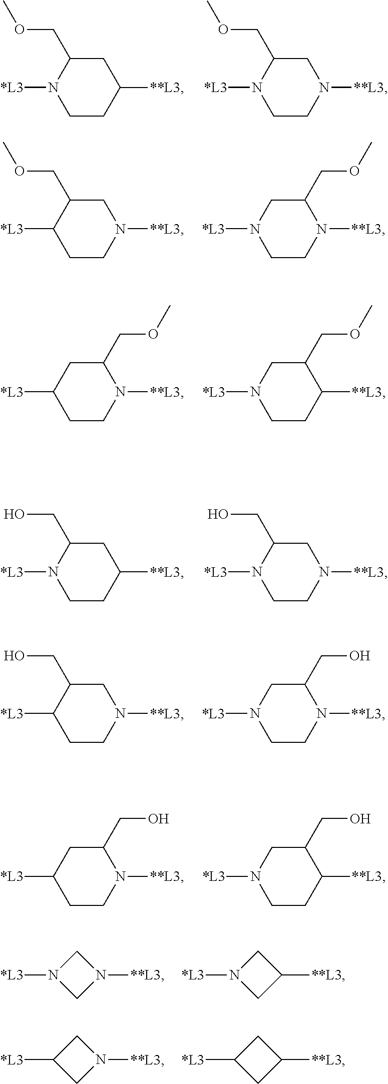

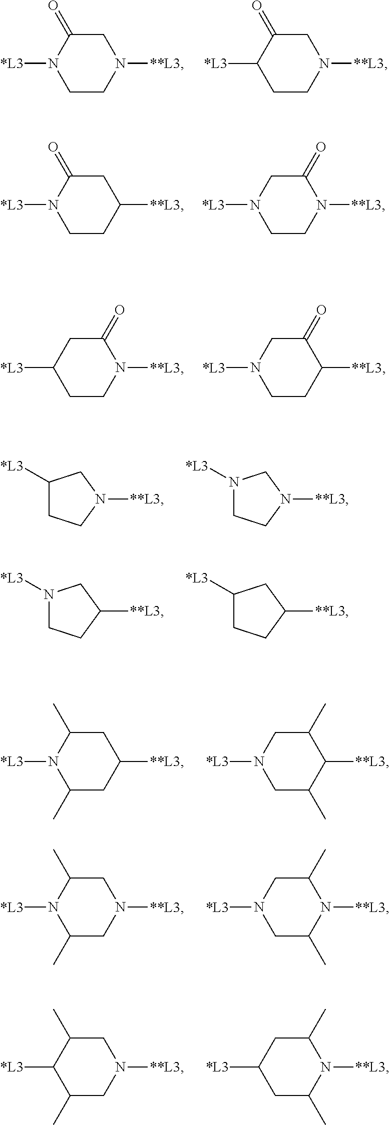

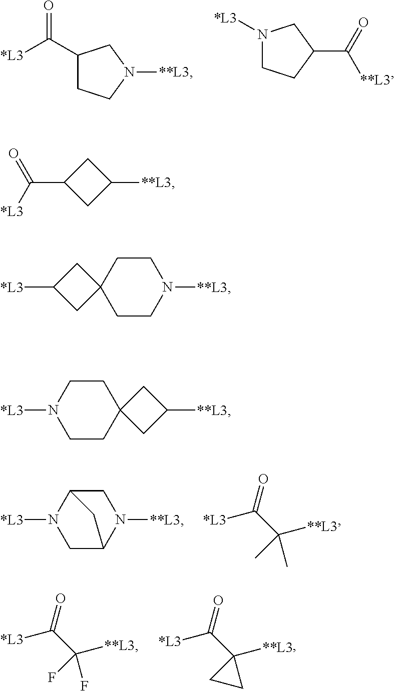

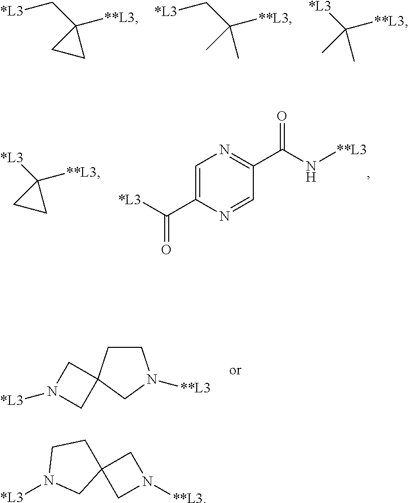

16. The compound of any one of claims 9-15, wherein L² is a single bond; or L³ is a single bond; or L² is a single bond and L³ is a single bond.

17. The compound of any one of claims 9-16, wherein 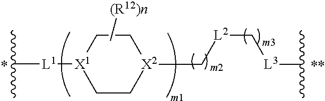
is selected from

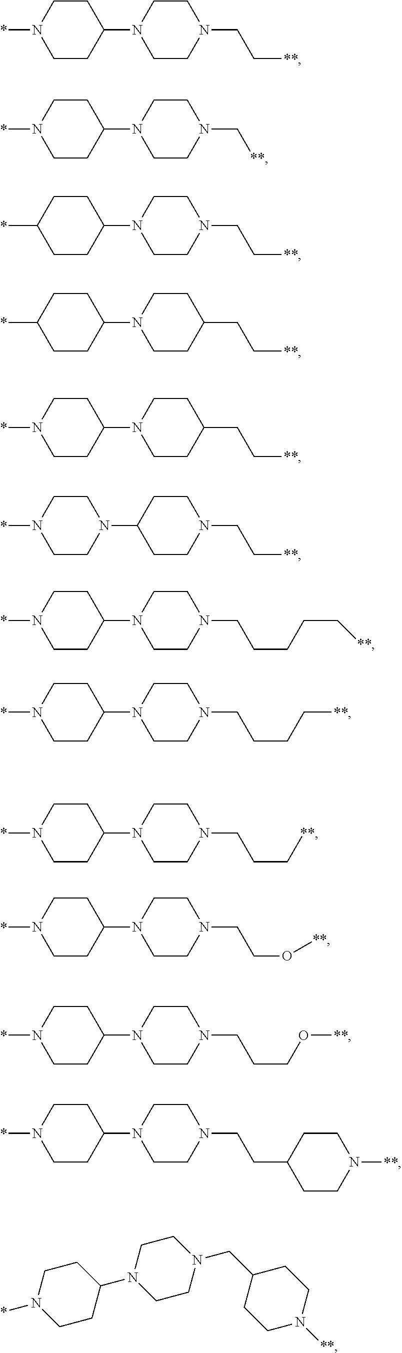

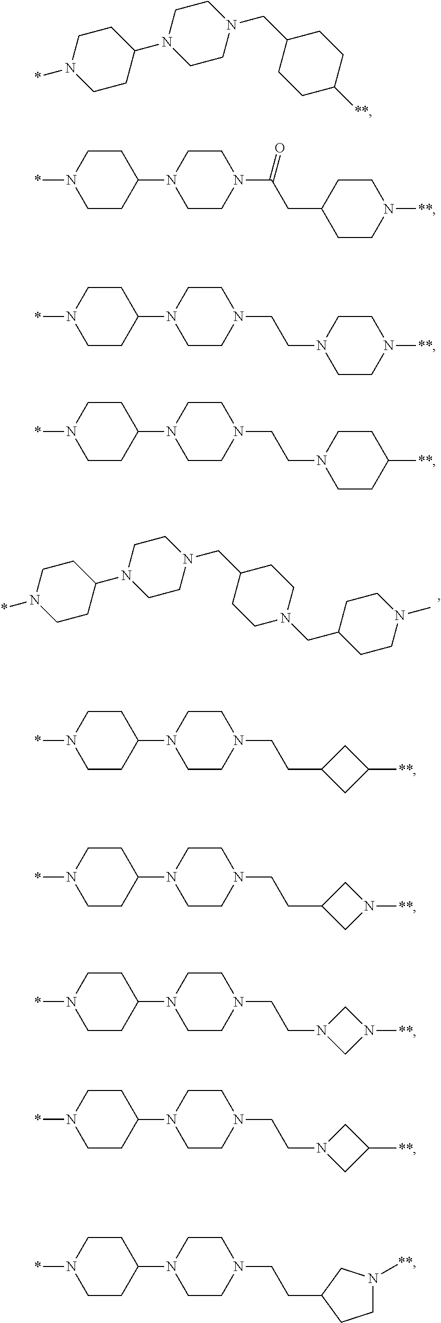

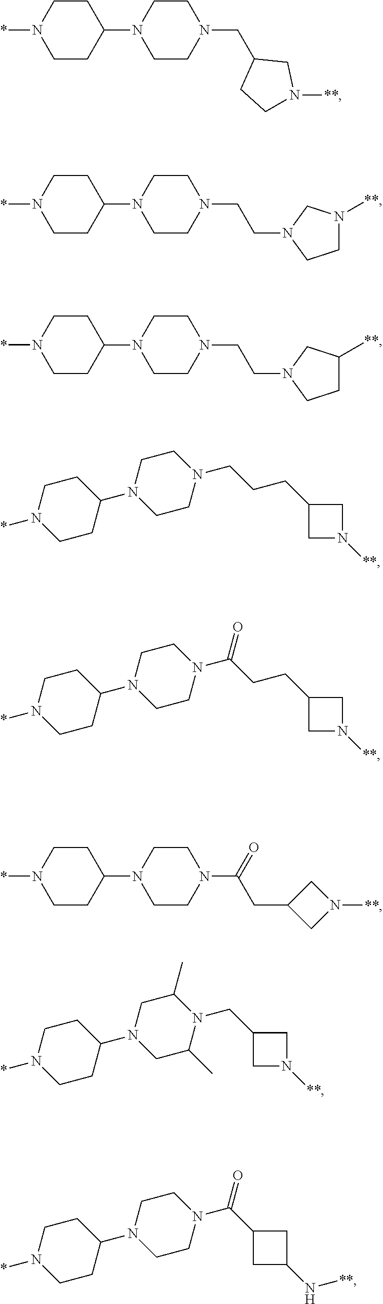

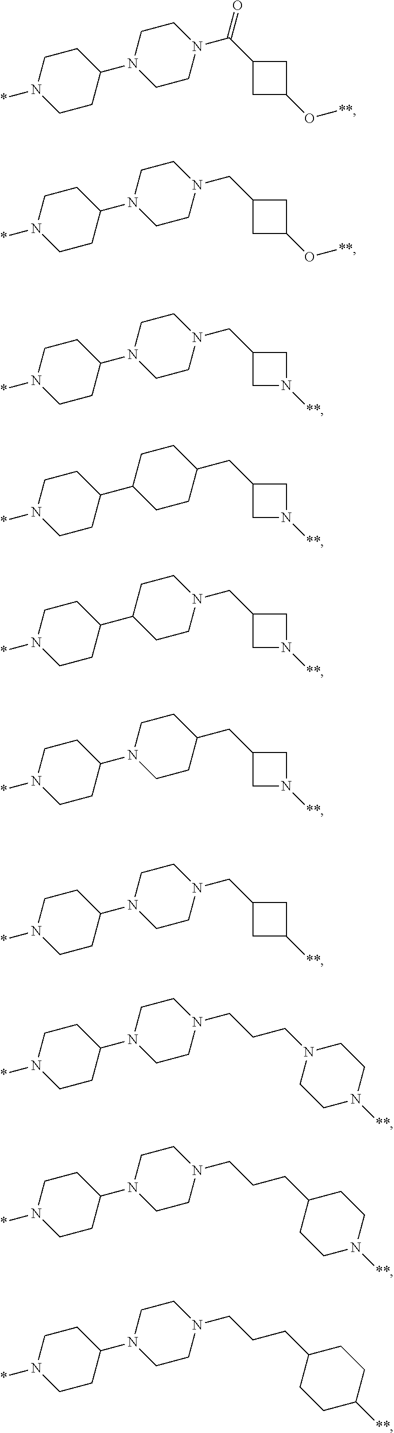

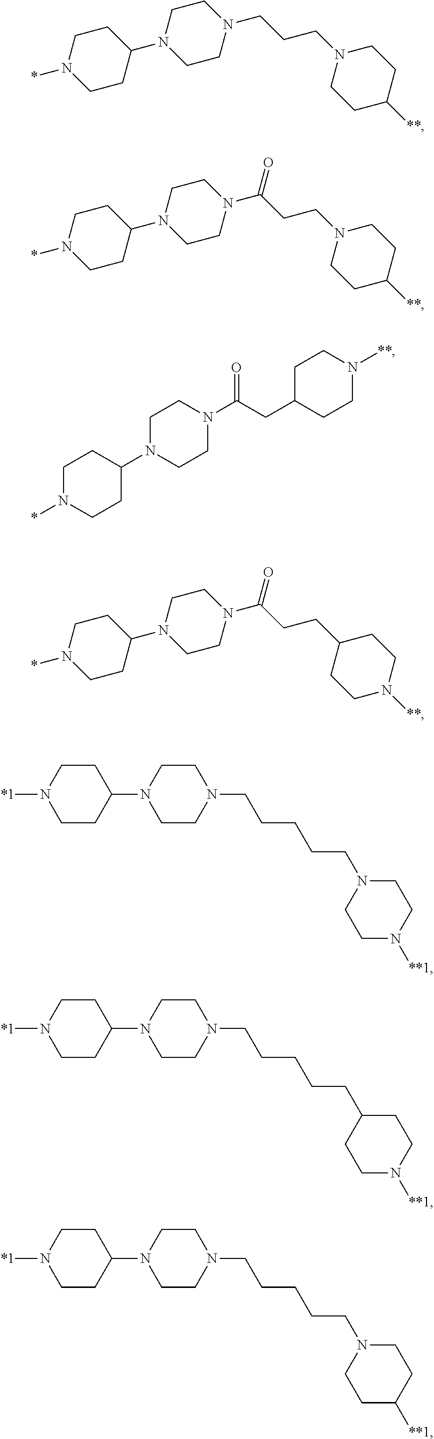

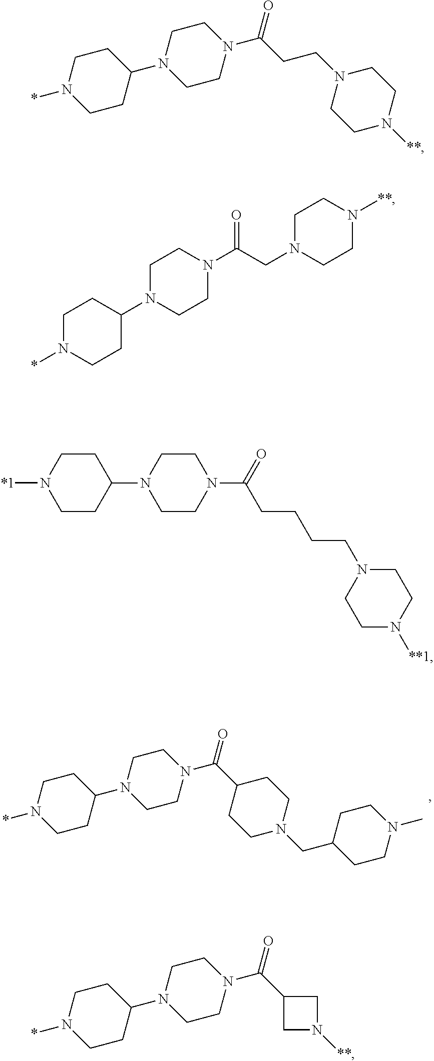

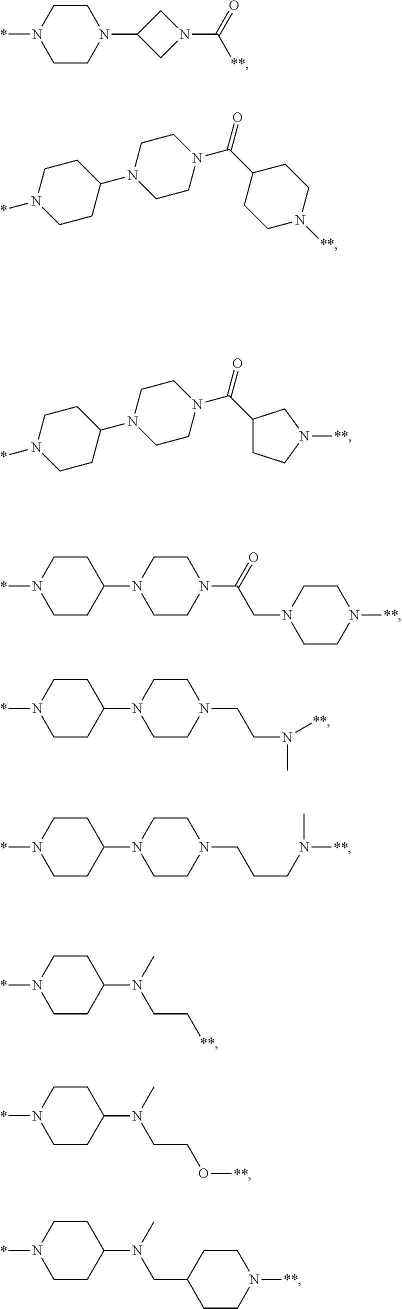

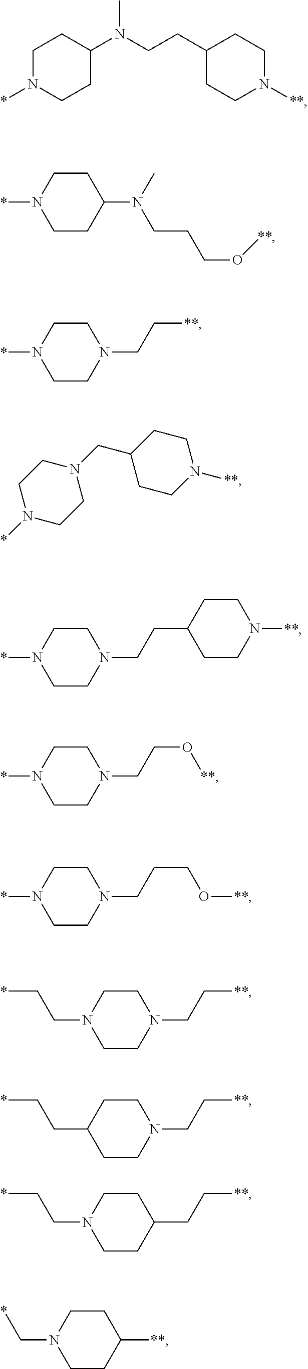

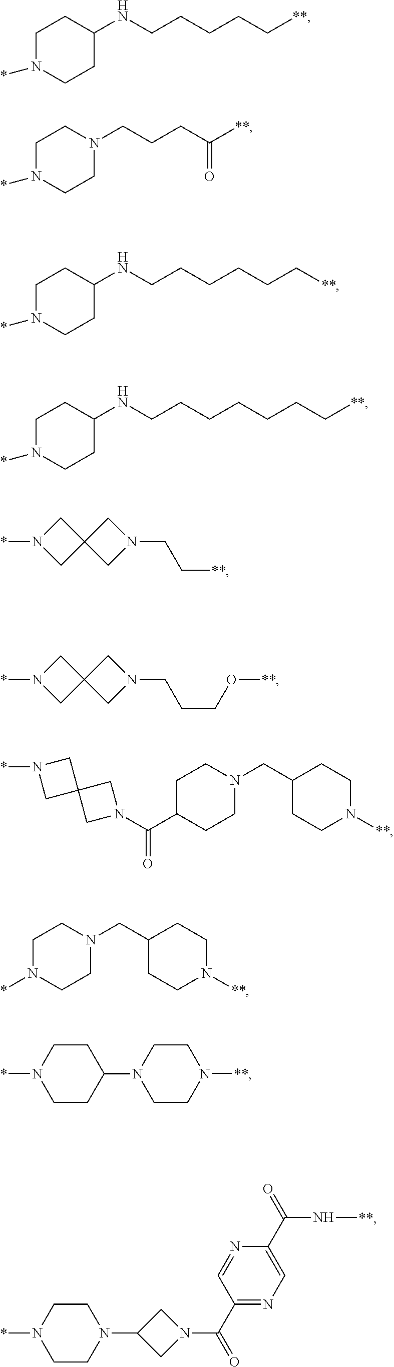

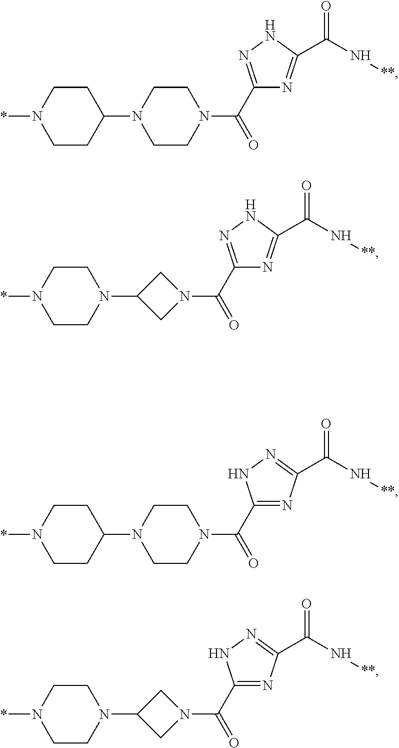

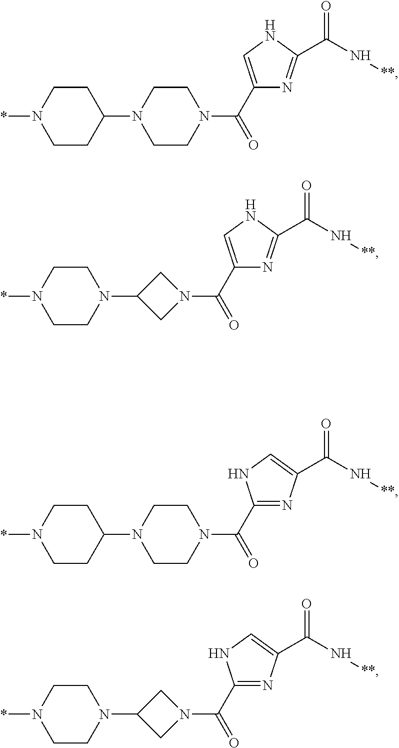

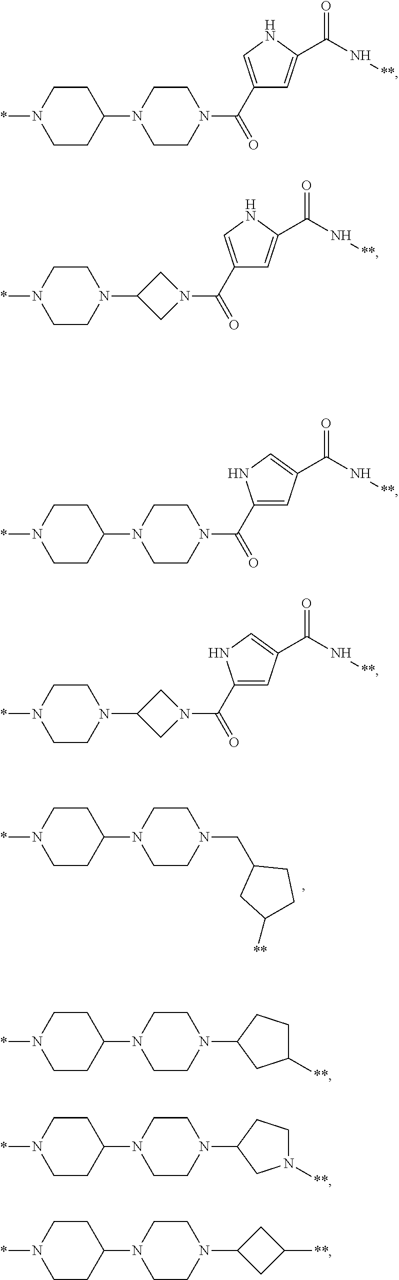

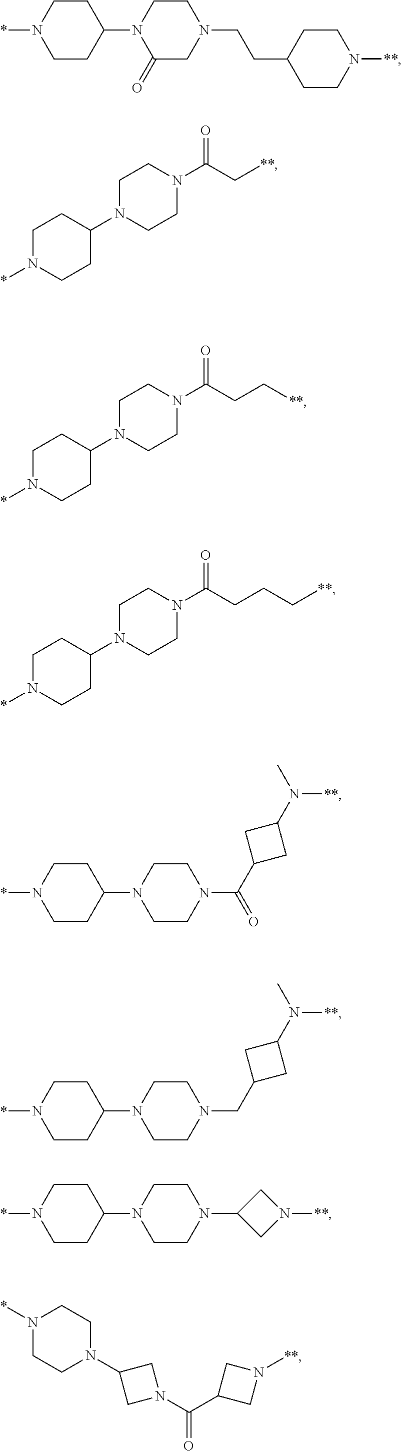

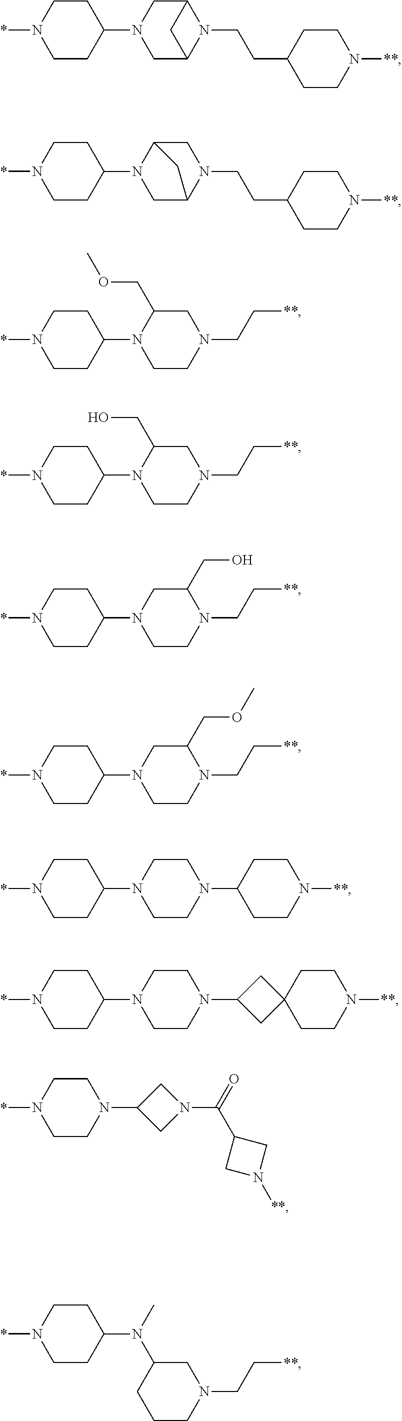

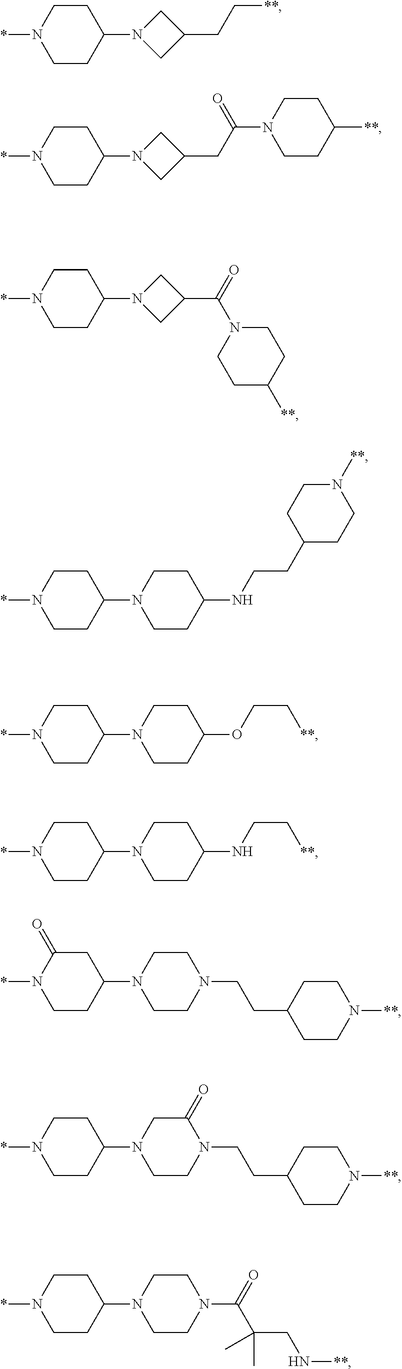

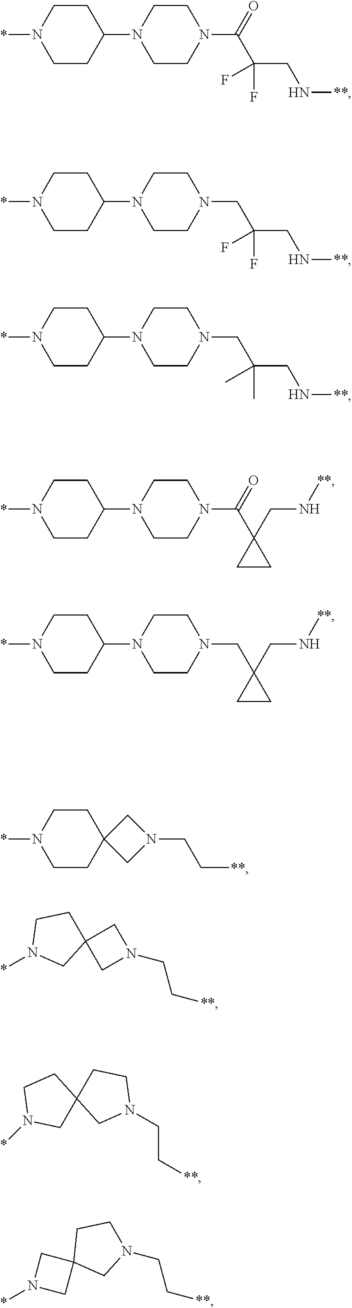

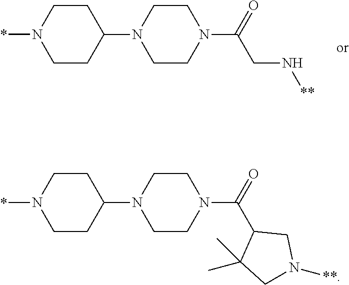

18. The compound of any one of claims 1-17, wherein Warhead is a moiety which binds to a target protein, wherein said target protein is selected from the group consisting of structural proteins, receptors, enzymes, cell surface proteins, proteins pertinent to the integrated function of a cell, including proteins involved in catalytic activity, aromatase activity, motor activity, helicase activity, metabolic processes (anabolism and catabolism), antioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity and translation regulator activity.

19. The compound of any one of claims 1-18, wherein Warhead is a moiety which binds to a target protein, wherein said target protein is selected from the group consisting of ErbB receptors, B7.1 and B7, TINFR1m, TNFR2, NADPH oxidase, Bcl-Bax and other partners in the apotosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases (including Bruton's Tyrosine Kinase), CD23, CD124, tyrosine kinase p561ck, CD4, CD5, IL-2 receptor, IL-1 receptor, TNF-alphaR, ICAM1, Cat+ channels, VCAM, VLA-4 integrin, selectins, CD40/CD40L, newokinins and receptors, inosine monophosphate dehydrogenase, p38 MAP Kinase, RAS-RAF-MEK-ERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, adenosine receptors, adenosine kinase and AMP deaminase, purinergic receptors (P2Y1, P2Y2, P2Y4, P2Y6, P2X 1-7), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase, ecdysone 20-monooxygenase, ion channel of the GABA gated chloride channel, acetylcholinesterase, voltage-sensitive sodium channel protein, calcium release channel, chloride channels, Acetyl-CoA carboxylase, adenylosuccinate synthetase, protoporphyrinogen oxidase, L-1 receptor associated kinase-3 (IRAK-3 or IRAK-M) or enolpyruvyl-shikimate-phosphate synthase.

20. The compound of any one of claims 1-19, wherein Warhead is 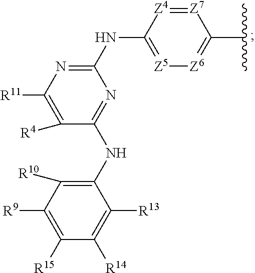
wherein R¹³ is selected from —P(O)R^{13a}R^{13b}, —SO₂R^{13a}, —SO₂—NR^{13a}R^{13b} or —N(R^{13a})—SO₂R^{13b};
R^{13a} and R^{13b} are each independently selected from hydrogen, —C₁-C₈alkyl or C₃-C₈cycloalkyl, said —C₁-C₈alkyl or C₃-C₈cycloalkyl is optionally substituted with at least one halogen;
R¹⁴ and R¹⁵ are each independently selected from hydrogen, halogen, —C₁-C₈alkyl, —C₃-C₈cycloalkyl, 3- to 8-membered heterocyclyl, C₆-C₁₂aryl, 5- to 12-membered heteroaryl, —CN, —OR^{14a}, —SO₂R^{14a}, —SO₂NR^{14a}R^{14b}, COR^{14a}, —CO₂R^{14a}, —CONR^{14a}R^{14b}, —NR^{14a}R^{14b}, —NR^{14a}COR^{14b}, —NR^{14a}CO₂R^{14b}, or —NR^{14a}SO₂R^{14b}; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{14d}, or
R¹⁴ and R¹⁵ together with the carbon atoms to which they are attached, form a 5 or 6-membered unsaturated or saturated ring, said ring comprising 0-3 heteroatoms independently selected from nitrogen, oxygen or sulfur; said ring is optionally substituted with at least one substituent R^{14c};
R^{14e}, at each occurrence, is independently hydrogen, halogen, —C₁-C₈alkyl, —C₁-C₈alkoxy, —C₃-C₈cycloalkyl, 3- to 8-membered heterocyclyl, C₆-C₁₂aryl, 5- to 12-membered heteroaryl, oxo (═O), —OR^{14a}, thioxo (═S), —SR^{14a}, —CN, —SO₂R^{14a}, —SO₂NR^{14a}R^{14b}, —COR^{14a}, —CO₂R^{14a}, —CONR^{14a}R^{14b}, —NR^{14a}R^{14b}, —NR^{14a}COR^{14b}, —NR^{14a}CO₂R^{14b} or —NR^{14a}SO₂R^{14b}; each of —C₁-C₈alkyl, —C₁-C₈alkoxy, C₃-C₈cycloalkyl, 3- to 8-membered heterocyclyl, C₆-C₁₂aryl or 5- to 12-membered heteroaryl is optionally substituted with at least one substituent R^{14d};
R^{14a} and R^{14b} are each independently selected from hydrogen, —C₁-C₈alkyl, —C₁-C₈haloalkyl, —C₂-C₈alkenyl, —C₂-C₈alkynyl, C₁-C₈alkoxy-C₁-C₈alkyl-, C₃-C₈cycloalkyl, 3- to 8-membered heterocyclyl, C₆-C₁₂aryl or 5- to 12-membered heteroaryl;
R^{14d}, at each occurrence, is independently halogen, —OH, —CN, oxo, —C₁-C₈alkyl, —C₂-C₈alkenyl, —C₂-C₈alkynyl, —C₃-C₈cycloalkyl, 3- to 8-membered heterocyclyl, —C₆-C₁₂aryl, or 5- to 12-membered heteroaryl;
R⁴ is selected from hydrogen, halogen, —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl, 5- to 12-membered heteroaryl, —CN, —SO₂R^{4a}, —SO₂NR^{4a}R^{4b}, —COR^{4a}, —CO₂R^{4a}, —CONR^{4a}R^{4b}, —NR^{4a}R^{4b}, —NR^{4a}COR^{4b}, —NR^{4a}CO₂R^{4b} or —NR^{4a}SO₂R^{4b}; each of —C₁-C₈alkyl, —C₂-C₈alkenyl, —C₂-C₈alkynyl, —C₁-C₈alkoxy, —C₃-C₈cycloalkyl, 3- to 8-membered heterocyclyl, —C₆-C₁₂aryl or 5- to 12-membered heteroaryl is optionally substituted with halogen, —C₁-C₈alkyl, —C₂-C₈alkenyl, —C₂-C₈alkynyl, —C₃-C₈cycloalkyl, 3- to 8-membered heterocyclyl, C₆-C₁₂aryl, 5- to 12-membered heteroaryl, oxo, —CN, —OR^{4c}, —SO₂R^{4c}, —SO₂NR^{4c}R^{4d}, —COR^{4c}, —CO₂R^{4c}, —CONR^{4c}R^{4d}, —NR^{4c}R^{4d}, —NR^{4c}COR^{4d}, —NR^{4c}CO₂R^{4d} or —NR^{4c}SO₂R^{4d};
R^{4a}, R^{4b}, R^{4c} and R^{4d} are each independently hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl;
R⁹, R¹⁰ and R¹¹ are each independently selected from hydrogen, halogen, —C₁-C₈alkyl, —NR^{9a}R^{9b}, —OR^{9a}, —C₃-C₈cycloalkyl, 3- to 8-membered heterocyclyl, —C₆-C₁₂aryl, 5- to 12-membered heteroaryl, oxo or —CN; each of —C₁-C₈alkyl, C₃-C₈cycloalkyl, 3- to 8-membered heterocyclyl, C₆-C₁₂aryl or 5- to 12-membered heteroaryl is optionally substituted with at least one substituent R^{9c};
R^{9a} and R^{9b} are each independently selected from hydrogen, —C₁-C₈alkyl, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl; each of said —C₁-C₈alkyl, —C₂-C₈alkenyl, —C₂-C₈alkynyl, C₃-C₈cycloalkyl, 3- to 8-membered heterocyclyl, C₆-C₁₂aryl or 5- to 12-membered heteroaryl is optionally substituted with at least one substituent R^{9d}; or
R^{9c} and R^{9d} are each independently halogen, hydroxy, —C₁-C₈alkyl, —C₁-C₈alkoxy, —C₂-C₈alkenyl, —C₂-C₈alkynyl, —C₃-C₈cycloalkyl, 3- to 8-membered heterocyclyl, —C₆-C₁₂aryl or 5- to 12-membered heteroaryl;
Z⁴, Z⁵, Z⁶ and Z⁷ are each independently selected from —CR^{Z4}, or N;
R^{Z4}, at each occurrence, is independently selected from hydrogen, halogen, —C₁-C₈alkyl, —NR^{Z4a}R^{Z4b}, —OR^{Z4a}, —SR^{Z4a}, C₃-C₈cycloalkyl, 3- to 8-membered heterocyclyl, C₆-C₁₂aryl, 5- to 12-membered heteroaryl, or CN; each of —C₁-C₈alkyl, C₃-C₈cycloalkyl, 3- to 8-membered heterocyclyl, C₆-C₁₂aryl, or 5- to 12-membered heteroaryl is optionally substituted with at least one R^{Z4c};
R^{Z4a} and R^{Z4b} are each independently selected from hydrogen, —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl, each of said —C₁-C₈alkyl, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl is optionally substituted with at least one substituent R^{Z4d};
R^{Z4c} and R^{Z4d} are each independently halogen, hydroxy, —C₁-C₈alkyl, —C₁-C₈alkoxy, —C₂-C₈alkenyl, —C₂-C₈alkynyl, C₃-C₈cycloalkyl, 3- to 8-membered heterocyclyl, C₆-C₁₂aryl, or 5- to 12-membered heteroaryl.

21. The compound of claim 20, wherein R¹³ is selected from —P(O)R^{13a}R^{13b} or —N(R^{13a})—SO₂R^{13b}, wherein R^{13a} and R^{13b} are each independently selected from hydrogen, —C₁-C₈alkyl (preferably —CH₃, —C₂H₅, —C₃H₇, —C₄H₉ or —C₅H₁₁; more preferably —CH₃, —CH₂CH₃, —CH₂CH₂CH₃, -iso-C₃H₇, —CH₂CH₂CH₂CH₃, -iso-C₄H₉, -sec-C₄H₉ or -tert-C₄H₉) or C₃-C₈cycloalkyl (preferably cyclopropyl, cyclobutyl or cyclopentyl).

22. The compound of claim 20, wherein R¹³ is selected from —P(O)(CH₃)₂, —NH—SO₂CH₃ or —N(CH₃)—SO₂CH₃.

23. The compound of claim 20, wherein R¹³ is —P(O)(CH₃)₂.

24. The compound of claim 20, wherein R¹⁴ and R¹⁵ are each independently selected from hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, C₆-C₁₂aryl, 5- to 12-membered heteroaryl, —CN, —OR^{14a}, —SO₂R^{14a}, —SO₂NR^{14a}R^{14b}, COR^{14a}, —CO₂R^{14a}, —CONR^{14a}R^{14b}, —NR^{14a}R^{14b}, —NR^{14a}COR^{14b}, —NR^{14a}CO₂R^{14b}, or —NR^{14a}SO₂R^{14b}; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, C₆-C₁₂aryl or 5- to 12-membered heteroaryl is optionally substituted with at least one substituent R^{14d}, or
R¹⁴ and R¹⁵ together with the carbon atoms to which they are attached, form a 5 or 6-membered unsaturated or saturated ring, said ring comprising 0, 1, 2 or 3 heteroatoms independently selected from nitrogen, oxygen or sulfur; said ring is optionally substituted with at least one substituent R^{14e};
R^{14e}, at each occurrence, is independently —H, —F, —Cl, —Br, —I, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, phenyl, 3- to 8-membered heterocyclyl, 5- to 12-membered heteroaryl, oxo, —CN, CF₃, CHF₂, CH₂F, thioxo, —SCF₃, —SCHF₂, —SCH₂F, —SCH₂CF₃, —SCF₂CH₃, —SCF₂CF₃, —SO₂R^{14a}, —SO₂NR^{14a}R^{14b}, —COR^{14a}, —CO₂R^{14a}, —CONR^{14a}R^{14b}, —NR^{14a}R^{14b}, —NR^{14a}COR^{14b}, —NR^{14a}CO₂R^{14b} or —NR^{14a}SO₂R^{14b}; each of methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, phenyl, 3- to 8-membered heterocyclyl, 5- to 12-membered heteroaryl is optionally substituted with at least one substituent R^{14d};
R^{14a} and R^{14b} are each independently selected from hydrogen, methyl, ethyl, propyl, butyl, pentyl, hexyl, heptyl, octyl, methoxy, ethoxy, propoxy, butoxy, pentoxy, hexoxy, heptyloxy, octyloxy, C₁-C₈alkoxy-C₁-C₈alkyl-, cyclopropyl, cyclobutyl, cyclopentyl, cyclohexyl, cycloheptyl, cyclooctyl, 3- to 8-membered heterocyclyl, phenyl or 5- to 12-membered heteroaryl;
R^{14d}, at each occurrence, is independently halogen, —OH, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

25. The compound of claim 20, wherein R¹⁴ and R¹⁵ together with the carbon atoms to which they are attached, form a 5 or 6-membered unsaturated (preferred aromatic) or saturated ring, said ring comprising 1 or 2 nitrogen heteroatoms; said ring is optionally substituted with at least one substituent —H, —F, —Cl, —Br, —I, methyl, ethyl, propyl (n- or iso-), butyl, pentyl, methoxy, ethoxy, propoxy, butoxy, pentoxy, cyclopropyl, cyclobutyl, cyclopentyl, cyclohexyl, cycloheptyl, cyclooctyl, —CH₂OH, —SCH₃, —SC₂H₅, oxo, thioxo, —CF₃, —CHF₂, —CH₂F, —SCF₃, —OMe, —OC₂H₅, —CN, —C(O)CH₃,

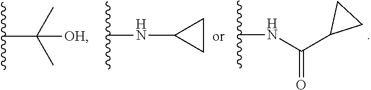

26. The compound of claim 20, wherein R¹⁴ and R¹⁵ together with the carbon atoms to which they are attached, form a 6-membered unsaturated (preferred aromatic), said ring comprising 1 or 2 nitrogen heteroatoms; said ring is optionally substituted with one substituent —H, —F, —Cl, —Br, —I, methyl, ethyl or cyclopropyl.

27. The compound of claim 20, wherein R⁴ is hydrogen, —F, —Cl, —Br, —I, methyl, ethyl, propyl, butyl, pentyl, hexyl, heptyl, octyl, cyclopropyl, cyclobutyl, cyclopentyl, cyclohexyl, cycloheptyl, cyclooctyl, —C₂-C₈alkenyl, —C₂-C₈alkynyl or —C₁-C₈alkoxy; each of methyl, ethyl, propyl, butyl, pentyl, hexyl, heptyl, octyl, cyclopropyl, cyclobutyl, cyclopentyl, cyclohexyl, cycloheptyl, cyclooctyl, —C₂-C₈alkenyl or —C₂-C₈alkynyl is optionally substituted with —F, —Cl, —Br, —I, oxo, or —CN.

28. The compound of claim 20, wherein R⁴ is hydrogen, —F, —Cl, —Br, —I, —CH₃, —CF₃, —CH₂F, or —CHF₂.

29. The compound of claim 20, wherein R⁴ is hydrogen, —F, —Cl, —Br or —I.

30. The compound of claim 20, wherein R⁹, R¹⁰ and R¹¹ are each independently selected from hydrogen, —F, —Cl, —Br, —I, methyl, ethyl, propyl, butyl, pentyl, hexyl, heptyl, octyl, —NR^{9a}R^{9b}, —OR^{9a}, cyclopropyl, cyclobutyl, cyclopentyl, cyclohexyl, cycloheptyl, cyclooctyl, 3- to 8-membered heterocyclyl, phenyl, 5- to 12-membered heteroaryl, oxo, or —CN; each of - methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9c};
R^{9a} and R^{9b} are each independently selected from hydrogen, methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{9d};
R^{9c} and R^{9d} are each independently —F, —Cl, —Br, —I, hydroxy, methyl, ethyl, propyl, butyl, pentyl, hexyl, heptyl, octyl, methoxy, ethoxy, propoxy, butoxy, pentoxy, hexoxy, heptyloxy, octyloxy, cyclopropyl, cyclobutyl, cyclopentyl, cyclohexyl, cycloheptyl, cyclooctyl, 3- to 8-membered heterocyclyl, phenyl or 5- to 12-membered heteroaryl.

31. The compound of claim 20, wherein R⁹, R¹⁰ and R¹¹ are each independently selected from hydrogen, —F, —Cl, —Br, —I, methyl, ethyl, propyl, butyl, —NH₂, —NHCH₃, —OH, —OCH₃, —OC₂H₅, cyclopropyl, cyclobutyl, cyclopentyl, cyclohexyl, —CH₂OH, —CH₂OMe, oxo, or —CN.

32. The compound of claim 20, wherein R⁹, R¹⁰ and R¹¹ are each independently selected from hydrogen, —CH₃, —F, —Cl, —Br or —I.

33. The compound of claim 20, wherein Z⁴, Z⁵, Z⁶ and Z⁷ are each independently —CR^{4z};
R^{4Z}, at each occurrence, is independently selected from hydrogen, —F, —Cl, —Br, —I, methyl, ethyl, propyl, butyl, pentyl, hexyl, heptyl, octyl, —NR^{4Za}R^{4Zb}, —OR^{4Za}, —SR^{4Za} cyclopropyl, cyclobutyl, cyclopentyl, cyclohexyl, cycloheptyl, cyclooctyl, 3- to 8-membered heterocyclyl, phenyl, 5- to 12-membered heteroaryl, or CN; each of methyl, ethyl, propyl, butyl, pentyl, hexyl, heptyl, octyl, cyclopropyl, cyclobutyl, cyclopentyl, cyclohexyl, cycloheptyl, cyclooctyl, 3- to 8-membered heterocyclyl, phenyl, or 5- to 12-membered heteroaryl is optionally substituted with at least one R^{4Zc};
R^{4Za} and R^{4Zb} are each independently selected from hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl, each of said hydrogen, methyl, ethyl, propyl, butyl, pentyl, hexyl, heptyl, octyl, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl or 5- to 12-membered heteroaryl is optionally substituted with at least one substituent R^{4Zd};
R^{4Zc} and R^{4Zd} are each independently —F, —Cl, —Br, —I, hydroxy, methyl, ethyl, propyl, butyl, pentyl, hexyl, heptyl, octyl, —C₁-C₈alkoxy, —C₂-C₈alkenyl, —C₂-C₈alkynyl, cyclopropyl, cyclobutyl, cyclopentyl, cyclohexyl, cycloheptyl, cyclooctyl, 3- to 8-membered heterocyclyl, phenyl, or 5- to 12-membered heteroaryl.

34. The compound of claim 20, wherein R^{4z} is selected from H, —CH₃, —C₂H₅, F, —CH₂F, —CHF₂, —CF₃, —OCH₃, —OC₂H₅, —C₃H₇, —OCH₂F, —OCHF₂, —OCH₂CF₃, —OCF₃, —SCF₃, —CF₃, —CH(OH)CH₃,

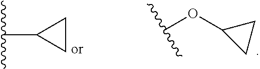

35. The compound of any one of claims 1-19, wherein Warhead is 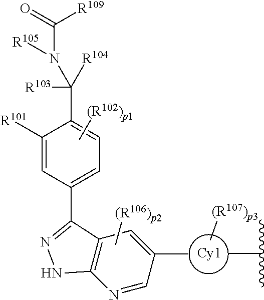
wherein 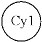
is a 5- or 6-membered aromatic ring comprising 0-3 heteroatoms selected from nitrogen, oxygen and sulfur;
R¹⁰¹, R¹⁰², R¹⁰³, R¹⁰⁴, R¹⁰⁵, R¹⁰⁶ and R¹⁰⁷ are each independently hydrogen, halogen, —C₁₋₈alkyl, —C₁₋₈alkoxy, —C₂₋₈alkenyl, —C₂₋₈alkynyl, cycloalkyl, heterocyclyl, aryl, heteroaryl, —CN, —NO₂, —OR^{10a}, —SO₂R^{10a}, —COR^{10a}, —CO₂R^{10a}, —CONR^{10a}R^{10b}, —C(═NR^{10a})NR^{10b}R^{10c}, —NR^{10a}R^{10b}, —NR^{10a}COR^{10b}, —NR^{10c}CONR^{10b}R^{10c}, —NR^{10a}CO₂R^{10b}, —NR^{10a}SONR^{10b}R^{10c}, —NR^{10a}SO₂NR^{10b}R^{10c}, or —NR^{10a}SO₂R^{10b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl;
R¹⁰⁹ is 5- or 6-membered aromatic ring comprising 0-3 heteroatoms selected from nitrogen, oxygen and sulfur; said aromatic ring is optionally substituted with halogen, —C₁₋₈alkyl, —C₂₋₈alkenyl, —C₂₋₈alkynyl, —C₁₋₈alkoxy, cycloalkyl, heterocyclyl, aryl, heteroaryl, —CN, —NO₂, —OR^{10a}, —SO₂R^{10a}, —COR^{10a}, —CO₂R^{10a}, —CONR^{10a}R^{10b}, —C(═NR^{10a})NR^{10b}R^{10c}, —NR^{10a}R^{10b}, —NR^{10a}COR^{10b}, —NR^{10a}CONR^{10b}R^{10c}, —NR^{10a}CO₂R^{10b}, —NR^{10a}SONR^{10b}R^{10c}, —NR^{10a}SO₂NR^{10b}R^{10c}, or —NR^{10a}SO₂R^{10b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl;
p1, p2 and p3 are each independently 0, 1, 2, 3 or 4;
R^{10a}, R^{10b} and R^{10c} are each independently hydrogen, —C₁₋₈alkyl, —C₂₋₈alkenyl, —C₂₋₈alkynyl, cycloalkyl, heterocyclyl, aryl, or heteroaryl.

36. The compound of claim 35, wherein 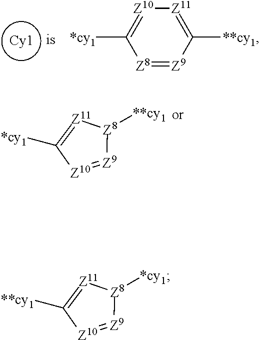
wherein Z⁸, Z⁹, Z¹⁰ and Z¹¹ are each independently selected from CH or N; wherein *cy1 refers to the position attached to the 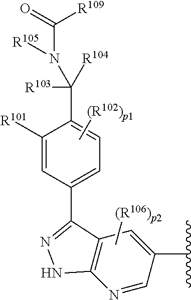
moiety, and **cy1 refers to the position attached to L¹.

37. The compound of claim 35, wherein 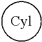
is selected from

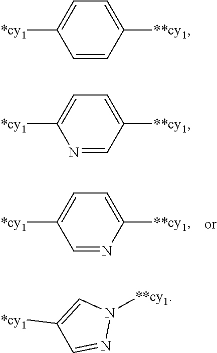

38. The compound of claim 35, wherein p3 is 0, 1, or 2, and each R¹⁰⁷ is independently selected from halogen, —C₁₋₈alkyl, or —C₁₋₈alkoxy, preferably F, Cl, Br, I, CH₃, or —OCH₃.

39. The compound of claim 35, wherein R^{10a} and R^{10b} are independently selected from hydrogen or CH₃; and n1 is 1 or 2.

40. The compound of claim 35, wherein R¹⁰¹ is methyl, —CH₂OH, —OCH₃, —CH₂OCH₃ or halogen; p1 is 0 or 1, and R¹⁰² is halogen.

41. The compound of claim 35, wherein R¹⁰³ and R¹⁰⁵ are hydrogen; and R¹⁰⁴ is selected from hydrogen or methyl.

42. The compound of claim 35, wherein R¹⁰⁹ is 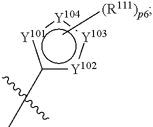
Y¹⁰¹, Y¹⁰², Y¹⁰³ and Y¹⁰⁴ are selected from CH, O, S or N; R¹¹¹ is selected from hydrogen, halogen, —C₁₋₈alkyl, —C₁₋₈alkoxy, —C₂₋₈alkenyl, —C₂₋₈alkynyl, cycloalkyl, heterocyclyl, aryl, heteroaryl, —CN, —NO₂, —OR^{10a}, —SO₂R^{10a}, —COR^{10a}, —CO₂R^{10a}, —CONR^{10a}R^{10b}, —C(═NR^{10a})NR^{10b}R^{10c}, —NR^{10a}R^{10b}, —NR^{10a}COR^{10b}, —NR^{10a}CONR^{10b}R^{10c}, —NR^{10a}CO₂R^{10b}, —NR^{10a}SONR^{10b}R^{10c}, —NR^{10a}SO₂NR^{10b}R^{10c}, or —NR^{10a}SO₂R^{10b}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl; R^{10a}, R^{10b}, and R^{10c} are each independently hydrogen, —C₁₋₈alkyl, —C₂₋₈alkenyl, —C₂₋₈alkynyl, cycloalkyl, heterocyclyl, aryl, or heteroaryl; and p6 is 0, 1, 2, 3 or 4.

43. The compound of claim 35, wherein Y¹⁰¹ is CH, S, N or O; Y¹⁰² is CH, O or N; Y¹⁰³ is O, S or N; and Y¹⁰⁴ is S, CH or N.

44. The compound of any one of claims 1-19, wherein Warhead is 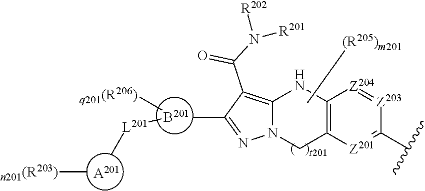
ring A²⁰¹ and B²⁰¹ are each independently an aromatic ring comprising 0-3 heteroatoms selected from nitrogen, sulfur and oxygen as ring member(s);
Z²⁰¹, Z²⁰³ and Z²⁰⁴ are each independently N or CR^{20z};
L²⁰¹ is independently a bond, —C₁₋₈alkylene-, —N(R²⁰⁴)—, —O—, —S—, *^{L201}-C_{1- 8}alkylene-O-**^{L201}, *^{L201}-O—C₁₋₈alkylene-**^{L201}, *^{L201}-N(R²⁰⁴)CO-**^{L201}, *^{L201}-CON(R²⁰⁴)-**^{L201}, *^{L201}-N(R²⁰⁴)CO—C₁₋₈alkylene-**^{L201}, *^{L201}-CON(R²⁰⁴)—C₁₋₈alkylene-**^{L201}, *^{L201}-N(R²⁰⁴)—C₁₋₈alkylene-**^{L201}, *^{L201}-C_{1- 8}alkylene-N(R²⁰⁴)-**^{L201}, -heterocyclene-, or -heteroarylene-, wherein each of said —C₁₋₈alkylene-, *^{L201}-C_{1- 8}alkylene-O-**^{L201}, *^{L201}-O—C₁₋₈alkylene-**^{L201}, *^{L201}-N(R²⁰⁴)CO—C₁₋₈alkylene-**^{L201}, *^{L201}-CON(R²⁰⁴)—C₁₋₈alkylene-**^{L201}, *^{L201}-N(R²⁰⁴)—C₁₋₈alkylene-**^{L201}, *^{L201}-C_{1- 8}alkylene-N(R²⁰⁴)-**^{L201}, -heterocyclene- and -heteroarylene- is optionally substituted with at least one substituent R^{20L};
wherein *^{L201} refers to the position attached to ring A, and **^{L201} refers to the position attached to ring B;
m201, n201 and q201 are each independently 0, 1, 2, 3 or 4;
t201 is 0, 1 or 2;
R²⁰¹, R²⁰², and R²⁰⁴ are each independently hydrogen, —C₁₋₈alkyl, —C₂₋₈alkenyl, —C₂₋₈alkynyl, cycloalkyl, heterocyclyl, aryl, or heteroaryl, wherein each of said —C₁₋₈alkyl, —C₂₋₈alkenyl, —C₂₋₈alkynyl, cycloalkyl, heterocyclyl, aryl, or heteroaryl is optionally substituted with halogen, hydroxy, —C₁₋₈alkyoxy, cycloalkyl, heterocyclyl, aryl, or heteroaryl;
R^{20L}, R²⁰³, R²⁰⁵ and R²⁰⁶ are each independently hydrogen, halogen, —C₁₋₈alkyl, —C₂₋₈alkenyl, —C₂₋₈alkynyl, cycloalkyl, heterocyclyl, aryl, heteroaryl, —CN, —NO₂, —OR^{20a}, —SO₂R^{20a}, —COR^{20a}, —CO₂R^{20a}, —CONR^{20a}R^{20b}, —C(═NR^{20a})NR^{20b}R^{20c}, —NR^{20a}R^{20b}, —NR^{20a}COR^{20b}, —NR^{20a}CONR^{20b}R^{20c}, —NR^{20a}CO₂R^{20b}, —NR^{20a}SONR^{20b}R^{20c}, —NR^{20a}SO₂NR^{20b}R^{20c}, or —NR^{20a}SO₂R^{20b}, wherein each of said —C₁₋₈alkyl, —C₂₋₈alkenyl, —C₂₋₈alkynyl, cycloalkyl, heterocyclyl, aryl, or heteroaryl is optionally substituted with at least one halogen, hydroxy, —C₁₋₈alkyl, —C₁₋₈alkyoxy, cycloalkyl, heterocyclyl, aryl, or heteroaryl;
R^{20z} is selected from hydrogen, halogen, —C₁₋₈alkyl, —C₂₋₈alkenyl, —C₂₋₈alkynyl, cycloalkyl, heterocyclyl, aryl, heteroaryl, —CN, —NO₂, —OR^{20a}, —SO₂R^{20a}, —COR^{20a}, —CO₂R^{20a}, —CONR^{20a}R^{20b}, —C(═NR^{20a})NR^{20b}R^{20c}, —NR^{20a}R^{20b}, —NR^{20a}COR^{20b}, —NR^{20a}CONR^{20b}R^{20c}, —NR^{20a}CO₂R^{20b}, —NR^{20a}SONR^{20b}R^{20c}, —NR^{20a}SO₂NR^{20b}R^{20c}, or —NR^{20a}SO₂R^{20b}, each of said —C₁₋₈alkyl, —C₂₋₈alkenyl, —C₂₋₈alkynyl, cycloalkyl, heterocyclyl, aryl, or heteroaryl is optionally substituted with at least one halogen, hydroxy, —C₁₋₈alkyl, —C₁₋₈alkyoxy, cycloalkyl, heterocyclyl, aryl, or heteroaryl;
R^{20a}, R^{20b}, and R^{20c} are each independently hydrogen, —C₁₋₈alkyl, —C₂₋₈alkenyl, —C₂₋₈alkynyl, cycloalkyl, heterocyclyl, aryl, or heteroaryl;
or two R^{20L}, together with the atom(s) to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{203f}, —SO₂R^{203f}, -SO₂NR^{203f}R^{203g}, —COR^{203f}, —CO₂R^{203f}, —CONR^{203f}R^{203g}, —C(═NR²⁰³f)NR^{203g}R^{203h}, —NR^{203f}R^{203g}, —NR^{203f}COR^{203g}, —NR^{203f}CONR^{203g}R^{203h}, —NR^{203f}CO₂R^{203f}, NR^{203f}SONR^{203f}R^{203g}, NR^{203f}SO₂NR^{203g}R^{203h}, or —NR^{203f}SO₂R^{203g}, each of said —C₁₋₈alkyl, —C₂₋₈alkenyl, —C₂₋₈alkynyl, cycloalkyl, heterocyclyl, aryl, or heteroaryl is optionally substituted with at least one substituent selected from halogen, —C₁₋₈alkyl, —OR²⁰³ⁱ, —NR²⁰³ⁱR^{203j}, cycloalkyl, heterocyclyl, aryl, or heteroaryl;
or two R²⁰³, together with the atoms to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{203f}, —SO₂R^{203f}, -SO₂NR^{203f}R^{203g}, —COR^{203f}, —CO₂R^{203f}, —CONR^{203f}R^{203g}, —C(═NR²⁰³f)NR^{203g}R^{203h}, —NR^{203f}R^{203g}, —NR^{203f}COR^{203g}, —NR^{203f}CONR^{203g}R^{203h}, —NR^{203f}CO₂R^{203f}, —NR^{203f}SONR^{203f}R^{203g}, NR^{203f}SO₂NR^{203g}R^{203h}, or —NR^{203f}SO₂R^{203g}, each of said —C₁₋₈alkyl, —C₂₋₈alkenyl, —C₂₋₈alkynyl, cycloalkyl, heterocyclyl, aryl, or heteroaryl is optionally substituted with at least one substituent selected from halogen, —C₁₋₈alkyl, —OR²⁰³ⁱ, —NR²⁰³ⁱR^{203j}, cycloalkyl, heterocyclyl, aryl, or heteroaryl;
or R⁴ and one of R³, together with the atoms to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN, —NO₂, —OR^{203f}, —SO₂R^{203f}, -SO₂NR^{203f}R^{203g}, —COR^{203f}, —CO₂R^{203f}, —CONR^{203f}R^{203g}, —C(═NR²⁰³f)NR^{203g}R^{203h}, —NR^{203f}R^{203g}, —NR^{203f}COR^{203g}, —NR^{203f}CONR^{203g}R^{203h}, —NR^{203f}CO₂R^{203f}, —NR^{203f}SONR^{203f}R^{203g}, —NR^{203f}SO₂NR^{203g}R^{203h}, or -NR^{203f}SO₂R^{203g}, each of said —C₁₋₈alkyl, —C₂₋₈alkenyl, —C₂₋₈alkynyl, cycloalkyl, heterocyclyl, aryl, or heteroaryl is optionally substituted with at least one substituent selected from halogen, —C₁₋₈alkyl, —OR²⁰³ⁱ, —NR²⁰³ⁱR^{203j}, cycloalkyl, heterocyclyl, aryl, or heteroaryl;
R^{203f}, R^{203g}, R^{203h}, R²⁰³ⁱ, and R^{203j} are each independently hydrogen, —C₁₋₈alkyl, C₁₋₈alkoxy-C₁₋₈alkyl-, —C₂₋₈alkenyl, —C₂₋₈alkynyl, cycloalkyl, heterocyclyl, aryl, or heteroaryl.

45. The compound according to any one of claim 44, wherein L²⁰¹ is a bond, —CH₂—, —C₂H₄—, —C₃H₆—, —C₄H₈—, —C₅H₁₀—, —O—, —NH—, *^{L201}-NHCH₂-**^{L201}, *^{L201}-NHC₂H₄-**^{L201}, *^{L201}-NHC₃H₆-**^{L201}, *^{L201}-NHC₄H₈-**^{L201}, *^{L201}-NHC₅H₁₀-**^{L201}, *^{L201}-OCH₂-**^{L201}, *^{L201}-OC₂H₄-**^{L201}, *^{L201}-OC₃H₆-**^{L201}, *^{L201}-OC₄H₈-**^{L201}, *^{L201}-OC₅H₁₀-**^{L201}, *^{L201}-CH₂O-**^{L201}, *^{L201}-C₂H₄O-**^{L201}, *^{L201}-C₃H₆O-**^{L201}, *^{L201}-C4H₈-**^{L201}, *^{L201}-C₅H₁₀-**^{L201}, *^{L201}-CONH-**^{L201}, *^{L201}-NHCO-**^{L201}, *^{L201}-CONHCH₂-**^{L201}, *^{L201}-CONHC₂H4-**^{L201}, *^{L201}-CONHC₃H₆-**^{L201}, *^{L201}-CONHC₄H₈-**^{L201}, *^{L201}-CONHC₅H₁₀-**^{L201}, 3- to 8-membered -heterocyclene- or 5- to 6-membered -heteroarylene-; wherein each of said —CH₂—, —C₂H4-, —C₃H₆—, —C₄H8-, —C₅H₁₀—, —O—, —NH—, *^{L201}-NHCH₂**^{L201}, *^{L201}-NHC₂H4-**^{L201}, *^{L201}-NHC₃H₆-**^{L201}, *^{L201}-NHC₄H₈-**^{L201}, *^{L201}-NHC₅H₁₀-**^{L201}, *^{L201}-OCH₂-**^{L201}, *^{L201}-OC₂H₄-**^{L201}, *^{L201}-OC₃H₆-**^{L201}, *^{L201}-OC₄H₈-**^{L201}, *^{L201}-OC₅H₁₀-**^{L201}, *^{L201}-CH₂O-**^{L201}, *^{L201}-C₂H4O-**^{L201}, *^{L201}-C₃H6O-**^{L201}, *^{L201}-C₄H₈O-**^{L201}, *^{L201}-C₅H₁₀O-**^{L201}, *^{L201}-CONH-**^{L201}, *^{L201}-NHCO-**^{L201}, *^{L201}-CONHCH₂**^{L201}, *^{L201}-CONHC₂H₄-**^{L201}, *^{L201}-CONHC₃H₆-**^{L201}, *^{L201}-CONHC₄H₈-**^{L201}, *^{L201}-CONHC₅H₁₀-**^{L201}, 3- to 8-membered heterocyclene- and 5- to 6-membered heteroarylene- is optionally substituted with at least one substituent R^{20L}; wherein R^{20L} is defined as above.

46. The compound according to claim 44, wherein L²⁰¹ is a bond, —O—, *^{L201}-OCH₂-**^{L201}, *^{L201}-CH₂O-**^{L201}, —NH—, *^{L201}-CONH-**^{L201}, *^{L201}-NHCO-**^{L201}, *^{L201}-CONHCH₂-**^{L201}, *^{L201}-CONHCH₂CH₂-**^{L201}, *^{L201}-CONHCH₂CH₂CH₂-**^{L201}, *^{L201}-CONHCH(CH₃)-**^{L201}, *^{L201}-CONHCH(C₂H₅)-**^{L201}, *^{L201}-NHCH₂-**^{L201}, *^{L201}-NHCH₂CH₂-**^{L201}, *^{L201}-NHCH₂CH₂CH₂-**^{L201}, *^{L201}-NHCH(CH₃)-**^{L201} or *^{L}-NHCH(C₂H₅)-**^{L}.

47. The compound according to claim 44, wherein L²⁰¹ is *^{L201}-N(R²⁰⁴)CO-**^{L201}, R²⁰³ and R²⁰⁴, together with the atoms to which they are attached, form a 5-, 6- or 7-membered ring, said ring comprising 0, 1 or 2 additional heteroatoms independently selected from nitrogen, oxygen or optionally oxidized sulfur as ring member(s), said ring is optionally substituted with at least one substituent independently selected from —F, —Cl, —Br, —I, —CH₃, —C₂H₅, —C₃H₇, —C₄H₉, —C₅H₁₁, cycloalkyl, heterocyclyl, aryl, heteroaryl, or oxo.

48. The compound according to claim 44, wherein 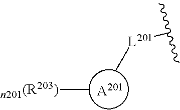
moiety is 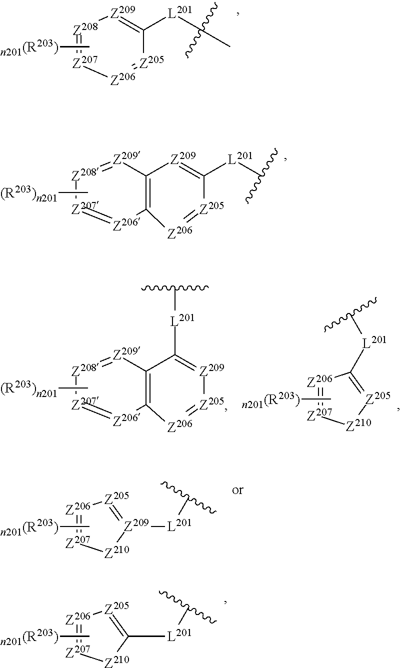
wherein Z²⁰⁵, Z²⁰⁶, Z²⁰⁷, Z²⁰⁸, Z²⁰⁹, Z²⁰⁶′, Z²⁰⁷′,% Z²⁰⁸′ and Z²⁰⁹′ are each independently N or C(H); Z²¹⁰ is N(H), O or S.

49. The compound according to claim 44, wherein ring A201 is a 5- to 6-membered aromatic ring comprising 0-3 heteroatoms selected from nitrogen, sulfur and oxygen as ring member(s).

50. The compound according to claim 44, wherein ring A201 is phenyl, naphthalenyl, quinoxalinyl, pyridinyl, pyridazinyl, pyrimidinyl, imidazolyl, thiazolyl, oxazolyl, oxadiazole, pyridyl, pyrazinyl, pyridazinyl, pyrrolyl, imidazolyl, pyrazolyl, triazolyl, furanyl, pyrimidinyl, pyrazinyl, pyrrolopyridinyl or dihydropyrrolopyrazinyl.

51. The compound according to claim 44, wherein the 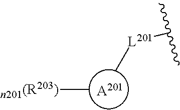
moiety is

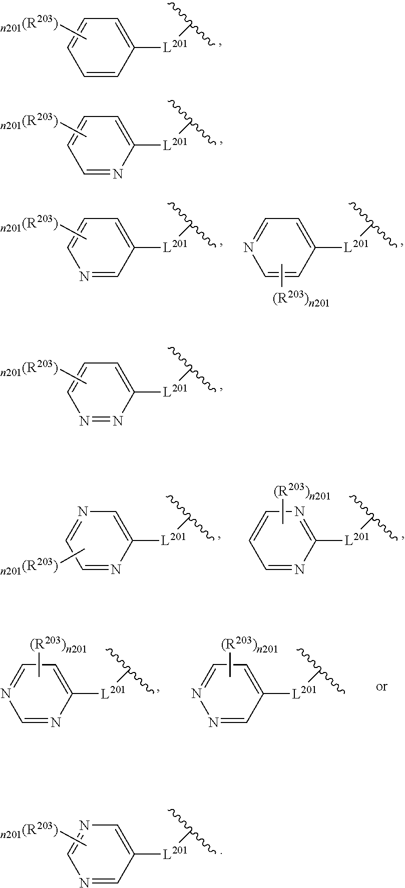

52. The compound according to claim 44, wherein R²⁰³ is hydrogen, oxo, —F, —Cl, —Br, —I, —CH₃, —C₂H₅, —C₃H₇, —C₄H₉, —C₅H₁₁, —OCH₃, —OC₂H₅, —OC₃H₇, —OC₄H9, —OC₅H₁₁, cyclopropyl, cyclobutyl, cyclopentyl, tetrahydropyrrolyl or phenyl, wherein each of said —CH₃, —C₂H₅, —C₃H₇, —C₄H₉, —C₅H₁₁, —OCH₃, —OC₂H₅, —OC₃H₇, —OC₄H9, —OC₅H₁₁, cyclopropyl, cyclobutyl, cyclopentyl, tetrahydropyrrolyl or phenyl, is optionally substituted with at least one —F, —Cl, —Br, —I, —CH₃, —C₂H₅, —C₃H₇, —C₄H₉, —C₅H₁₁, —OCH₃, —OC₂H₅, —OC₃H₇, —OC₄H9, —OC₅H11, —OH, cyclopropyl, cyclobutyl or cyclopentyl.

53. The compound according to claim 44, wherein R²⁰³ is hydrogen, oxo, —F, —Cl, —Br, —I, methyl, ethyl, n-propyl, iso-propyl, n-butyl, iso-butyl, sec-butyl, tert-butyl, trifluoromethyl, difluoromethyl, fluoromethyl, —OMe, —OEt, —OPr, —OBu, cyclopropyl, cyclobutyl, tetrahydropyrrolyl or phenyl.

54. The compound according to claim 44, wherein two R²⁰³, together with the atoms to which they are attached, form a 4-, 5-, 6-, 7- or 8-membered ring, said ring comprising 0, 1 or 2 additional heteroatoms independently selected from nitrogen, oxygen or optionally oxidized sulfur as ring member(s), said ring is optionally substituted with at least one substituent independently selected from —F, —Cl, —Br, —I, —CH₃, —C₂H₅, —C₃H₇, —C₄H₉, —C₅H₁₁, —OCH₃, —OC₂H₅, —OC₃H₇, —OC₄H₉, —OC₅H₁₁, —OH, —CN, cyclopropyl, cyclobutyl or cyclopentyl.

55. The compound according to claim 44, wherein the 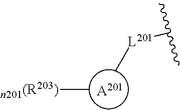
moiety is

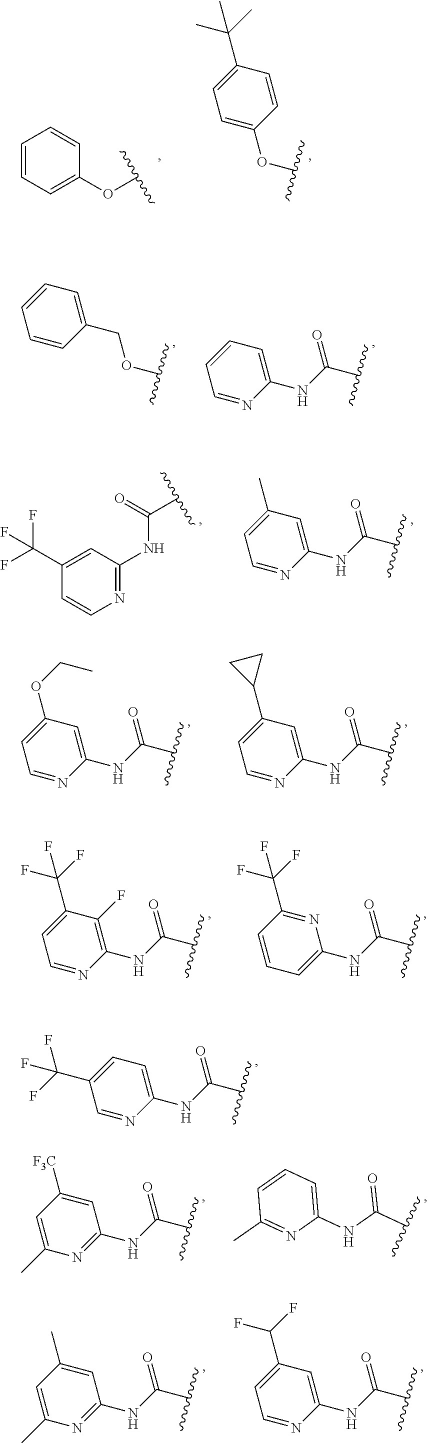

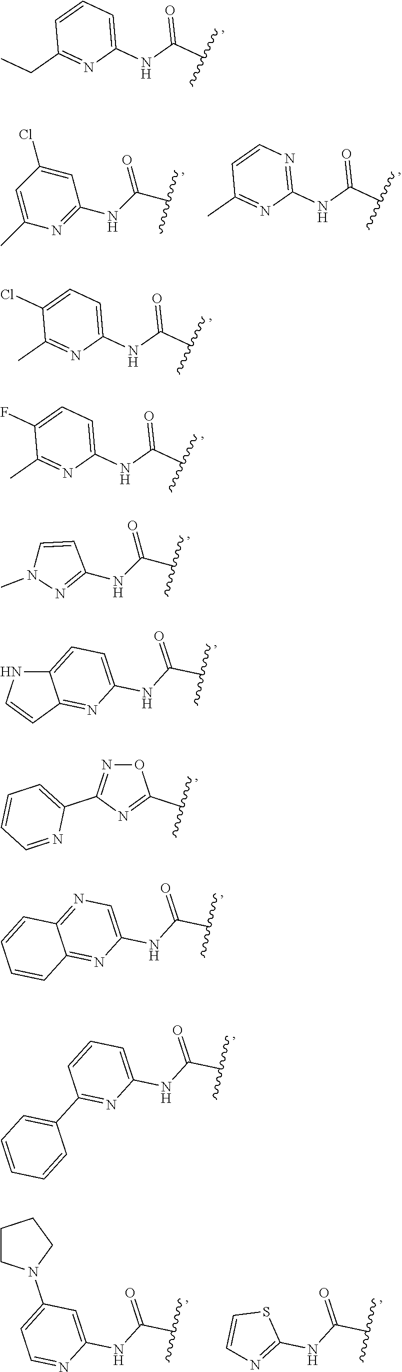

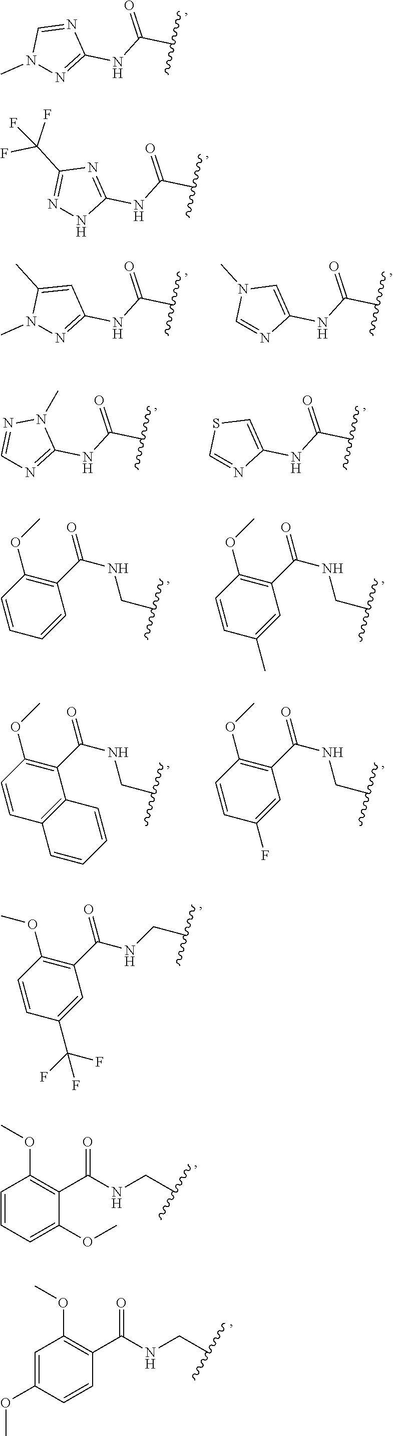

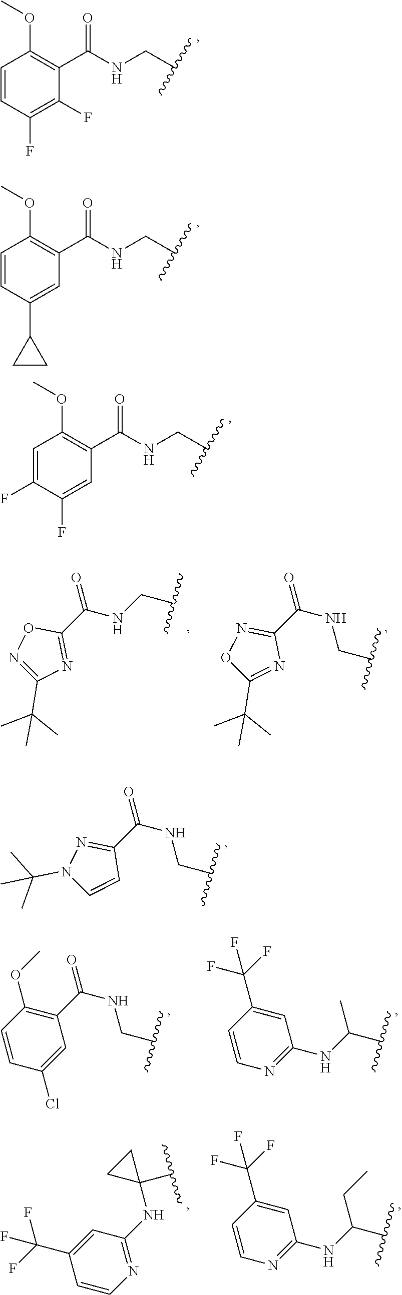

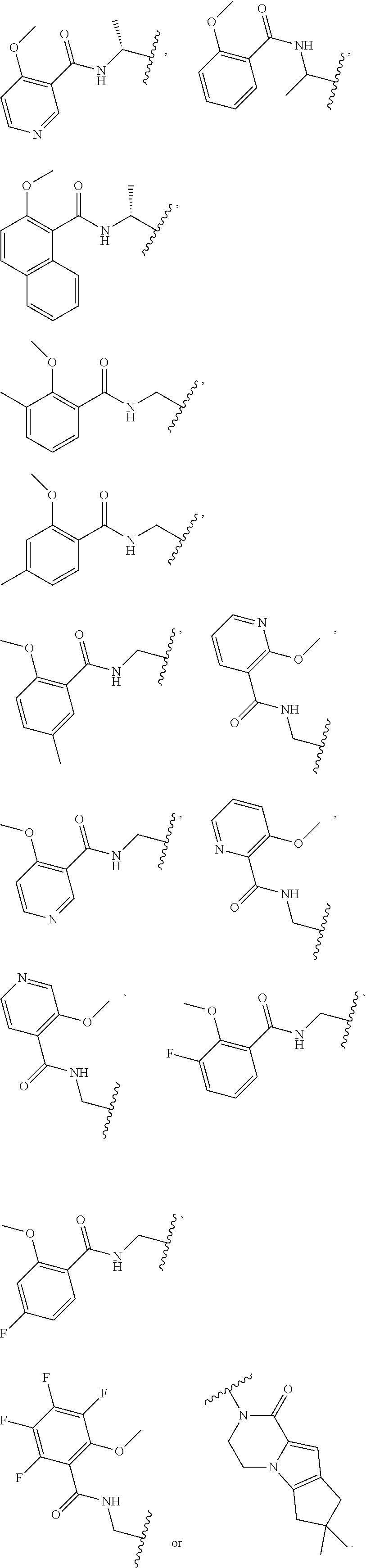

56. The compound according to claim 44, wherein the 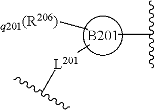
moiety is 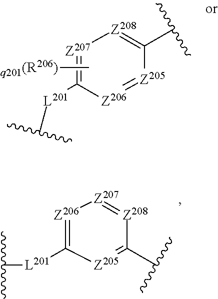
wherein Z²⁰⁵, Z²⁰⁶, Z²⁰⁷ and Z²⁰⁸ are defined as above.

57. The compound according to claim 44, wherein the 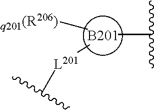
moiety is

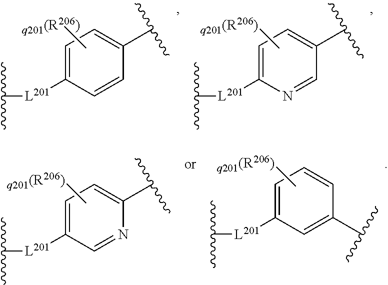

58. The compound according to claim 44, wherein ring B201 is phenyl, pyridinyl, imidazolyl, thiazolyl, oxazolyl, pyridyl, pyrimidinyl, pyrazinyl, pyridazinyl, pyrrolyl, imidazolyl, pyrazolyl, thiazolyl, oxazolyl, triazolyl, thiophenyl, furanyl, pyrimidinyl or pyrazinyl, each of which is optionally substituted with (R²⁰⁶)_{q201}.

59. The compound according to claim 44, wherein R²⁰⁶ is hydrogen, —F, —Cl, —Br, —I, —CH₃, —C₂H₅, —C₃H₇, —C₄H₉, —C₅H₁₁, —CN, —OCH₃, —OC₂H₅, —OC₃H₇, —OC₄H₉ or —OC₅H₁₁, wherein each of said —CH₃, —C₂H₅, —C₃H₇, —C₄H₉, —C₅H₁₁, —OCH₃, —OC₂H₅, —OC₃H₇, —OC₄H₉ or —OC₅H₁₁ is optionally substituted with —F, —Cl, —Br, —I, hydroxy, —C₁₋₈alkyoxy, cycloalkyl, heterocyclyl, aryl, or heteroaryl.

60. The compound according to claim 44, wherein the 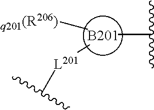
moiety is

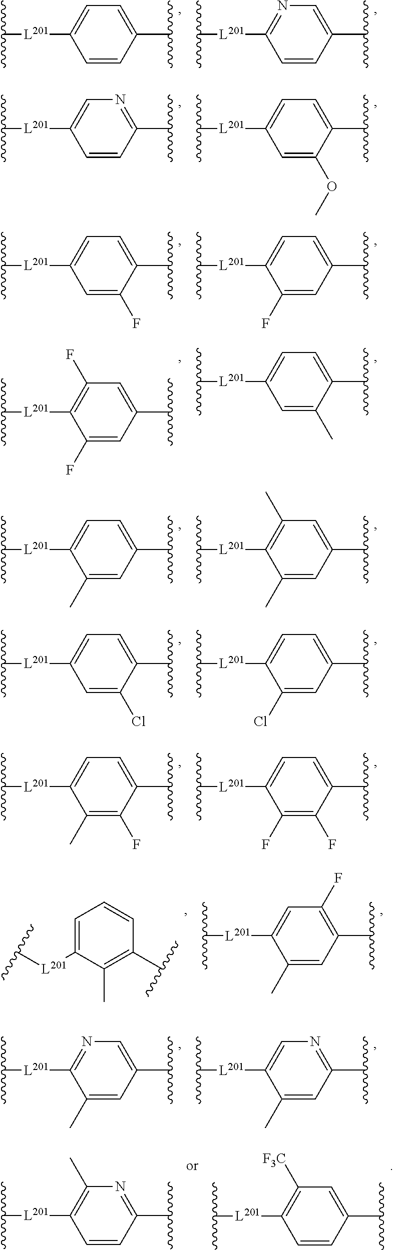

61. The compound according to claim 44, wherein R²⁰¹ and R²⁰² are each independently hydrogen, —CH₃, —C₂H₅, —C₃H₇, —C₄H₉, —C₅H₁₁, —C₂₋₈alkenyl, —C₂₋₈alkynyl or aryl.

62. The compound according to claim 44, wherein R²⁰¹ and R²⁰² are both H.

63. The compound according to any one of claims 1-27, wherein R²⁰⁵ is independently hydrogen, —F, —Cl, —Br, —I, —CH₃, —C₂H₅, —C₃H₇, —C₄H₉, —C₅H₁₁, —C₂₋₈alkenyl, —C₂₋₈alkynyl or aryl.

64. The compound according to claim 44, wherein R^{20z} is hydrogen, —F, —Cl, —Br, —I, —CH₃, —C₂H₅, —C₃H₇, —C₄Hg or —C5H₁₁.

65. The compound according to any one of claims 44 to 64, wherein the 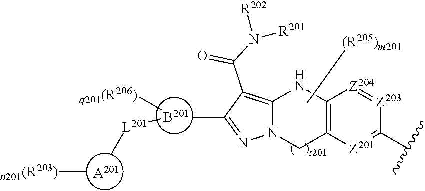
moiety is

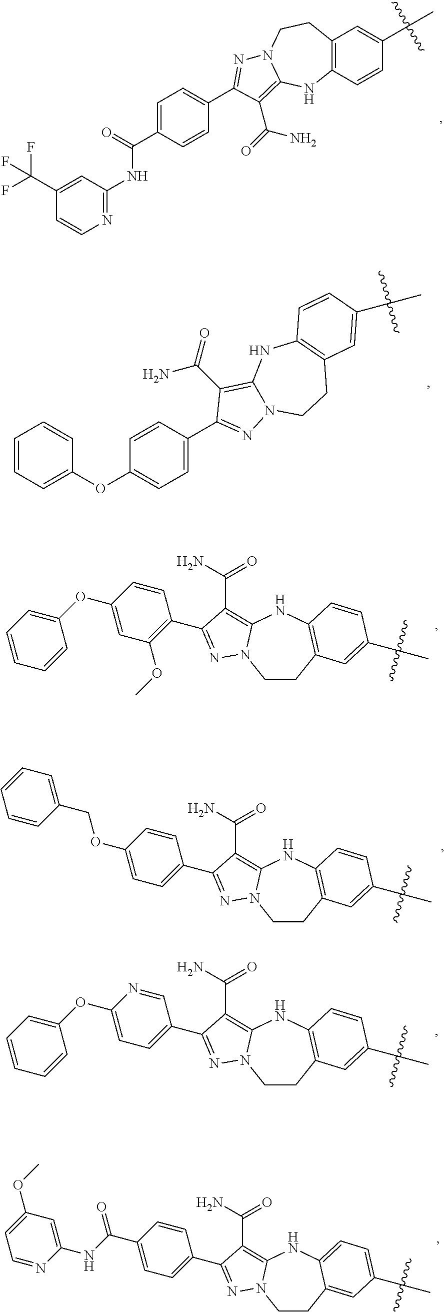 [SMILES: *c1ccc2c(c1)CCn1nc(-c3ccc(C(=O)Nc4cc(C(F)(F)F)ccn4)cc3)c(C(N)=O)c1N2 ; *c1ccc2c(c1)CCn1nc(-c3ccc(Oc4ccccc4)cc3)c(C(N)=O)c1N2 ; *c1ccc2c(c1)CCn1nc(-c3ccc(Oc4ccccc4)cc3OC)c(C(N)=O)c1N2 ; *c1ccc2c(c1)CCn1nc(-c3ccc(OCc4ccccc4)cc3)c(C(N)=O)c1N2 ; *c1ccc2c(c1)CCn1nc(-c3ccc(Oc4ccccc4)nc3)c(C(N)=O)c1N2 ; *c1ccc2c(c1)CCn1nc(-c3ccc(C(=O)Nc4cc(OC)ccn4)cc3)c(C(N)=O)c1N2]

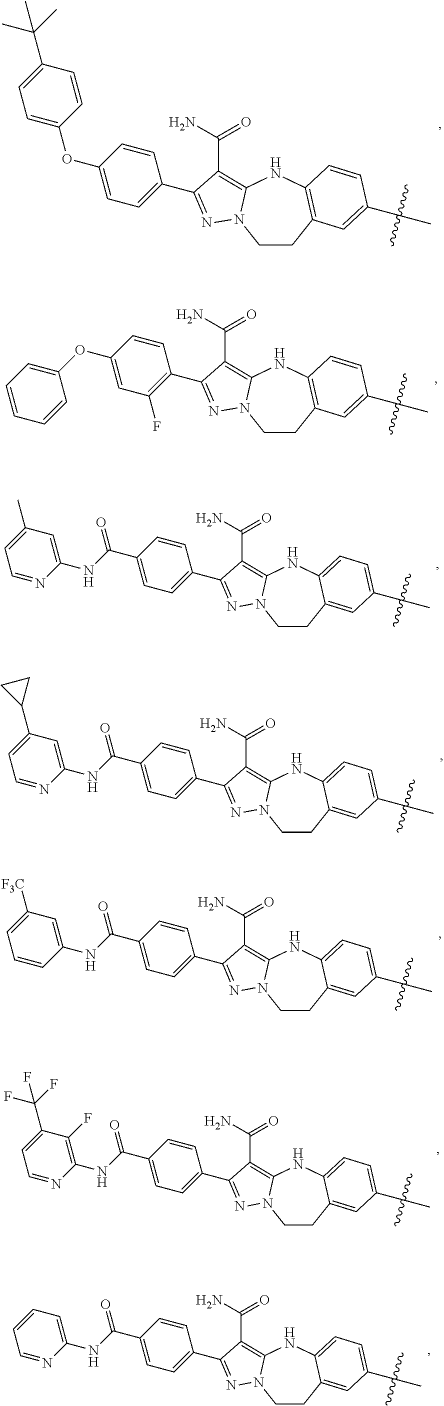

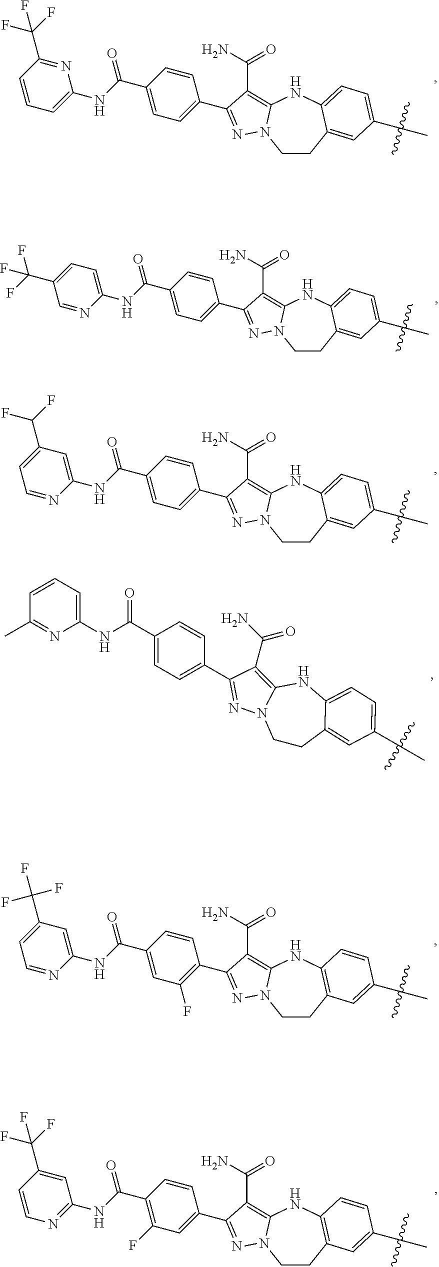 [SMILES: *c1ccc2c(c1)CCn1nc(-c3ccc(C(=O)Nc4cccc(C(F)(F)F)n4)cc3)c(C(N)=O)c1N2 ; *c1ccc2c(c1)CCn1nc(-c3ccc(C(=O)Nc4ccc(C(F)(F)F)cn4)cc3)c(C(N)=O)c1N2 ; *c1ccc2c(c1)CCn1nc(-c3ccc(C(=O)Nc4cc(C(F)F)ccn4)cc3)c(C(N)=O)c1N2 ; *c1ccc2c(c1)CCn1nc(-c3ccc(C(=O)Nc4cccc(C)n4)cc3)c(C(N)=O)c1N2 ; *c1ccc2c(c1)CCn1nc(-c3ccc(C(=O)Nc4cc(C(F)(F)F)ccn4)cc3F)c(C(N)=O)c1N2 ; *c1ccc2c(c1)CCn1nc(-c3ccc(C(=O)Nc4cc(C(F)(F)F)ccn4)c(F)c3)c(C(N)=O)c1N2]

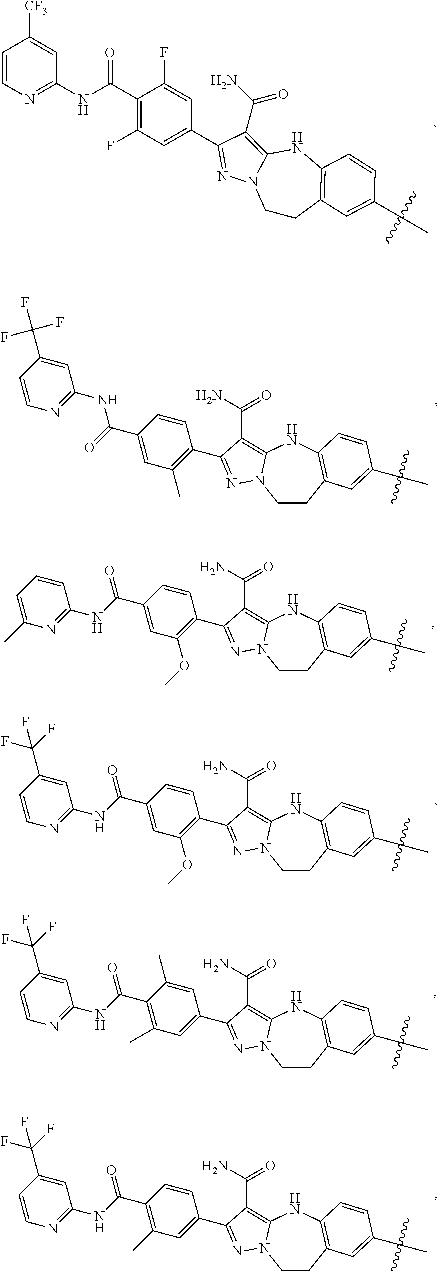 [SMILES: *c1ccc2c(c1)CCn1nc(-c3cc(F)c(C(=O)Nc4cc(C(F)(F)F)ccn4)c(F)c3)c(C(N)=O)c1N2 ; *c1ccc2c(c1)CCn1nc(-c3ccc(C(=O)Nc4cc(C(F)(F)F)ccn4)cc3C)c(C(N)=O)c1N2 ; *c1ccc2c(c1)CCn1nc(-c3ccc(C(=O)Nc4cccc(C)n4)cc3OC)c(C(N)=O)c1N2 ; *c1ccc2c(c1)CCn1nc(-c3ccc(C(=O)Nc4cc(C(F)(F)F)ccn4)cc3OC)c(C(N)=O)c1N2 ; *c1ccc2c(c1)CCn1nc(-c3cc(C)c(C(=O)Nc4cc(C(F)(F)F)ccn4)c(C)c3)c(C(N)=O)c1N2 ; *c1ccc2c(c1)CCn1nc(-c3ccc(C(=O)Nc4cc(C(F)(F)F)ccn4)c(C)c3)c(C(N)=O)c1N2]

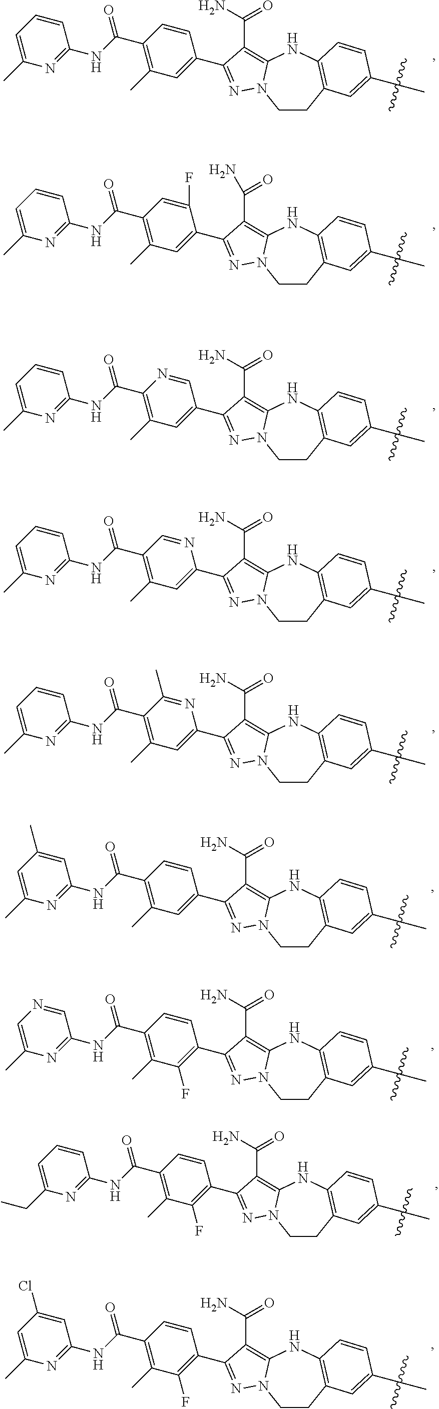

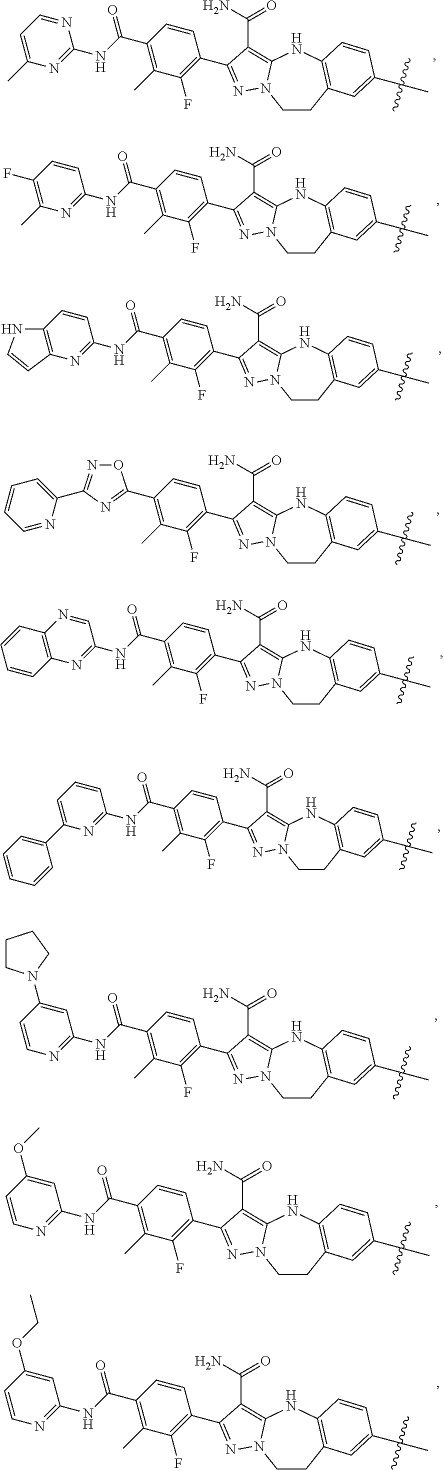

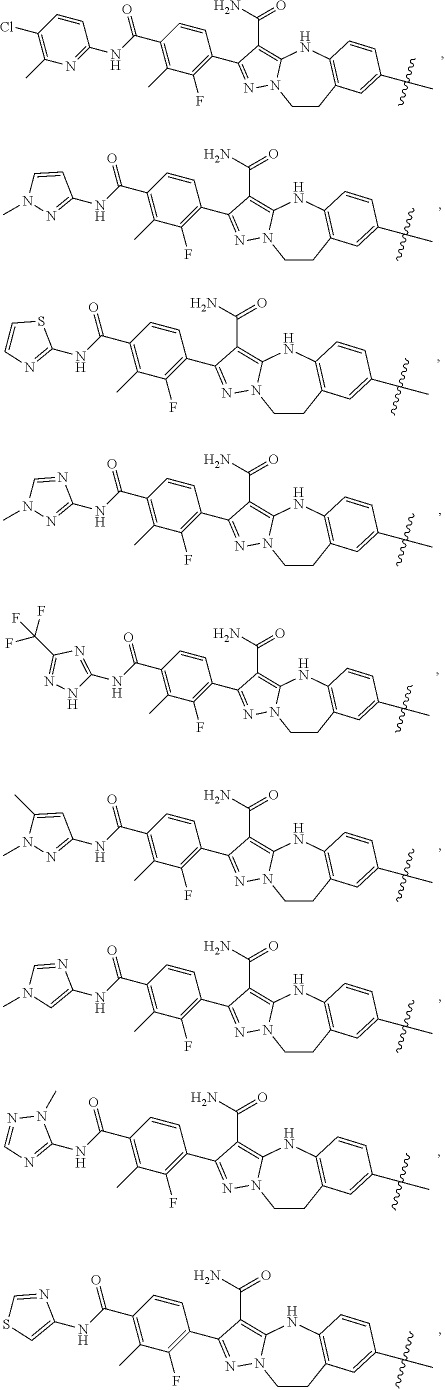

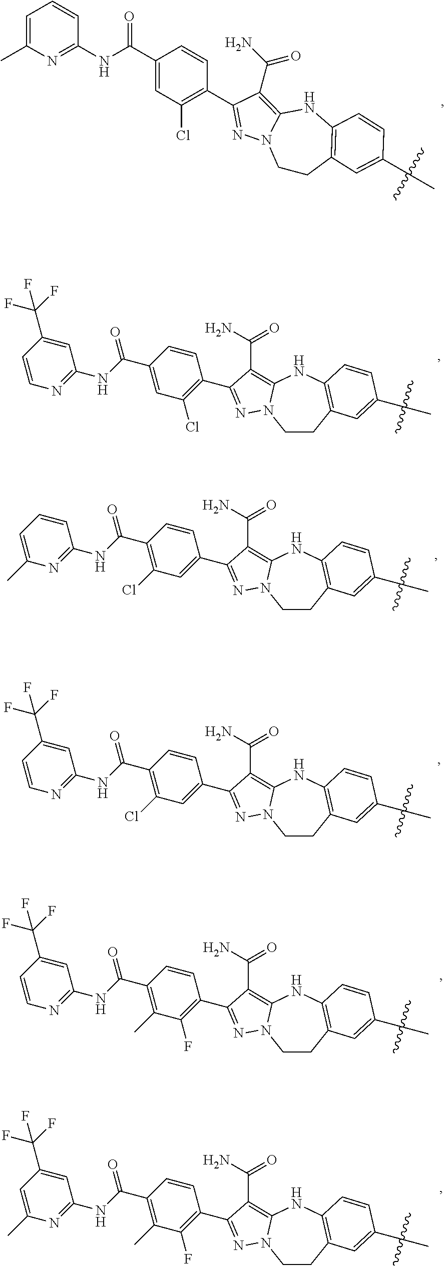 [SMILES: *c1ccc2c(c1)CCn1nc(-c3ccc(C(=O)Nc4cccc(C)n4)cc3Cl)c(C(N)=O)c1N2 ; *c1ccc2c(c1)CCn1nc(-c3ccc(C(=O)Nc4cc(C(F)(F)F)ccn4)cc3Cl)c(C(N)=O)c1N2 ; *c1ccc2c(c1)CCn1nc(-c3ccc(C(=O)Nc4cccc(C)n4)c(Cl)c3)c(C(N)=O)c1N2 ; *c1ccc2c(c1)CCn1nc(-c3ccc(C(=O)Nc4cc(C(F)(F)F)ccn4)c(Cl)c3)c(C(N)=O)c1N2 ; *c1ccc2c(c1)CCn1nc(-c3ccc(C(=O)Nc4cc(C(F)(F)F)ccn4)c(C)c3F)c(C(N)=O)c1N2 ; *c1ccc2c(c1)CCn1nc(-c3ccc(C(=O)Nc4cc(C(F)(F)F)cc(C)n4)c(C)c3F)c(C(N)=O)c1N2]

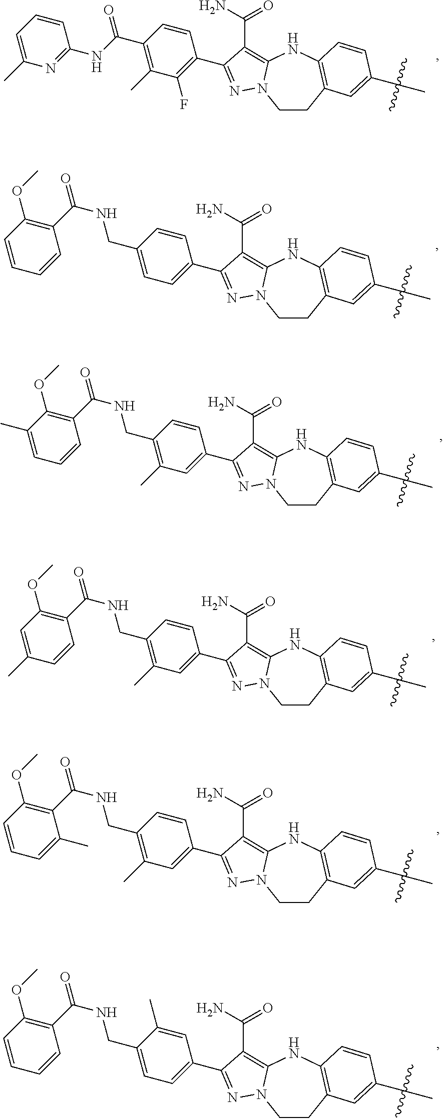 [SMILES: *c1ccc2c(c1)CCn1nc(-c3ccc(C(=O)Nc4cccc(C)n4)c(C)c3F)c(C(N)=O)c1N2 ; *c1ccc2c(c1)CCn1nc(-c3ccc(CNC(=O)c4ccccc4OC)cc3)c(C(N)=O)c1N2 ; *c1ccc2c(c1)CCn1nc(-c3ccc(CNC(=O)c4cccc(C)c4OC)c(C)c3)c(C(N)=O)c1N2 ; *c1ccc2c(c1)CCn1nc(-c3ccc(CNC(=O)c4ccc(C)cc4OC)c(C)c3)c(C(N)=O)c1N2 ; *c1ccc2c(c1)CCn1nc(-c3ccc(CNC(=O)c4c(C)cccc4OC)c(C)c3)c(C(N)=O)c1N2 ; *c1ccc2c(c1)CCn1nc(-c3ccc(CNC(=O)c4ccccc4OC)c(C)c3)c(C(N)=O)c1N2]

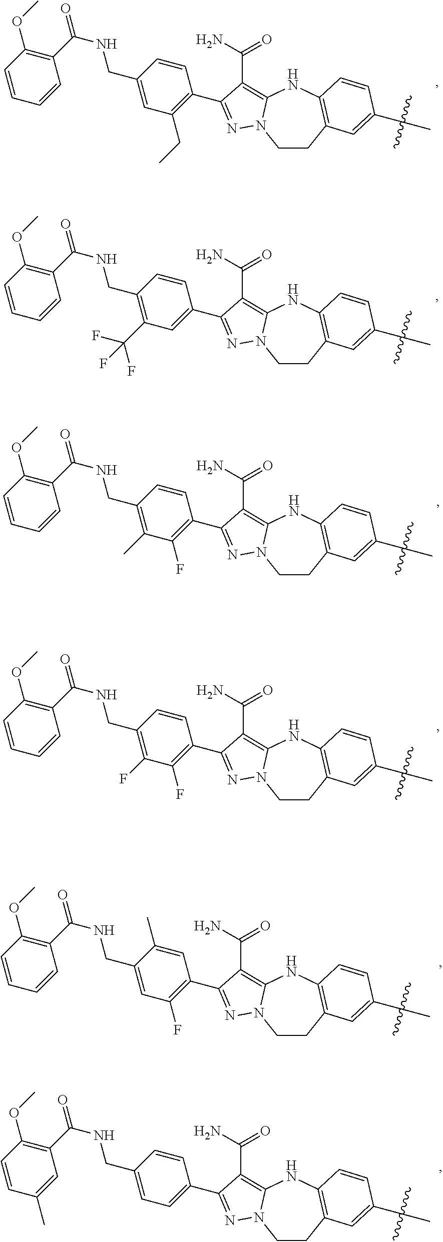 [SMILES: *c1ccc2c(c1)CCn1nc(-c3ccc(CNC(=O)c4ccccc4OC)cc3CC)c(C(N)=O)c1N2 ; *c1ccc2c(c1)CCn1nc(-c3ccc(CNC(=O)c4ccccc4OC)c(C(F)(F)F)c3)c(C(N)=O)c1N2 ; *c1ccc2c(c1)CCn1nc(-c3ccc(CNC(=O)c4ccccc4OC)c(C)c3F)c(C(N)=O)c1N2 ; *c1ccc2c(c1)CCn1nc(-c3ccc(CNC(=O)c4ccccc4OC)c(F)c3F)c(C(N)=O)c1N2 ; *c1ccc2c(c1)CCn1nc(-c3cc(C)c(CNC(=O)c4ccccc4OC)cc3F)c(C(N)=O)c1N2 ; *c1ccc2c(c1)CCn1nc(-c3ccc(CNC(=O)c4cc(C)ccc4OC)cc3)c(C(N)=O)c1N2]

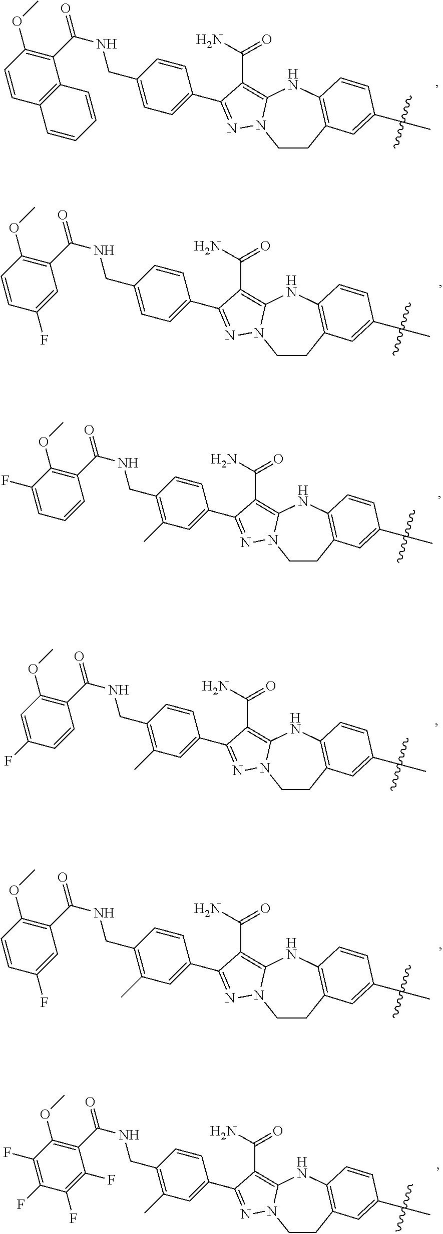 [SMILES: *c1ccc2c(c1)CCn1nc(-c3ccc(CNC(=O)c4c(OC)ccc5ccccc45)cc3)c(C(N)=O)c1N2 ; *c1ccc2c(c1)CCn1nc(-c3ccc(CNC(=O)c4cc(F)ccc4OC)cc3)c(C(N)=O)c1N2 ; *c1ccc2c(c1)CCn1nc(-c3ccc(CNC(=O)c4cccc(F)c4OC)c(C)c3)c(C(N)=O)c1N2 ; *c1ccc2c(c1)CCn1nc(-c3ccc(CNC(=O)c4ccc(F)cc4OC)c(C)c3)c(C(N)=O)c1N2 ; *c1ccc2c(c1)CCn1nc(-c3ccc(CNC(=O)c4cc(F)ccc4OC)c(C)c3)c(C(N)=O)c1N2 ; *c1ccc2c(c1)CCn1nc(-c3ccc(CNC(=O)c4c(F)c(F)c(F)c(F)c4OC)c(C)c3)c(C(N)=O)c1N2]

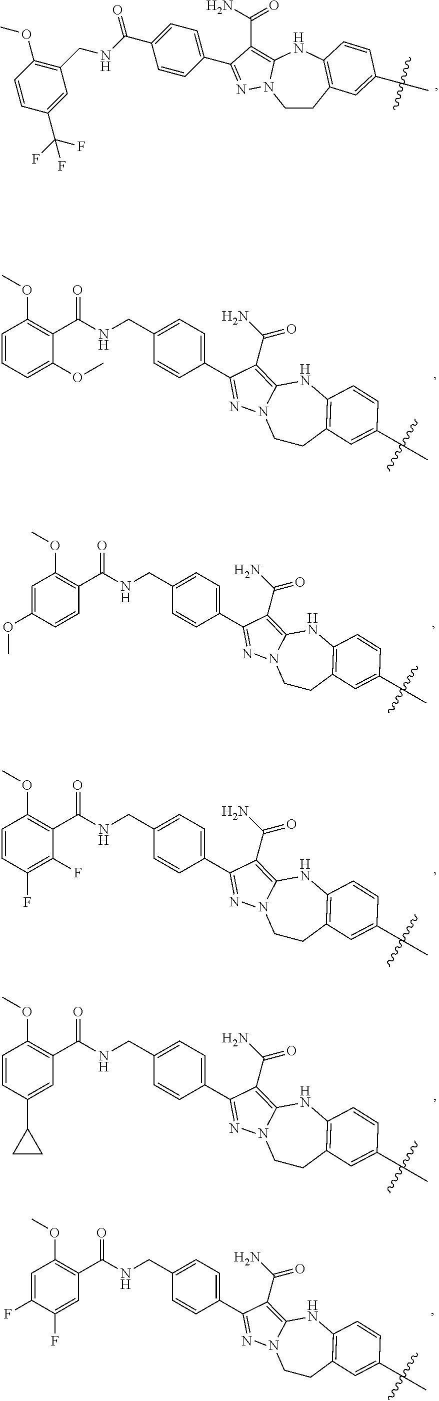 [SMILES: *c1ccc2c(c1)CCn1nc(-c3ccc(C(=O)NCc4cc(C(F)(F)F)ccc4OC)cc3)c(C(N)=O)c1N2 ; *c1ccc2c(c1)CCn1nc(-c3ccc(CNC(=O)c4c(OC)cccc4OC)cc3)c(C(N)=O)c1N2 ; *c1ccc2c(c1)CCn1nc(-c3ccc(CNC(=O)c4ccc(OC)cc4OC)cc3)c(C(N)=O)c1N2 ; *c1ccc2c(c1)CCn1nc(-c3ccc(CNC(=O)c4c(OC)ccc(F)c4F)cc3)c(C(N)=O)c1N2 ; *c1ccc2c(c1)CCn1nc(-c3ccc(CNC(=O)c4cc(C5CC5)ccc4OC)cc3)c(C(N)=O)c1N2 ; *c1ccc2c(c1)CCn1nc(-c3ccc(CNC(=O)c4cc(F)c(F)cc4OC)cc3)c(C(N)=O)c1N2]

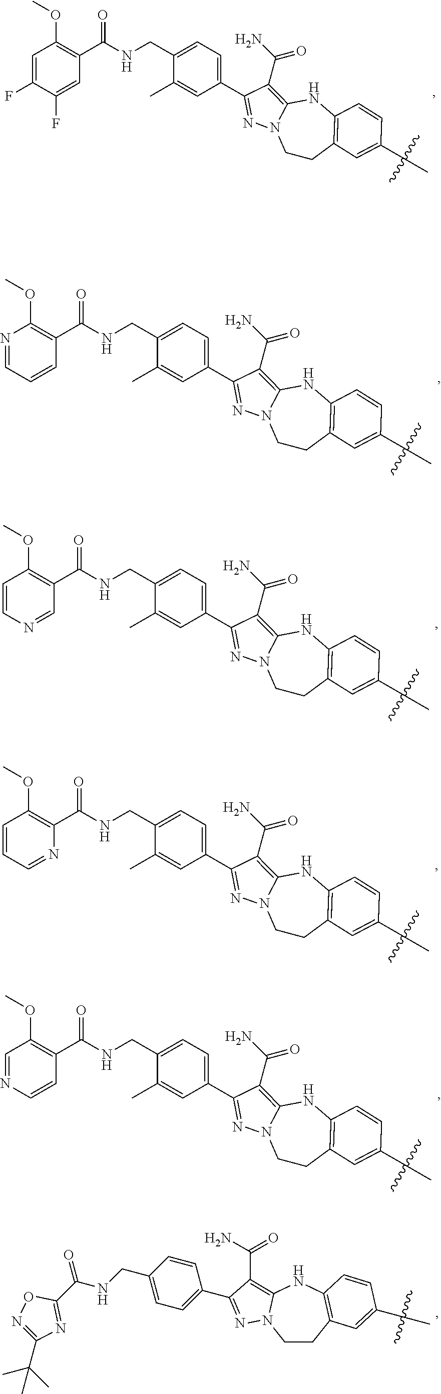 [SMILES: *c1ccc2c(c1)CCn1nc(-c3ccc(CNC(=O)c4cc(F)c(F)cc4OC)c(C)c3)c(C(N)=O)c1N2 ; *c1ccc2c(c1)CCn1nc(-c3ccc(CNC(=O)c4cccnc4OC)c(C)c3)c(C(N)=O)c1N2 ; *c1ccc2c(c1)CCn1nc(-c3ccc(CNC(=O)c4cnccc4OC)c(C)c3)c(C(N)=O)c1N2 ; *c1ccc2c(c1)CCn1nc(-c3ccc(CNC(=O)c4ncccc4OC)c(C)c3)c(C(N)=O)c1N2 ; *c1ccc2c(c1)CCn1nc(-c3ccc(CNC(=O)c4ccncc4OC)c(C)c3)c(C(N)=O)c1N2 ; *c1ccc2c(c1)CCn1nc(-c3ccc(CNC(=O)c4nc(C(C)(C)C)no4)cc3)c(C(N)=O)c1N2]

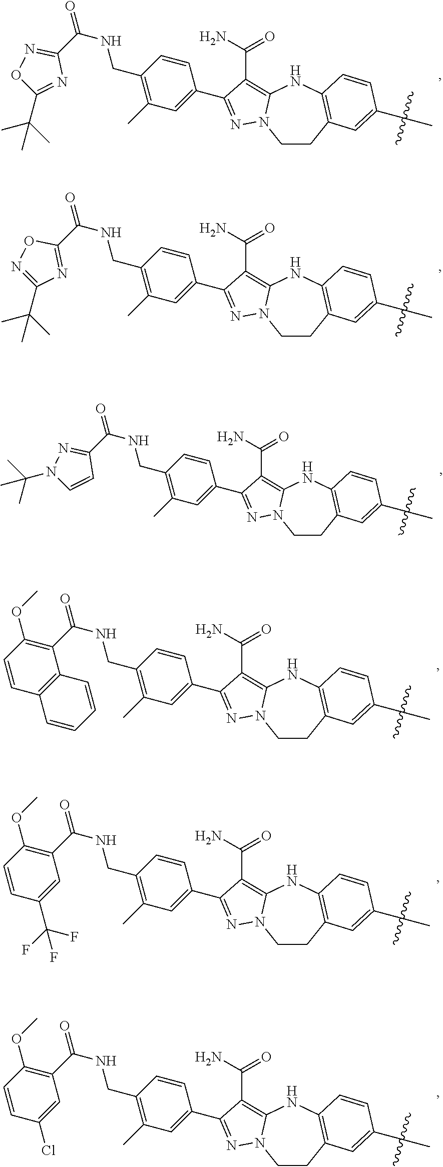 [SMILES: *c1ccc2c(c1)CCn1nc(-c3ccc(CNC(=O)c4noc(C(C)(C)C)n4)c(C)c3)c(C(N)=O)c1N2 ; *c1ccc2c(c1)CCn1nc(-c3ccc(CNC(=O)c4nc(C(C)(C)C)no4)c(C)c3)c(C(N)=O)c1N2 ; *c1ccc2c(c1)CCn1nc(-c3ccc(CNC(=O)c4ccn(C(C)(C)C)n4)c(C)c3)c(C(N)=O)c1N2 ; *c1ccc2c(c1)CCn1nc(-c3ccc(CNC(=O)c4c(OC)ccc5ccccc45)c(C)c3)c(C(N)=O)c1N2 ; *c1ccc2c(c1)CCn1nc(-c3ccc(CNC(=O)c4cc(C(F)(F)F)ccc4OC)c(C)c3)c(C(N)=O)c1N2 ; *c1ccc2c(c1)CCn1nc(-c3ccc(CNC(=O)c4cc(Cl)ccc4OC)c(C)c3)c(C(N)=O)c1N2]

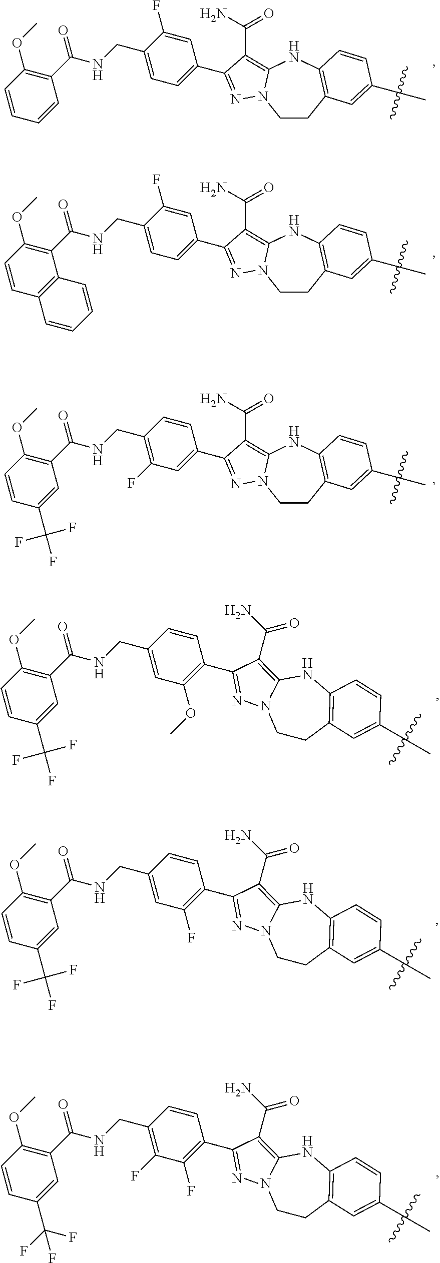 [SMILES: *c1ccc2c(c1)CCn1nc(-c3ccc(CNC(=O)c4ccccc4OC)c(F)c3)c(C(N)=O)c1N2 ; *c1ccc2c(c1)CCn1nc(-c3ccc(CNC(=O)c4c(OC)ccc5ccccc45)c(F)c3)c(C(N)=O)c1N2 ; *c1ccc2c(c1)CCn1nc(-c3ccc(CNC(=O)c4cc(C(F)(F)F)ccc4OC)c(F)c3)c(C(N)=O)c1N2 ; *c1ccc2c(c1)CCn1nc(-c3ccc(CNC(=O)c4cc(C(F)(F)F)ccc4OC)cc3OC)c(C(N)=O)c1N2 ; *c1ccc2c(c1)CCn1nc(-c3ccc(CNC(=O)c4cc(C(F)(F)F)ccc4OC)cc3F)c(C(N)=O)c1N2 ; *c1ccc2c(c1)CCn1nc(-c3ccc(CNC(=O)c4cc(C(F)(F)F)ccc4OC)c(F)c3F)c(C(N)=O)c1N2]

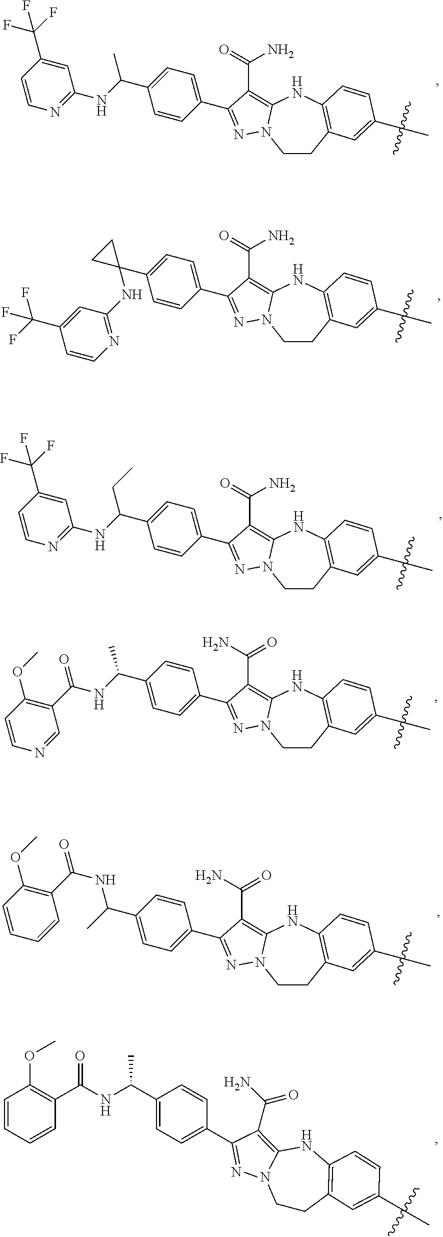 [SMILES: *c1ccc2c(c1)CCn1nc(-c3ccc(C(C)Nc4cc(C(F)(F)F)ccn4)cc3)c(C(N)=O)c1N2 ; *c1ccc2c(c1)CCn1nc(-c3ccc(C4(Nc5cc(C(F)(F)F)ccn5)CC4)cc3)c(C(N)=O)c1N2 ; *c1ccc2c(c1)CCn1nc(-c3ccc(C(CC)Nc4cc(C(F)(F)F)ccn4)cc3)c(C(N)=O)c1N2 ; *c1ccc2c(c1)CCn1nc(-c3ccc([C@@H](C)NC(=O)c4cnccc4OC)cc3)c(C(N)=O)c1N2 ; *c1ccc2c(c1)CCn1nc(-c3ccc(C(C)NC(=O)c4ccccc4OC)cc3)c(C(N)=O)c1N2 ; *c1ccc2c(c1)CCn1nc(-c3ccc([C@@H](C)NC(=O)c4ccccc4OC)cc3)c(C(N)=O)c1N2]

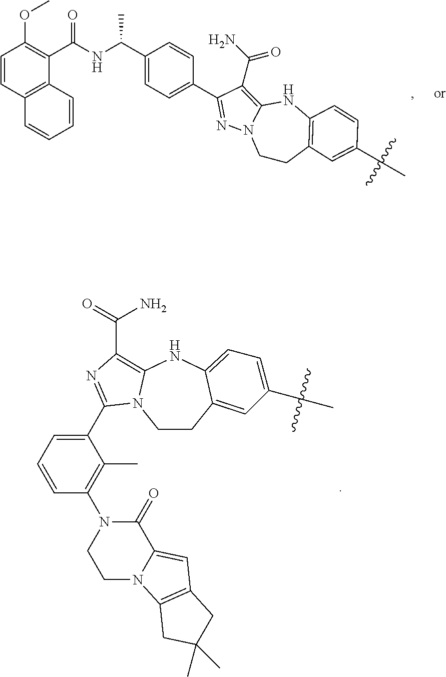 [SMILES: *c1ccc2c(c1)CCn1nc(-c3ccc([C@@H](C)NC(=O)c4c(OC)ccc5ccccc45)cc3)c(C(N)=O)c1N2 ; *c1ccc2c(c1)CCn1c(-c3cccc(N4CCn5c(cc6c5CC(C)(C)C6)C4=O)c3C)nc(C(N)=O)c1N2]

66. The compound ofany one of claims 1-19, wherein Warhead is 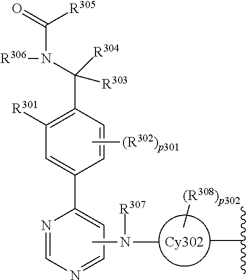
wherein:
Cy302 is a 5- or 6-membered saturated ring or unsaturated ring (preferably aromatic ring) comprising 0-3 heteroatoms selected from nitrogen, oxygen and sulfur as ring member(s);
each of occurrence, R³⁰¹, R³⁰², R³⁰³, R³⁰⁴ and R³⁰⁸ are each independently hydrogen, halogen, —C₁₋₈alkyl, —C₁₋₈alkoxy, —C₂₋₈alkenyl, —C₂₋₈alkynyl, cycloalkyl, heterocyclyl, aryl, heteroaryl, oxo (═O), —CN, —NO₂, —OR^{30c}, —SO₂R^{30c}, —COR^{30c}, —CO₂R^{30c}, —CONR^{30c}R^{30d}, —C(═NR^{30c})NR^{30d}R^{30e}, —NR^{30c}R^{30d}, —NR^{30c}COR^{30d}, —NR^{30c}CONR^{30d}R^{30e}, —NR^{30c}CO₂R^{30d}, —NR^{30c}SONR^{30d}R^{30e}, —NR^{30c}SO₂NR^{30d}R^{30e}, or —NR^{30c}SO₂R^{30d}, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl;
R³⁰⁶ and R³⁰⁷ are each independently hydrogen, —C₁₋₈alkyl, —C₂₋₈alkenyl, —C₂₋₈alkynyl, cycloalkyl, heterocyclyl, aryl or heteroaryl, each of said —C₁₋₈alkyl, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl;
R³⁰⁵ is 5- or 6-membered aromatic ring comprising 0-3 heteroatoms selected from nitrogen, oxygen and sulfur as ring member(s); said aromatic ring is optionally substituted with halogen, —C₁₋₈alkyl, —C₂₋₈alkenyl, —C₂₋₈alkynyl, —C₁₋₈alkoxy, cycloalkyl, heterocyclyl, aryl, heteroaryl, —CN, —NO₂, —OR^{30c}, —SO₂R^{30c}, —COR^{30c}, —CO₂R^{30c}, —CONR^{30c}R^{30d}, —C(═NR^{30c})NR^{30d}R^{30e}, —NR^{30c}R^{30d}, —NR^{30c}COR^{30d}, —NR^{30c}CONR^{30d}R^{30e}, —NR^{30c}CO₂R^{30d}, —NR^{30c}SONR^{30d}R^{30e}, —NR^{30c}SO₂NR^{30d}R^{30e}, or —NR^{30c}SO₂R^{30d}, each of said —C₁₋₈alkyl, —C₂₋₈alkenyl, —C₂₋₈alkynyl, cycloalkyl, heterocyclyl, aryl, or heteroaryl is optionally substituted with —C₁₋₈alkyl, halogen, hydroxy, -haloC₁₋₈alkyl, —C₁₋₈alkyoxy, cycloalkyl, heterocyclyl, aryl, or heteroaryl;
p301 and p302 are each independently 0, 1, 2, 3 or 4;
R^{30c}, R^{30d} and R^{30e} are each independently hydrogen, —C₁₋₈alkyl, —C₂₋₈alkenyl, —C₂₋₈alkynyl, cycloalkyl, heterocyclyl, aryl, or heteroaryl; or
(R^{30c} and R^{30d}) or (R^{30d} and R^{30e}) together with the atom(s) to which they are attached, form a 3- to 12-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂.

67. The compound of claim 66, wherein
R³⁰¹ is hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN, —NO₂, —OR^{30c}, —SO₂R^{30c}, —COR^{30c}, —CO₂R^{30c}, —CONR^{30c}R^{30d}, —C(═NR^{30c})NR^{30d}R^{30e}, —NR^{30c}R^{30d}, —NR^{30c}COR^{30d}, —NR^{30c}CONR^{30d}R^{30e}, —NR^{30c}CO₂R^{30d}, —NR^{30c}SONR^{30d}R^{30e}, —NR^{30c}SO₂NR^{30d}R^{30e}, or —NR^{30c} SO₂R^{30d}, each of said methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with F, Cl, Br, I, hydroxy, -haloC₁₋₈alkyl, - methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, heterocyclyl, aryl, or heteroaryl;
R^{30c}, R^{30d} and R^{30e} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, or heteroaryl; or
(R^{30c} and R^{30d}) or (R^{30d} and R^{30e}) together with the atom(s) to which they are attached, form a 3-, 4-, 5-, 6-, 7- or 8-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂.

68. The compound of any claim of claims 66-67, wherein R³⁰¹ is hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN or —NO₂; preferably RI is hydrogen, F, Cl, Br, I, methyl, ethyl, propyl, butyl, cyclopropyl, cyclobutyl.

69. The compound of any claim of claims 66-68, wherein
R³⁰² is hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN, —NO₂, —OR^{30c}, —SO₂R^{30c}, —COR^{30c}, —CO₂R^{30c}, —CONR^{30c}R^{30d}, —C(═NR^{30c})NR^{30d}R^{30e}, —NR^{30c}R^{30d}, —NR^{30c}COR^{30d}, —NR^{30c}CONR^{30d}R^{30e}, —NR^{30c}CO₂R^{30d}, —NR^{30c}SONR^{30d}R^{30e}, —NR^{30c}SO₂NR^{30d}R^{30e}, or —NR^{30c} SO₂R^{30d}, each of said methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with F, Cl, Br, I, hydroxy, -haloC₁₋₈alkyl, - methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, heterocyclyl, aryl, or heteroaryl;
R^{30c}, R^{30d} and R^{30e} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, or heteroaryl; or
(R^{30c} and R^{30d}) or (R^{30d} and R^{30e}) together with the atom(s) to which they are attached, form a 3-, 4-, 5-, 6-, 7- or 8-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂.

70. The compound of any claim of claims 66-69, wherein R³⁰² is hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN or —NO₂; preferably R³⁰² is hydrogen, F, Cl, Br, I, methyl, ethyl, propyl, butyl, methoxy, ethoxy, propoxy, butoxy, cyclopropyl, cyclobutyl, cyclopentyl, —CN or —NO₂.

71. The compound of any claim of claims 66-70, wherein
R³⁰³ and R³⁰⁴ are each independently hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN, —NO₂, —OR^{30c}, —SO₂R^{30c}, —COR^{30c}, —CO₂R^{30c}, —CONR^{30c}R^{30d}, —C(═NR^{30c})NR^{30d}R^{30e}, —NR^{30c}R^{30d}, —NR^{30c}COR^{30d}, —NR^{30c}CONR^{30d}R^{30e}, —NR^{30c}CO₂R^{30d}, —NR^{30c}SONR^{30d}R^{30e}, —NR^{30c}SO₂NR^{30d}R^{30e}, or —NR^{30c}SO₂R^{30d}, each of said methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with F, Cl, Br, I, hydroxy, -haloC₁₋₈alkyl, - methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, heterocyclyl, aryl, or heteroaryl;
R^{30c}, R^{30d} and R^{30e} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, or heteroaryl; or
(R^{30c} and R^{30d}) or (R^{30d} and R^{30e}) together with the atom(s) to which they are attached, form a 3-, 4-, 5-, 6-, 7- or 8-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂.

72. The compound of any claim of claims 66-71, wherein R³⁰³ and R³⁰⁴ are each independently hydrogen, F, Cl, Br, I, methyl, ethyl, propyl, butyl, pentyl, hexyl, heptyl, octyl, methoxy, ethoxy, propoxy, cyclopropyl, cyclobutyl, cyclopentyl, cyclohexyl, cycloheptyl, cyclooctyl, heterocyclyl, aryl, heteroaryl or —CN; preferably R³⁰³ and R³⁰⁴ are each independently hydrogen, methyl, ethyl, propyl, butyl, cyclopropyl, cyclobutyl or cyclopentyl.

73. The compound of any claim of claims 66-72, wherein the 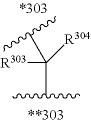
moiety is 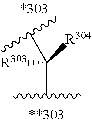
wherein *303 refers to the position attached to the 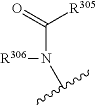
moiety, and **303 refers to the position attached to 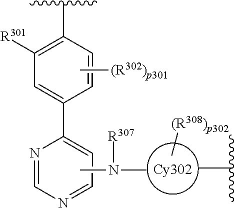
moiety.

74. The compound of any claim of claims 66-73, wherein R³⁰⁶ and R³⁰⁷ are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl, each of said methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with F, Cl, Br, I, hydroxy, -haloC₁₋₈alkyl, methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, heterocyclyl, aryl or heteroaryl; preferably, R³⁰⁶ and R³⁰⁷ are each independently H, methyl, ethyl, propyl butyl, pentyl, hexyl, cyclopropyl, cyclobutyl, cyclopentyl or cyclohexyl.

75. The compound of any claim of claims 66-74, wherein R³⁰⁵ is 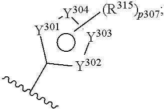
Y³⁰¹, Y³⁰², Y³⁰³ and Y³⁰⁴ are each independently selected from CH, O, S or N; R³¹⁵ is each independently selected from hydrogen, halogen, —C₁₋₈alkyl, —C₁₋₈alkoxy, —C₂₋₈alkenyl, —C₂₋₈alkynyl, cycloalkyl, heterocyclyl, aryl, heteroaryl, —CN, —NO₂, —OR^{30c}, —SO₂R^{30c}, —COR^{30c}, —CO₂R^{30c}, —CONR^{30c}R^{30d}, —C(═NR^{30c})NR^{30d}R^{30e}, —NR^{30c}R^{30d}, —NR^{30c}COR^{30d}, —NR^{30c}CONR^{30d}R^{30e}, —NR^{30c}CO₂R^{30d}, —NR^{30c}SONR^{30d}R^{30e}, —NR^{30c}SO₂NR^{30d}R^{30e}, or —NR^{30c}SO₂R^{30d}, each of said —C₁₋₈alkyl, —C₁₋₈alkoxy, —C₂₋₈alkenyl, —C₂₋₈alkynyl, cycloalkyl, heterocyclyl, aryl, or heteroaryl is optionally substituted with halogen, hydroxy, -haloC₁₋₈alkyl, —C₁₋₈alkyoxy, cycloalkyl, heterocyclyl, aryl, or heteroaryl; R^{30c}, R^{30d}, and R^{30e} are each independently defined as in claim 66; and p307 is 0, 1, 2, 3 or 4.

76. The compound of claim 75, wherein Y³⁰¹ is CH, S, N or O; Y³⁰² is CH, S, O or N; Y³⁰³ is CH, O, S or N; and Y³⁰⁴ is CH, O, S or N.

77. The compound of claim 75, wherein 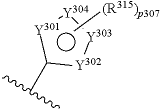
is selected from

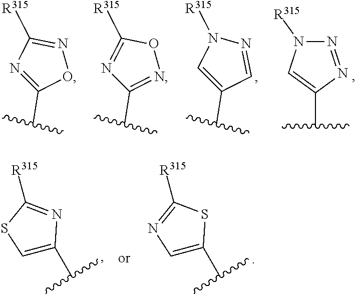

78. The compound according to claim 75, wherein, R³¹⁵ is selected from —H, —F, —Cl, —Br, —I, —CH₃, —C₂H₅, —C₃H₇, —C₄H₉, —C₅H₁₁, —C₆H₁₃, methoxy, ethoxy, propoxy, butoxy, pentoxy, hexoxy, cyclopropyl, cyclobutyl, cyclopentyl, cyclohexyl, —C₂₋₈alkenyl, —C₂₋₈alkynyl, —CH₂OH, —CH₂CH₂OH, —CH(OH)CH₃, —CH₂CH₂CH₂OH, —CH(OH)CH₂CH₃, —CH₂CH(OH)CH₃, —CH₂OCH₃, —CFH₂, —CF₂H, —CF₃, —CH₂CF₃, —CH₂CH₂CF₃, each of said —CH₃, —C₂H₅, —C₃H₇, —C₄H₉, —C₅H₁₁, —C₆H₁₃, methoxy, ethoxy, propoxy, butoxy, pentoxy, hexoxy, cyclopropyl, cyclobutyl, cyclopentyl, cyclohexyl, —C₂₋₈alkenyl, —C₂₋₈alkynyl is optionally substituted with at least one F, Cl, Br, I, hydroxy, -haloC₁₋₈alkyl, —C₁₋₈alkyoxy, cycloalkyl, heterocyclyl, aryl, or heteroaryl.

79. The compound according to claim 75, wherein, R³¹⁵ is selected from

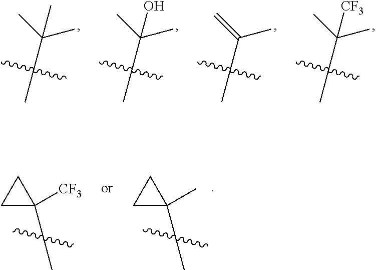

80. The compound of any claim of claims 66-79, wherein the 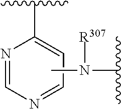
moiety is

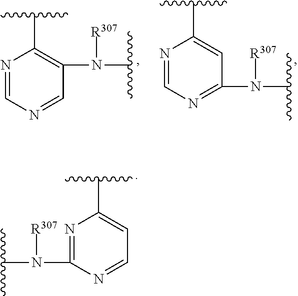

81. The compound of any claim of claims 66-80, wherein Cy302 is a 5- or 6-membered aromatic ring comprising 0-3 heteroatoms selected from nitrogen, oxygen and sulfur as ring member(s).

82. The compound of any claim of claims 66-81, wherein 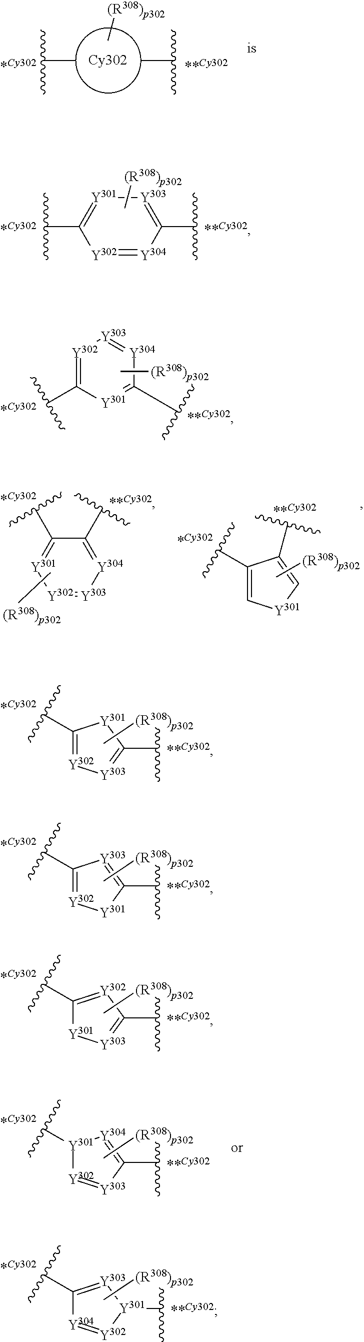
Y³⁰¹, Y³⁰², Y³⁰³ and Y³⁰⁴ are each independently defined as in claim 66;
wherein *Cy302 refers to the position attached to —N(R³⁰⁷) in the 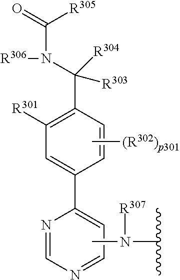
moiety, and **Cy302 refers to the position attached to 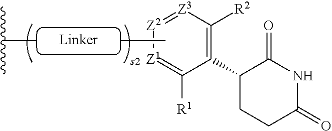
moiety.

83. The compound of any claim of claims 66-82, wherein

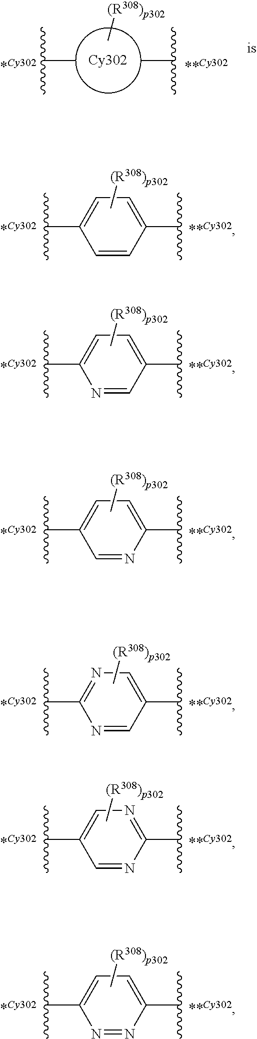

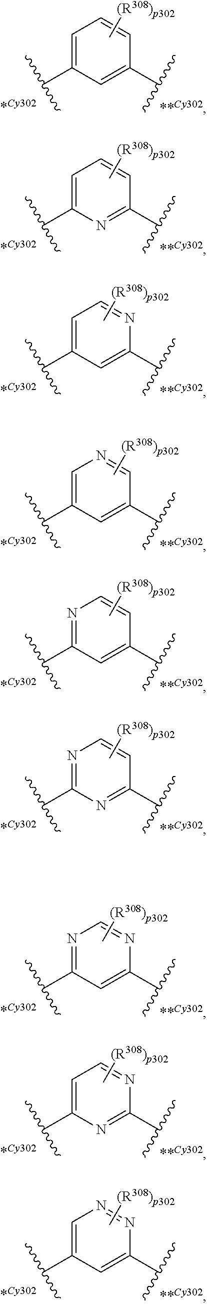

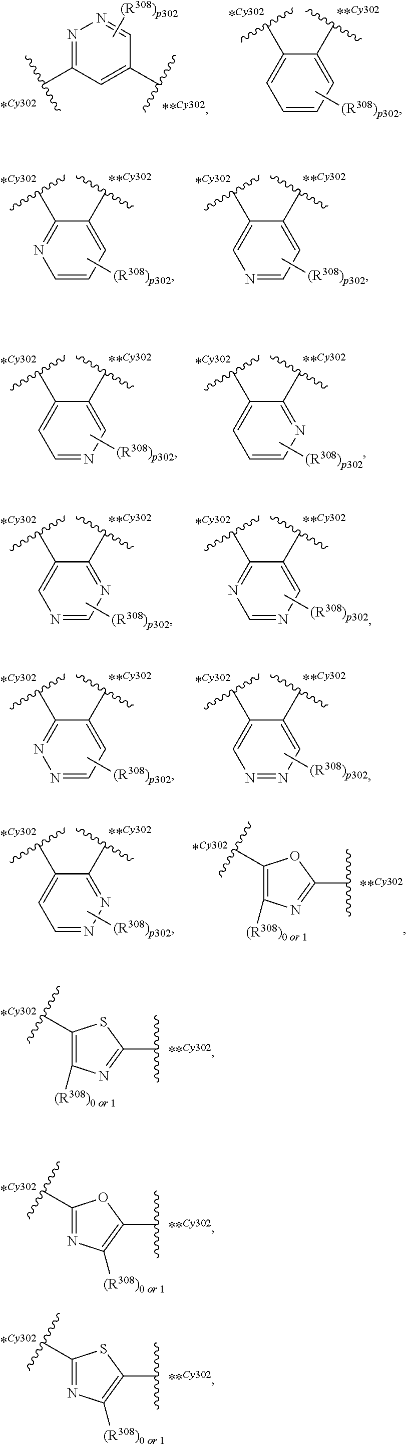

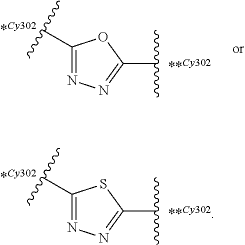 [SMILES: *c1nnc(*)o1 ; *c1nnc(*)s1]

84. The compound of any claim of claims 66-83, wherein
R³⁰⁸ is hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN, —NO₂, —OR^{30c}, —SO₂R^{30c}, —COR^{30c}, —CO₂R^{30c}, —CONR^{30c}R^{30d}, —C(═NR^{30c})NR^{30d}R^{30e}, —NR^{30c}R^{30d}, —NR^{30c}COR^{30d}, —NR^{30c}CONR^{30d}R^{30e}, —NR^{30c}CO₂R^{30d}, —NR^{30c}SONR^{30d}R^{30e}, —NR^{30c}SO₂NR^{30d}R^{30e}, or —NR^{30c}SO₂R^{30d}, each of said methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl or heteroaryl is optionally substituted with F, Cl, Br, I, hydroxy, -haloC₁₋₈alkyl, - methoxy, ethoxy, propoxy, butoxy, pentoxy, hexoxy, heptoxy, octoxy, cyclopropyl, cyclobutyl, cyclopentyl, cyclohexyl, cycloheptyl, cyclooctyl, heterocyclyl, aryl, or heteroaryl;
R^{30c}, R^{30d} and R^{30e} are each independently hydrogen, methyl, ethyl, propyl, butyl, pentyl, hexyl, heptyl, octyl, —C₂₋₈alkenyl, —C₂₋₈alkynyl, cyclopropyl, cyclobutyl, cyclopentyl, cyclohexyl, cycloheptyl, cyclooctyl, heterocyclyl, aryl, or heteroaryl; or
(R^{30c} and R^{30d}) or (R^{30d} and R^{30e}) together with the atom(s) to which they are attached, form a 3-, 4-, 5-, 6-, 7- or 8-membered ring, said ring comprising 0, 1 or 2 additional heteroatoms independently selected from nitrogen, oxygen or optionally oxidized sulfur as ring member(s), said ring is optionally substituted with at least one substituent independently selected from halogen, —C₁₋₈alkyl, —C₂₋₈alkenyl, —C₂₋₈alkynyl, cycloalkyl, heterocyclyl, aryl, heteroaryl, oxo, —CN or —NO₂.

85. The compound of any claim of claims 66-84, wherein R³⁰⁸ is hydrogen, F, Cl, Br, I, methyl, ethyl, propyl, butyl, pentyl, hexyl, heptyl, octyl, methoxy, ethoxy, propoxy, butoxy, pentoxy, hexoxy, heptoxy, octoxy, —C₂₋₈alkenyl, —C₂₋₈alkynyl, cyclopropyl, cyclobutyl, cyclopentyl, cyclohexyl, cycloheptyl, cyclooctyl, heterocyclyl, aryl, heteroaryl, —CN or —NO₂; preferably R2 is hydrogen, F, Cl, Br, I, methyl, ethyl, propyl, butyl, methoxy, ethoxy, propoxy, butoxy, cyclopropyl, cyclobutyl, cyclopentyl, —CN or —NO₂.

86. The compound of any claim of claims 66-85, wherein

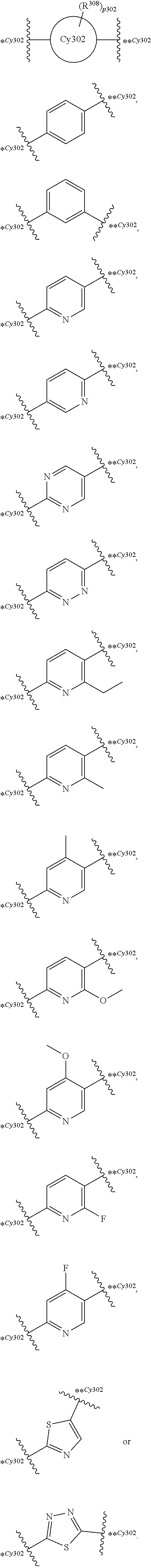

87. The compound of any claim of claims 66-85 wherein the 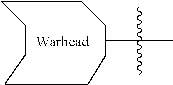
moiety is selected from

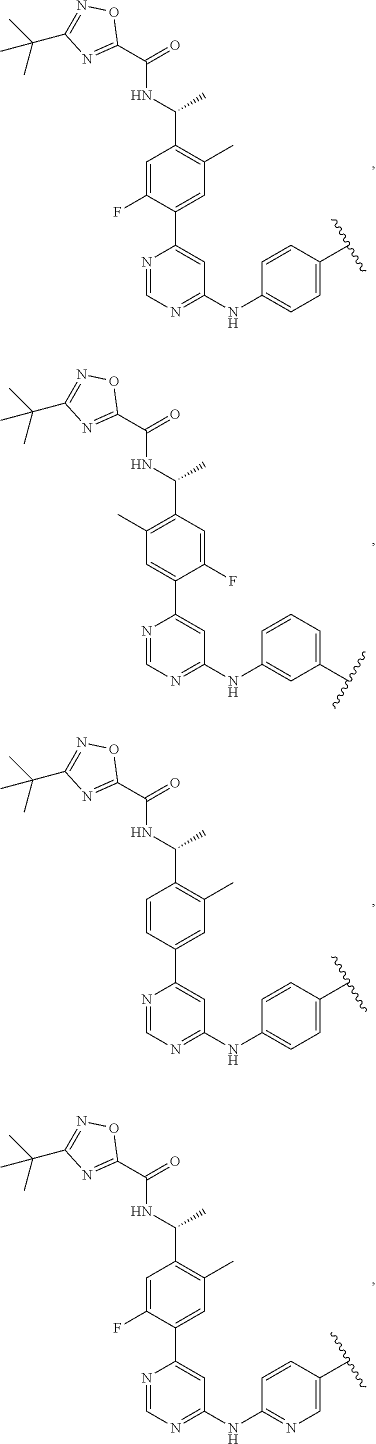 [SMILES: *c1ccc(Nc2cc(-c3cc(C)c([C@@H](C)NC(=O)c4nc(C(C)(C)C)no4)cc3F)ncn2)cc1 ; *c1cccc(Nc2cc(-c3cc(C)c([C@@H](C)NC(=O)c4nc(C(C)(C)C)no4)cc3F)ncn2)c1 ; *c1ccc(Nc2cc(-c3ccc([C@@H](C)NC(=O)c4nc(C(C)(C)C)no4)c(C)c3)ncn2)cc1 ; *c1ccc(Nc2cc(-c3cc(C)c([C@@H](C)NC(=O)c4nc(C(C)(C)C)no4)cc3F)ncn2)nc1]

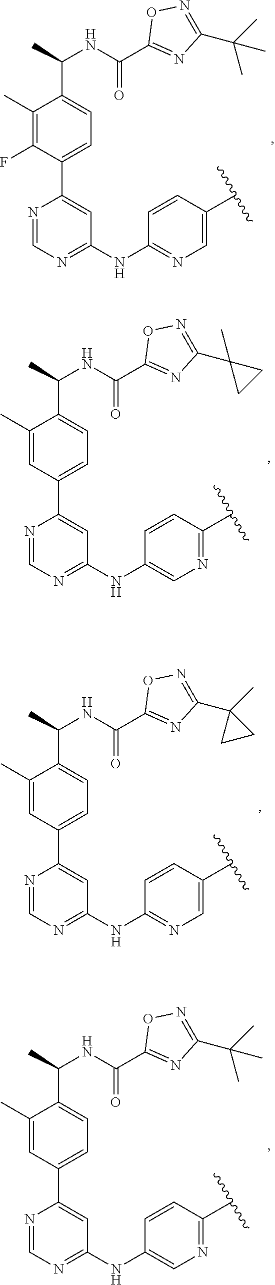

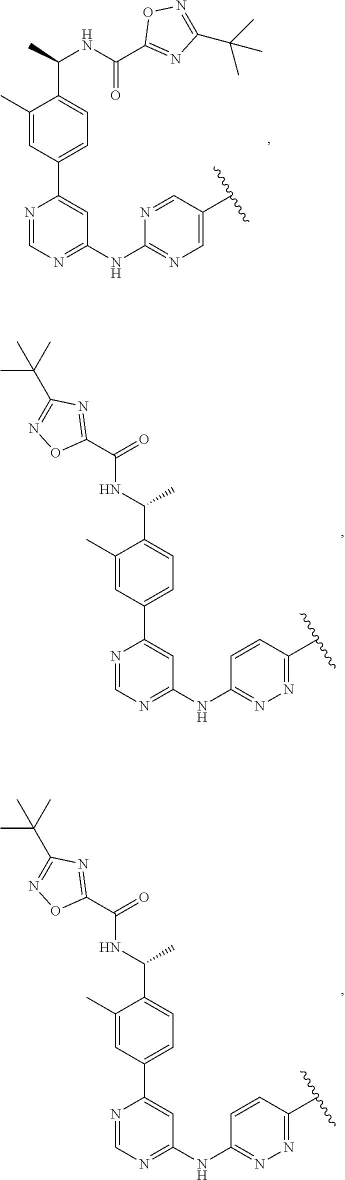

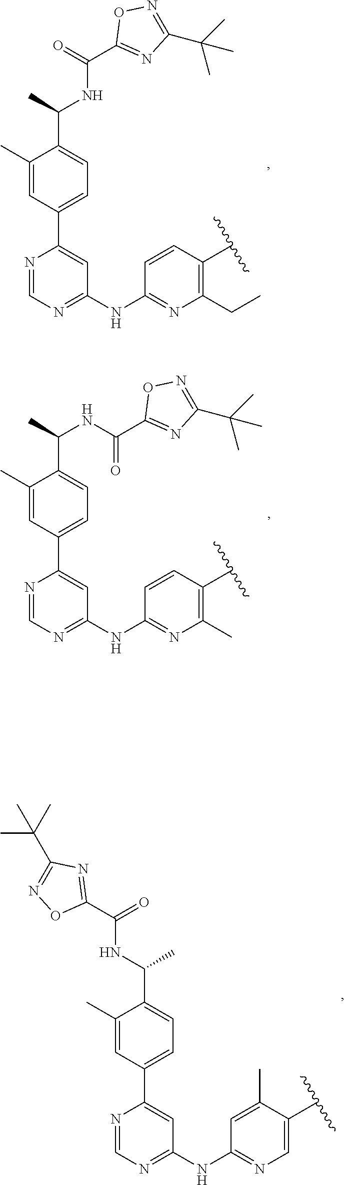

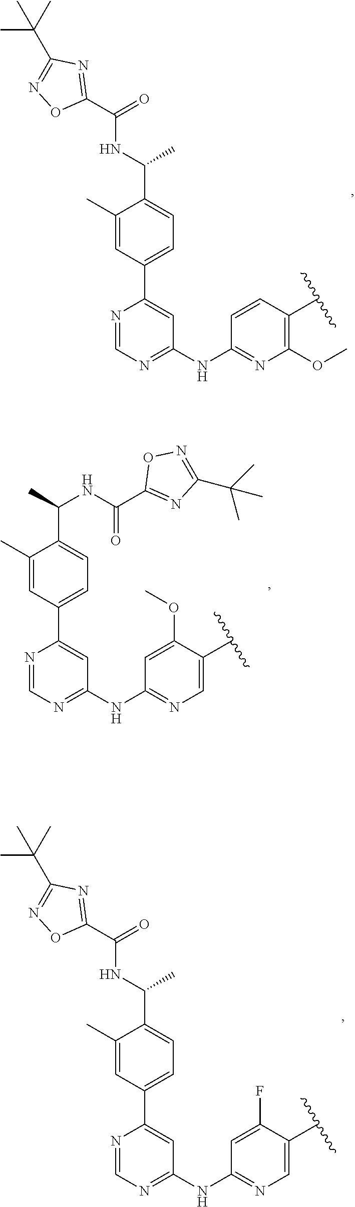

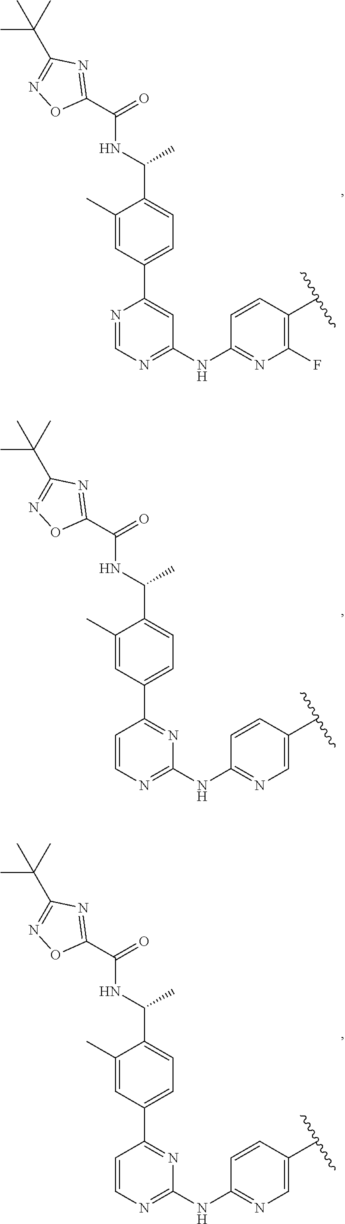 [SMILES: *c1ccc(Nc2cc(-c3ccc([C@@H](C)NC(=O)c4nc(C(C)(C)C)no4)c(C)c3)ncn2)nc1F ; *c1ccc(Nc2nccc(-c3ccc([C@@H](C)NC(=O)c4nc(C(C)(C)C)no4)c(C)c3)n2)nc1 ; *c1ccc(Nc2nccc(-c3ccc([C@@H](C)NC(=O)c4nc(C(C)(C)C)no4)c(C)c3)n2)nc1]

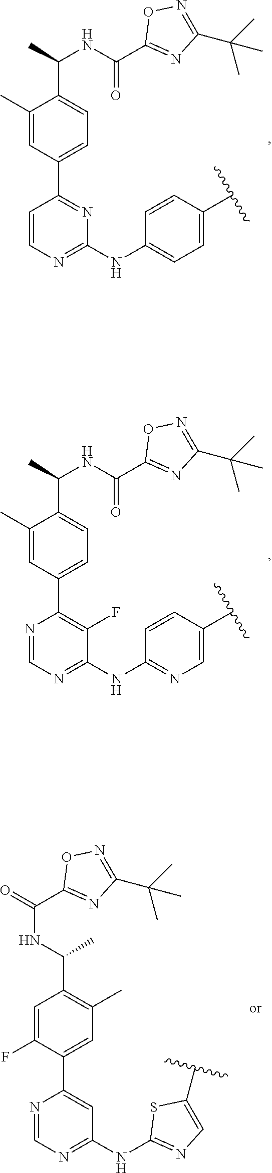

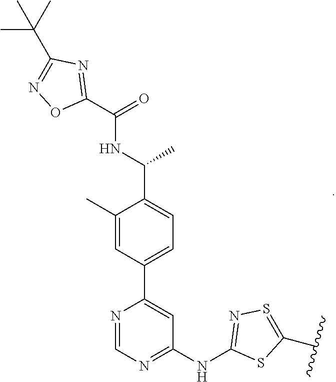

88. A pharmaceutical composition comprising a compound of any one of claims 1-87 or a pharmaceutically acceptable salt, tautomer or prodrug thereof, together with a pharmaceutically acceptable excipient.

89. A method of treating a disease that can be treated by degrading the target protein that the Warhead can combine.

90. The method of claim 89, wherein said target protein is selected from the group consisting of structural proteins, receptors, enzymes, cell surface proteins, proteins pertinent to the integrated function of a cell, including proteins involved in catalytic activity, aromatase activity, motor activity, helicase activity, metabolic processes (anabolism and catabolism), antioxidant activity, proteolysis, biosynthesis, proteins with kinase activity, oxidoreductase activity, transferase activity, hydrolase activity, lyase activity, isomerase activity, ligase activity, enzyme regulator activity, signal transducer activity, structural molecule activity, binding activity (protein, lipid carbohydrate), receptor activity, cell motility, membrane fusion, cell communication, regulation of biological processes, development, cell differentiation, response to stimulus, behavioral proteins, cell adhesion proteins, proteins involved in cell death, proteins involved in transport (including protein transporter activity, nuclear transport, ion transporter activity, channel transporter activity, carrier activity, permease activity, secretion activity, electron transporter activity, pathogenesis, chaperone regulator activity, nucleic acid binding activity, transcription regulator activity, extracellular organization and biogenesis activity and translation regulator activity.

91. The method of claim 89, wherein Warhead is a moiety which binds to a target protein, wherein said target protein is selected from the group consisting of ErbB receptors, B7.1 and B7, TINFR1m, TNFR2, NADPH oxidase, Bcl-Bax and other partners in the apotosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases (including Bruton's Tyrosine Kinase), CD23, CD124, tyrosine kinase p561ck, CD4, CD5, IL-2 receptor, IL-1 receptor, TNF-alphaR, ICAM1, Cat+ channels, VCAM, VLA-4 integrin, selectins, CD40/CD40L, newokinins and receptors, inosine monophosphate dehydrogenase, p38 MAP Kinase, RAS-RAF-MEK-ERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, adenosine receptors, adenosine kinase and AMP deaminase, purinergic receptors (P2Y1, P2Y2, P2Y4, P2Y6, P2X 1-7), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase, ecdysone 20-monooxygenase, ion channel of the GABA gated chloride channel, acetylcholinesterase, voltage-sensitive sodium channel protein, calcium release channel, chloride channels, Acetyl-CoA carboxylase, adenylosuccinate synthetase, protoporphyrinogen oxidase, L-1 receptor associated kinase-3 (IRAK-3 or IRAK-M) or enolpyruvyl-shikimate-phosphate synthase.

92. The method of claim 89, wherein the disease is cancer.

93. A compound of Formula (I): 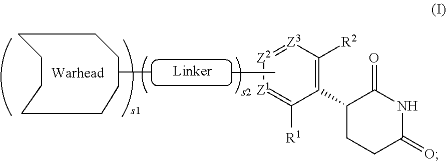
or a pharmaceutically acceptable salt thereof, or a deuterated analog thereof, or a prodrug thereof, wherein:
Warhead, Linker, s2, Z¹, Z², Z³, R¹ and R² are as defined as any preceding claims;
s1 is 0.

94. A method of binding and altering the specificity of cereblon complex to induce the degradation of a complex-associated protein by using the compound of claim 93, wherein the protein is selected from ErbB receptors, B7.1 and B7, TINFR1m, TNFR2, NADPH oxidase, Bcl-Bax and other partners in the apotosis pathway, C5a receptor, HMG-CoA reductase, PDE V phosphodiesterase type, PDE IV phosphodiesterase type 4, PDE I, PDEII, PDEIII, squalene cyclase inhibitor, CXCR1, CXCR2, nitric oxide (NO) synthase, cyclo-oxygenase 1, cyclo-oxygenase 2, 5HT receptors, dopamine receptors, G Proteins, i.e., Gq, histamine receptors, 5-lipoxygenase, tryptase serine protease, thymidylate synthase, purine nucleoside phosphorylase, GAPDH trypanosomal, glycogen phosphorylase, Carbonic anhydrase, chemokine receptors, JAW STAT, RXR and similar, HIV 1 protease, HIV 1 integrase, influenza, neuramimidase, hepatitis B reverse transcriptase, sodium channel, multi drug resistance (MDR), protein P-glycoprotein (and MRP), tyrosine kinases (including Bruton's Tyrosine Kinase), CD23, CD124, tyrosine kinase p561ck, CD4, CD5, IL-2 receptor, IL-1 receptor, TNF-alphaR, ICAM1, Cat+ channels, VCAM, VLA-4 integrin, selectins, CD40/CD40L, newokinins and receptors, inosine monophosphate dehydrogenase, p38 MAP Kinase, RAS-RAF-MEK-ERK pathway, interleukin-1 converting enzyme, caspase, HCV, NS3 protease, HCV NS3 RNA helicase, glycinamide ribonucleotide formyl transferase, rhinovirus 3C protease, herpes simplex virus-1 (HSV-I), protease, cytomegalovirus (CMV) protease, poly (ADP-ribose) polymerase, cyclin dependent kinases, vascular endothelial growth factor, oxytocin receptor, microsomal transfer protein inhibitor, bile acid transport inhibitor, 5 alpha reductase inhibitors, angiotensin 11, glycine receptor, noradrenaline reuptake receptor, endothelin receptors, neuropeptide Y and receptor, adenosine receptors, adenosine kinase and AMP deaminase, purinergic receptors (P2Y1, P2Y2, P2Y4, P2Y6, P2X 1-7), farnesyltransferases, geranylgeranyl transferase, TrkA a receptor for NGF, beta-amyloid, tyrosine kinase Flk-IIKDR, vitronectin receptor, integrin receptor, Her-21 neu, telomerase inhibition, cytosolic phospholipaseA2 and EGF receptor tyrosine kinase, ecdysone 20-monooxygenase, ion channel of the GABA gated chloride channel, acetylcholinesterase, voltage-sensitive sodium channel protein, calcium release channel, chloride channels, Acetyl-CoA carboxylase, adenylosuccinate synthetase, protoporphyrinogen oxidase, L-1 receptor associated kinase-3 (IRAK-3 or IRAK-M), enolpyruvyl-shikimate-phosphate synthase or neo-substrates (like IKZF1, IKZF3, and CK1a).

95. A method of treating an CRBN-mediated disorder, disease, or condition in a patient comprising administering to said patient the pharmaceutical composition of claim 93, preferably, the disorder disease, or condition is selected from proliferative disorders, neurological disorders and disorder associated with transplantation.
